(19) United States
(12) Patent Application Publication
(10) Pub. No.: US 2023/0192700 A1
(43) Pub. Date: Jun. 22, 2023

(54) PYRAZOLOPYRAZINES ACTING ON CANCERS VIA INHIBITION OF CDK12

(71) Applicants: Bayer Aktiengesellschaft, Leverkusen (DE); Deutsches Krebsforschungszentrum, Heidelberg (DE)

(72) Inventors: Markus BERGER, Berlin (DE); Bernd BUCHMANN, Hohen Neuendorf (DE); Kai THEDE, Berlin (DE); Philipp BUCHGRABER, Berlin (DE); Gerhard SIEMEISTER, Berlin (DE); Patrick STEIGEMANN, Berlin (DE); Antje Margret WENGNER, Berlin (DE); Ulf BÖMER, Glienicke (DE); Naomi BARAK, Berlin (DE); Philip LIENAU, Berlin (DE)

(73) Assignees: Bayer Aktiengesellschaft, Leverkusen (DE); Deutsches Krebsforschungszentrum, Heidelberg (DE)

(21) Appl. No.: 17/909,636

(22) PCT Filed: Mar. 5, 2021
(86) PCT No.: PCT/EP2021/055576

§ 371 Date: Sep. 6, 2022

(30) Foreign Application Priority Data
Mar. 6, 2020 (EP) 20161679.4

(51) Int. Cl. C07D 487/04; A61K 45/06; A61P 35/00; C07D 519/00

(52) U.S. Cl. CPC C07D 487/04; A61K 45/06; A61P 35/00; C07D 519/00

(57) ABSTRACT

The present invention provides compounds of general formula (I) in which X, R¹, R² and R³ are as described and defined herein, methods of preparing said compounds, intermediate compounds useful for preparing said compounds, pharmaceutical compositions and combinations comprising said compounds, and the use of said compounds for manufacturing pharmaceutical compositions for the treatment and/or prophylaxis of diseases, in particular of hyperproliferative disorders such as cancer disorders, as a sole agent or in combination with other active ingredients.

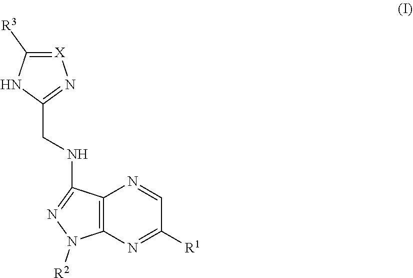

### BACKGROUND

The present invention provides compounds of general formula (I) which impair the activity of CDK12. In particular, the present invention provides compositions and methods for the treatment of cancer and other CDK12-dependant diseases. More particularly, the present invention provides compounds which induce the proteolytic degradation of CDK12 and/or Cyclin K in the cell. Thus, the present invention provides compounds capable of degrading CDK12 and/or Cyclin K for the treatment of breast cancer, liver cancer, lung cancer, ovarian cancer, endometrial cancer, cervical cancer, colorectal cancer, gastric cancer, esophageal cancer, bladder cancer, prostate cancer, Ewing sarcoma, glioblastoma and acute myeloid leukemia. Even more particularly, the present invention provides compounds capable of degrading CDK12 and/or Cyclin K for the treatment of lung cancer, breast cancer, liver cancer, colorectal cancer, gastric cancer, prostate cancer and leukemia.

Cyclin-dependent kinase (CDK) 12 (CDK12, gene id 51755) is a member of the subset of the CDK serine/threonine kinase family that phosphorylates the C-terminal domain (CTD) of RNA polymerase II. CDK12 in complex with Cyclin K (CCNK, gene id 8812) regulates transcriptional, co- and posttranscriptional processes by phosphorylation of Ser2 and Ser5 of the CTD of RNA polymerase II complexes which are important in the elongation phase of pre-mRNA synthesis. CDK12/Cyclin K has been reported to regulate transcriptional elongation and mRNA processing, in particular co- and post-transcriptional pre-mRNA splicing, alternative splicing, 3′end processing, and suppression of intronic polyadenlyation. CDK13 (CDK13, gene id 8621), a kinase which is closely related to CDK12, also forms a complex with Cyclin K and regulates the transcription of a different set of genes (Bartkowiak et al. Genes Dev. 2010; 24:2303-16. Dubbury et al. Nature. 2018; 564:141-5. Greenleaf, Transcription. 2018; 10:91-110. Greifenberg et al. Cell Rep. 2016; 14:320-31. Liang et al. Mol. Cell. Biol. 2015; 35:928-38. Lui et al. J. Clin. Pathol. 2018; 71:957-62. Tien et al. Nuc. Acids Res. 2017; 45:6698-716). The transcription of genes encoding components of DNA damage signaling and repair pathways, such as the homologous recombination and replication stress response genes BRCA1, FANCD2, FANCI, and ATR, as well as encoding components of other stress response pathways, such as NF-κB and oxidative stress response, has been reported to be specifically regulated by CDK12/Cyclin K as demonstrated by gene knock-down and chemoproteomics studies (Blazek et al. Genes Dev. 2011; 25:2158-72. Henry et al. Sci. Signal. 2018; 11:eaam8216. Li et al. Sci. Rep. 2016; 6:21455.). In addition, CDK12/Cyclin K has been reported to control the translation of a subset of mRNAs, including the CHK1 mRNA, by directly phosphorylating the mRNA 5′ cap-binding translational repressor 4E-BP1 leading to its release from the mRNA cap (Choi et al. Genes Dev. 2019; 33:418-35). The recent discovery of rare bi-allelic CDK12 inactivating mutations in high-grade serous ovarian cancer and in primary and castration-resistant prostate cancer leading to a special type of genomic instability which is characterized by the occurance of numerous tandem duplications, indicating gross defects in DNA repair, underscores the role of CDK12 in DNA damage response and the maintenance of the genome (Ekumi et al. Nucl. Acids Res. 2015; 43:2575-89. Grasso et al. Nature. 2012; 487:239-43. Joshi et al. J. Biol. Chem. 2014; 289:9247-53. Menghi et al. Cancer Cell. 2018; 34:197-210.e5. Popova et al. Cancer Res. 2016; 76:1882-91. Quigley et al. Cell. 2018; 174:758-69.e9. Robinson et al. 2015; 162:454. Viswanathan et al. Cell. 2018; 174:433-47.e19. Wu et al. Cell. 2018; 173:1770-82.e14). The CDK12 gene is located on chromosome 17 about 200 kb proximal to the ERBB2 gene and is often coamplified in breast cancer. Furthermore, CDK12 gene amplification has been observed in other cancer types such as stomach cancer, esophageal cancer, pancreatic cancer, uterine cancer, endometrial cancer, prostate cancer, and bladder cancer (Lui et al. J Clin Pathol. 2018; 71:957-62. Gupta et al. Clin. Cancer Res. 2017; 23:1346-57). CDK12 amplification and high expression levels suggest a tumor promoting role of CDK12 which is, at least partially, based on alterantively spliced mRNAs, increased DNA repair capabilities and increased stress tolerance (Lui et al. J Clin Pathol. 2018; 71:957-62. Tien et al. Nucl. Acids Res. 2017; 45:6698-716). Taken together these data validated CDK12 as a potential target to develop drugs for the treatment of cancer and other diseases such as myotonic dystrophy type 1.

Some inhibitors of CDK12 kinase activity are known:

Flavopiridol, a micromolar non-selective inhibitor of CDK12 which inhibits other kinases such as CDK9, CDK1, CDK4 etc. (Bösken et al. Nat. Comm. 2014; 5:3505). Dinaciclib, a pan CDK inhibitor (Johnson et al. Cell Rep. 2016; 17:2367-81). THZ531, a dual inhibitor of CDK12 and CDK13 (Zhang et al. Nat. Chem. Biol. 2016; 12:876-84). SR-3029 and related purine compounds (Johannes et al. Chem. Med. Chem. 2018; 13:231-5). SR-4835, a dual inhibitor of CDK12 and CDK13 (Quereda et al. Cancer Cell 2019; 36:1-14). Compound 919278, a micromolar CDK12 inhibitor (Henry et al. Science Signal. 2018; 11:eaam8216). Arylurea derivatives (Ito et al. J. Med. Chem. 2018; 61:7710-28).

There is a need for development of compounds selectively impairing the function of CDK12/Cyclin K for the treatment of cancer and other diseases, e.g. by inducing the proteolytic degradation of CDK12 and/or Cyclin K protein in the cell. Restricted selectivity of inhibitors targeting the ATP pocket is an issue which may lead to undesired side effects and limited clinical utility (Sawa. Mini-Rev. Med. Chem 2008; 8:1291-7). Surprisingly, the compounds described in the present invention induce the proteolytic degradation of CDK12 and/or Cyclin K protein in the cell. CDK12 inhibitors with low kinase inhibition potential at physiological ATP concentrations but strong CDK12 degrading potency are selective against other kinases. In addition, by degradation of CDK12 and/or Cyclin K functions of the CDK12/CyclinK protein complex which are independent from the sole kinase activity, such as scaffolding functions for other proteins e.g. in the RNA polymerase II complex or the pre-mRNA splicing complex will be impaired as well. Thus, there is a need to provide compounds which impair the activity of CDK12 and/or Cyclin K in the cell and which exhibit a good degree of selectivity towards the targeting of other CDKs and other kinases, such as, for example, casein kinases.

### SUMMARY

The present invention provides compounds of general formula (I):

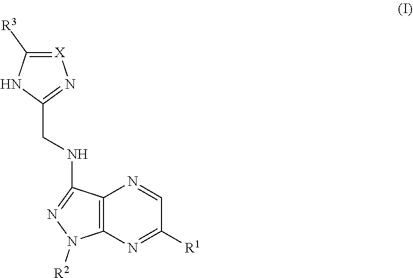

in which X, R¹, R² and R³ are as described and defined herein, methods of preparing said compounds, intermediate compounds useful for preparing said compounds, pharmaceutical compositions and combinations comprising said compounds, and the use of said compounds for manufacturing pharmaceutical compositions for the treatment and/or prophylaxis of diseases, in particular of hyperproliferative disorders such as cancer disorders, as a sole agent or in combination with other active ingredients.

### DESCRIPTION OF THE INVENTION

It has now been found that the compounds of the present invention effectively impair the activity of CDK12 and/or Cyclin K for which data are given in the biological experimental section and may therefore be used for the treatment and/or prophylaxis of hyperproliferative disorders, such as cancer disorders. In particular, the compounds of the present invention are CDK12 inhibitors with low kinase inhibition potential at physiological ATP concentrations but strong proteolytic CDK12 and/or Cyclin K degrading potency in cells and are therefore selective against other kinases while maintaining an impairing effect towards CDK12/Cyclin K.

In accordance with a first aspect, the present invention provides compounds of general formula (I):

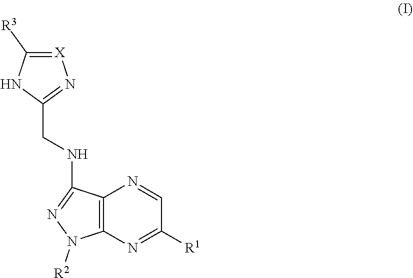

wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group,a heterocycloalkyl group, a 7- to 9-membered bridged compound and a 7- to 11-membered spiro compound,
   wherein said heterocycloalkyl group, 7- to 9-membered bridged compound or 7- to 11-membered spiro compound is connected to the rest of the molecule via a carbon atom of said heterocycloalkyl group,
   wherein said 7- to 9-membered bridged compound or 7- to 11-membered spiro compound each optionally contains one heteroatom independently selected from nitrogen and oxygen,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

### DETAILED DESCRIPTION

Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3, more particularly 1 or 2, and even more particularly 1.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one, two or three substituents, more particularly with one substituent.

The terms “oxo”, “an oxo group” or “an oxo substituent” mean a doubly bonded oxygen atom ═O. Oxo may be attached to atoms of suitable valency, for example to a saturated carbon atom or to a sulfur atom. For example, but without limitation, one oxo group can be attached to a carbon atom, resulting in the formation of a carbonyl group C(═O), or two oxo groups can be attached to one sulfur atom, resulting in the formation of a sulfonyl group —S(═O)₂.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

If within the present text any item is referred to as “supra” within the description it indicates any of the respective disclosures made within the specification in any of the preceding pages, or above on the same page.

If within the present text any item is referred to as “infra” within the description it indicates any of the respective disclosures made within the specification in any of the subsequent pages, or below on the same page.

The terms as mentioned in the present text have the following meanings:

The term “halogen atom” means a fluorine, chlorine, bromine or iodine atom, particularly a fluorine, chlorine or bromine atom, more particularly a fluorine atom.

The term “C₁-C₆-alkyl” means a linear or branched, saturated, monovalent hydrocarbon group having 1, 2, 3, 4, 5 or 6 carbon atoms, e.g. a methyl-, ethyl-, propyl-, isopropyl-, butyl-, sec-butyl-, isobutyl-, tert-butyl-, pentyl-, isopentyl-, 2-methylbutyl-, 1-methylbutyl-, 1-ethylpropyl-, 1,2-dimethylpropyl-, neo-pentyl-, 1,1-dimethylpropyl-, hexyl-, 1-methylpentyl-, 2-methylpentyl-, 3-methylpentyl-, 4-methyl pentyl-, 1-ethylbutyl-, 2-ethylbutyl-, 1,1-dimethylbutyl-, 2,2-dimethylbutyl-, 3,3-dimethylbutyl-, 2,3-dimethylbutyl-, 1,2-dimethylbutyl- or a 1,3-dimethylbutyl- group, or an isomer thereof. Particularly, said group has 1, 2, 3 or 4 carbon atoms (“C₁-C₄-alkyl”), e.g. a methyl-, ethyl-, propyl-, isopropyl-, butyl-, sec-butyl-, isobutyl- or a tent-butyl group, more particularly 1, 2 or 3 carbon atoms (“C₁-C₃-alkyl”), e.g. a methyl-, ethyl-, n-propyl- or an isopropyl group.

The term “C₁-C₆-hydroxyalkyl” means a linear or branched, saturated, monovalent hydrocarbon group in which the term “C₁-C₆-alkyl” is defined supra, and in which one or more hydrogen atoms are replaced with a hydroxy group, e.g. a hydroxymethyl-, 1-hydroxyethyl-, 2-hydroxyethyl-, 1,2-dihydroxyethyl-, 3-hydroxypropyl-, 2-hydroxypropyl-, 1-hydroxypropyl-, 1-hydroxypropan-2-yl-, 2-hydroxypropan-2-yl-, 2,3-dihydroxypropyl-, 1,3-dihydroxypropan-2-yl-, 3-hydroxy-2-methyl-propyl-, 2-hydroxy-2-methyl-propyl- or a 1-hydroxy-2-methyl-propyl- group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl-, ethylsulfanyl-, propylsulfanyl-, isopropylsulfanyl-, butylsulfanyl-, sec-butylsulfanyl-, isobutylsulfanyl-, tert-butylsulfanyl-, pentylsulfanyl-, isopentylsulfanyl- or a hexylsulfanyl- group.

The term “C₁-C₆-haloalkyl” means a linear or branched, saturated, monovalent hydrocarbon group in which the term “C₁-C₆-alkyl” is as defined supra and in which one or more of the hydrogen atoms are replaced, identically or differently, with a halogen atom. Preferably, said halogen atom is a fluorine atom. Said C₁-C₆-haloalkyl, particularly a C₁-C₃-haloalkyl group is, for example, fluoromethyl-, difluoromethyl-, trifluoromethyl-, 2-fluoroethyl-, 2,2-difluoroethyl-, 2,2,2-trifluoroethyl-, pentafluoroethyl-, 3,3,3-trifluoropropyl- or a 1,3-difluoropropan-2-yl group.

The term “C₁-C₆-alkoxy” means a linear or branched, saturated, monovalent group of formula (C₁-C₆-alkyl)-O—, in which the term “C₁-C₆-alkyl” group is as defined supra, e.g. methoxy-, ethoxy-, n-propoxy-, isopropoxy-, n-butoxy-, sec-butoxy-, isobutoxy-, tert-butoxy-, pentyloxy-, isopentyloxy- or a n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Preferably, said halogen atom in “C₁-C₆-haloalkoxy-” is fluorine, resulting in a group referred herein as “C₁-C₆-fluoroalkoxy-”. Representative C₁-C₆-fluoroalkoxy- groups include, for example, —OCF₃, —OCHF₂, —OCH₂F, —OCF₂CF₃ and —OCH₂CF₃.

The term “C₂-C₆-alkenyl” means a linear or branched, monovalent hydrocarbon group, which contains one or more double bonds and which has 2, 3, 4, 5 or 6 carbon atoms, preferably 2, 3 or 4 carbon atoms (“C₂-C₄-alkenyl-”) or 2 or 3 carbon atoms (“C₂-C₃-alkenyl-”), it being understood that in the case in which said alkenyl- group contains more than one double bond, then said double bonds may be isolated from, or conjugated with, each other. Representative alkenyl groups include, for example, an ethenyl-, prop-2-enyl-, (E)-prop-1-enyl-, (Z)-prop-1-enyl-, iso-propenyl-, but-3-enyl-, (E)-but-2-enyl-, (Z)-but-2-enyl-, (E)-but-1-enyl-, (Z)-but-1-enyl-, 2-methylprop-2-enyl-, 1-methylprop-2-enyl-, 2-methylprop-1-enyl-, (E)-1-methylprop-1-enyl-, (Z)-1-methylprop-1-enyl-, buta-1,3-dienyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, (E)-3,3-dimethylprop-1-enyl-, (Z)-3,3-dimethylprop-1-enyl-, penta-1,4-dienyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, hexa-1,5-dienyl- and a 1-(1,1-dimethylethyl-)ethenyl group. Particularly, said group is an ethenyl- or a prop-2-enyl group.

The same definitions can be applied should the alkenyl group be placed within a chain as a bivalent “C₂-C₆-alkenylene” moiety. All names as mentioned above then will bear a “ene” added to their end, thus e.g., a “pentenyl” becomes a bivalent “pentenylene” group.

The term “C₂-C₆-haloalkenyl-” means a linear or branched hydrocarbon group in which one or more of the hydrogen atoms of a “C₂-C₆-alkenyl” as defined supra are each replaced, identically or differently, by a halogen atom. Preferably, said halogen atom is fluorine, resulting in a group referred herein as “C₂-C₆-fluoroalkenyl-”. Representative C₂-C₆-fluoroalkenyl- groups include, for example, —CH═CF₂, —CF═CH₂, —CF═CF₂, —C(CH₃)═CF₂, —CH═C(F)—CH₃, —CH₂—CF═CF₂ and —CF₂—CH═CH₂.

The term “C₂-C₆-alkynyl-” means a linear or branched, monovalent hydrocarbon group which contains one or more triple bonds, and which contains 2, 3, 4, 5 or 6 carbon atoms, preferably 2, 3 or 4 carbon atoms (“C₂-C₄-alkynyl-”) or 2 or 3 carbon atoms (“C₂-C₃-alkynyl-”). Representative C₂-C₆-alkynyl- groups include, for example, an ethynyl-, prop-1-ynyl-, prop-2-ynyl-, but-1-ynyl-, but-2-ynyl-, but-3-ynyl-, pent-1-ynyl-, pent-2-ynyl, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- and a 3,3-dimethylbut-1-ynyl- group. Particularly, said alkynyl- group is an ethynyl-, a prop-1-ynyl- or a prop-2-ynyl group.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl-, cyclobutyl-, cyclopentyl-, cyclohexyl-, cycloheptyl- or cyclooctyl- group, or a bicyclic hydrocarbon ring, e.g. a bicyclo[4.2.0]octyl- or a octahydropentalenyl- group.

The term “C₃-C₆-halocycloalkyl” means a saturated, monovalent, mono- or bicyclic hydrocarbon ring which contains 3, 4, 5 or 6 carbon atoms in which the term “C₃-C₆-cycloalkyl” is as defined supra and in which one or more of the hydrogen atoms of the hydrocarbon ring are replaced, identically or differently, with a halogen atom. Preferably, said halogen atom is a fluorine atom. The “C₃-C₆-cycloalkyl” group as defined supra in which one or more of the hydrogen atoms are replaced, identically or differently, with a halogen atom, preferably a fluorine atom, is for example, a monocyclic hydrocarbon ring, e.g. a cyclopropyl-, cyclobutyl-, cyclopentyl- or cyclohexyl- group, or a bicyclic hydrocarbon ring.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g., a cyclobutenyl-, cyclopentenyl-, cyclohexenyl-, cycloheptenyl- or a cyclooctenyl group, or a bicyclic hydrocarbon ring, e.g., a bicyclo[2.2.1]hept-2-enyl- or a bicyclo[2.2.2]oct-2-enyl group.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy-, cyclobutyloxy-, cyclopentyloxy-, cyclohexyloxy-, cycloheptyloxy- or a cyclooctyloxy- group.

Unless stated otherwise, the term “heterocycloalkyl” means a saturated, monovalent, monocyclic, bicyclic, bridged or spiro compound that contains one or more identical or different ring heteroatoms selected from nitrogen, oxygen and sulfur or one group selected from —S(═O)—, —S(═O)₂— and —S(═O)(═NH)—. Thus, exemplarily, but without being limited thereto, the term “heterocycloalkyl” preferably refers to a 4- to 9-membered monocyclic or 5- to 11-membered bicyclic compound, a 7- to 9-membered bridged compound or a 7- to 12-membered spiro compound containing one or two identical or different ring heteroatoms selected from nitrogen, oxygen and sulfur or one group selected from —S(═O)—, —S(═O)₂— and —S(═O)(═NH)—.

If the term “heterocycloalkyl” is used without specifying a number of atoms it is meant to be a “4- to 10-membered heterocycloalkyl-” group, more particularly a 5- to 6-membered heterocycloalkyl group. The terms “4- to 7-membered heterocycloalkyl”, “4- to 6-membered heterocycloalkyl” and “5- to 7-membered heterocycloalkyl” mean a monocyclic, saturated heterocycle with “4, 5, 6 or 7” or, respectively, “4, 5 or 6” or “5, 6 or 7” ring atoms in total, which are saturated or partially unsaturated monocycles, bicycles or polycycles that contain one or two identical or different ring heteroatoms selected from nitrogen, oxygen and sulfur or one group selected from —S(═O)—, —S(═O)₂— and —S(═O)(═NH)—. It is possible for said heterocycloalkyl group to be attached to the rest of the molecule via any one of the carbon atoms or, if present, a nitrogen atom.

Exemplarily, without being limited thereto, said “4- to 7-membered heterocycloalkyl”, can be a 4-membered ring, a “4-membered heterocycloalkyl-” group, such as an azetidinyl- or an oxetanyl group; or a 5-membered ring, a “5-membered heterocycloalkyl-” group, such as a tetrahydrofuranyl-, dioxolinyl-, pyrrolidinyl-, imidazolidinyl-, pyrazolidinyl- or a pyrrolinyl group; or a 6-membered ring, a “6-membered heterocycloalkyl-” group, such as a tetrahydropyranyl-, piperidinyl-, morpholinyl-, 3-oxomorpholin-4-yl, dithianyl-, thiomorpholinyl- or a piperazinyl group; or a 7-membered ring, a “7-membered heterocycloalkyl-” group, such as an azepanyl-, diazepanyl- or an oxazepanyl group, for example. The heterocycloalkyl groups may be substituted one or more times independently with C₁-C₃-alkyl, C₁-C₃-alkoxy, hydroxy, halogen or a carbonyl group.

Particularly, “4- to 6-membered heterocycloalkyl” means a 4- to 6-membered heterocycloalkyl as defined supra containing one ring nitrogen atom and optionally one further ring heteroatom selected from nitrogen, oxygen and sulfur. Particularly, “5- to 7-membered heterocycloalkyl” means a 5- to 7-membered heterocycloalkyl as defined supra containing one ring nitrogen atom and optionally one further ring heteroatom selected from nitrogen, oxygen and sulfur. More particularly, “5- or 6-membered heterocycloalkyl” means a monocyclic, saturated heterocycle with 5 or 6 ring atoms in total, containing one ring nitrogen atom and optionally one further ring heteroatom selected from nitrogen and oxygen.

The term “heteroaryl-” means a monocyclic, bicyclic or tricyclic aromatic ring system having 5, 6, 7, 8, 9, 10, 11, 12, 13 or 14 ring atoms (a “5- to 14-membered heteroaryl-” group), preferably 5, 6, 9 or 10 ring atoms and which contains 1, 2, 3 or 4 heteroatoms which may be identical or different, said heteroatoms being selected from oxygen, nitrogen and sulfur. Said heteroaryl- group can be a 5-membered heteroaryl group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl- or a tetrazolyl group; or a 6-membered heteroaryl group, such as, for example, a pyridyl-, pyridazinyl-, pyrimidyl-, pyrazinyl- or a triazinyl group; or a benzo-fused 5-membered heteroaryl- group, such as, for example, a benzofuranyl-, benzothienyl-, benzoxazolyl-, benzisoxazolyl-, benzimidazolyl-, benzothiazolyl-, benzotriazolyl-, indazolyl-, indolyl- or a isoindolyl group; or a benzo-fused 6-membered heteroaryl group, such as, for example, a quinolinyl-, quinazolinyl-, isoquinolinyl-, cinnolinyl-, phthalazinyl- or quinoxalinyl-; or another bicyclic group, such as, for example, indolizinyl-, purinyl- or a pteridinyl group.

Preferably, “heteroaryl-” is a monocyclic aromatic ring system having 5 or 6 ring atoms and which contains at least one heteroatom, if more than one, they may be identical or different, said heteroatom being selected from oxygen, nitrogen and sulfur, a (“5- to 6-membered monocyclic heteroaryl-”) group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl-, pyridyl-, pyridazinyl-, pyrimidyl-, pyrazinyl- or a triazinyl group.

In general, and unless otherwise mentioned, said heteroaryl- groups include all the possible isomeric forms thereof, e.g., the positional isomers thereof. Thus, for some illustrative non-restricting example, the term pyridyl- includes pyridin-2-yl-, pyridin-3-yl- and pyridin-4-yl-; the term thienyl- includes thien-2-yl- and thien-3-yl-, and a heteroarylene group may be inserted into a chain also in the inverse way such as e.g. a 2,3-pyridinylene includes pyridine-2,3-yl as well as pyridine-3,2-yl. Furthermore, said heteroaryl- groups can be attached to the rest of the molecule via any one of the carbon atoms, or, if applicable, a nitrogen atom, e.g., a pyrrol-1-yl-, a pyrazol-1-yl- or an imidazol-1-yl- group.

Particularly, the heteroaryl group is a pyridyl- or pyrimidyl group or a imidazolyl group, including a hydroxy substitution of the pyridyl group leading, e.g., to a 2-hydroxy-pyridine which is the tautomeric form to a 2-oxo-2(1H)-pyridine. In some embodiments, the heteroaryl group is an oxazolyl group.

Further, as used herein, the term “C₃-C₈”, as used throughout this text, e.g., in the context of the definition of “C₃-C₈-cycloalkyl-”, is to be understood as meaning e.g. a cycloalkyl- group having a whole number of carbon atoms of 3 to 8, i.e., 3, 4, 5, 6, 7 or 8 carbon atoms. It is to be understood further that said term “C₃-C₈” is to be interpreted as disclosing any sub-range comprised therein, e.g., C₃-C₆, C₄-C₅, C₃-C₅, C₃-C₄, C₄-C₆, C₅-C₇; preferably C₃-C₆.

Similarly, as used herein, the term “C₂-C₆”, as used throughout this text, e.g., in the ontext of the definitions of “C₂-C₆-alkenyl-” and “C₂-C₆-alkynyl-”, is to be understood as meaning an alkenyl- group or an alkynyl- group having a whole number of carbon atoms from 2 to 6, i.e., 2, 3, 4, 5 or 6 carbon atoms. It is to be understood further that said term “C₂-C₆” is to be interpreted as disclosing any sub-range comprised therein, e.g., C₂-C₆, C₃-C₅, C₃-C₄, C₂-C₃, C₂-C₄, C₂-C₅, preferably C₂-C₃.

The term “C₁-C₆”, as used throughout this text, e.g., in the context of the definition of “C₁-C₆-alkyl-”, “C₁-C₆-haloalkyl-”, “C₁-C₆-alkoxy-” or “C₁-C₆-haloalkoxy-” is to be understood as meaning an alkyl group having a whole number of carbon atoms from 1 to 6, i.e., 1, 2, 3, 4, 5 or 6 carbon atoms. It is to be understood further that said term “C₁-C₆” is to be interpreted as disclosing any sub-range comprised therein, e.g. C₁-C₆, C₂-C₅, C₃-C₄, C₁-C₂, C₁-C₃, C₁-C₄, C₁-C₅, C₁-C₆, preferably C₁-C₂, C₁-C₃, C₁-C₄, C₁-C₅, C₁-C₆ more preferably C₁-C₄, in the case of “C₁-C₆-haloalkyl-” or “C₁-C₆-haloalkoxy-” even more preferably C₁-C₂.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;

“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄C₅, C₅-C₈, C₅-C₇, C₅-C₆, C₆-C₈, C₆-C₇ and C₇-C₈;

“C₃-C₆” encompasses C₃, C₄, C₅, C₆, C₃-C₆, C₃-C₅, C₃-C₄, C₄-C₆, C₄-C₅, and C₅-C₆;

“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;

“C₄-C₇” encompasses C₄, C₅, C₆, C₇, C₄-C₇, C₄-C₆, C₄-C₅, C₅-C₇, C₅-C₆ and C₆-C₇;

“C₄-C₆” encompasses C₄, C₅, C₆, C₄-C₆, C₄-C₅ and C₅-C₆;

“C₅-C₁₀” encompasses C₅, C₆, C₇, C₈, C₉, C₁₀, C₅-C₁₀, C₅-C₉, C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁₀;

“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” refers to an atom or a group of atoms that is displaced in a chemical reaction as stable species taking with it the bonding electrons, e.g., typically forming an anion. Preferably, a leaving group is selected from the group comprising: halo, in particular a chloro, bromo or iodo, (methylsulfonyl)oxy-, [(4-methylphenyl)sulfonyl]oxy-, [(trifluoromethyl)sulfonyl]oxy-, [(nonafluorobutyl)sulfonyl]oxy-[(4-bromophenyl)sulfonyl]oxy-, [(4-nitrophenyl)sulfonyl]oxy-, [(2-nitro-phenyl)sulfonyl]oxy-, [(4-isopropyl phenyl)sulfonyl]oxy-, [(2,4,6-triisopropylphenyl)sulfonyl]oxy-, [(2,4,6-trimethylphenyl)sulfonyl]oxy-, [(4-tert-butylphenyl)sulfonyl]oxy-, (phenylsulfonyl)oxy-, and a [(4-methoxyphenyl)sulfonyl]oxy group.

As used herein, the term “protective group” is a protective group attached to an oxygen or nitrogen atom in intermediates used for the preparation of compounds of the general formula (I). Such groups are introduced e.g., by chemical modification of the respective hydroxy or amino group in order to obtain chemoselectivity in a subsequent chemical reaction. Protective groups for hydroxy and amino groups are described for example in T. W. Greene and P. G. M. Wuts in Protective Groups in Organic Synthesis, 4^{th} edition, Wiley 2006; more specifically, protective groups for amino groups can be selected from substituted sulfonyl groups, such as a mesyl-, tosyl- or a phenylsulfonyl group, acyl groups such as a benzoyl-, acetyl- or a tetrahydropyranoyl group, or carbamate based groups, such as a tert-butoxycarbonyl group (Boc). Protective groups for hydroxy groups can be selected from acyl groups such as a benzoyl-, acetyl-, pivaloyl- or a tetrahydropyranoyl group, or can include silicon, as in e.g., a tert-butyldimethylsilyl-, tert-butyldiphenylsilyl-, triethylsilyl- or a triisopropylsilyl group.

The term “substituent” refers to a group “substituted” on, e.g., an alkyl-, haloalkyl-, cycloalkyl-, heterocyclyl-, heterocycloalkenyl-, cycloalkenyl-, aryl-, or a heteroaryl group at any atom of that group, replacing one or more hydrogen atoms therein. In one aspect, the substituent(s) on a group are independently any one single, or any combination of two or more of the permissible atoms or groups of atoms delineated for that substituent. In another aspect, a substituent may itself be substituted with any one of the above substituents. Further, as used herein, the phrase “optionally substituted” means unsubstituted (e.g., substituted with an H) or substituted.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon.

By “subject” is meant a mammal, including, but not limited to, a human or non-human mammal, such as a bovine, equine, canine, ovine, rodent, or feline.

It is possible for the compounds of general formula (I) to exist as isotopic variants. The invention therefore includes one or more isotopic variant(s) of the compounds of general formula (I), particularly deuterium-containing compounds of general formula (I).

The invention also includes all suitable isotopic variations of a compound of the invention.

The term “isotopic variant” of a compound or a reagent is defined as a compound exhibiting an unnatural proportion of one or more of the isotopes that constitute such a compound.

The expression “unnatural proportion” in relation to an isotope means a proportion of such isotope which is higher than its natural abundance. The natural abundances of isotopes to be applied in this context are described in “Isotopic Compositions of the Elements 1997”, Pure Appl. Chem., 70(1), 217-235, 1998.

An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

With respect to the treatment and/or prophylaxis of the disorders specified herein, the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful, e.g., in drug and/or substrate tissue distribution studies. These isotopes are particularly preferred for the ease of their incorporation and detectability. Positron-emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it may be possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

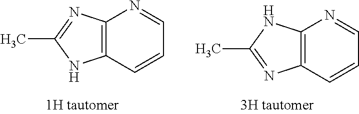

The present invention includes all possible tautomers of the compounds of the present invention as single tautomers, or as any mixture of said tautomers, in any ratio.

Further, in the context of the present invention, it may be possible for the compounds of formula (I) to exist as tautomers. For example, as depicted below, the compounds of formula (I) according to the present invention can exist as a 1H tautomer, or a 3H tautomer, or even a mixture in any amount of two or more of the possible tautomers:

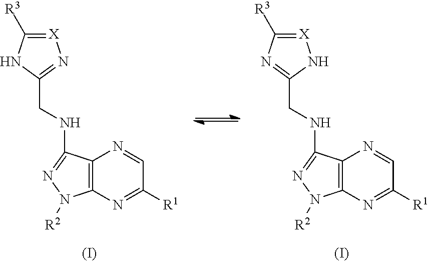

The present invention includes all possible tautomers of the compounds of formula (I) of the present invention as single tautomers, or as any mixture of any two or more of any possible tautomers, in any ratio.

Further, in the context of the present invention, it may be possible for the compounds of formula (I) where X is a nitrogen atom to exist as tautomers. For example, as depicted below, the compounds of formula (I) according to the present invention where X is a nitrogen atom can exist as a 1H tautomer, or a 4H tautomer, or even a mixture in any amount of two or more of the possible tautomers:

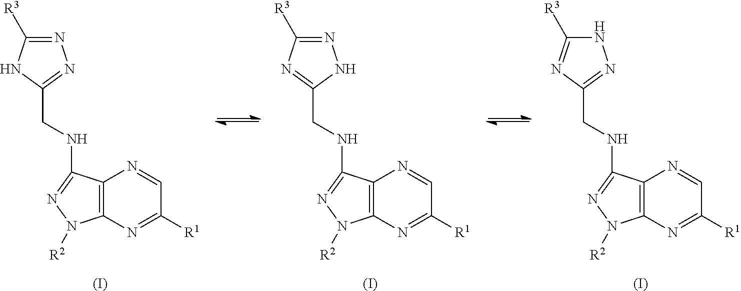

The present invention includes all possible tautomers of the compounds of formula (I) of the present invention where X is a nitrogen atom as single tautomers, or as any mixture of any two or more possible tautomers, in any ratio.

Further, in the context of the present invention, it may be possible for the compounds of formula (I) where X is a CR⁴ group to exist as tautomers. For example, as depicted below, the compounds of formula (I) according to the present invention where X is a CR₄ group can exist as two different 1H tautomers, or even a mixture in any amount of two or more of the possible tautomers:

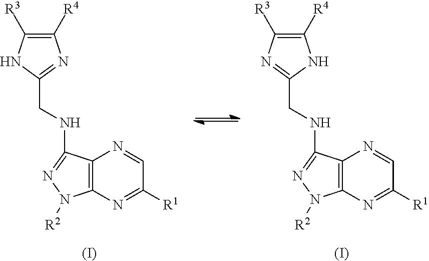

The present invention includes all possible tautomers of the compounds of formula (I) of the present invention where X is a CR⁴ group as single tautomers, or as any mixture of any two or more possible tautomers, in any ratio.

Further, in the context of the present invention, it may be possible for the pirazolopirazine core of the compounds of formula (I) to exhibit tautomerism and for said compounds to exist as single tautomers or even as a mixture in any amount of two or more of the possible tautomers:

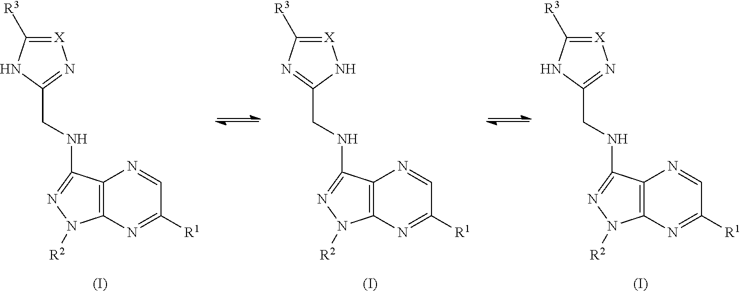

Further, the compounds of the present invention can exist as N-oxides, which are defined in that at least one nitrogen of the compounds of the present invention is oxidised. The present invention includes all such possible N-oxides.

The present invention also provides useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

Physiologically acceptable salts of the compounds according to the invention encompass acid addition salts of mineral acids, carboxylic acids and sulfonic acids, for example salts of hydrochloric acid, hydrobromic acid, hydroiodic acid, sulfuric acid, bisulfuric acid, phosphoric acid, nitric acid or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalenedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) and ammonium salts derived from ammonia or organic amines with 1 to 16 C atoms, such as, by way of example and by preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base, and 1-amino-2,3,4-butanetriol.

Additionally, the compounds according to the invention may form salts with a quaternary ammonium ion obtainable, e.g., by quaternisation of a basic nitrogen-containing group with agents such as lower alkylhalides such as methyl-, ethyl-, propyl-, and butylchlorides, -bromides and -iodides; dialkylsulfates such as dimethyl-, diethyl-, dibutyl- and diamylsulfates, long chain halides such as decyl-, lauryl-, myristyl- and stearylchlorides, -bromides and -iodides, aralkylhalides such as benzyl- and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

Solvates and hydrates of disclosed intermediates or example compounds, or salts thereof, which have been obtained, by the preparation and/or purification processes described herein, may be formed in any ratio.

Furthermore, the present invention includes all possible crystalline forms, or polymorphs, of the compounds of the present invention, either as a single polymorph, or as a mixture of more than one polymorph, in any ratio.

Moreover, the present invention also includes prodrugs of the compounds according to the invention. The term “prodrugs” designates compounds which themselves can be biologically active or inactive, but are converted (for example metabolically or hydrolytically) into compounds according to the invention during their residence time in the body. For example, a prodrug may be in the form of an in vivo hydrolysable ester of the specified compound. Derivatives of the compounds of formula (I) and the salts thereof which are converted into a compound of formula (I) or a salt thereof in a biological system (bioprecursors or pro-drugs) are covered by the invention. Said biological system may be, for example, a mammalian organism, particularly a human subject. The bioprecursor is, for example, converted into the compound of formula (I) or a salt thereof by metabolic processes.

Further, in the context of the present invention, when the inhibitory and/or degradatory activity of the compounds of formula (I) according to the present invention is referred to, the following terms are defined as follows:

As used herein and in the context of the present invention, the term “IC50 CDK12 hATP” refers to the IC₅₀ values obtained according to the assay described in section 2.2 of the Experimental Section herein below, i.e. the IC₅₀ values for the inhibition of CDK12 at high ATP.

As used herein and in the context of the present invention, the term “DC50 CDK12” refers to the DC₅₀ values obtained according to the assay described in section 6 of the Experimental Section herein below, i.e. the DC₅₀ values for the degradation of CDK12.

As used herein and in the context of the present invention, the term “DC50 Cyclin K” refers to the DC₅₀ values obtained according to the assay described in section 7 of the Experimental Section herein below, i.e. the DC₅₀ values for the degradation of Cyclin K.

Description

Further Embodiments of the First Aspect of the Present Invention

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
   wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group;
   R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃COO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group, a heterocycloalkyl group, a 7- to 9-membered bridged compound and a 7- to 11-membered spiro compound,
   wherein said heterocycloalkyl group, 7- to 9-membered bridged compound or 7- to 11-membered spiro compound is connected to the rest of the molecule via a carbon atom of said heterocycloalkyl group,
   wherein said 7- to 9-membered bridged compound or 7- to 11-membered spiro compound each optionally contains one heteroatom independently selected from nitrogen and oxygen,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₄-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-haloalkyl group, a cyano group and a phenyl group,
wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
   wherein said heterocycloalkyl group is connected to the rest of the molecule via a carbon atom of said heterocycloalkyl group,
   wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-haloalkyl group, a cyano group and a phenyl group,
wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₃-C₈-cycloalkyl group, a phenyl group and a heterocycloalkyl group,
   wherein said heterocycloalkyl group is connected to the rest of the molecule via a carbon atom of said heterocycloalkyl group,
   wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group and a heterocycloalkyl group,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a heterocycloalkyl group, a 7- to 9-membered bridged compound and a 7- to 11-membered spiro compound,
   wherein said heterocycloalkyl group, 7- to 9-membered bridged compound or 7- to 11-membered spiro compound is connected to the rest of the molecule via a carbon atom of said heterocycloalkyl group,
   wherein said 7- to 9-membered bridged compound or 7- to 11-membered spiro compound each optionally contains one heteroatom independently selected from nitrogen and oxygen,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-haloalkyl group, a cyano group and a phenyl group,
wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₃-C₈-cycloalkyl group and a heterocycloalkyl group,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
R² is a heterocycloalkyl group,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
   R² is a heterocycloalkyl group,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
   R⁴ is selected from a hydrogen atom, a C₁-C₃-alkyl group and a C₁-C₃-haloalkyl group;
      or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-hydroxyalkyl group, a (C₁-C₃-alkoxy)-(C₁-C₅-alkyl)- group, a (C₃-C₈-cycloalkyl)-(C₁-C₂-alkyl)- group, a (R⁵R⁶N—)—(C₁-C₅-alkyl)- group, a heterocycloalkyl group, a 7- to 9-membered bridged compound and a 7- to 11-membered spiro compound,
   wherein said heterocycloalkyl group, 7- to 9-membered bridged compound or 7- to 11-membered spiro compound is connected to the rest of the molecule via a carbon atom of said heterocycloalkyl group,
   wherein said 7- to 9-membered bridged compound or 7- to 11-membered spiro compound each optionally contains one heteroatom independently selected from nitrogen and oxygen,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₄-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
   wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
   R² is a heterocycloalkyl group selected from tetrahydrofuranyl-, pyrrolidinyl-, tetrahydropyranyl- and piperidinyl-,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, C₃-C₈-cycloalkoxy group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is a CR⁴ group;
      and R³ and R⁴, together with the carbon atoms to which they are attached form a phenyl group,
   wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-haloalkyl group, a C₃-C₈-cycloalkyl group, a phenyl group, a heteroaryl group and a heterocycloalkyl group,
wherein said heterocycloalkyl group is connected to the rest of the molecule via a carbon atom of said heterocycloalkyl group,
wherein said C₃-C₈-cycloalkyl group, C₃-C₈-cycloalkoxy group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-haloalkyl group, a C₃-C₈-cycloalkyl group, a phenyl group, a heteroaryl group and a heterocycloalkyl group,
wherein said heterocycloalkyl group is connected to the rest of the molecule via a carbon atom of said heterocycloalkyl group,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
   wherein said heterocycloalkyl group is connected to the rest of the molecule via a carbon atom of said heterocycloalkyl group,
   wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
   R² is selected from a C₁-C₆-alkyl group, a C₃-C₈-cycloalkyl group, a phenyl group and a 4- to 7-membered heterocycloalkyl group,
   wherein said 4- to 7-membered heterocycloalkyl group is connected to the rest of the molecule via a carbon atom of said 4- to 7-membered heterocycloalkyl group,
   wherein said phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a 4- to 7-membered heterocycloalkyl group, a 7- to 9-membered bridged compound and a 7- to 11-membered spiro compound,
   wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group, 7- to 9-membered bridged compound or 7- to 11-membered spiro compound,
   wherein said 7- to 9-membered bridged compound or 7- to 11-membered spiro compound each optionally contains one heteroatom independently selected from nitrogen and oxygen,
   wherein said C₃-C₈-cycloalkyl group, phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a C₃-C₈-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₄-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
   R² is selected from a C₁-C₆-alkyl group, a C₃-C₈-cycloalkyl group, a phenyl group and a heterocycloalkyl group,
   wherein said heterocycloalkyl group is connected to the rest of the molecule via a carbon atom of said heterocycloalkyl group,
   wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
   R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a 4- to 7-membered heterocycloalkyl group, a 7- to 9-membered bridged compound and a 7- to 11-membered spiro compound,
   wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group, 7- to 9-membered bridged compound or 7- to 11-membered spiro compound,
   wherein said 7- to 9-membered bridged compound or 7- to 11-membered spiro compound each optionally contains one heteroatom independently selected from nitrogen and oxygen,
   wherein said C₃-C₈-cycloalkyl group, phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one, two or three times, each substituent independently selected from a halogen atom and a C₁-C₃-haloalkyl group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-haloalkyl group, a cyano group and a phenyl group,
   wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₃-C₈-cycloalkyl group, a phenyl group and a heterocycloalkyl group,
   wherein said heterocycloalkyl group is connected to the rest of the molecule via a carbon atom of said heterocycloalkyl group,
   wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-haloalkyl group, a cyano group and a phenyl group,
wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
   wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
   R² is a heterocycloalkyl group selected from tetrahydrofuranyl-, pyrrolidinyl-, tetrahydropyranyl- and piperidinyl-,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C1-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
   R⁴ is selected from a hydrogen atom, a C₁-C₃-alkyl group and a C₁-C₃-haloalkyl group;
      or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a heterocycloalkyl group, a 7- to 9-membered bridged compound and a 7- to 11-membered spiro compound,
wherein said heterocycloalkyl group is selected from tetrahydrofuranyl-, pyrrolidinyl-, tetrahydropyranyl- and piperidinyl-,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
R³ and R⁴, together with the carbon atoms to which they are attached form a 5- to 7-membered cycloalkyl, heterocycloalkyl, phenyl or heteroaryl group,
   wherein said heterocycloalkyl or heteroaryl group contains one or two heteroatoms independently selected from nitrogen, oxygen and sulfur,
   and wherein said cycloalkyl, heterocycloalkyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
R³ and R⁴, together with the carbon atoms to which they are attached form a 5- to 7-membered cycloalkyl, heterocycloalkyl, phenyl or heteroaryl group,
   wherein said heterocycloalkyl or heteroaryl group contains one or two heteroatoms independently selected from nitrogen, oxygen and sulfur,
   and wherein said cycloalkyl, heterocycloalkyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one or two times, each substituent independently selected from a halogen atom and a C₁-C₃-haloalkyl group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a heterocycloalkyl group, a 7- to 9-membered bridged compound and a 7- to 11-membered spiro compound,
wherein said heterocycloalkyl group is selected from tetrahydrofuranyl-, pyrrolidinyl-, tetrahydropyranyl- and piperidinyl-,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₄-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a heterocycloalkyl group, a 7- to 9-membered bridged compound and a 7- to 11-membered spiro compound,
wherein said heterocycloalkyl group is selected from tetrahydrofuranyl-, pyrrolidinyl-, tetrahydropyranyl- and piperidinyl-,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
   and R³ and R⁴, together with the carbon atoms to which they are attached form a phenyl group,
   wherein said phenyl group is optionally substituted one or two times, each substituent independently selected from a halogen atom and a C₁-C₃-haloalkyl group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group,
   and wherein, when substituted, said phenyl group is preferably substituted in one or more of the ortho- and/or meta-positions with respect to the point of attachment of said phenyl group to the rest of the molecule;
   R⁴ is selected from a hydrogen atom, a C₁-C₃-alkyl group and a C₁-C₃-haloalkyl group;
      or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
R² is selected from a C₁-C₆-alkyl group, a C₁-C₆-haloalkyl group, a C₃-C₈-cycloalkyl group, a phenyl group, a heteroaryl group and a heterocycloalkyl group,
   wherein said heterocycloalkyl group is connected to the rest of the molecule via a carbon atom of said heterocycloalkyl group,
   wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
   R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group,
   and wherein, when substituted, said phenyl group is preferably substituted in one or more of the ortho- and/or meta-positions with respect to the point of attachment of said phenyl group to the rest of the molecule;
   R⁴ is selected from a hydrogen atom, a C₁-C₃-alkyl group and a C₁-C₃-haloalkyl group;
      or, where X is a CR⁴ group, R³ and R⁴, together with the carbon atoms to which they are attached form a 5- to 7-membered cycloalkenyl, heterocycloalkenyl, phenyl or heteroaryl group,
   wherein said heterocycloalkenyl or heteroaryl group contains one or two heteroatoms independently selected from nitrogen, oxygen and sulfur,
   and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
   wherein said heterocycloalkyl group is connected to the rest of the molecule via a carbon atom of said heterocycloalkyl group,
   wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group,
   and wherein, when substituted, said phenyl group is preferably substituted in one or more of the ortho- and/or meta-positions with respect to the point of attachment of said phenyl group to the rest of the molecule;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

The present invention provides the compounds of general formula (I) which are disclosed in the Example Section of this text, infra.

In some embodiments, the present invention includes compounds of general formula (I) selected from:

N-[(1H-benzimidazol-2-yl)methyl]-6-cyclopropyl-1-(1-methylpiperidin-4-yl)-1H-pyrazolo[3,4-b]pyrazin-3-amine,

6-cyclopropyl-N-[(4-methoxy-1H-benzimidazol-2-yl)methyl]-1-(1-methylpiperidin-4-yl)-1H-pyrazolo[3,4-b]pyrazin-3-amine,

6-cyclopropyl-N-[(4-fluoro-1H-benzimidazol-2-yl)methyl]-1-(1-methylpiperidin-4-yl)-1H-pyrazolo[3,4-b]pyrazin-3-amine,

6-cyclopropyl-N-[(5-fluoro-1H-benzimidazol-2-yl)methyl]-1-(1-methylpiperidin-4-yl)-1H-pyrazolo[3,4-b]pyrazin-3-amine,

6-cyclopropyl-1-(1-methylpiperidin-4-yl)-N-{[4-(trifluoromethoxy)-1H-benzimidazol-2-yl]methyl}-1H-pyrazolo[3,4-b]pyrazin-3-amine,

6-cyclopropyl-N-[(5-methyl-1H-benzimidazol-2-yl)methyl]-1-(1-methylpiperidin-4-yl)-1H-pyrazolo[3,4-b]pyrazin-3-amine,

6-cyclopropyl-1-(1-methylpiperidin-4-yl)-N-{[4-(trifluoromethyl)-1H-benzimidazol-2-yl]methyl}-1H-pyrazolo[3,4-b]pyrazin-3-amine,

6-cyclopropyl-N-[(4-methyl-1H-benzimidazol-2-yl)methyl]-1-(1-methylpi peridi n-4-yl)-1H-pyrazolo[3,4-b]pyrazin-3-amine,

6-cyclopropyl-N-[(4,5-difluoro-1H-benzimidazol-2-yl)methyl]-1-(1-methylpiperidin-4-yl)-1H-pyrazolo[3,4-b]pyrazin-3-amine,

(±)-N-[(1H-benzimidazol-2-yl)methyl]-6-cyclopropyl-1-[oxolan-3-yl]-1H-pyrazolo[3,4-b]pyrazin-3-amine,

tert-butyl 4-(3-{[(1H-benzimidazol-2-yl)methyl]amino}-6-cyclopropyl-1H-pyrazolo[3,4-b]pyrazin-1-yl)piperidine-1-carboxylate,

N-[(1H-benzimidazol-2-yl)methyl]-6-cyclopropyl-1-(piperidin-4-yl)-1H-pyrazolo[3,4-b]pyrazin-3-amine,

formic acid-N-[(1H-benzim idazol-2-yl)methyl]-6-cyclopropyl-1-phenyl-1H-pyrazolo[3,4-b]pyrazin-3-amine (1/1),

N-[(1H-benzimidazol-2-yl)methyl]-6-cyclopropyl-1-(propan-2-yl)-1H-pyrazolo[3,4-b]pyrazin-3-amine,

N-[(1H-benzi midazol-2-yl)methyl]-6-cyclopropyl-1-methyl-1H-pyrazolo[3,4-b]pyrazin-3-amine,

N-[(1H-benzimidazol-2-yl)methyl]-6-cyclopropyl-1-(oxan-4-yl)-1H-pyrazolo[3,4-b]pyrazin-3-amine,

(+) or (−)-N-[(1H-benzimidazol-2-yl)methyl]-6-cyclopropyl-1-(oxolan-3-yl)-1H-pyrazolo[3,4-b]pyrazin-3-amine,

(−) or (+) -N-[(1H-benzimidazol-2-yl)methyl]-6-cyclopropyl-1-[oxolan-3-yl]-1H-pyrazolo[3,4-b]pyrazin-3-amine,

6-cyclopropyl-N-[(5,6-dichloro-1H-benzimidazol-2-yl)methyl]-1-(1-methylpiperidin-4-yl)-1H-pyrazolo[3,4-b]pyrazin-3-amine,

N-[(4-bromo-1H-benzimidazol-2-yl)methyl]-6-cyclopropyl-1-(1-methylpiperidin-4-yl)-1H-pyrazolo[3,4-b]pyrazin-3-amine,

N-[(5-chloro-4-methyl-1H-benzimidazol-2-yl)methyl]-6-cyclopropyl-1-(1-methylpiperidin-4-yl)-1H-pyrazolo[3,4-b]pyrazin-3-amine,

N-[(5-chloro-1H-benzimidazol-2-yl)methyl]-6-cyclopropyl-1-(1-methylpiperidin-4-yl)-1H-pyrazolo[3,4-b]pyrazin-3-amine,

6-cyclopropyl-N-[(4-fluoro-1H-benzimidazol-2-yl)methyl]-1-(oxan-4-yl)-1H-pyrazolo[3,4-b]pyrazin-3-amine,

6-cyclopropyl-N-[(4-methoxy-1H-benzimidazol-2-yl)methyl]-1-(oxan-4-yl)-1H-pyrazolo[3,4-b]pyrazin-3-amine,

6-cyclopropyl-1-(1-methylpiperidin-4-yl)-N-{[5-(trifluoromethyl)-1H-benzimidazol-2-yl]methyl}-1H-pyrazolo[3,4-b]pyrazin-3-amine,

6-cyclopropyl-N-[(5-fluoro-4-methyl-1H-benzimidazol-2-yl)methyl]-1-(1-methylpiperidin-4-yl)-1H-pyrazolo[3,4-b]pyrazin-3-amine,

6-cyclopropyl-1-(1-methylpiperidin-4-yl)-N-({5-[(trifluoromethyl)sulfanyl]-1H-benzimidazol-2-yl}methyl)-1H-pyrazolo[3,4-b]pyrazin-3-amine,

6-cyclopropyl-N-[(4,5-dimethyl-1H-benzimidazol-2-yl)methyl]-1-(1-methylpiperidin-4-yl)-1H-pyrazolo[3,4-b]pyrazin-3-amine,

N-[(5-chloro-6-fluoro-1H-benzimidazol-2-yl)methyl]-6-cyclopropyl-1-(1-methylpiperidin-4-yl)-1H-pyrazolo[3,4-b]pyrazin-3-amine,

6-cyclopropyl-N-[(5,6-difluoro-1H-benzimidazol-2-yl)methyl]-1-(1-methylpiperidin-4-yl)-1H-pyrazolo[3,4-b]pyrazin-3-amine,

N-[(4-chloro-5-fluoro-1H-benzimidazol-2-yl)methyl]-6-cyclopropyl-1-(1-methylpiperidin-4-yl)-1H-pyrazolo[3,4-b]pyrazin-3-amine,

6-cyclopropyl-N-[(4,5-difluoro-1H-benzimidazol-2-yl)methyl]-1-(oxan-4-yl)-1H-pyrazolo[3,4-b]pyrazin-3-amine,

6-cyclopropyl-N-[(4-methyl-1H-benzimidazol-2-yl)methyl]-1-(oxan-4-yl)-1H-pyrazolo[3,4-b]pyrazin-3-amine,

(±)-N-[(1H-benzimidazol-2-yl)methyl]-6-cyclopropyl-1-(1-methylazepan-4-yl)-1H-pyrazolo[3,4-b]pyrazin-3-amine,

(±)-N-[(1H-benzimidazol-2-yl)methyl]-6-cyclopropyl-1-(1-methylpyrrolidin-3-yl)-1H-pyrazolo[3,4-b]pyrazin-3-amine,

N-[(1H-benzimidazol-2-yl)methyl]-6-cyclopropyl-1-(4,4-difluorocyclohexyl)-1H-pyrazolo[3,4-b]pyrazin-3-amine,

(+) or (−)-N-[(1H-benzimidazol-2-yl)methyl]-6-cyclopropyl-1-[(4R*)-1-methylazepan-4-yl]-1H-pyrazolo[3,4-b]pyrazin-3-amine,

(—) or (+)-N-[(1H-benzimidazol-2-yl)methyl]-6-cyclopropyl-1-[(4R*)-1-methylazepan-4-yl]-1H-pyrazolo[3,4-b]pyrazin-3-amine,

N-[(1H-benzimidazol-2-yl)methyl]-1-cyclopentyl-6-cyclopropyl-1H-pyrazolo[3,4-b]pyrazin-3-amine,

cis-3-(3-{[(1H-benzim idazol-2-yl)methyl]amino}-6-cyclopropyl-1H-pyrazolo[3,4-b]pyrazin-1-yl)cyclobutan-1-ol,

(±)-cis/trans-N-[(1H-benzimidazol-2-yl)methyl]-6-cyclopropyl-1-(1,2-dimethylpiperidin-4-yl)-1H-pyrazolo[3,4-b]pyrazin-3-amine,

(±)-cis/trans-N-[(1H-benzimidazol-2-yl)methyl]-6-cyclopropyl-1-(3-methyloxan-4-yl)-1H-pyrazolo[3,4-b]pyrazin-3-amine,

trans-3-(3-{[(1H-benzimidazol-2-yl)methyl]amino}-6-cyclopropyl-1H-pyrazolo[3,4-b]pyrazin-1-yl)cyclobutan-1-ol,

(±)-cis/trans-N-[(1H-benzimidazol-2-yl)methyl]-6-cyclopropyl-1-(2-methyloxan-4-yl)-1H-pyrazolo[3,4-b]pyrazin-3-amine,

exo/endo-N-[(1H-benzim idazol-2-yl)methyl]-6-cyclopropyl-1-(8-oxabicyclo[3.2.1]octan-3-yl)-1H-pyrazolo[3,4-b]pyrazin-3-amine,

[trans-3-(3-{[(1H-benzimidazol-2-yl)methyl]amino}-6-cyclopropyl-1H-pyrazolo[3,4-b]pyrazin-1-yl)cyclobutyl]methanol,

[cis-3-(3-{[(1H-benzimidazol-2-yl)methyl]amino}-6-cyclopropyl-1H-pyrazolo[3,4-b]pyrazin-1-yl)cyclobutyl]methanol,

2-[cis/trans-3-(3-{[(1H-benzimidazol-2-yl)methyl]amino}-6-cyclopropyl-1H-pyrazolo[3,4-b]pyrazin-1-yl)cyclobutyl]propan-2-ol,

N-[(1H-benzimidazol-2-yl)methyl]-1-cyclobutyl-6-cyclopropyl-1H-pyrazolo[3,4-b]pyrazin-3-amine,

trans-4-(3-{[(1H-benzimidazol-2-yl)methyl]amino}-6-cyclopropyl-1H-pyrazolo[3,4-b]pyrazin-1-yl)cyclohexan-1-ol,

cis-4-(3-{[(1H-benzimidazol-2-yl)methyl]amino}-6-cyclopropyl-1H-pyrazolo[3,4-b]pyrazin-1-yl)cyclohexan-1-ol,

N-[(1H-benzimidazol-2-yl)methyl]-6-cyclopropyl-1-(2,2,6,6-tetramethyloxan-4-yl)-1H-pyrazolo[3,4-b]pyrazin-3-amine,

6-cyclopropyl-N-[(5-fluoro-1H-benzimidazol-2-yl)methyl]-1-(oxan-4-yl)-1H-pyrazolo[3,4-b]pyrazin-3-amine,

N-[(1H-benzimidazol-2-yl)methyl]-6-cyclopropyl-1-(2-oxaspiro[3.3]heptan-6-yl)-1H-pyrazolo[3,4-b]pyrazin-3-amine,

N-[(1H-benzimidazol-2-yl)methyl]-6-cyclopropyl-1-(oxetan-3-yl)-1H-pyrazolo[3,4-b]pyrazin-3-amine,

trans-4-(6-cyclopropyl-3-{[(5-fluoro-1H-benzimidazol-2-yl)methyl]amino}-1H-pyrazolo[3,4-b]pyrazin-1-yl)cyclohexan-1-ol,

(±)-N-[(1H-benzimidazol-2-yl)methyl]-6-cyclopropyl-1-(4-methoxybutan-2-yl)-1H-pyrazolo[3,4-b]pyrazin-3-amine,

(−)-N-[(1H-benzimidazol-2-yl)methyl]-6-cyclopropyl-1-[4-methoxybutan-2-yl]-1H-pyrazolo[3,4-b]pyrazin-3-amine,

(+)-N-[(1H-benzimidazol-2-yl)methyl]-6-cyclopropyl-1-[4-methoxybutan-2-yl]-1H-pyrazolo[3,4-b]pyrazin-3-amine,

3-(3-{[(1H-benzimidazol-2-yl)methyl]amino}-6-cyclopropyl-1H-pyrazolo[3,4-b]pyrazin-1-yl)propan-1-ol,

N-[(1H-benzimidazol-2-yl)methyl]-6-cyclopropyl-1-(3-methoxypropyl)-1H-pyrazolo[3,4-b]pyrazin-3-amine,

(±)-N-[(1H-benzimidazol-2-yl)methyl]-6-cyclopropyl-1-[4-(dimethylamino)butan-2-yl]-1H-pyrazolo[3,4-b]pyrazin-3-amine,

(+) or (−)-N-[(1H-benzimidazol-2-yl)methyl]-6-cyclopropyl-1-[4-(dimethylamino)butan-2-yl]-1H-pyrazolo[3,4-b]pyrazin-3-amine,

(−) or(+)-N-[(1H-benzimidazol-2-yl)methyl]-6-cyclopropyl-1-[4-(dimethylamino)butan-2-yl]-1H-pyrazolo[3,4-b]pyrazin-3-amine,

N-[(1H-benzimidazol-2-yl)methyl]-6-cyclopropyl-1-(2-methoxyethyl)-1H-pyrazolo[3,4-b]pyrazin-3-amine,

2-(3-{[(1H-benzimidazol-2-yl)methyl]amino}-6-cyclopropyl-1H-pyrazolo[3,4-b]pyrazin-1-yl)ethan-1-ol,

N-[(1H-benzimidazol-2-yl)methyl]-6-cyclopropyl-1-(3-methoxy-3-methylbutyl)-1H-pyrazolo[3,4-b]pyrazin-3-amine,

N-[(1H-benzimidazol-2-yl)methyl]-6-cyclopropyl-1-[2-(dimethylamino)ethyl]-1H-pyrazolo[3,4-b]pyrazin-3-amine,

1-(3-{[(1H-benzimidazol-2-yl)methyl]amino}-6-cyclopropyl-1H-pyrazolo[3,4-b]pyrazin-1-yl)-2-methylpropan-2-ol,

trans-3-[(3-{[(1H-benzimidazol-2-yl)methyl]amino}-6-cyclopropyl-1H-pyrazolo[3,4-b]pyrazin-1-yl)methyl]cyclobutan-1-ol,

cis-3-[(3-{[(1H-benzimidazol-2-yl)methyl]amino}-6-cyclopropyl-1H-pyrazolo[3,4-b]pyrazin-1-yl)methyl]cyclobutan-1-ol,

1-[(3-{[(1H-benzimidazol-2-yl)methyl]amino}-6-cyclopropyl-1H-pyrazolo[3,4-b]pyrazin-1-yl)methyl]cyclobutan-1-ol,

1-[(3-{[(1H-benzimidazol-2-yl)methyl]amino}-6-cyclopropyl-1H-pyrazolo[3,4-b]pyrazin-1-yl)methyl]cyclopentan-1-ol,

{1-[(3-{[(1H-benzimidazol-2-yl)methyl]amino}-6-cyclopropyl-1H-pyrazolo[3,4-b]pyrazin-1-yl)methyl]cyclobutyl}methanol,

N-[(1H-benzimidazol-2-yl)methyl]-1-(cyclobutylmethyl)-6-cyclopropyl-1H-pyrazolo[3,4-b]pyrazin-3-amine,

4-(3-{[(1H-benzimidazol-2-yl)methyl]amino}-6-cyclopropyl-1H-pyrazolo[3,4-b]pyrazin-1-yl)-2-methylbutan-2-ol,

N-[(1H-benzimidazol-2-yl)methyl]-6-methyl-1-(1-methylpiperidin-4-yl)-1H-pyrazolo[3,4-b]pyrazin-3-amine,

(+) or (−)-N-[(1H-benzimidazol-2-yl)methyl]-6-cyclopropyl-1-(1-methylpyrrolidin-3-yl)-1H-pyrazolo[3,4-b]pyrazin-3-amine and

(−) or (+)-N-[(1H-benzimidazol-2-yl)methyl]-6-cyclopropyl-1-(1-methylpyrrolidin-3-yl)-1H-pyrazolo[3,4-b]pyrazin-3-amine.

In some embodiments, the present invention includes compounds of general formula (I), supra, which show a ratio (IC50 CDK12 hATP)/(DC50 CDK12) which is equal or greater than 5.

In some embodiments, the present invention includes compounds of general formula (I), supra, which show a ratio (IC50 CDK12 hATP)/(DC50 CDK12) which is greater than 5.

In some embodiments, the present invention includes compounds of general formula (I), supra, which show a ratio (IC50 CDK12 hATP)/(DC50 CDK12) which is equal or greater than 10.

In some embodiments, the present invention includes compounds of general formula (I), supra, which show a ratio (IC50 CDK12 hATP)/(DC50 CDK12) which is greater than 10.

In some embodiments, the present invention includes compounds of general formula (I), supra, which show a ratio (IC50 CDK12 hATP)/(DC50 CDK12) which is equal or greater than 20.

In some embodiments, the present invention includes compounds of general formula (I), supra, which show a ratio (IC50 CDK12 hATP)/(DC50 CDK12) which is greater than 20.

In some embodiments, the present invention includes compounds of general formula (I), supra, which show a ratio (IC50 CDK12 hATP)/(DC50 CDK12) which is equal or greater than 30.

In some embodiments, the present invention includes compounds of general formula (I), supra, which show a ratio (IC50 CDK12 hATP)/(DC50 CDK12) which is greater than 30.

In some embodiments, the present invention includes compounds of general formula (I), supra, which show a ratio (IC50 CDK12 hATP)/(DC50 CDK12) which is equal or greater than 50.

In some embodiments, the present invention includes compounds of general formula (I), supra, which show a ratio (IC50 CDK12 hATP)/(DC50 CDK12) which is greater than 50.

In some embodiments, the present invention includes compounds of general formula (I), supra, which show a ratio (IC50 CDK12 hATP)/(DC50 CDK12) which is equal or greater than 100.

In some embodiments, the present invention includes compounds of general formula (I), supra, which show a ratio (IC50 CDK12 hATP)/(DC50 CDK12) which is greater than 100.

In some embodiments, the present invention includes compounds of general formula (I), supra, which show a ratio (IC50 CDK12 hATP)/(DC50 CDK12) which is equal or greater than 500.

In some embodiments, the present invention includes compounds of general formula (I), supra, which show a ratio (IC50 CDK12 hATP)/(DC50 CDK12) which is greater than 500.

In some embodiments, the present invention includes compounds of general formula (I), supra, which show a ratio (IC50 CDK12 hATP)/(DC50 CDK12) which is equal or greater than 1000.

In some embodiments, the present invention includes compounds of general formula (I), supra, which show a ratio (IC50 CDK12 hATP)/(DC50 CDK12) which is greater than 1000.

In some embodiments, the present invention includes compounds of general formula (I), supra, which show a ratio (IC50 CDK12 hATP)/(DC50 CDK12) which is greater than 5 and a (DC50 CDK12) value which is lower than 200 nM.

In some embodiments, the present invention includes compounds of general formula (I), supra, which show a ratio (IC50 CDK12 hATP)/(DC50 CDK12) which is greater than 5 and a (DC50 CDK12) value which is lower than 20 nM.

In some embodiments, the present invention includes compounds of general formula (I), supra, which show a ratio (IC50 CDK12 hATP)/(DC50 CDK12) which is greater than 5 and a (DC50 CDK12) value which is lower than 2 nM.

In some embodiments, the present invention includes compounds of general formula (I), supra, which show a ratio (IC50 CDK12 hATP)/(DC50 CDK12) which is greater than 10 and a (DC50 CDK12) value which is lower than 200 nM.

In some embodiments, the present invention includes compounds of general formula (I), supra, which show a ratio (IC50 CDK12 hATP)/(DC50 CDK12) which is greater than 10 and a (DC50 CDK12) value which is lower than 20 nM.

In some embodiments, the present invention includes compounds of general formula (I), supra, which show a ratio (IC50 CDK12 hATP)/(DC50 CDK12) which is greater than 10 and a (DC50 CDK12) value which is lower than 2 nM.

In some embodiments, the present invention includes compounds of general formula (I), supra, which show a ratio (IC50 CDK12 hATP)/(DC50 CDK12) which is equal or greater than 20 and a (DC50 CDK12) value which is equal or lower than 200 nM.

In some embodiments, the present invention includes compounds of general formula (I), supra, which show a ratio (IC50 CDK12 hATP)/(DC50 CDK12) which is greater than 20 and a (DC50 CDK12) value which is lower than 200 nM.

In some embodiments, the present invention includes compounds of general formula (I), supra, which show a ratio (IC50 CDK12 hATP)/(DC50 CDK12) which is greater than 20 and a (DC50 CDK12) value which is lower than 20 nM.

In some embodiments, the present invention includes compounds of general formula (I), supra, which show a ratio (IC50 CDK12 hATP)/(DC50 CDK12) which is greater than 20 and a (DC50 CDK12) value which is lower than 2 nM.

In some embodiments, the present invention includes compounds of general formula (I), supra, which show a ratio (IC50 CDK12 hATP)/(DC50 CDK12) which is equal or greater than 100 and a (DC50 CDK12) value which is equal or lower than 200 nM.

In some embodiments, the present invention includes compounds of general formula (I), supra, which show a ratio (IC50 CDK12 hATP)/(DC50 CDK12) which is greater than 100 and a (DC50 CDK12) value which is lower than 200 nM.

In some embodiments, the present invention includes compounds of general formula (I), supra, which show a ratio (IC50 CDK12 hATP)/(DC50 CDK12) which is greater than 100 and a (DC50 CDK12) value which is lower than 20 nM.

In some embodiments, the present invention includes compounds of general formula (I), supra, which show a ratio (IC50 CDK12 hATP)/(DC50 CDK12) which is greater than 100 and a (DC50 CDK12) value which is lower than 2 nM.

In some embodiments, the present invention includes compounds of general formula (I), supra, which show a ratio (IC50 CDK12 hATP)/(DC50 CDK12) which is equal or greater than 500 and a (DC50 CDK12) value which is equal or lower than 200 nM.

In some embodiments, the present invention includes compounds of general formula (I), supra, which show a ratio (IC50 CDK12 hATP)/(DC50 CDK12) which is greater than 500 and a (DC50 CDK12) value which is lower than 200 nM.

In some embodiments, the present invention includes compounds of general formula (I), supra, which show a ratio (IC50 CDK12 hATP)/(DC50 CDK12) which is greater than 500 and a (DC50 CDK12) value which is lower than 20 nM.

In some embodiments, the present invention includes compounds of general formula (I), supra, which show a ratio (IC50 CDK12 hATP)/(DC50 CDK12) which is greater than 500 and a (DC50 CDK12) value which is lower than 2 nM.

Further Embodiments of the First Aspect of the Present Invention:

In some embodiments, the present invention provides compounds of formula (I), supra, in which R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group,
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom and a C₁-C₆-alkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom and a C₁-C₆-alkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₆-alkyl group, a C₁-C₆-haloalkyl group, a C₃-C₈-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₄-haloalkyl group, a C₃-C₄-cycloalkyl group, a a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₃-alkyl group, a C₁-C₃-haloalkyl group, a C₃-C₆-cycloalkyl group, and a C₃-C₆-halocycloalkyl group,
wherein said C₁-C₃-alkyl group or C₃-C₆-cycloalkyl group is each optionally substituted with one or more substituents independently selected from a halogen atom, a C₁-C₂-alkyl group and a C₁-C₂-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₃-alkyl group, a C₁-C₃-haloalkyl group, a C₃-C₆-cycloalkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₃-alkyl group, a C₁-C₃-haloalkyl group, a C₃-C₆-cycloalkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group, a C₁-C₃-haloalkyl group, a C₃-C₆-cycloalkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group, a C₁-C₃-haloalkyl group and a C₃-C₆-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group and a C₃-C₆-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group and a C₁-C₃-haloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group and a trifluoromethyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group, a C₁-C₃-alkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a cyano group, a C₁-C₃-alkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a cyano group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom and a cyano group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₆-alkyl group, a C₁-C₆-haloalkyl group, a C₃-C₈-cycloalkyl group, a (C₃-C₈-cycloalkyl)-(C₁-C₆-alkyl)- group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₃-alkyl group, a C₁-C₃-haloalkyl group, a C₃-C₆-cycloalkyl group and a C₃-C₆-halocycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₃-alkyl group and a C₃-C₆-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a C₁-C₃-alkyl group and a C₃-C₆-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is a C₃-C₆-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₆-haloalkyl group, a cyano group and a phenyl group,
wherein said phenyl group is optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R² is selected is selected from a C₁-C₆-alkyl group, a C₁-C₆-haloalkyl group, a C₃-C₈-cycloalkyl group, a phenyl group, a heteroaryl group and a heterocycloalkyl group,
wherein said heterocycloalkyl group is connected to the rest of the molecule via a carbon atom of said heterocycloalkyl group,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R² is selected is selected from a C₁-C₆-alkyl group, a C₁-C₆-haloalkyl group a C₃-C₈-cycloalkyl group, a phenyl group, a heteroaryl group and a 4- to 7-membered heterocycloalkyl group,
wherein said 4- to 7-membered heterocycloalkyl group is connected to the rest of the molecule via a carbon atom of said 4- to 7-membered heterocycloalkyl group,
wherein said C₃-C₈-cycloalkyl group, phenyl group, heteroaryl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₃-C₈-cycloalkyl group, a phenyl group and a 4- to 7-membered heterocycloalkyl group,
wherein said 4- to 7-membered heterocycloalkyl group is connected to the rest of the molecule via a carbon atom of said 4- to 7-membered heterocycloalkyl group,
wherein said phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group, a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group, a 4- to 7-membered heterocycloalkyl group, a 7- to 9-membered bridged compound and a 7- to 11-membered spiro compound,
wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a C₁-C₃-alkyl group, a C₁-C₃-haloalkyl group, a C₁-C₃-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group, a 4- to 7-membered heterocycloalkyl group, a 7- to 9-membered bridged compound and a 7- to 11-membered spiro compound,
wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a 4- to 7-membered heterocycloalkyl group, a 7- to 9-membered bridged compound and a 7- to 11-membered spiro compound,
wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group,
wherein said 7- to 9-membered bridged compound or 7- to 11-membered spiro compound each optionally contains one heteroatom independently selected from nitrogen and oxygen,
wherein said 4- to 7-membered heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a heterocycloalkyl group, a 7- to 9-membered bridged compound and a 7- to 11-membered spiro compound,
wherein said heterocycloalkyl group is selected from tetrahydrofuranyl-, pyrrolidinyl-, tetrahydropyranyl- and piperidinyl-,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which
R² is selected from a 7- to 9-membered bridged compound and a 7- to 11-membered spiro compound,
   wherein said 7- to 9-membered bridged compound or 7- to 11-membered spiro compound each optionally contains one heteroatom independently selected from nitrogen and oxygen,
      or a tautomer, or an N-oxide, or a salt thereof, or a salt of a tautomer, or a salt of an N-oxide, or a mixture of same.

In some embodiments, the present invention provides compounds of formula (I), supra, in which
R² is a 7- to 9-membered bridged compound which optionally contains one heteroatom independently selected from nitrogen and oxygen,
   or a tautomer, or an N-oxide, or a salt thereof, or a salt of a tautomer, or a salt of an N-oxide, or a mixture of same.

In some embodiments, the present invention provides compounds of formula (I), supra, in which
R² is a 7- to 11-membered spiro compound which optionally contains one heteroatom independently selected from nitrogen and oxygen,
   or a tautomer, or an N-oxide, or a salt thereof, or a salt of a tautomer, or a salt of an N-oxide, or a mixture of same.

In some embodiments, the present invention provides compounds of formula (I), supra, in which X is selected from a nitrogen atom and a CR⁴ group or a tautomer, or an N-oxide, or a salt thereof, or a salt of a tautomer, or a salt of an N-oxide, or a mixture of same.

In some embodiments, the present invention provides compounds of formula (I), supra, in which X is a nitrogen atom or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
and wherein, when substituted, said phenyl group is preferably substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
and wherein, when substituted, said phenyl group is substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
and wherein, when substituted, said phenyl group is preferably substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
and wherein, when substituted, said phenyl group is substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a C₁-C₃-alkyl group and a C₁-C₃-haloalkyl group;

or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group;

or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a C₁-C₃-alkyl group and a C₁-C₃-haloalkyl group or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl, phenyl or heteroaryl group,
wherein said heteroaryl group contains one or two heteroatoms independently selected from nitrogen, oxygen and sulfur,
and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one, two or three times, each substituent independently selected from a halogen atom, a C₁-C₃-alkyl group, a C₁-hydroxyalkyl group, a C₁-C₃-haloalkyl group, a C₁-C₃-alkoxy group, a C₁-C₆-haloalkoxy group and a C₃-C₅-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one or two times, each substituent independently selected from a halogen atom, a C₁-C₃-alkyl group, a C₁-hydroxyalkyl group, a C₁-C₃-haloalkyl group, a C₁-C₃-alkoxy group and a C₁-C₆-haloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl group,
wherein said 6-membered cycloalkenyl group is each optionally substituted one or two times, each substituent independently selected from a halogen atom, a C₁-C₃-alkyl group, a C₁-hydroxyalkyl group, a C₁-C₃-haloalkyl group, a C₁-C₃-alkoxy group and a C₁-C₆-haloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl or a heteroaryl group,
wherein said heteroaryl group contains one or two heteroatoms independently selected from nitrogen, oxygen and sulfur,
and wherein said phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group and a C₃-C₅-cycloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-alkoxy group, a C₁-C₆-hydroxyalkyl group, a C₁-C₆-haloalkyl group, and a C₃-C₅-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a C₁-C₆-alkyl group, a C₁-C₆-alkoxy group, a C₁-C₆-hydroxyalkyl group, a C₁-C₆-haloalkyl group, and a C₃-C₅-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom, a C₁-C₆-alkyl group, a C₁-C₆-alkoxy group, a C₁-C₆-hydroxyalkyl group and a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom, a C₁-C₃-alkyl group, a C₁-C₃-alkoxy group, a C₁-C₃-hydroxyalkyl group and a C₁-C₃-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom, a C₁-C₃-alkoxy group and a C₁-C₃-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom and a C₁-C₃-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵and R⁶ are each independently selected from a hydrogen atom, a C₁-C₃-alkyl group, a C₃-C₈-cycloalkyl group, a C₁-C₃-haloalkyl group, a (C₃-C₈-cycloalkyl)-(C₁-C₃-alkyl)- group, a C₁-C₃-hydroxyalkyl group, a (C₁-C₃-alkoxy)-(C₁-C₃-alkyl)-group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵and R⁶ are each independently selected from a hydrogen atom, a C₁-C₃-alkyl group, a C₃-C₈-cycloalkyl group, a C₁-C₃-haloalkyl group, a (C₃-C₈-cycloalkyl)-(C₁-C₃-alkyl)- group, a C₁-C₃-hydroxyalkyl group, a (C₁-C₃-alkoxy)-(C₁-C₃-alkyl)- group, or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₃-alkyl group, a C₃-C₆-cycloalkyl group, a C₂-C₃-haloalkyl group, a (C₃-C₆-cycloalkyl)-(C₁-C₃-alkyl)- group, a C₂-C₃-hydroxyalkyl group, a (C₁-C₃-alkoxy)-(C₁-C₃-alkyl)- group, or a tautomer, or an N-oxide, or a salt thereof, or a salt of a tautomer, or a salt of an N-oxide, or a mixture of same.

In some embodiments, the present invention provides compounds of formula (I), supra, in which R⁵and R⁶ are each independently selected from a hydrogen atom, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom and a C₁-C₃-alkyl group, or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each a C₁-C₃-alkyl group, or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R⁷ is selected from a hydrogen atom and a C₁-C₄-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ is selected from a hydrogen atom and a C₁-C₃-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R⁷ is a C₁-C₄-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ is a C₁-C₃-alkyl group or a tautomer, or an N-oxide, or a salt thereof, or a salt of a tautomer, or a salt of an N-oxide, or a mixture of same.

In further embodiments, the present invention includes compounds of formula (I), or a tautomer, an N-oxide, or a salt thereof, or a salt of a tautomer or an N-oxide, or a mixture of same.

In further embodiments, the present invention includes compounds of formula (I), or a salt thereof.

In further embodiments, the present invention includes compounds of formula (I), or a tautomer, or a salt thereof, or a salt of a tautomer, or a mixture of same.

In further embodiments, the present invention includes compounds of formula (I), which are a salt.

In further embodiments, the present invention includes compounds of formula (I), which are a tautomer or a salt thereof, or a salt of a tautomer, or a mixture of same.

In further embodiments, the present invention includes compounds of formula (I), which are an N-oxide, or a salt thereof, or a salt of an N-oxide, or a mixture of same.

In further embodiments of the first aspect, the present invention provides combinations of two or more of the above mentioned embodiments under the heading “further embodiments of the first aspect of the present invention”.

Furthermore it is understood that the invention includes any subcombination of the disclosed single embodiments herein for certain residues or subcombination of residues of formula (I).

The present invention includes any sub-combination within any embodiments or aspects of the present invention of compounds of general formula (I), supra.

The present invention includes any sub-combination within any embodiments or aspects of the present invention of compounds of general formula (I) or intermediate compounds. The present invention includes the compounds of general formula (I) which are disclosed in the Example Section of this text, infra.

General Synthesis of Compounds of General Formula (I) of the Present Invention

The following paragraphs outline a variety of synthetic approaches suitable to prepare compounds of the general formula (I), and intermediates useful for their synthesis.

In addition to the routes described below, also other routes may be used to synthesise the target compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R², R³ or R⁴ can be achieved before and/or after the exemplified transformations. These modifications can be such as the introduction of protective groups, cleavage of protective groups, reduction or oxidation of functional groups, halogenation, metallation, metal catalysed coupling reactions, exemplified by but not limited to Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999).

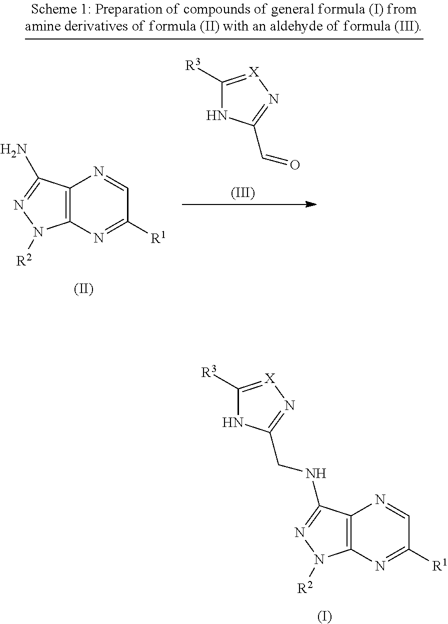

Compounds of general formula (I) can be assembled from amine derivatives of formula (II), in which R¹ and R² are as defined for the compounds of general formula (I), and an aldehyde of formula (III), in which R² and X are as defined for the compounds of general formula (I), by means of a reductive amination well known to the person skilled in the art, according to Scheme 1. Said reductive amination reaction can be performed by reaction of compounds of the formulae (II) and (III) in the presence of a suitable acid, such as acidic acid, trifluoroacetic acid or titanium(IV) isopropoxide, and a reducing reagent such as sodium borohydride or sodium cyanoborohydride in an appropriate solvent.

Preferred herein is the performance of said reductive aminination reaction using acetic acid or additional drops of trifluoroacetic acid and sodium cyanoborohydride as a reducing reagent in methanol as a solvent, within a temperature range from 20° C. to 60° C.

Also preferred herein is the performance of said reductive aminination reaction using titanium(IV) isopropoxide as acid component and sodium borohydride as a reducing reagent in methanol as a solvent, within a temperature range from 20° C. to 60° C.

Compounds of the general formula (I) with different NH or OH groups in the molecule may be protected before and may be deprotected after the reductive amination step if necessary well known to the person skilled in the art.

In the case of R¹ being a halogen atom, a reaction sequence first introducing a methoxy group wherein said methoxy group is substituted with a halogen atom at the end of the synthetic procedure leading to the compound of formula (I) might be necessary and is available to a person skilled in the art using methodologies and procedures known in the art.

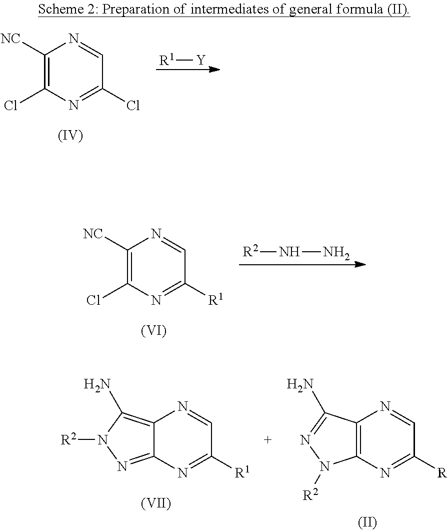

Intermediate amine derivatives of formula (II) are available for example by the sequence depicted in scheme 2. This approach started with commercially available cyanide (IV), using metallo organic chemistry well known to the person skilled in the art at the more reactive chloro atom, which can be substituted by R¹, for example using a Suzuki reaction. In these cases the Y in (V) is B(OH)₂ or any other boronic acid derivative useful for such a reaction in the presence of a Pd-catalyst for Suzuki reactions well known to the person skilled in the art for the production of compounds of formula (VI). In principle also other reagents for the introduction of R¹ are possible such as Sn- or Zn-reagents for Y or other different metal-catalyzed reactions well known to the person skilled in the art.

The reaction of compounds of formula (VI) with a hydrazine derivative of formula (VII) or its salt in the presence of a base such as sodium carbonate or potassium carbonate give the desired compounds of formula (II) together with its isomer (VIII), which can be separated easily by chromatography.

Hydrazine derivative of formula (VII) or its salt are commercially available or can be easily prepared by its aldehyde or ketone precursors using a reaction sequence of addition of tert-butyl hydrazinecarboxylate to the carbonyl forming an imine which will be reduced with e.g. sodium cyanoborohydride to afford the corresponding Boc-protected hydrazine followed by a deprotection step using an acid such as hydrochloric acid or others for such a deprotection well known to the person skilled in the art to form the desired hydrazine derivative.

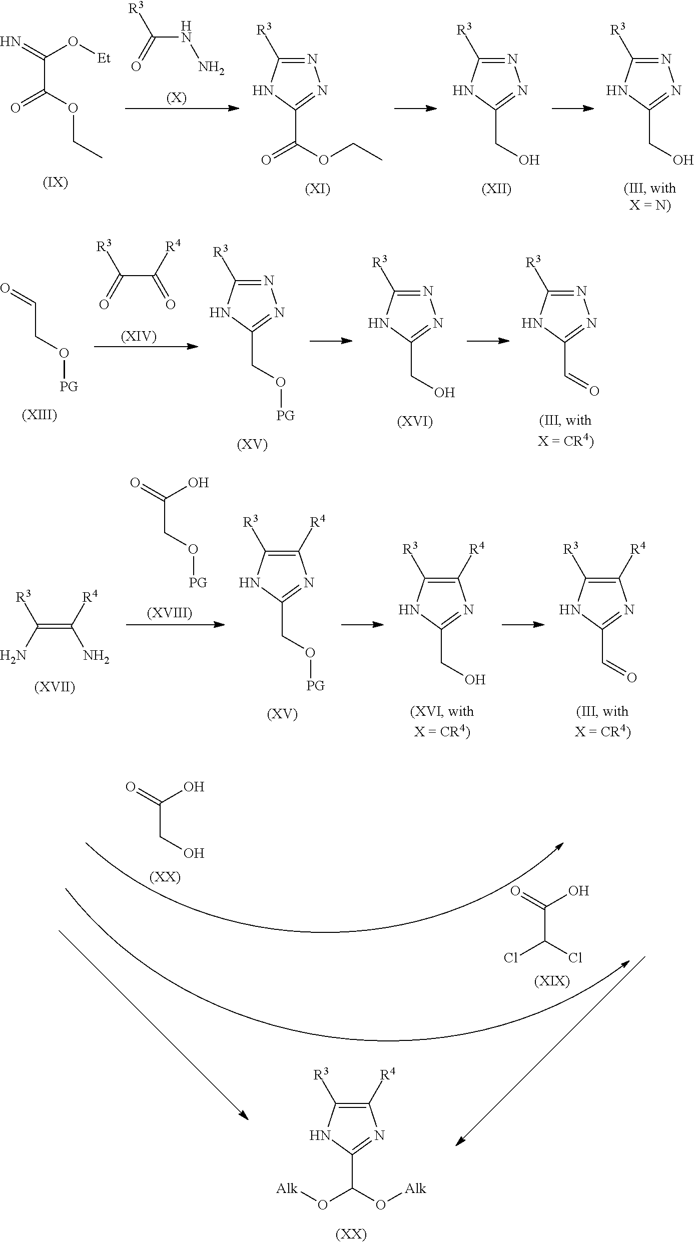

The synthesis of different types of aldehydes of the general formula (III) is depicted in scheme 3. In the case of aldehydes with X═N and R³ as defined for the compounds of general formula (I) in the first step commercially available ethyl 2-ethoxy-2-iminoacetate (XVII) reacts with acylhydrazides of the gerenal formula (X) in which R³ is as defined for the compounds of general formula (I) to yield the ester (XI) which in the subsequent step is reduced using e.g. lithiumaluminiumhydride (according to US2018/141954) or other conditions known by the person skilled in the art to give alcohols of formula (XII) with X═N. The used acylhydrazides (X) are commercially available or can be easily prepared using the corresponding acid or ester via known procedures for person skilled in the art.

For aldehydes (III) with X═CR⁴ and R⁴ as defined for the compounds of general formula (I) known protected hydroxyacetaldehydes (XIII) react with 1,2-diketones (XIV) (for preparation see Landais, Y.; Vincent, J. M., Science of Synthesis, (2005) 26, 647) in the presence of ammonium acetate in appropriate solvent such as butanol according to Journal of Medicinal Chemistry, 2012, 2894 to yield the protected alcohols (XV) which in the subsequent step is deprotected unsing conditions known by the person skilled in the art to give alcohols of formula (XVI) with X═CR⁴.

These aldehydes can be also prepared starting with 1,2-diamino compounds (XVII) by the reaction with known protected glycolic acid derivatives of formula (XVIII) using 1-ethyl-3-(3-dimethylaminopropyl)carbodiimide hydrochloride and hydroxybenzotriazole mono hydrate followed by acetic acid analogous to Bioorganic and Medicinal Chemistry Letters, 2013, 4374 to yield the protected aldehyde (XV) which in the subsequent step are deprotected using conditions known by the person skilled in the art to give alcohols of formula (XVI) with X═CR⁴.

Alternatively, the 1,2-diamino compounds (XVII) can react with glycolic acid (XIX) using hydroxyapatite according to CN105198819 (2017) to give alcohols of formula (XVI) with X═CR⁴.

The alcohols of the formula (XII) or (XVI) can then oxidized using manganese dioxide or Swern oxidation or hypervalent iodide reagents to the aldehydes of formula (III) well known by the person skilled in the art.

A direct approach to aldehydes of formula (III) is the reaction of diamines (XVII) with dichloroacetic acid (XIX) under acidic conditions such as hydrochloric acid according to WO2006/62465 to give aldehydes of formula (III).

In a twostep sequence first diamines derivatives of formula (XVII) react with commercially available dialkoxyacetic acid ester such as ethyl diethoxyacetate to the protected aldehydes of formula (XX, alkyl=methyl or ethyl) which in the following deprotection step under acidic conditions well known by the person skilled in the art gives the aldehydes of formula (III).

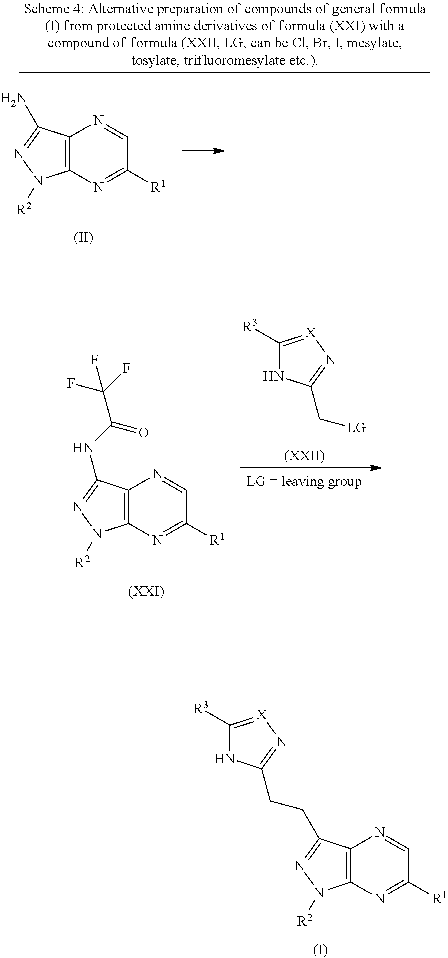

Via an alternative approach compounds of general formula (I) can be assembled from trifluoroacetamide derivatives of formula (XXI), in which R¹ and R² are as defined for the compounds of general formula (I), and a compound of formula (III), in which R² and X are as defined for the compounds of general formula (I) and LG is a leaving group such as Cl, Br, I, mesylate, tosylate, trifluoromesylate etc., by means of an alkylation well known to the person skilled in the art followed by deprotection/saponification, according to Scheme 1. Said alkylation reaction can be performed by reaction of compounds of the formulae (XXI) and (XXII) in the presence of a suitable base, such as sodium hydride, lithium diisopropylamide and similar bases in an appropriate solvent such as DMF.

An intermediately protection of NH-groups in compounds of formula (XXII) could be necessary and in these cases protecting groups such as tetrahydropyranyl, benzyl, para-methoxy-benzyl, 2-(trimethylsilyl)ethoxymethyl or others well known by the person skilled in the art are used and after the alkylation step could be deprotected.

Compounds of the formula (XXII) can be easily prepared using the prepared alcohols (XII and XVI, see scheme 3) by using methodologies well known by the person skilled in the art, such as the reaction of the alcohol with tosyl chloride, mesyl chloride or mesyl anhydride in the presence of a base, or CBr₄/PPh₃ or similar types of such a transformation.

The used trifluoroacetamide derivatives of formula (XXI) can be easily prepared by the reaction of amines derivatives of formula (II) with trifluoracetic acid anhydride in the presence of a base such as triethylamine, diisopropylethylamine etc. in an inert solvent such as DMF.

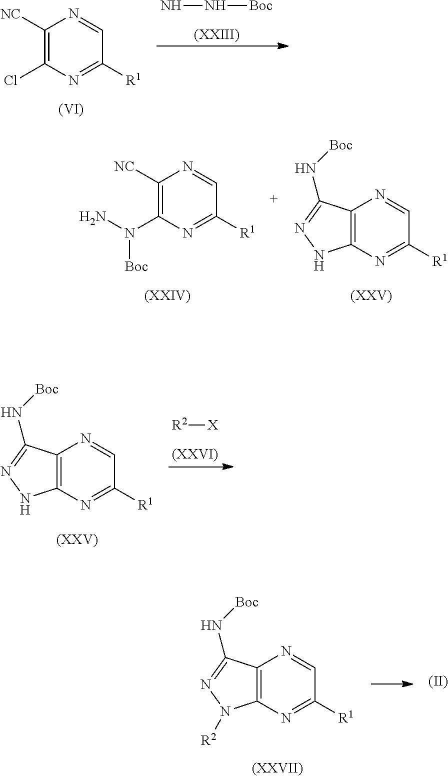

In addition to the synthesis of intermediate amine derivatives of formula (II) depicted in scheme 2 an alternative approach is shown in scheme 5. This approach started with cyanide derivatives of formula (VI) in which R¹ is as defined for the compounds of general formula (I). Reaction of compounds (VI) with mono-Boc-protected hydrazine (XXIII) using strong bases such as sodium hydride, lithium diisopropylamide etc. in an inert solvent such as N,N-dimethylformamide yielded compounds (XXIV) together with a small amount of the cyclized compounds of formula (XXV) including a shift of the protecting group. Compounds of the formula (XXIV) or a mixture of (XXIV) and (XXV) can be completely transformed into compounds such as (XXV) using again a strong base such as sodium hydride or lithium diisopropylamide in N,N-dimethylformamide. The compounds (XXV) can then react with reagents of formula (XXVI), in which R² is as defined for the compounds of general formula (I) and X is a typical leaving group such as chloride, bromide, iodide or sulfonates known to the person skilled in the art using one or better two equivalents of a base such as sodium hydride or lithium diisopropylamide in an inert solvent such as tetrahydrofuran yielding alkylated compounds of formula (XXVII). These compounds can be easily deprotected using acidic conditions well known to the person skilled in the art to get compounds of formula (II).

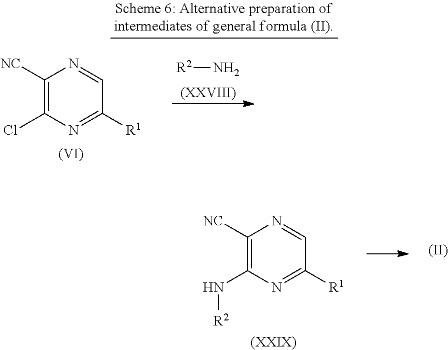

Another alternative synthesis of intermediate amine derivatives of formula (II) is depicted in scheme 6. In this approach compounds of formula (VI) reacts with an amine of formula (XXVIII) in which R² is as defined for the compounds of general formula (I) under basic condition using e.g. potassium carbonate in an inert solvent such as N,N-dimethylformamide at higher temperature for example 100° C. to the compounds of formula (XXIX). These amines (XXIX) then reacts under basic conditions using e.g. lithium diisopropylamide or lithium bis(trimethylsilyl)amide with an activated hydroxylamine derivative such as O-(diphenylphosphinoyl)hydroxylamine or corresponding sulfonates known to the person skilled in the art in an inert solvent such as tetrahydrofuran to produce an intermediately formed hydrazine which cyclized directly to compounds of formula (II).

### Methods and Administration

Compounds of general formula (I) of the present invention demonstrate a valuable pharmacological spectrum of action and pharmacokinetic profile, both of which could not have been predicted. Compounds of the present invention have surprisingly been found to effectively impair the activity of CDK12, showing a strong CDK12 degrading potency which induce the proteolytic degradation of CDK12 protein in the cell resulting in an increased selectivity against other kinases.Therefore, it is possible that said compounds can be used for the treatment and/or prophylaxis of diseases, preferably hyperproliferative disorders in humans and animals.

Further, CDK12 has been identified as a druggable target for addressing the RNA-based disease myotonic dystrophy type 1 (DM1) (Ketley et al., Sci. Transl. Med. 12, eaaz2415 (2020)). Thus, it is possible that compounds of general formula (I) of the present invention can be used for the treatment and/or prophylaxis of diseases in which CDK12 is involved, such as myotonic dystrophy type 1 (DM1).

As used herein, “prophylaxis” includes a use of the compound that, in a statistical sample, reduces the occurrence of the disorder or condition in the treated sample relative to an untreated control sample, or delays the onset or reduces the severity of one or more symptoms of the disorder or condition relative to the untreated control sample, when administered to prior to the onset of the disorder or condition.

Compounds of the present invention can be utilized to inhibit, block, reduce, decrease, etc., cell proliferation and/or cell division, and/or produce apoptosis, which are all types of “treatment”. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalamic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, basal cell carcinoma, squamous cell carcinoma, Kaposi's sarcoma, malignant melanoma, Merkel cell skin cancer and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to, sarcoma of the soft tissue, osteosarcoma, malignant fibrous histiocytoma, lymphosarcoma, and rhabdomyosarcoma.

Leukemias include, but are not limited to acute lymphoblastic leukemia, acute myeloid leukemia, (acute) T-cell leukemia, acute lymphoblastic leukemia, acute lymphocytic leukemia (ALL) , acute monocytic leukemia (AML), acute promyelocytic leukemia (APL), bisphenotypic B myelomonocytic leukemia, chronic lymphocytic leukemia (CLL), chronic myelogenous leukemia, chronic myeloid leukemia (CML), chronic myelomonocytic leukemia (CMML), large granular lymphocytic leukemia, plasma cell leukemia and also myelodysplastic syndrome (MDS), which can develop into an acute myeloid leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Ophthalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving and/or improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemo-therapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In some embodiments, a compound of general formula (I) of the present invention is administered to a cell prior to the radiation or other induction of DNA damage in the cell. In some embodiments of the invention, a compound of general formula (I) of the present invention is administered to a cell concomitantly with the radiation or other induction of DNA damage in the cell. In yet some embodiments of the invention, a compound of general formula (I) of the present invention is administered to a cell after radiation or other induction of DNA damage in the cell has begun. In yet some embodiments of the invention, a compound of general formula (I) of the present invention is administered to a cell immediately after radiation or other induction of DNA damage in the cell has begun.

In some embodiments, the cell is in vitro. In another embodiment, the cell is in vivo.

Thus in some embodiments, the present invention includes a method of inhibiting proliferation of a cell and/or the induction of apoptosis in a cell, comprising contacting the cell with a compound of formula (I).

Another aspect of the invention is a method for treating, preventing or prophylaxing cancer (i.e. a method for the treatment, prevention or prophylaxis of cancer) in a subject (e.g., human, other mammal, such as rat, etc.) by administering an effective amount of at least one compound of general formula (I), or a pharmaceutically acceptable salt, polymorph, metabolite, hydrate, solvate or ester thereof to the subject.

In some embodiments, the subject may be administered a medicament, comprising at least one compound of general formula (I) and one or more pharmaceutically acceptable carriers, excipients and/or diluents.

Furthermore in some embodiments, the present invention includes a method of using a compound of general formula (I) for the treatment of diseases.

Particularly in some embodiments, the present invention includes a method of treating a hyperproliferative disease, more particularly cancer, comprising administering an effective amount of at least one compound of general formula (I) to a subject in need thereof.

Particularly in some embodiments, the present invention includes a method of treating a hyperproliferative disease, more particularly cancer, comprising administering an effective amount of at least one compound of general formula (I) having a ratio (IC50 CDK12 hATP)/(DC50 CDK12) which is equal or greater than 20 and/or a (DC50 CDK12) value which is equal or lower than 200 nM to a subject in need thereof.

In some embodiments, the method of treatment and/or prophylaxis of a hyperproliferative disorder in a subject may comprise administering to the subject an effective amount of a compound of general formula (I). The hyperproliferative disorder may be, for example, cancer (e.g., lung cancer, breast cancer, acute myeloid leukemia, lymphoma, glioblastoma, prostate cancer, etc.).

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, ovarian cancer, comprising administering an effective amount of at least one compound of formula (I) to a subject in need thereof.

Furthermore in some embodiments, the present invention includes a method of treating cancer, particularly multiple myeloma, ovarian carcinoma, acute monocytic leukemia, melanoma and lung cancer, comprising administering an effective amount of at least one compound of formula (I) to a subject in need thereof.

Furthermore in some embodiments, the present invention includes a method of treating cancer, particularly breast cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; ovarian cancer; and pancreas cancer, comprising administering an effective amount of at least one compound of formula (I) to a subject in need thereof. GC-DLBCL means Germinal B-cell Diffuse Large B-Cell Lymphoma and ** ABC-DLBCL means Activated B-cell Diffuse Large B-Cell Lymphoma.

Furthermore in some embodiments, the present invention includes a method of treating cancer, particularly breast cancer, lung cancer, diffuse large B-cell lymphoma subtype including GC-DLBCL* and ABC-DLBCL** subtypes, mantle cell lymphoma, acute monocytic leukemia, melanoma, ovarian cancer, and pancreas cancer comprising administering an effective amount of at least one compound of formula (I) to a subject in need thereof. Furthermore in some embodiments, the present invention provides a compound of formula (I) for use of treating diseases.

Furthermore in some embodiments, the present invention includes a method of treating cancer, particularly breast cancer; lymphoma, leukemia, multiple myeloma; and ovarian cancer, comprising administering an effective amount of at least one compound of formula (I) to a subject in need thereof.

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, and ovarian cancer, comprising administering an effective amount of at least one compound of formula (I) to a subject in need thereof.

Furthermore in some embodiments, the present invention includes a method of treating cancer, particularly breast cancer, lymphoma (including non-Hodgkin-lymphoma type, diffuse large B-cell lymphoma subtype, mantle cell lymphoma), leukemia (including acute monocytic leukemia), liver cancer, multiple myeloma, melanoma, non-small cell lung cancer, small cell lung cancer, ovarian cancer, ovarian carcinoma, stomach cancer, and squamous cell carcinoma, comprising administering an effective amount of at least one compound of formula (I) to a subject in need thereof.

Furthermore in some embodiments, the present invention includes a method of treating cancer, particularly breast cancer, diffuse large B-cell lymphoma subtype, mantle cell lymphoma, acute monocytic leukemia, liver cancer, multiple myeloma, melanoma, non-small cell lung cancer, small cell lung cancer, ovarian cancer, ovarian carcinoma, prostate cancer, stomach cancer, and squamous cell carcinoma, comprising administering an effective amount of at least one compound of formula (I) to a subject in need thereof.

Furthermore in some embodiments, the present invention includes a method of treating cancer, particularly bladder cancer, bone cancer, brain cancer, breast cancer, colon cancer (colorectal cancer), endometrial (uterine) cancer, gastric cancer, head and neck cancer, kidney cancer, leukemia, liver cancer, lung cancer, lymphoma, lung cancer, myeloma, neuroblastoma, ovarian cancer, pancreatic cancer, rhabdoid tumor, sarcoma and skin cancer, comprising administering an effective amount of at least one compound of formula (I) to a subject in need thereof.

Furthermore in some embodiments, the present invention includes a method of treating cancer, particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma and acute myeloid leukemia comprising administering an effective amount of at least one compound of formula (I) to a subject in need thereof.

Furthermore in some embodiments, the present invention includes a method of treating cancer, particularly lung cancer, breast cancer, liver cancer, colorectal cancer, gastric cancer, prostate cancer and leukemia comprising administering an effective amount of at least one compound of formula (I) to a subject in need thereof.

Furthermore in some embodiments, the present invention includes a method of treating myotonic dystrophy type 1 (DM1) comprising administering an effective amount of at least one compound of general formula (I) to a subject in need thereof.

In accordance with some embodiments, the present invention provides compounds of general formula (I), as described supra, or stereoisomers, tautomers, N-oxides, hydrates, solvates, and salts thereof, particularly pharmaceutically acceptable salts thereof, or mixtures of same, for use in the treatment and/or prophylaxis of diseases, in particular hyperproliferative disorders.

In accordance with some embodiments, the present invention provides compounds of general formula (I) having a ratio (IC50 CDK12 hATP)/(DC50 CDK12) which is equal or greater than 20 and/or a (DC50 CDK12) value which is equal or lower than 200 nM, as described supra, or stereoisomers, tautomers, N-oxides, hydrates, solvates, and salts thereof, particularly pharmaceutically acceptable salts thereof, or mixtures of same, for use in the treatment and/or prophylaxis of diseases, in particular hyperproliferative disorders.

Furthermore in accordance with a further aspect, the present invention provides a compound of formula (I) for use of treating diseases.Furthermore in accordance with a further aspect, the present invention provides a compound of formula (I) having a ratio (IC50 CDK12 hATP)/(DC50 CDK12) which is equal or greater than 20 and/or a (DC50 CDK12) value which is equal or lower than 200 nM for use of treating diseases.

In in accordance with a further aspect, the present invention includes a compound of general formula (I) for use in a method of inhibiting proliferation of a cell and/or the induction of apoptosis in a cell, comprising contacting the cell with a compound of formula (I).

Particularly in some embodiments, the present invention includes compounds of general formula (I) for use in a method of treating a hyperproliferative disease, more particularly wherein the hyperproliferative disease is cancer, and yet even more particularly wherein the cancer disease is selected from lymphoma, non-Hodgkin-lymphoma type, diffuse large B-cell lymphoma subtype, ovarian cancer, multiple myeloma, acute leukemia, and acute myeloid leukemia.

More particularly in some embodiments, the present invention includes compounds of general formula (I) for use in a method of treating a hyperproliferative disease, more particularly wherein the hyperproliferative disease is cancer, and yet even more particularly wherein the cancer disease is selected from breast cancer; lymphoma, leukemia, multiple myeloma; and ovarian cancer.

Particularly in some embodiments, the present invention includes compounds of general formula (I) for use in a method of treating a hyperproliferative disease, more particularly wherein the hyperproliferative disease is cancer, and yet even more particularly wherein the cancer is selected from breast cancer; esophageal cancer; liver cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; melanoma; ovarian cancer; or pancreas cancer.

More particularly in some embodiments, the present invention includes compounds of general formula (I) for use in a method of treating cancer wherein the cancer disease is selected from breast cancer; lymphoma, leukemia, multiple myeloma; and ovarian cancer.

More particularly in some embodiments, the present invention includes compounds of general formula (I) for use in a method of treating cancer wherein the cancer disease is selected from breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

More particularly in some embodiments, the present invention includes compounds of general formula (I) for use in a method of treating cancer wherein the cancer disease is selected from lung cancer, breast cancer, liver cancer, colorectal cancer, gastric cancer, prostate cancer, and leukemia.

Furthermore in some embodiments, the present invention includes compounds of general formula (I) for use in a method of treating myotonic dystrophy type 1 (DM1).

In some embodiments, the present invention includes the use of the compounds of general formula (I) for the manufacture of a medicament for the treatment and/or prophylaxis of a hyperproliferative disease.

In some embodiments, the present invention includes the use of the compounds of general formula (I) for the manufacture of a medicament for the treatment and/or prophylaxis of a hyperproliferative disease, wherein the hyperproliferative disease is cancer.

In some embodiments, the present invention includes the use of the compounds of general formula (I) having a ratio (IC50 CDK12 hATP)/(DC50 CDK12) which is equal or greater than 20 and/or a (DC50 CDK12) value which is equal or lower than 200 nM for the manufacture of a medicament for the treatment and/or prophylaxis of a hyperproliferative disease.

In some embodiments, the present invention includes the use of the compounds of general formula (I) having a ratio (IC50 CDK12 hATP)/(DC50 CDK12) which is equal or greater than 20 and/or a (DC50 CDK12) value which is equal or lower than 200 nM for the manufacture of a medicament for the treatment and/or prophylaxis of a hyperproliferative disease, wherein the hyperproliferative disease is cancer.

In some embodiments, the present invention includes the use of the compounds of general formula (I) for the manufacture of a medicament for the treatment of a hyperproliferative disease, particularly cancer and more particularly lymphoma, non-Hodgkin-lymphoma type, diffuse large B-cell lymphoma subtype, ovarian cancer, multiple myeloma, acute leukemia, and acute myeloid leukemia type.

In some embodiments, the present invention includes the use of the compounds of general formula (I) for the manufacture of a medicament for the treatment of a hyperproliferative disease, particularly cancer and more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

In some embodiments, the present invention includes the use of the compounds of general formula (I) for the manufacture of a medicament for the treatment of a hyperproliferative disease, particularly cancer and more particularly lung cancer, breast cancer, liver cancer, colorectal cancer, gastric cancer, prostate cancer, and leukemia.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

Furthermore in some embodiments, the present invention includes the use of the compounds of general formula (I) for the manufacture of a medicament for the treatment of myotonic dystrophy type 1 (DM 1).

In some embodiments, the present invention provides a method of treatment and/or prophylaxis of diseases, in particular hyperproliferative disorders, particularly cancer, comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment and/or prophylaxis of diseases, in particular hyperproliferative disorders, particularly cancer, more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

Furthermore in some embodiments, the present invention provides a method of treatment of myotonic dystrophy type 1 (DM1) comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore provides pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat), solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine), disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In some embodiments, the present invention provides pharmaceutical combinations, in particular medicaments, comprising at least one compound of general formula (I) of the present invention and at least one or more further active ingredients, in particular for the treatment and/or prophylaxis of a hyperproliferative disorder, particularly cancer.

Particularly, the present invention provides a pharmaceutical combination, which comprises:
one or more first active ingredients, in particular compounds of general formula (I) as defined supra, and
one or more further active ingredients, in particular for the treatment and/or prophylaxis of a hyperproliferative disorder, particularly cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also provides such pharmaceutical combinations. For example, the compounds of the present invention can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin 11, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (1231), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib , regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib , valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid and zorubicin.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

### Experimental Section—NMR Spectra

To the extent NMR peak forms and multiplicities are specified, they are stated as they appear in the spectra, possible higher order effects have not been considered.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight” <1%.

### Experimental Section—Abbreviations

The following table lists the abbreviations used in this paragraph and in the Intermediates and Examples section as far as they are not explained within the text body. Other abbreviations have their meanings customary per se to the skilled person. A comprehensive list of the abbreviations utilized by organic chemists of ordinary skill in the art appears presented in the first issue of each volume of the Journal of Organic Chemistry; this list is typically presented in a table titled “Standard List of Abbreviations”. In case of doubt, the abbreviations and/or their meaning according to the following table shall prevail.

TABLE 1
Abbreviations

| Abbreviation | Meaning |
|---|---|
| DMF | N,N-Dimethylformamide |
| DMSO | dimethylsulfoxide |
| ESI | electrospray (ES) ionisation |
| h, hr (hrs) | hour(s) |
| HPLC | high performance liquid chromatography |
| LC-MS | liquid chromatography-mass spectrometry |
| m | multiplet (NMR) |
| Min | minute(s) |
| MS | mass spectrometry |
| NMR | nuclear magnetic resonance spectroscopy: chemical shifts |
| | (δ) are given in ppm. The chemical shifts were corrected |
| | by setting the DMSO signal to 2.50 ppm using dmso-d6 |
| | unless otherwise stated. |
| rt, RT | room temperature |
| Rt, Rt | retention time |
| THF | tetrahydrofuran |
| UPLC | ultra performance liquid chromatography |
| UV | ultraviolet |
| δ | chemical shift |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art. Reactions were set up and started, e.g. by the addition of reagents, at temperatures as specified in the protocols; if no temperature is specified, the respective working step was performed at ambient temperature, i.e. between 18 and 25° C.

“Silicone filter” or “water resistant filter” refers to filter papers which are made hydrophobic (impermeable to water) by impregnation with a silicone. With the aid of these filters, water can be separated from water-immiscible organic solvents by means of a filtration (i.e. filter paper type MN 617 WA, Macherey-Nagel).

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent or solvent mixture. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/ethanol. In flash column chromatography, unmodified (“regular”) silica gel may be used as well as aminophase functionalized silica gel. As used herein, “Biotage SNAP cartridge silica” refers to the use of regular silica gel; “Biotage SNAP cartridge NH₂ silica” refers to the use of aminophase functionalized silica gel. If reference is made to flash column chromatography or to flash chromatography in the experimental section without specification of a stationary phase, regular silica gel was used.

In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid, diethylamine or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

UPLC-MS Standard Procedures

Analytical UPLC-MS was performed as described below. The masses (m/z) are reported from the positive mode electrospray ionisation unless the negative mode is indicated (ESI−).

Analytical UPLC Methods:

Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 3:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 4:

Instrument: SHIMADZU LCMS-2020 SingleQuad; Column: Chromolith©Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 mL/min; temperature: 50° C.; PDA: 220 nm & 254 nm.

Method 5:

Instrument: Agilent 1100\G1956A SingleQuad; Column: Kinetex@ 5 pm EVO C18 30*2.1 mm; eluent A: water+0.0375 vol % trifluoroacetic acid, eluent B: acetonitrile+0.01875 vol % trifluoroacetic acid; gradient: 0-0.8 min 5-95% B, 0.8-1.2 min 95% B; flow 1.5 mL/min; temperature: 50° C.; PDA: 220 nm & 254 nm.

Preparative HPLC Methods:

Method HT Acidic:

Instrument: Waters Autopurificationsystem; Column: Waters XBrigde C18 5 μ 100×30 mm; eluent A: water+0.1 vol % formic acid (99%), eluent B: acetonitrile; gradient; DAD scan: 210-400 nm.

Method HT Basic:

Instrument: Waters Autopurificationsystem; Colum: Waters XBrigde C18 5 μ 100×30 mm; eluent A: water+0.2 vol % aqueous ammonia (32%), eluent B: acetonitrile; gradient; DAD scan: 210-400 nm.

Specific Optial Rotation Methods:

Method O1: Instrument: JASCO P2000 Polarimeter; wavelength 589 nm; temperature: 20° C.; integration time 10 s; path length 100 mm.

### Intermediate 1

### 3-chloro-5-cyclopropylpyrazine-2-carbonitrile

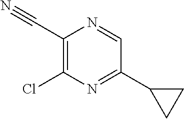 [SMILES: N#Cc1ncc(C2CC2)nc1Cl]

A mixture of 3,5-dichloropyrazine-2-carbonitrile (5.00 g, 28.7 mmol), cyclopropylboronic acid (2.47 g, 28.7 mmol), [1,1′-bis (diphenylphosphino)ferrocene]dichloropalladium(II) (2.10 g, 2.87 mmol), potassium carbonate (7.94 g, 57.5 mmol) in a 5.0 mL water and 50 mL dioxane was stirred over night at 80° C. After cooling the mixture was filtered, the filtercake was washed with ethyl acetate and then the combined organic phase was evaporated to dryness. The residue was purified via Biotage chromatography system (100 g snap KP-Sil column, hexane/0-45% ethyl acetate) yielding 2.2 g (38%, including about 15% of 3,5-dicyclopropylpyrazine-2-carbonitrile) of the desired title compound.

LC-MS (Method 1): Rₜ=1.12 min; MS (ESIpos): m/z=180 [M+H]⁺

¹H-NMR (400 MHz, DMSO-d6) δ [ppm]=1.05-1.11 (m, 2H), 1.25-1.32 (m, 2H), 2.34-2.42 (m, 1H), 8.84 (s, 1H).

### Intermediate 2

### 6-cyclopropyl-1-(1-methylpiperidin-4-yl)-1H-pyrazolo[3,4-b]pyrazin-3-amine

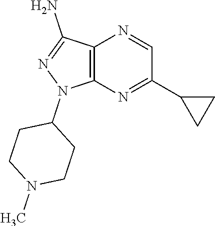 [SMILES: CN1CCC(n2nc(N)c3ncc(C4CC4)nc32)CC1]

To a solution of 3-chloro-5-cyclopropylpyrazine-2-carbonitrile (2.10 g, 11.7 mmol, intermediate 1) in 28 mL 1-butanol was added potassium carbonate (6.46 g, 46.8 mmol) followed by 4-hydrazinyl-1-methylpiperidine bis hydrogen chloride salt (3.54 g, 17.5 mmol, CAS-RN:[53242-78-7], commercially available e.g. Enamine Ltd. or Apollo). The mixture was stirred for 4 hours at 120° C. and over night at room temperature. The reaction mixture was diluted with 150 mL ethyl acetate, extracted with water twice, brine, dried using a hydrophobic filter and evaporated to dryness. The residue was adsored on Isolute® and this absorbed material was then purified by two subsequent column chromatographies using a Biotage chromatography system (1.110 g snap NH column, hexane/0-100% ethyl acetate, 2.55 g snap NH column, hexane/0-100% ethyl acetate). Using this methodology we got the desired 6-cyclopropyl-1-(1-methylpiperidin-4-yl)-1H-pyrazolo[3,4-b]pyrazin-3-amine. Yield: 653 mg (18% yield)

LC-MS (Method 2): Rₜ=0.96 min; MS (ESIpos): m/z=274 [M+H]⁺

¹H-NMR (400 MHz, DMSO-d6) δ [ppm]=1.02-1.05 (m, 2H), 1.06-1.11 (m, 2H), 1.70-1.78 (m, 2H), 1.99-2.17 (m, 4H), 2.20 (s, 3H), 2.26-2.33 (m, 1H), 2.81-2.90 (m, 2H), 4.34 (tt, 1H), 5.73 (s, 2H), 8.31 (s, 1H).

### Intermediate 3

### tert-butyl 4-(3-amino-6-cyclopropyl-1H-pyrazolo[3,4-b]pyrazin-1-yl)piperidine-1-carboxylate

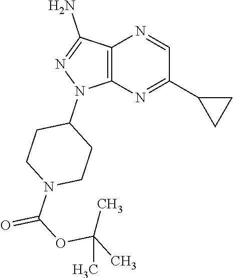 [SMILES: CC(C)(C)OC(=O)N1CCC(n2nc(N)c3ncc(C4CC4)nc32)CC1]

In analogy to intermediate 2 3-chloro-5-cyclopropylpyrazine-2-carbonitrile (1.00 g, 5.57 mmol) and tert-butyl 4-hydrazinopiperidine-1-carboxylate hydrochloride salt (2.10 g, 8.35 mmol, CAS-RN:[1258001-18-1], commercially available, e.g. ABCR) reacted to the desired titled compound. Yield: 900 mg (38% yield)

LC-MS (Method 2): Rₜ=1.23 min; MS (ESIpos): m/z=359 [M+H]⁺

### Intermediate 4

### (±)-6-cyclopropyl-1-[(3)-oxolan-3-yl]-1H-pyrazolo[3,4-b]pyrazin-3-amine

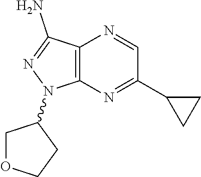 [SMILES: Nc1nn(C2CCOC2)c2nc(C3CC3)cnc12]

In analogy to intermediate 2 3-chloro-5-cyclopropylpyrazine-2-carbonitrile (300 mg, 1.67 mmol) and (±)-[oxolan-3-yl]hydrazine hydrogen chloride salt (347 mg, 2.51 mmol, CAS-RN:[158001-24-2], commercially available e.g. Enamine Ltd.) reacted to the desired titled compound. Yield: 190 mg (42% yield)

LC-MS (Method 1): Rₜ=0.88 min; MS (ESIpos): m/z=246 [M+H]⁺

¹H-NMR (400 MHz, DMSO-d6) δ [ppm]=1.02-1.12 (m, 4H), 2.20-2.38 (m, 3H), 3.77-3.88 (m, 2H), 3.92-4.05 (m, 2H), 5.18-5.30 (m, 1H), 5.82 (s, 2H), 8.34 (s, 1H).

### Intermediate 5

### 4-fluoro-1H-benzimidazole-2-carbaldehyde—hydrogen chloride (1/1)

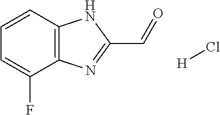

A mixture of 3-fluorobenzene-1,2-diamine (300 mg, 2.38 mmol) and dichloroacetic acid (390 μL, 4.8 mmol) in 7.9 mL 4N HCl was stirred for 16 hours at 100° C. The reaction mixture was filtered and the filter cake was washed with water. The filtrate was extracted three times with chloroform and the aqueous phase was evaporated to dryness to obtained 435 mg of the titled compound as raw material which was used in the next step without further purification.

### Intermediate 6

### 5-fluoro-1H-benzimidazole-2-carbaldehyde

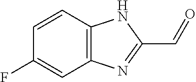 [SMILES: O=Cc1nc2cc(F)ccc2[nH]1]

In analogy to intermediate 5 4-fluorobenzene-1,2-diamine (300 mg, 2.38 mmol) and dichloroacetic acid (390 μL, 4.8 mmol) reacted to the desired titled compound (380 mg) as raw material which was used without any further purification in the next step.

### Intermediate 7

### 4-(trifluoromethoxy)-1H-benzimidazole-2-carbaldehyde

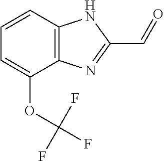 [SMILES: O=Cc1nc2c(OC(F)(F)F)cccc2[nH]1]

To a solution of 3-(trifluoromethoxy)benzene-1,2-diamine (150 mg, 781 μmol) in 1.2 mL EtOH was added ethyl diethoxyacetate (170 μL, 940 μmol) and sodium ethanolate (58.4 mg, 859 μmol). This reaction mixture was heated under reflux for 16 hours. Then an addtional portion of ethyl diethoxyacetate (170 μL, 940 μmol) and sodium ethanolate (58.4 mg, 859 μmol) was added and the heating was continued for 5 hours. After cooling to room temperature ethanol was concentrated in vacuum and then water was added. This mixture was extracted threetimes with methylene chloride, the combined organic phases were filtered using a hydrophobic filter and evaporated to dryness. The residue was purified via Biotage chromatography system (snap NH column, hexane/10-100 ethyl acetate) yielding 151 mg (44% yield) 2-(diethoxymethyl)-4-(trifluoromethoxy)-1H-benzimidazole.

LC-MS (Method 2): Rₜ=1.15 min; MS (ESIpos): m/z=306 [M+H]⁺

151 mg 2-(diethoxymethyl)-4-(trifluoromethoxy)-1H-benzimidazole was stirred in 372 μL 4N HCl for three hours at 60° C. The reaction was cooled to room temperature an then evaporated to dryness. The residue was the desired titled compound (150 mg) as raw material which was used without any further purification in the next step.

### Intermediate 8

### 5-methyl-1H-benzimidazole-2-carbaldehyde

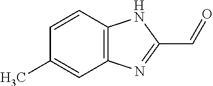

In analogy to intermediate 7: 4-methylbenzene-1,2-diamine (150 mg, 1.22 mmol) and ethyl diethoxyacetate (263 μL, 1.47 mmol) reacted in a two step procedure to obtain the desired titled compound (200 mg) as raw material which was used without any further purification in the next step.

### Intermediate 9

### 4-(trifluoromethyl)-1H-benzimidazole-2-carbaldehyde

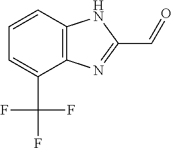 [SMILES: O=Cc1nc2c(C(F)(F)F)cccc2[nH]1]

In analogy to intermediate 5: 3-(trifluoromethyl)benzene-1,2-diamine (300 mg, 1.70 mmol) and dichloroacetic acid (280 μL, 3.4 mmol) reacted to the desired titled compound (113 mg) as raw material which was used without any further purification in the next step.

### Intermediate 10

### 4-methyl-1H-benzimidazole-2-carbaldehyde

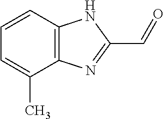

In analogy to intermediate 5: 3-methylbenzene-1,2-diamine (300 mg, 2.46 mmol) and dichloroacetic acid (400 μL, 4.9 mmol) reacted to the desired titled compound (885 mg) as raw material which was used without any further purification in the next step.

### Intermediate 11

### 4,5-difluoro-1H-benzimidazole-2-carbaldehyde

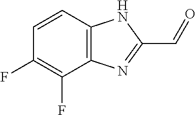 [SMILES: O=Cc1nc2c(F)c(F)ccc2[nH]1]

In analogy to intermediate 5: 3-methylbenzene-1,2-diamine (300 mg, 2.46 mmol) and dichloroacetic acid (400 μL, 4.9 mmol) reacted to the desired titled compound (606 mg) as raw material which was used without any further purification in the next step.

### Intermediate 12

### 6-cyclopropyl-1-phenyl-2H-pyrazolo[3,4-b]pyrazin-3-amine

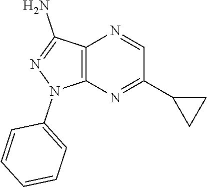 [SMILES: Nc1nn(-c2ccccc2)c2nc(C3CC3)cnc12]

To a solution of 3-chloro-5-cyclopropylpyrazine-2-carbonitrile (1.00 g, 5.57 mmol, intermediate 1) in 1-butanol (10 mL, 110 mmol) were added phenylhydrazine (903 mg, 8.35 mmol) and potassium carbonate (2.31 g, 16.7 mmol) at room temperature. The reaction mixture was heated to 120° C. and stirred at 120° C. for 1.5 hours. The reaction mixture was cooled to room temperature and evaporated under reduced pressure to give a residue. The residue was dissolved in ethyl acetate and washed with water. The organic layer was evaporated under reduced pressure to give a residue. The residue was purified by flash column (petroleum ether:ethyl acetate=1:0 then 1:1) to give 6-cyclopropyl-2-phenyl-2H-pyrazolo[3,4-b]pyrazin-3-amine as a brown solid and \hat{ }the desired titled compound (120 mg, 85% purity, 7% yield) as a yellow solid.

LC-MS (Method 5): Rₜ=0.626 min; MS (ESI pos): m/z=252.2 [M+H]⁺.

### Intermediate 13

### 6-cyclopropyl-1-(propan-2-yl)-1H-pyrazolo[3,4-b]pyrazin-3-amine

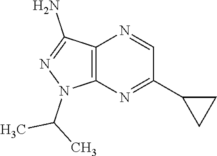 [SMILES: CC(C)n1nc(N)c2ncc(C3CC3)nc21]

To a solution of 3-chloro-5-cyclopropylpyrazine-2-carbonitrile (200 mg, 93% purity, 1.04 mmol, intermediate 1) in 1-butanol (2.0 mL, 22 mmol) were added isopropylhydrazine (115 mg, 1.55 mmol) and potassium carbonate (429 mg, 3.11 mmol) at room temperature. The reaction mixture was heated to 120° C. and stirred at 120° C. for 1.5 hours. After cooling the solution to room temperature, water was added to the reaction mixture. The solution was extracted with ethyl acetate. The organic layer was evaporated under reduced pressure to give a residue. The residue was purified by preparative TLC (petroleum ether: ethyl acetate =1: 1) to give 6-cyclopropyl-1-isopropyl-1H-pyrazolo[3,4-b]pyrazin-3-amine (70 mg, 99% purity, 31% yield) as a yellow solid.

LC-MS (Method 5): Rₜ=0.761 min; MS (ESIpos): m/z=281.2 [M+H]⁺.

¹H-NMR (400 MHz, DMSO-d6): δ [ppm]=1.15-1.01 (m, 4H), 1.39-1.31 (m, 6H), 2.38-2.23 (m, 1H), 4.85-4.70 (m, 1H), 5.71 (s, 2H), 8.31 (s, 1H).

### Intermediate 14

### 6-cyclopropyl-1-methyl-1H-pyrazolo[3,4-b]pyrazin-3-amine

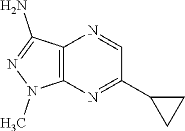

To a solution of 3-chloro-5-cyclopropylpyrazine-2-carbonitrile (200 mg, 93% purity, 1.04 mmol, intermediate 1) in 1-butanol (2.0 mL) were added methylhydrazine (40% in water) (0.210 mL, 40% purity, 1.6 mmol) and potassium carbonate (429 mg, 3.11 mmol) at room temperature. The reaction mixture was heated to 120° C. and stirred at 120° C. for 2 hours. The reaction mixture was cooled to room temperature. Water was added to the reaction mixture, the solution was extracted with ethyl acetate. The organic layer was evaporated under reduced pressure to give a residue. The residue was purified by preparative-TLC (petroleum ether:ethyl acetate=1:1) to give the desired titled compound (160 mg, 82% yield) as a yellow solid.

LC-MS (Method 4): Rₜ=0.734 min; MS (ESIpos): m/z=375.2 [M+H]⁺.

¹H-NMR (400 MHz, DMSO-d6): δ [ppm]=1.18-0.99 (m, 4H), 2.33-2.21 (m, 1H), 3.69 (s, 3H), 5.69 (s, 2H), 8.32 (s, 1H).

### Intermediate 15

### tert-butyl 2-(oxan-4-ylidene)hydrazine-1-carboxylate

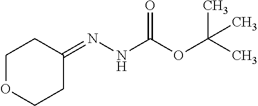

A mixture of dihydro-2H-pyran-4(3H)-one (25.0 g, 250 mmol) and tert-butyl hydrazinecarboxylate (39.6 g, 300 mmol) in methanol (500 mL) was stirred at 20° C. for 16 hours. The reaction mixture was evaporated in vacuum to give the desired titled compound (50.0 g, 93% yield) as a yellow solid. The crude product was used directly to next step without further purification.

### Intermediate 16

### tert-butyl 2-(oxan-4-yl)hydrazine-1-carboxylate

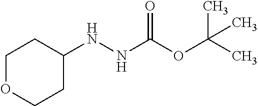

A mixture of tert-butyl 2-(oxan-4-ylidene)hydrazine-1-carboxylate (50.0 g, 233 mmol, intermediate 15) in the mixed solvent of water (450 mL) and acetic acid (150 mL) was stirred at 20° C. for 1 hour. Sodium cyanoborohydride (17.6 g, 280 mmol) was added dropwise to the reaction mixture above at 0° C. The mixture was stirred at 20° C. for 16 hours. The solution was neutralized with sodium hydroxide (3 M) to pH-7 and extracted with dichloromethane. The combined organic layers were washed with brine, dried over anhydrous sodium sulfate and filtered. The filtrate was evaporated in vacuum to give a residue. The residue was purified by silica gel chromatography (200-300 mesh, petroleum ether:ethyl acetate=10:1 to 1:1) to afford the desired titled compound (40.0 g, 79% yield) as white solid.

¹H NMR (400 MHz, CDCl₃): δ [ppm]=1.44-1.36 (m, 2H), 1.46 (s, 9H), 1.80-1.73 (m, 2H), 3.12-3.02 (m, 1H), 3.44-3.35 (m, 2H), 4.00-3.92 (m, 2H), 6.14 (br.s, 1H).

### Intermediate 17

### (oxan-4-yl)hydrazine-hydrogen chloride (1/1)

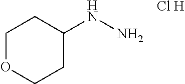

A mixture of tert-butyl 2-(oxan-4-yl)hydrazine-1-carboxylate (28.0 g, 129 mmol, intermediate 16) and hydrochloric acid/dioxane (300 mL, 4.0 M, 1.2 mol) was stirred 20° C. for 0.5 hour. The resulting suspension was filtered and the filter cake was washed with ethyl acetate, dried in vacuum to give the desired titled compound (23.0 g, 90% purity) as a white solid.

¹H NMR (400 MHz, DMSO-d₆): δ [ppm]=1.54-1.41 (m, 2H), 1.93-1.86 (m, 2H), 3.18-3.09 (m, 1H), 3.31-3.22 (m, 2H), 3.90-3.83 (m, 2H), 5.81 (br.s, 4H).

### Intermediate 18

### 6-cyclopropyl-1-(oxan-4-yl)-1H-pyrazolo[3,4-b]pyrazin-3-amine

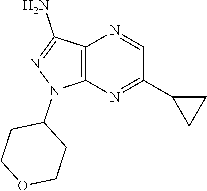 [SMILES: Nc1nn(C2CCOCC2)c2nc(C3CC3)cnc12]

To a solution of 3-chloro-5-cyclopropylpyrazine-2-carbonitrile (700 mg, 80% purity, 3.12 mmol, intermediate 1) in 1-butanol (8.0 mL) were added potassium carbonate (1.29 g, 9.35 mmol) and (oxan-4-yl)hydrazine-hydrogen chloride (1/1) (793 mg, 90% purity, 4.68 mmol, intermediate 17) at room temperature. The reaction mixture was stirred at 120° C. for 2 hours. The mixture was filtered off and the filtrate was concentrated to give a residue. The residue was purified by column chromatography (100-200 mesh, petroleum ether:ethyl acetate=20:1 to 1:1) to give 6-cyclopropyl-2-(oxan-4-yl)-1H-pyrazolo[3,4-b]pyrazin-3-amine as a yellow solid and the desired titled compound (210 mg, 87% purity, 23% yield) as a brown solid.

LC-MS (Method 4): Rₜ=0.708 min; MS (ESIpos): m/z=260.1 [M+H]⁺.

¹H NMR (400 MHz, DMSO-d6): δ [ppm]=1.10-1.04 (m, 4H), 1.8-1.73 (m, 2H), 2.15-2.03 (m, 2H), 2.33-2.28 (m, 1H), 3.50 (t, 2H), 3.95 (dd, 2H), 4.68-4.58 (m, 1H), 5.74 (s, 2H), 8.32 (s, 1H).

### Intermediate 19

### 5,6-dichloro-1H-benzimidazole-2-carbaldehyde

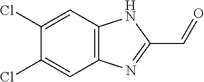 [SMILES: O=Cc1nc2cc(Cl)c(Cl)cc2[nH]1]

In analogy to intermediate 7: 4,5-dichlorobenzene-1,2-diamine (300 mg, 1.69 mmol) and ethyl diethoxyacetate (360 μL, 2.00 mmol) reacted in a two step procedure to obtain the desired titled compound (199 mg) as raw material which was used without any further purification in the next step.

### Intermediate 20

### 4-bromo-1H-benzimidazole-2-carbaldehyde

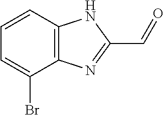 [SMILES: O=Cc1nc2c(Br)cccc2[nH]1]

In analogy to intermediate 7: 3-bromobenzene-1,2-diamine (150 mg, 802 μmol) and ethyl diethoxyacetate (170 μL, 960 μmol) reacted in a two step procedure to obtain the desired titled compound (168 mg) as raw material which was used without any further purification in the next step.

### Intermediate 21

### 5-chloro-4-methyl-1H-benzimidazole-2-carbaldehyde

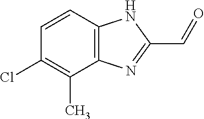

In analogy to intermediate 7: 4-chloro-3-methylbenzene-1,2-diamine (150 mg, 958 μmol) and ethyl diethoxyacetate (200 μL, 1.1 mmol) reacted in a two step procedure to obtain the desired titled compound (125 mg) as raw material which was used without any further purification in the next step.

### Intermediate 22

### 5-chloro-1H-benzimidazole-2-carbaldehyde

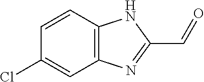 [SMILES: O=Cc1nc2cc(Cl)ccc2[nH]1]

In analogy to intermediate 7) 4-chlorobenzene-1,2-diamine (150 mg, 1.05 mmol) and ethyl diethoxyacetate (230 μL, 1.3 mmol) reacted in a two step procedure to obtain the desired titled compound (120 mg) as raw material which was used without any further purification in the next step.

### Intermediate 23

### 5-(trifluoromethyl)-1H-benzimidazole-2-carbaldehyde

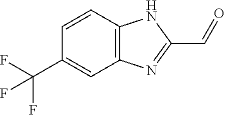 [SMILES: O=Cc1nc2cc(C(F)(F)F)ccc2[nH]1]

In analogy to intermediate 7) 4-(trifluoromethyl)benzene-1,2-diamine (150 mg, 852 μmol) and ethyl diethoxyacetate (360 μL, 2.0 mmol) reacted in a two step procedure to obtain the desired titled compound (138 mg) as raw material which was used without any further purification in the next step.

### Intermediate 24

### 5-fluoro-4-methyl-1H-benzimidazole-2-carbaldehyde

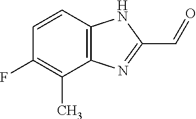

In analogy to intermediate 7) 4-fluoro-3-methylbenzene-1,2-diamine (150 mg, 1.07 mmol) and ethyl diethoxyacetate (460 μL, 2.6 mmol) reacted in a two step procedure to obtain the desired titled compound (207 mg) as raw material which was used without any further purification in the next step.

### Intermediate 25

### 5-[(trifluoromethyl)sulfanyl]-1H-benzimidazole-2-carbaldehyde

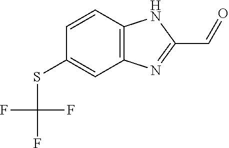 [SMILES: O=Cc1nc2cc(SC(F)(F)F)ccc2[nH]1]

In analogy to intermediate 7) 4-[(trifluoromethyl)sulfanyl]benzene-1,2-diamine (150 mg, 720 μmol) and ethyl diethoxyacetate (310 μL, 1.7 mmol) reacted in a two step procedure to obtain the desired titled compound (152 mg) as raw material which was used without any further purification in the next step.

### Intermediate 26

### 4,5-dimethyl-1H-benzim idazole-2-carbaldehyde

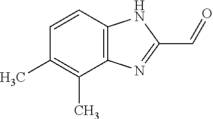

In analogy to intermediate 7) 3,4-dimethylbenzene-1,2-diamine (150 mg, 1.10 mmol) and ethyl diethoxyacetate (470 μL, 2.6 mmol) reacted in a two step procedure to obtain the desired titled compound (97 mg) as raw material which was used without any further purification in the next step.

### Intermediate 27

### 5-chloro-6-fluoro-1H-benzimidazole-2-carbaldehyde

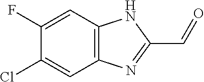 [SMILES: O=Cc1nc2cc(Cl)c(F)cc2[nH]1]

In analogy to intermediate 7) 4-chloro-5-fluorobenzene-1,2-diamine (150 mg, 934 μmol) and ethyl diethoxyacetate (400 μL, 2.2 mmol) reacted in a two step procedure to obtain the desired titled compound (111 mg) as raw material which was used without any further purification in the next step.

### Intermediate 28

### 5,6-difluoro-1H-benzimidazole-2-carbaldehyde

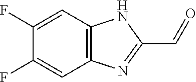 [SMILES: O=Cc1nc2cc(F)c(F)cc2[nH]1]

In analogy to intermediate 7) 4,5-difluorobenzene-1,2-diamine (450 mg, 3.12 mmol) and ethyl diethoxyacetate (1.3 mL, 7.5 mmol) reacted in a two step procedure to obtain the desired titled compound (381 mg) as raw material which was used without any further purification in the next step.

### Intermediate 29

### 4-chloro-5-fluoro-1H-benzimidazole-2-carbaldehyde

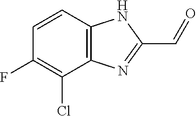 [SMILES: O=Cc1nc2c(Cl)c(F)ccc2[nH]1]

In analogy to intermediate 7) 3-chloro-4-fluorobenzene-1,2-diamine (190 mg, 1.18 mmol) and ethyl diethoxyacetate (510 μL, 2.8 mmol) reacted in a two step procedure to obtain the desired titled compound (168 mg) as raw material which was used without any further purification in the next step.

### Intermediate 30

### tert-butyl 2-(4,4-difluorocyclohexylidene)hydrazine-1-carboxylate

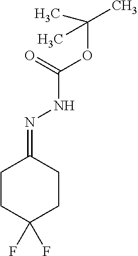 [SMILES: CC(C)(C)OC(=O)NN=C1CCC(F)(F)CC1]

To A solution of 4,4-difluorocyclohexan-1-one (3.50 g, 26.1 mmol, CAS-RN:[22515-18-0], commercially available e.g. ABCR) in 12 mL methanol a solution of tert-butyl hydrazinecarboxylate (3.28 g, 24.8 mmol) in 12 mL methanol was added dropwise. The mixture was then stirred for 1 h at room temperature and directly used in the next step.

### Intermediate 31

### tert-butyl 2-(4,4-difluorocyclohexyl)hydrazine-1-carboxylate

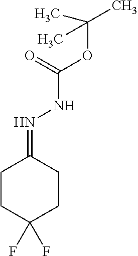 [SMILES: CC(C)(C)OC(=O)NN=C1CCC(F)(F)CC1]

In two independent reactions tert-butyl 2-(4,4-difluorocyclohexylidene)hydrazine-1-carboxylate (3.20 g/3.20g, 12.9 mmol/12.9 mmol, intermediate 30) was mixed with 26 mL water and 8.6 mL acetic acid. After the mixtures were stirred for 1 h at 0° C. sodium cyanoborohydride (972 mg/972 mg, 15.5 mmol/15.5 mmol) was added. The reactions were stirred over night at room temperature. To the mixtures an aqueous sodiumhydroxide solution (c=4 mol/L) was added till pH=7 was achieved. The mixtures were extracted with 50 mL dichloromethane for three times. The organic layers were washed with brine, filtered over a hydrophobic filter and evaporated till most of the solvent was gone. The residues of both reactions were combined to give 8.1 g (113% yield) of the desired titled compound, which was used without any further purification.

¹H-NMR (400 MHz, DMSO-d6) δ [ppm]=1.38 (s, 9H), 1.40-1.50 (m, 2H), 1.60-1.84 (m, 4H), 1.94-2.10 (m, 2H), 2.92 (br s, 1H), 4.42 (br s, 1H), 8.22 (br s, 1H).

### Intermediate 32

### (4,4-difluorocyclohexyl)hydrazine—hydrogen chloride (1/1)

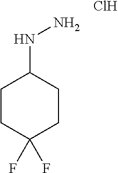

Tert-butyl 2-(4,4-difluorocyclohexyl)hydrazine-1-carboxylate (1.00 g, 4.00 mmol, intermediate 31) was suspended in 10 mL 4M hydrogen chloride in dioxane and stirred for 1 h at 20° C. The mixture was filtered and washed with dioxane. The filtercake was dried to give the desired product as raw material (1.5 g). The raw material was directly used in the next step.

¹H-NMR (400 MHz, DMSO-d6) δ [ppm]=1.51-1.63 (m, 2H), 1.77-2.11 (m, 6H), 3.07 (br t, 1H), 7.45 (br s, 4H).

### Intermediate 33

### 6-cyclopropyl-1-(4,4-difluorocyclohexyl)-1H-pyrazolo[3,4-b]pyrazin-3-amine

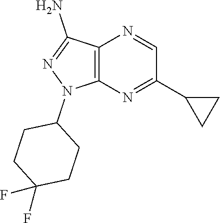 [SMILES: Nc1nn(C2CCC(F)(F)CC2)c2nc(C3CC3)cnc12]

To a solution of 3-chloro-5-cyclopropylpyrazine-2-carbonitrile (478 mg, 2.66 mmol, intermediate 1) in 7.2 mL 1-butanol were added potassium carbonate (1.47 g, 10.6 mmol) and (4,4-difluorocyclohexyl)hydrazine-hydrogen chloride (1/1) (745 mg, 3.99 mmol, intermediate 32). The mixture was stirred for 4 h at 120° C. and over night at room temperature. The mixture was diluted with 150 mL ethyl acetate and extracted with 100 mL water twice. The organic layer was washed with brine and evaporated to dryness. The residue was adsorbed on Isolute® and purified using a Biotage chromatography system (55 g snap NH column hexane/0-100% ethyl acetate, ethyl acetate/0-100% ethanol) followed by a HPLC (water (0.2% ammonia)/15-55% acetonitrile). Using this methodology 45.0 mg of the desired titled compound were obtained (76% purity, 4% yield).

LC-MS (Method 2): Rₜ=1.10 min; MS (ESIpos): m/z=294 [M+H]⁺

¹H-NMR (400 MHz, DMSO-d6) δ [ppm]: 0.867 (0.49), 1.025 (0.57), 1.038 (1.43), 1.044 (1.88), 1.050 (2.12), 1.056 (1.88), 1.063 (1.23), 1.077 (1.87), 1.083 (2.68), 1.090 (1.37), 1.095 (1.90), 1.103 (2.45), 1.110 (1.35), 1.122 (0.83), 1.231 (0.58), 1.255 (0.45), 1.264 (1.00), 1.272 (0.80), 1.284 (1.14), 1.292 (0.85), 1.575 (0.48), 1.611 (0.41), 1.750 (0.40), 1.782 (0.64), 1.795 (1.04), 1.814 (1.66), 1.827 (2.30), 1.839 (1.95), 1.850 (1.74), 1.873 (1.59), 1.923 (1.10), 1.954 (1.84), 1.966 (1.74), 1.984 (1.55), 2.021 (1.56), 2.043 (1.31), 2.060 (1.42), 2.099 (2.18), 2.115 (1.64), 2.145 (2.61), 2.275 (0.56), 2.287 (0.71), 2.295 (0.92), 2.307 (1.10), 2.319 (0.91), 2.322 (1.04), 2.327 (1.62), 2.331 (1.02), 2.337 (0.72), 2.354 (0.45), 2.370 (0.53), 2.382 (0.57), 2.518 (6.78), 2.522 (4.56), 2.539 (16.00), 2.664 (0.79), 2.669 (1.13), 2.673 (0.82), 2.994 (1.38), 3.970 (2.28), 5.759 (3.45), 5.776 (3.09), 6.035 (0.72), 8.324 (6.70), 8.411 (0.50), 8.837 (2.88), 11.781 (1.19).

### Intermediate 34

### tert-butyl 2-(1-methylazepan-4-ylidene)hydrazine-1-carboxylate

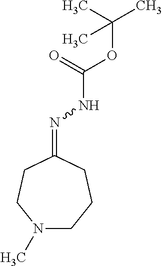 [SMILES: CN1CCCC(=NNC(=O)OC(C)(C)C)CC1]

To a solution of 1-methylazepan-4-one (3.40 g, 26.7 mmol, CAS-RN:[1859-33-2], commercially available e.g. Combi-Blocks Inc.) in 12 mL methanol a solution of tert-butyl hydrazinecarboxylate (3.36 g, 25.4 mmol) in 12 mL methanol was added dropwise. The mixture was then stirred for 1 h at room temperature and directly used in the next step.

### Intermediate 35

### (±)-tert-butyl 2-(1-methylazepan-4-yl)hydrazine-1-carboxylate

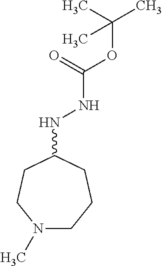 [SMILES: CN1CCCC(NNC(=O)OC(C)(C)C)CC1]

Tert-butyl 2-(1-methylazepan-4-ylidene)hydrazine-1-carboxylate (3.23 g, 13.4 mmol, intermediate 34) was mixed with 33 mL water and 11 mL acetic acid. After the mixture was stirred for 1 h at 0° C. sodium cyanoborohydride (1.01 g, 16.0 mmol) was added. The reaction was stirred over night at room temperature. To the mixture an aqueous sodiumhydroxide solution (c=4 mol/L) was added till pH=7 was achieved. The mixture was extracted with 50 mL dichloromethane for three times. The organic layer was washed with brine, filtered over a hydrophobic filter and evaporated till most of the solvent was gone to give 1.63 g of the desired titled compound (91% purity, 46% yield).

¹H-NMR (400 MHz, DMSO-d6) δ [ppm]: 0.877 (0.60), 0.893 (0.64), 1.385 (16.00), 1.399 (1.20), 1.746 (2.15), 1.790 (0.42), 1.906 (1.10), 2.725 (5.08), 3.053 (0.54), 5.758 (2.17), 8.266 (0.47).

### Intermediate 36

### (±)-4-hydrazinyl-1-methylazepane—hydrogen chloride (1/1)

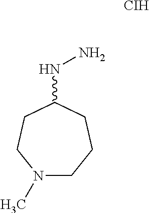

(±)-Tert-butyl 2-(1-methylazepan-4-yl)hydrazine-1-carboxylate (1.60 g, 6.57 mmol, intermediate 35) was suspended in 16 mL 4M hydrogen chloride in dioxane and stirred for 1 h at 20° C. The mixture was filtered and washed with dioxane. The filtercake was dried to give the desired product as raw material (1.44 g, 117% yield). The raw material was directly used in the next step.

¹H-NMR (500 MHz, DMSO-d6) δ [ppm]: 1.162 (2.14), 1.176 (2.18), 1.453 (0.60), 1.561 (0.70), 1.588 (16.00), 1.607 (0.44), 1.710 (0.65), 1.736 (1.95), 1.747 (1.76), 1.761 (2.36), 1.786 (0.69), 1.866 (0.96), 1.881 (0.82), 1.904 (0.95), 1.920 (0.72), 1.940 (1.75), 1.954 (2.59), 1.972 (2.06), 1.992 (0.76), 2.069 (1.26), 2.085 (1.26), 2.097 (1.24), 2.108 (1.14), 2.171 (0.63), 2.179 (0.73), 2.185 (0.75), 2.194 (0.76), 2.202 (0.64), 2.210 (0.62), 2.217 (0.65), 2.225 (0.57), 2.264 (0.59), 2.284 (0.72), 2.313 (0.45), 2.515 (0.77), 2.518 (0.68), 2.522 (0.53), 2.688 (9.28), 2.698 (10.46), 2.703 (10.97), 2.713 (9.56), 2.750 (0.41), 2.759 (0.41), 2.930 (0.44), 2.947 (0.81), 2.952 (0.84), 2.973 (1.23), 2.995 (1.08), 3.013 (1.36), 3.030 (1.01), 3.041 (0.75), 3.058 (0.81), 3.078 (0.55), 3.207 (0.59), 3.234 (1.61), 3.251 (1.74), 3.272 (1.30), 3.282 (1.68), 3.292 (2.02), 3.302 (2.48), 3.310 (2.59), 3.323 (1.88), 3.339 (1.35), 3.396 (0.79), 3.412 (0.90), 3.424 (0.87), 3.440 (0.71), 4.609 (0.55), 6.511 (0.61), 11.025 (1.26).

### Intermediate 37

### (±)-6-cyclopropyl-1-(1-methylazepan-4-yl)-1H-pyrazolo[3,4-b]pyrazin-3-amine

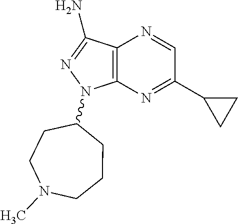 [SMILES: CN1CCCC(n2nc(N)c3ncc(C4CC4)nc32)CC1]

To a solution of 3-chloro-5-cyclopropylpyrazine-2-carbonitrile (960 mg, 5.34 mmol, intermediate 1) in 14 mL 1-butanol were added potassium carbonate (2.95 g, 21.4 mmol) and (±)-4-hydrazinyl-1-methylazepane-hydrogen chloride (1/1) (1.44 g, 8.01 mmol, intermediate 36). The mixture was stirred for 4 h at 120° C. and over night at room temperature. The mixture was diluted with 150 mL ethyl acetate and extracted with 100 mL water twice. The organic layer was washed with brine and evaporated to dryness. The residue was adsorbed on Isolute® and purified two times using a Biotage chromatography system (55 g snap NH column hexane/0-100% ethyl acetate then ethyl acetate/0-100% ethanol) (28 g snap NH column hexane/0-100% ethyl acetate then ethyl acetate/0-100% ethanol). Using this methodology 434 mg of the desired titled compound were obtained (41% purity, 12% yield).

LC-MS (Method 2): Rₜ=0.99 min; MS (ESIpos): m/z=287 [M+H]⁺

¹H-NMR (400 MHz, DMSO-d6) δ [ppm]: 0.978 (0.57), 0.985 (0.87), 0.988 (0.80), 0.993 (0.87), 0.996 (0.89), 1.005 (0.61), 1.011 (0.46), 1.024 (0.64), 1.031 (0.88), 1.036 (0.93), 1.042 (0.83), 1.049 (0.51), 1.052 (0.48), 1.061 (0.55), 1.066 (0.70), 1.073 (1.01), 1.081 (0.83), 1.092 (0.96), 1.099 (0.54), 1.128 (0.44), 1.149 (0.57), 1.154 (4.92), 1.171 (10.35), 1.189 (5.12), 1.903 (0.45), 1.987 (16.00), 2.139 (0.41), 2.268 (0.95), 2.273 (0.90), 2.281 (0.66), 2.292 (3.94), 2.301 (0.50), 2.304 (0.44), 2.445 (0.41), 2.518 (0.54), 2.523 (0.51), 2.557 (0.43), 2.564 (0.42), 2.578 (0.54), 3.215 (2.37), 3.957 (0.41), 3.998 (1.21), 4.016 (3.55), 4.034 (3.40), 4.052 (1.12), 5.710 (1.18), 7.991 (1.06), 7.994 (0.94), 8.298 (2.79), 8.637 (1.32).

### Intermediate 38

### (±)-6-cyclopropyl-1-(1-methylpyrrolidin-3-yl)-1H-pyrazolo[3,4-b]pyrazin-3-amine

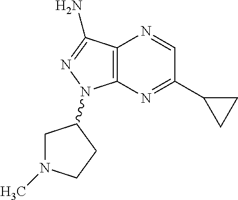 [SMILES: CN1CCC(n2nc(N)c3ncc(C4CC4)nc32)C1]

To a solution of 3-chloro-5-cyclopropylpyrazine-2-carbonitrile (1.18 g, 6.59 mmol, intermediate 1) in 16 mL 1-butanol were added potassium carbonate (3.65 g, 26.4 mmol) and (±)-3-hydrazinyl-1-methylpyrrolidine-hydrogen chloride (1/1) (1.50 g, 9.89 mmol). The mixture was stirred for 4 h at 120° C. and over night at room temperature. The mixture was diluted with 150 mL ethyl acetate and extracted with 100 mL water twice. The organic layer was washed with brine and evaporated to dryness. The residue was adsorbed on Isolute® and purified two times using a Biotage chromatography system (28 g snap NH column hexane/0-100% ethyl acetate then ethyl acetate/0-75% ethanol) (55 g snap NH column hexane/0-100% ethyl acetate then ethyl acetate/0-75% ethanol). Using this methodology 560 mg of the desired titled compound were obtained (40% purity, 13% yield).

LC-MS (Method 2): Rₜ=0.91 min; MS (ESIpos): m/z=260 [M+H]⁺

¹H-NMR (400 MHz, DMSO-d6) δ [ppm]: 0.947 (0.49), 0.965 (0.62), 0.982 (1.42), 0.986 (1.49), 0.994 (1.64), 0.999 (1.71), 1.005 (1.67), 1.011 (1.54), 1.019 (1.80), 1.033 (2.55), 1.040 (3.31), 1.044 (3.35), 1.051 (3.67), 1.057 (3.12), 1.062 (2.25), 1.067 (2.55), 1.073 (3.66), 1.080 (2.61), 1.086 (2.08), 1.093 (3.00), 1.100 (1.60), 1.112 (0.64), 1.258 (0.80), 1.986 (0.44), 2.090 (0.62), 2.102 (0.70), 2.111 (1.09), 2.123 (1.24), 2.131 (1.07), 2.143 (1.10), 2.155 (0.72), 2.164 (0.43), 2.173 (0.88), 2.191 (1.11), 2.197 (0.98), 2.209 (0.92), 2.216 (0.98), 2.223 (0.63), 2.235 (0.62), 2.238 (0.55), 2.242 (0.55), 2.248 (0.67), 2.254 (0.48), 2.269 (16.00), 2.280 (2.14), 2.287 (1.26), 2.296 (1.39), 2.299 (1.45), 2.309 (1.39), 2.318 (0.99), 2.327 (0.61), 2.331 (0.62), 2.387 (0.53), 2.405 (0.58), 2.518 (1.47), 2.523 (0.99), 2.533 (1.63), 2.539 (0.59), 2.550 (1.55), 2.555 (2.38), 2.573 (2.21), 2.594 (0.64), 2.682 (0.65), 2.693 (0.67), 2.701 (0.88), 2.714 (0.84), 2.722 (0.56), 2.734 (0.46), 2.923 (1.04), 2.943 (1.36), 2.966 (0.95), 3.136 (1.15), 3.177 (0.65), 3.244 (0.97), 3.261 (1.56), 3.682 (0.61), 3.874 (0.53), 3.882 (0.53), 3.895 (0.51), 3.901 (0.56), 3.932 (0.52), 3.960 (1.41), 4.523 (0.56), 5.128 (0.48), 5.142 (0.55), 5.147 (0.53), 5.152 (0.54), 5.167 (0.47), 5.758 (3.51), 5.794 (3.92), 5.860 (1.26), 7.933 (0.58), 7.940 (0.40), 8.000 (0.70), 8.004 (2.41), 8.008 (0.62), 8.014 (0.77), 8.326 (7.65), 8.348 (0.60), 8.355 (0.52), 8.369 (2.42).

### Intermediate 39

### 3-(cyclopentylamino)-5-cyclopropylpyrazine-2-carbonitrile

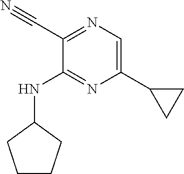 [SMILES: N#Cc1ncc(C2CC2)nc1NC1CCCC1]

To a solution of 3-chloro-5-cyclopropylpyrazine-2-carbonitrile (500 mg, 2.78 mmol, intermediate 1) in 8.3 mL N,N-dimethylformamide were added cyclopentanamine (300 pL, 3.1 mmol) and potassium carbonate (1.15 g, 8.35 mmol). The reaction mixture was stirred for 16h at 100° C. Water was added and the mixture was extracted with ethyl acetate for three times. The combined organic layers were filtered through a hydrophobic filter, evaporated under reduced pressure and purified using a Biotage chromatography system to give 200 mg (98% purity, 31% yield) of the desired compound as a yellow solid.

LC-MS (Method 2): Rₜ=1.41 min; MS (ESIneg): m/z=227 [M−H]⁻

¹H-NMR (400 MHz, DMSO-d6) δ [ppm]: 0.974 (1.10), 0.979 (1.08), 0.985 (3.55), 0.992 (5.85), 0.996 (3.84), 0.998 (3.56), 1.003 (5.18), 1.011 (2.76), 1.033 (1.01), 1.047 (2.48), 1.055 (4.64), 1.062 (2.81), 1.067 (3.28), 1.074 (5.56), 1.081 (3.15), 1.086 (1.66), 1.093 (1.50), 1.231 (0.41), 1.491 (1.14), 1.497 (1.48), 1.503 (2.35), 1.518 (5.21), 1.522 (4.68), 1.529 (4.56), 1.539 (2.95), 1.542 (2.92), 1.547 (2.40), 1.553 (2.00), 1.560 (2.34), 1.573 (1.15), 1.589 (0.49), 1.633 (0.55), 1.642 (0.90), 1.648 (1.19), 1.655 (1.41), 1.669 (2.00), 1.675 (2.85), 1.679 (2.91), 1.718 (0.55), 1.850 (0.45), 1.863 (1.21), 1.870 (1.56), 1.880 (2.42), 1.888 (2.26), 1.891 (2.02), 1.898 (2.20), 1.907 (1.58), 1.929 (0.91), 2.075 (0.71), 2.087 (1.50), 2.095 (1.62), 2.099 (0.96), 2.106 (2.83), 2.115 (1.27), 2.118 (1.60), 2.126 (1.49), 2.138 (0.72), 2.327 (0.52), 2.332 (0.40), 2.522 (4.18), 2.664 (0.49), 2.669 (0.63), 2.673 (0.50), 3.190 (1.23), 3.363 (0.63), 3.905 (1.54), 4.136 (0.85), 4.154 (1.60), 4.171 (1.59), 4.188 (0.82), 7.219 (2.09), 7.236 (2.17), 7.494 (0.54), 7.894 (16.00), 7.907 (0.46).

### Intermediate 40

### 1-cyclopentyl-6-cyclopropyl-1H-pyrazolo[3,4-b]pyrazin-3-amine

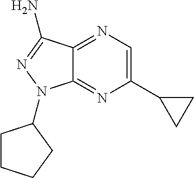 [SMILES: Nc1nn(C2CCCC2)c2nc(C3CC3)cnc12]

A solution of 3-(cyclopentylamino)-5-cyclopropylpyrazine-2-carbonitrile (100 mg, 438 pmol, intermediate 39) in 0.49 mL N,N-dimethylformamide was cooled to 0° C. To that solution a solution of lithium bis(trimethylsilyl)amide (660 μL, 1.0 M, 660 μmol) in tetrahydrofuran was added dropwise. The mixture was stirred for 10 min at 0° C. and then O-(diphenylphosphinoyl)hydroxylamine (138 mg, 591 μmol; CAS-RN:[72804-96-7]) was added. The mixture was stirred for further 3 h at room temperature. Another 30.7 mg of O-(diphenylphosphinoyl)hydroxylamine (131 μmol) were added and the mixture was stirred for 16 h at room temperature. Another 30.7 mg of 0-(diphenylphosphinoyl)hydroxylamine (131 μmol) were added and the mixture was stirred for 1 h at room temperature. Water was added and the mixture was extracted with ethyl acetate three times. The combined organic layers were filtered through a hydrophobic filter, evaporated under reduced pressure and purified using a Biotage chromatography system to give 112 mg (90% purity, 95% yield) of the desired compound.

LC-MS (Method 2): Rₜ=1.19 min; MS (ESIpos): m/z=244 [M+H]⁺

¹H-NMR (400 MHz, DMSO-d6) δ [ppm]: 1.013 (0.98), 1.025 (3.28), 1.032 (4.44), 1.037 (4.86), 1.044 (4.17), 1.050 (2.40), 1.063 (2.68), 1.070 (4.03), 1.077 (2.37), 1.083 (2.81), 1.090 (4.71), 1.097 (2.47), 1.108 (1.16), 1.231 (0.52), 1.258 (0.43), 1.589 (0.45), 1.604 (0.92), 1.611 (1.24), 1.620 (1.82), 1.632 (2.41), 1.638 (1.97), 1.648 (1.87), 1.658 (0.95), 1.668 (0.59), 1.676 (0.50), 1.772 (0.48), 1.779 (0.56), 1.800 (1.44), 1.814 (2.04), 1.822 (1.99), 1.835 (1.51), 1.842 (1.23), 1.857 (1.17), 1.874 (1.07), 1.878 (1.04), 1.888 (1.54), 1.893 (1.65), 1.906 (2.42), 1.923 (2.13), 1.944 (1.79), 1.952 (2.02), 1.965 (2.23), 1.978 (1.34), 1.985 (1.65), 1.997 (0.96), 2.014 (0.40), 2.265 (0.60), 2.277 (1.24), 2.284 (1.32), 2.292 (1.28), 2.296 (2.14), 2.305 (0.98), 2.308 (1.26), 2.316 (1.29), 2.323 (0.66), 2.328 (1.09), 2.518 (2.70), 2.523 (1.73), 2.665 (0.51), 2.669 (0.69), 2.673 (0.48), 2.729 (1.69), 2.888 (2.03), 3.956 (1.37), 4.945 (0.58), 4.962 (1.37), 4.965 (1.38), 4.982 (2.31), 4.999 (1.27), 5.002 (1.22), 5.019 (0.47), 5.727 (7.34), 5.759 (0.60), 7.745 (0.56), 7.918 (0.63), 8.307 (16.00).

### Intermediate 41

### 5-cyclopropyl-3-{[(cis)-3-hydroxycyclobutyl]amino}pyrazine-2-carbonitrile

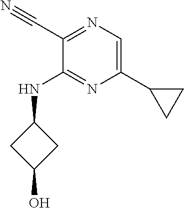 [SMILES: N#Cc1ncc(C2CC2)nc1N[C@H]1C[C@@H](O)C1]

In analogy to 39) 3-chloro-5-cyclopropylpyrazine-2-carbonitrile (600 mg, 3.34 mmol, intermediate 1) and cis-3-aminocyclobutan-1-ol-hydrogen chloride (1/1) (454 mg, 3.67 mmol, CAS-RN:[1219019-22-3], commercially available e.g. ABCR) reacted and give after purification using a Biotage chromatography 390 mg (95% purity, 48% yield) of the desired title compound.

LC-MS (Method 2): Rₜ=0.93 min; MS (ESIpos): m/z=231 [M+H]⁺

¹H-NMR (400 MHz, DMSO-d6) δ [ppm]: 0.965 (1.12), 0.970 (1.22), 0.976 (3.15), 0.983 (5.08), 0.987 (3.22), 0.989 (2.91), 0.994 (4.20), 1.001 (1.99), 1.007 (0.61), 1.025 (0.61), 1.042 (1.81), 1.049 (3.86), 1.057 (2.26), 1.062 (2.75), 1.070 (4.48), 1.077 (2.49), 1.081 (1.14), 1.087 (1.05), 1.874 (1.33), 1.881 (0.98), 1.896 (2.50), 1.903 (2.43), 1.909 (0.96), 1.916 (2.46), 1.923 (2.61), 1.938 (1.06), 1.945 (1.43), 1.952 (0.42), 1.987 (0.63), 2.064 (0.66), 2.075 (1.31), 2.083 (1.43), 2.088 (0.84), 2.095 (2.46), 2.104 (1.02), 2.107 (1.33), 2.115 (1.21), 2.126 (0.50), 2.518 (3.76), 2.523 (2.55), 2.526 (2.43), 2.530 (2.05), 2.537 (2.10), 2.541 (2.21), 2.543 (1.87), 2.548 (2.43), 2.555 (0.83), 2.558 (1.18), 2.565 (1.32), 3.714 (0.71), 3.719 (0.72), 3.730 (0.79), 3.737 (1.38), 3.742 (0.56), 3.747 (0.58), 3.752 (1.39), 3.759 (0.74), 3.770 (0.70), 3.775 (0.67), 3.828 (0.93), 3.832 (0.90), 3.848 (1.76), 3.863 (1.76), 3.880 (0.89), 3.883 (0.90), 3.897 (0.69), 5.037 (7.48), 5.052 (7.23), 5.758 (0.59), 7.585 (2.12), 7.599 (2.08), 7.760 (0.49), 7.902 (16.00).

### Intermediate 42

### cis-3-(3-amino-6-cyclopropyl-1H-pyrazolo[3,4-b]pyrazin-1-yl)cyclobutan-1-ol

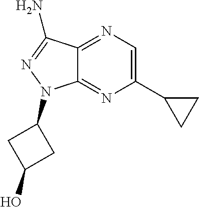 [SMILES: Nc1nn([C@H]2C[C@@H](O)C2)c2nc(C3CC3)cnc12]

A solution of 5-cyclopropyl-3-{[(cis)-3-hydroxycyclobutyl]amino}pyrazine-2-carbonitrile (280 mg, 1.22 mmol, intermediate 41) in 7.0 mL N,N-dimethylformamide was cooled to 0° C. To that solution a solution of lithium bis(trimethylsilyl)amide (2.7 mL, 1.0 M, 2.7 mmol) in tetrahydrofuran was added dropwise. The mixture was stirred for 10 min at 0° C. and then

O-(diphenylphosphinoyl)hydroxylamine (383 mg, 1.64 mmol, CAS-RN:[72804-96-7]) was added. The mixture was stirred for further 2 h at room temperature. Water was added and the mixture was extracted with ethyl acetate three times. The combined organic layers were filtered through a hydrophobic filter, evaporated under reduced pressure and purified using a Biotage chromatography system to give 357 mg (84% purity, 101% yield) of the desired compound.

LC-MS (Method 2): Rₜ=0.80 min; MS (ESIpos): m/z=246 [M+H]⁺

¹H-NMR (400 MHz, DMSO-d6) δ [ppm]: 1.011 (1.08), 1.024 (3.63), 1.030 (5.01), 1.036 (5.60), 1.042 (4.85), 1.049 (3.39), 1.060 (2.93), 1.066 (4.81), 1.073 (2.74), 1.079 (3.64), 1.086 (5.33), 1.093 (2.84), 1.105 (1.32), 1.153 (0.96), 1.171 (1.74), 1.189 (0.88), 1.986 (2.97), 2.261 (0.68), 2.273 (1.38), 2.281 (1.54), 2.293 (2.46), 2.305 (1.47), 2.313 (1.32), 2.326 (0.83), 2.397 (1.11), 2.403 (0.88), 2.418 (2.48), 2.425 (2.64), 2.440 (2.60), 2.446 (2.99), 2.461 (1.19), 2.468 (1.69), 2.475 (0.89), 2.518 (1.88), 2.523 (1.34), 2.546 (0.93), 2.552 (1.62), 2.560 (1.51), 2.570 (2.92), 2.576 (2.54), 2.580 (2.23), 2.587 (2.44), 2.591 (2.34), 2.598 (2.22), 2.608 (1.15), 2.615 (1.16), 2.664 (0.47), 2.669 (0.57), 2.673 (0.43), 3.159 (2.09), 3.172 (2.09), 3.953 (0.65), 3.967 (0.92), 3.970 (0.90), 3.987 (1.83), 4.002 (1.84), 4.016 (1.10), 4.019 (0.98), 4.022 (0.93), 4.034 (0.77), 4.096 (0.56), 4.110 (0.56), 4.566 (0.56), 4.584 (1.36), 4.588 (1.40), 4.607 (2.53), 4.625 (1.43), 4.629 (1.23), 4.648 (0.52), 5.213 (7.13), 5.228 (6.81), 5.795 (8.49), 5.833 (0.65), 7.759 (0.68), 8.321 (16.00), 8.327 (1.57), 8.334 (0.80).

### Intermediate 43

### (±)-tert-butyl 2-(1,2-dimethylpiperidin-4-ylidene)hydrazine-1-carboxylate

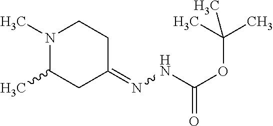 [SMILES: CC1CC(=NNC(=O)OC(C)(C)C)CCN1C]

To a solution of (±)-1,2-dimethylpiperidin-4-one (500 mg, 3.93 mmol, CAS-RN:[13669-32-4], commercially available e.g. Enamine) in 3.0 mL methanol a solution of tert-butyl hydrazinecarboxylate (494 mg, 3.73 mmol) in 3.0 mL methanol was added dropwise. The mixture was then stirred for 1 h at room temperature and directly used in the next step.

### Intermediate 44

### (±)-tert-butyl 2-(cis/trans-1,2-di methyl piperidin-4-yl)-hydrazine-1-carboxylate

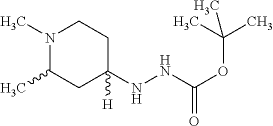 [SMILES: CC1CC(NNC(=O)OC(C)(C)C)CCN1C]

In analogy to intermediate 35) (±)-tert-butyl 2-(1,2-dimethylpiperidin-4-ylidene)hydrazine-1-carboxylate (950 mg, 3.94 mmol, intermediate 43) reacted and give without further purification 1.10 g (80% purity, 91% yield) of the desired title compound as raw product.

### Intermediate 45

### (±)-cis/trans-4-hydrazinyl-1,2-dimethylpiperidine—hydrogen chloride (1/1)

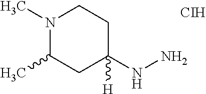

In analogy to intermediate 36) (±)-tert-butyl 2-(cis/trans-1,2-dimethylpiperidin-4-yl)-hydrazine-1-carboxylate (957 mg, 3.93 mmol, intermediate 44) reacted and give without further purification 750 mg (95% purity, 100% yield) of the desired title compound as hygroscopic raw product.

### Intermediate 46

### (±)-cis/trans-6-cyclopropyl-1-(1,2-dimethylpiperidin-4-yl)-1H-pyrazolo[3,4-b]pyrazin amine

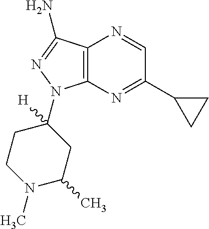 [SMILES: CC1CC(n2nc(N)c3ncc(C4CC4)nc32)CCN1C]

In analogy to example 37) 3-chloro-5-cyclopropylpyrazine-2-carbonitrile (350 mg, 1.95 mmol, intermediate 1) and (±)-cis/trans-4-hydrazinyl-1,2-dimethylpiperidine-hydrogen chloride (1/1) (700 mg, 3.90 mmol, intermediate 45) reacted and give after one purification using a Biotage chromatography system 277 mg (69% purity, 34% yield) of the desired title compound.

LC-MS (Method 2): Rₜ=1.02 min; MS (ESIpos): m/z=287 [M+H]⁺

¹H-NMR (400 MHz, DMSO-d6) δ [ppm]: 0.842 (2.85), 0.861 (6.60), 0.878 (3.64), 0.890 (0.41), 0.900 (0.71), 0.909 (0.78), 0.914 (2.03), 0.923 (0.80), 0.930 (2.24), 0.938 (0.66), 0.942 (0.46), 0.947 (0.59), 0.965 (3.89), 0.969 (1.42), 0.980 (5.75), 0.985 (4.81), 0.994 (3.14), 1.000 (4.29), 1.009 (1.96), 1.023 (10.35), 1.028 (5.57), 1.034 (6.01), 1.038 (10.81), 1.047 (2.94), 1.060 (1.68), 1.065 (2.33), 1.073 (3.33), 1.079 (2.50), 1.083 (1.90), 1.085 (2.24), 1.092 (3.56), 1.099 (2.06), 1.111 (1.04), 1.120 (0.58), 1.128 (0.80), 1.135 (0.71), 1.139 (0.64), 1.149 (1.01), 1.154 (4.59), 1.172 (9.46), 1.179 (0.64), 1.189 (4.87), 1.230 (0.49), 1.245 (0.45), 1.258 (0.95), 1.261 (1.04), 1.269 (0.54), 1.279 (1.37), 1.283 (1.29), 1.300 (1.36), 1.305 (0.77), 1.312 (0.73), 1.316 (1.25), 1.323 (0.73), 1.334 (0.54), 1.344 (0.42), 1.353 (0.78), 1.365 (0.75), 1.367 (1.15), 1.370 (1.45), 1.381 (1.22), 1.383 (0.98), 1.386 (1.33), 1.389 (1.09), 1.398 (0.61), 1.403 (1.04), 1.405 (0.93), 1.420 (0.56), 1.423 (0.52), 1.612 (0.41), 1.624 (0.51), 1.637 (3.35), 1.672 (1.11), 1.678 (0.88), 1.684 (0.91), 1.698 (0.81), 1.717 (1.74), 1.757 (2.21), 1.772 (3.25), 1.785 (2.59), 1.814 (1.77), 1.845 (0.80), 1.914 (0.53), 1.926 (0.79), 1.952 (0.63), 1.987 (15.78), 1.995 (0.90), 2.003 (0.86), 2.010 (1.06), 2.018 (0.97), 2.026 (1.22), 2.036 (1.02), 2.045 (0.98), 2.057 (1.06), 2.067 (0.89), 2.088 (1.30), 2.094 (6.58), 2.121 (6.18), 2.131 (2.59), 2.139 (1.19), 2.149 (1.86), 2.154 (1.92), 2.163 (3.55), 2.187 (16.00), 2.211 (1.34), 2.221 (0.49), 2.263 (0.56), 2.275 (1.04), 2.283 (1.21), 2.295 (1.79), 2.307 (1.21), 2.315 (1.19), 2.327 (1.13), 2.331 (0.67), 2.518 (1.77), 2.523 (1.24), 2.665 (0.42), 2.669 (0.68), 2.674 (0.55), 2.727 (0.48), 2.734 (0.41), 2.763 (0.50), 2.857 (1.31), 2.865 (0.99), 2.878 (0.89), 2.884 (1.06), 2.893 (0.63), 3.226 (0.40), 3.354 (1.74), 3.371 (1.68), 3.383 (1.56), 3.399 (0.72), 3.458 (0.60), 3.460 (0.45), 3.468 (0.61), 3.470 (0.66), 3.493 (0.40), 3.663 (0.55), 3.667 (0.46), 3.675 (0.58), 3.677 (0.72), 3.682 (1.38), 3.699 (0.69), 3.701 (0.63), 3.709 (0.43), 3.713 (0.54), 3.955 (1.16), 3.999 (1.18), 4.016 (3.52), 4.034 (3.44), 4.052 (1.12), 4.313 (0.49), 4.326 (1.07), 4.339 (0.50), 4.389 (0.51), 4.400 (0.41), 4.408 (0.51), 4.418 (0.95), 4.429 (0.58), 4.447 (0.46), 4.562 (0.52), 4.574 (0.52), 5.723 (4.71), 5.758 (3.24), 7.927 (0.55), 8.303 (11.04), 8.314 (0.70), 8.318 (0.51), 8.637 (1.89), 8.836 (0.72).

### Intermediate 47

### (±)-tert-butyl 2-(3-methyloxan-4-ylidene)hydrazine-1-carboxylate

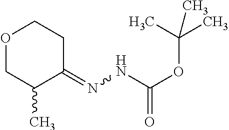 [SMILES: CC1COCCC1=NNC(=O)OC(C)(C)C]

To a solution of (±)-3-methyloxan-4-one (500 mg, 4.38 mmol, CAS-RN:[119124-53-7], commercially available e.g. ABCR) in 3.4 mL methanol a solution of tert-butyl hydrazinecarboxylate (550 mg, 4.16 mmol) in 3.4 mL methanol was added dropwise. The mixture was then stirred for 1 h at room temperature and directly used in the next step.

### Intermediate 48

### (±)-tert-butyl 2-(cis/trans-3-methyloxan-4-yl)hydrazine-1-carboxylate

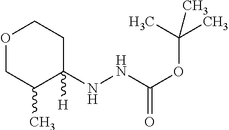 [SMILES: CC1COCCC1NNC(=O)OC(C)(C)C]

In analogy to intermediate 35) (±)-tert-butyl 2-(3-methyloxan-4-ylidene)hydrazine-1-carboxylate (1000 mg, 4.38 mmol, intermediate 47) reacted and give without further purification 1.15 g (114% yield) of the desired title compound as raw product.

### Intermediate 49

### (±)-(cis/trans-3-methyloxan-4-yl)hydrazine-hydrogen chloride (1/1)

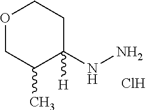

In analogy to intermediate 36) (±)-tert-butyl 2-(cis/trans-3-methyloxan-4-yl)hydrazine-1-carboxylate (1.01 g, 4.38 mmol, intermediate 48) reacted and give without further purification 759 mg (104% yield) of the desired title compound as hygroscopic raw product.

### Intermediate 50

### (±)-cis/trans-6-cyclopropyl-1-(3-methyloxan-4-yl)-1H-pyrazolo[3,4-b]pyrazin-3-amine

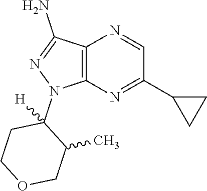 [SMILES: CC1COCCC1n1nc(N)c2ncc(C3CC3)nc21]

In analogy to example 37) 3-chloro-5-cyclopropylpyrazine-2-carbonitrile (700 mg, 3.90 mmol, intermediate 1) and (±)-(cis/trans-3-methyloxan-4-yl)hydrazine-hydrogen chloride (1/1) (1.30 g, 7.79 mmol, intermediate 49) reacted and give after one purification using a Biotage chromatography system 366 mg (34% yield) of the desired title compound.

LC-MS (Method 2): Rₜ=0.98 min; MS (ESIpos): m/z=274 [M+H]⁺

¹H-NMR (400 MHz, DMSO-d6) δ [ppm]: 0.424 (0.64), 0.440 (0.65), 0.641 (0.46), 0.659 (0.49), 0.779 (1.56), 0.789 (0.52), 0.797 (1.97), 0.802 (3.11), 0.812 (0.56), 0.818 (3.13), 0.831 (0.80), 0.850 (3.18), 0.860 (0.86), 0.866 (2.68), 0.875 (2.32), 0.893 (2.37), 0.910 (1.04), 0.915 (1.07), 0.926 (1.40), 0.929 (1.28), 0.932 (1.16), 0.938 (0.63), 0.945 (0.67), 0.965 (0.80), 0.983 (0.42), 0.990 (0.43), 0.997 (0.54), 1.001 (0.54), 1.006 (0.55), 1.009 (0.77), 1.014 (0.74), 1.018 (0.93), 1.021 (0.87), 1.026 (0.66), 1.029 (0.82), 1.034 (0.84), 1.041 (0.62), 1.067 (0.63), 1.076 (0.54), 1.083 (0.73), 1.127 (0.41), 1.147 (0.53), 1.154 (1.61), 1.171 (2.69), 1.175 (0.55), 1.182 (0.44), 1.189 (1.26), 1.359 (0.63), 1.363 (0.75), 1.382 (16.00), 1.401 (0.82), 1.413 (0.42), 1.429 (1.72), 1.691 (0.56), 1.697 (0.48), 1.703 (0.55), 1.709 (0.50), 1.723 (0.48), 1.729 (0.46), 1.735 (0.42), 1.986 (4.05), 2.518 (0.56), 2.835 (0.42), 2.862 (0.60), 2.890 (0.53), 2.898 (0.42), 2.902 (0.43), 3.237 (0.44), 3.244 (0.40), 3.256 (0.47), 3.262 (0.47), 3.267 (0.67), 3.273 (0.66), 3.296 (0.57), 3.302 (0.50), 3.314 (0.49), 3.342 (0.54), 3.351 (0.74), 3.355 (0.59), 3.675 (0.61), 3.682 (0.49), 3.692 (0.41), 3.701 (0.49), 3.756 (0.42), 3.774 (0.47), 3.784 (0.70), 3.799 (0.45), 3.817 (0.48), 4.017 (0.94), 4.034 (0.94), 4.542 (0.50), 4.553 (0.50), 4.665 (1.33), 4.679 (1.25), 5.757 (0.82), 8.321 (0.90), 8.331 (1.42), 8.333 (0.70), 8.395 (0.48), 8.556 (1.26).

### Intermediate 51

### 5-cyclopropyl-3-(trans-3-hydroxycyclobutyl)amino]pyrazine-2-carbonitrile

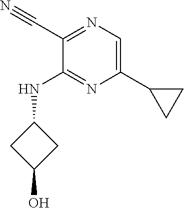 [SMILES: N#Cc1ncc(C2CC2)nc1N[C@H]1C[C@H](O)C1]

In analogy to 39) 3-chloro-5-cyclopropylpyrazine-2-carbonitrile (400 mg, 2.23 mmol, intermediate 1) and trans-3-aminocyclobutan-1-ol-hydrogen chloride (1/1) (303 mg, 2.45 mmol, CAS-RN:[1205037-95-1], commercially available e.g. Advanced ChemBlocks Inc.) reacted and give after purification using a Biotage chromatography 350 mg (97% purity, 66% yield) of the desired title compound.

LC-MS (Method 2): Rₜ=0.89 min; MS (ESIpos): m/z=231 [M+H]⁺

¹H-NMR (400 MHz, DMSO-d6) δ [ppm]: 0.959 (1.11), 0.970 (3.07), 0.977 (5.05), 0.981 (3.23), 0.984 (2.87), 0.988 (4.16), 0.995 (1.90), 1.031 (0.44), 1.046 (1.68), 1.054 (3.77), 1.061 (2.35), 1.066 (2.67), 1.074 (4.37), 1.081 (2.50), 1.092 (1.12), 2.067 (0.71), 2.078 (2.40), 2.088 (2.81), 2.099 (4.00), 2.110 (4.35), 2.119 (3.01), 2.130 (2.74), 2.140 (2.08), 2.258 (1.85), 2.266 (0.98), 2.273 (2.40), 2.275 (2.80), 2.283 (1.54), 2.290 (3.27), 2.297 (1.44), 2.304 (2.02), 2.314 (0.77), 2.322 (1.46), 2.518 (0.80), 2.523 (0.51), 2.727 (2.35), 2.888 (2.77), 3.895 (1.71), 4.241 (0.72), 4.248 (0.75), 4.251 (0.88), 4.258 (1.49), 4.263 (0.86), 4.269 (1.46), 4.276 (0.92), 4.278 (0.80), 4.286 (0.78), 4.341 (0.72), 4.345 (0.57), 4.356 (0.89), 4.360 (1.40), 4.368 (0.59), 4.374 (1.38), 4.380 (0.86), 4.391 (0.55), 4.394 (0.69), 4.986 (7.28), 4.999 (7.45), 7.511 (0.66), 7.574 (2.20), 7.588 (2.18), 7.912 (16.00).

### Intermediate 52

### trans-3-(3-amino-6-cyclopropyl-1H-pyrazolo[3,4-b]pyrazin-1-yl)cyclobutan-1-ol

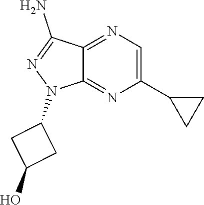 [SMILES: Nc1nn([C@H]2C[C@H](O)C2)c2nc(C3CC3)cnc12]

In analogy to 42) 5-cyclopropyl-3-(trans-3-hydroxycyclobutyl)amino]pyrazine-2-carbonitrile (350 mg, 1.52 mmol, intermediate 51) and O-(diphenylphosphinoyl)hydroxylamine (479 mg, 2.05 mmol) reacted and give after purification using a Biotage chromatography 266 mg (84% purity, 60% yield) of the desired title compound.

LC-MS (Method 2): Rₜ=0.76 min; MS (ESIpos): m/z=246 [M+H]⁺

¹H-NMR (400 MHz, DMSO-d6) δ [ppm]: 0.101 (1.64), 0.976 (0.47), 0.987 (0.45), 1.015 (0.77), 1.027 (2.89), 1.034 (3.93), 1.040 (4.25), 1.046 (3.89), 1.053 (2.79), 1.064 (2.35), 1.070 (3.76), 1.077 (2.11), 1.083 (2.77), 1.090 (4.25), 1.097 (2.18), 1.109 (1.04), 2.098 (0.42), 2.110 (0.41), 2.260 (1.47), 2.264 (1.19), 2.270 (1.60), 2.273 (1.19), 2.277 (1.90), 2.281 (2.11), 2.284 (2.53), 2.292 (3.86), 2.296 (2.89), 2.302 (2.36), 2.305 (1.94), 2.309 (1.92), 2.314 (2.22), 2.323 (1.99), 2.328 (0.87), 2.518 (0.92), 2.523 (0.65), 2.622 (1.51), 2.631 (0.79), 2.639 (2.24), 2.646 (1.37), 2.655 (2.81), 2.663 (1.45), 2.670 (2.15), 2.679 (0.81), 2.687 (1.36), 2.727 (0.44), 2.729 (0.41), 2.887 (0.47), 3.159 (1.06), 3.172 (0.89), 3.958 (1.26), 4.448 (0.56), 4.455 (0.72), 4.458 (0.72), 4.465 (1.18), 4.476 (1.18), 4.483 (0.75), 4.486 (0.73), 4.493 (0.59), 4.988 (0.71), 5.000 (0.65), 5.163 (6.86), 5.176 (6.70), 5.227 (0.89), 5.231 (0.87), 5.242 (0.70), 5.246 (1.58), 5.248 (1.55), 5.252 (0.67), 5.263 (0.85), 5.267 (0.83), 5.810 (6.46), 7.912 (1.47), 7.927 (0.57), 8.324 (16.00).

### Intermediate 53

### (±)-tert-butyl 2-(2-methyloxan-4-ylidene)hydrazine-1-carboxylate

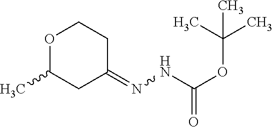 [SMILES: CC1CC(=NNC(=O)OC(C)(C)C)CCO1]

To a solution of (±)-2-methyloxan-4-one (2.30 g, 20.1 mmol, CAS-RN:[1193-20-0], commercially available e.g. Enamine) in 15 mL methanol a solution of tert-butyl hydrazinecarboxylate (2.53 g, 19.1 mmol) in 15 mL methanol was added dropwise. The mixture was then stirred for 1 h at room temperature and directly used in the next step.

### Intermediate 54

### (±)-tert-butyl 2-(cis/trans-2-methyloxan-4-yl)hydrazine-1-carboxylate

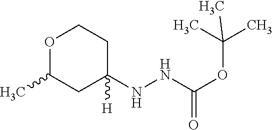 [SMILES: CC1CC(NNC(=O)OC(C)(C)C)CCO1]

In analogy to intermediate 35) (±)-tert-butyl 2-(2-methyloxan-4-ylidene)hydrazine-1-carboxylate (4.60 g, 20.1 mmol, intermediate 53) reacted and give without further purification 4.88 g (105% yield) of the desired title compound as raw product.

### Intermediate 55

### (±)-(cis/trans-2-methyloxan-4-yl)hydrazine—hydrogen chloride (1/1)

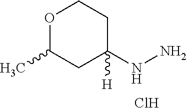

In analogy to intermediate 36) (±)-tert-butyl 2-(cis/trans-2-methyloxan-4-yl)hydrazine-1-carboxylate (1.50 g, 6.51 mmol, intermediate 54) reacted and give without further purification 1.10 g (101% yield) of the desired title compound as hygroscopic raw product.

### Intermediate 56

### (±)-6-cyclopropyl-1-(cis/trans-2-methyloxan-4-yl)-1H-pyrazolo[3,4-b]pyrazin-3-amine

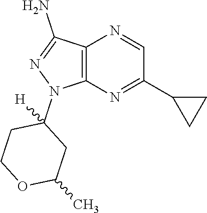 [SMILES: CC1CC(n2nc(N)c3ncc(C4CC4)nc32)CCO1]

In analogy to example 37) 3-chloro-5-cyclopropylpyrazine-2-carbonitrile (292 mg, 1.63 mmol, intermediate 1) and (±)-(cis/trans-2-methyloxan-4-yl)hydrazine-hydrogen chloride (1/1) (1.09 g, 6.51 mmol, intermediate 55) reacted and give after one purification using a Biotage chromatography system 310 mg (77% purity, 54% yield) of the desired title compound.

LC-MS (Method 2): Rₜ=0.98 min; MS (ESIpos): m/z=274 [M+H]⁺

¹H-NMR (400 MHz, DMSO-d6) δ [ppm]: 0.814 (0.44), 0.843 (2.17), 0.861 (4.55), 0.879 (2.62), 0.906 (0.47), 0.914 (0.58), 0.918 (0.66), 0.925 (1.15), 0.930 (0.63), 0.938 (0.68), 0.946 (0.79), 0.951 (1.33), 0.961 (0.49), 0.966 (0.77), 0.982 (1.51), 0.990 (1.01), 1.002 (7.36), 1.009 (1.58), 1.018 (7.72), 1.027 (1.23), 1.033 (2.62), 1.040 (2.42), 1.045 (2.47), 1.052 (2.43), 1.060 (9.64), 1.076 (11.53), 1.083 (2.69), 1.087 (3.05), 1.091 (4.02), 1.099 (2.86), 1.106 (2.68), 1.111 (1.65), 1.122 (7.47), 1.138 (6.00), 1.154 (1.80), 1.160 (0.85), 1.175 (1.15), 1.188 (0.62), 1.203 (0.60), 1.207 (0.85), 1.215 (0.60), 1.219 (0.91), 1.234 (0.98), 1.239 (0.77), 1.247 (0.91), 1.251 (0.66), 1.259 (1.19), 1.266 (1.45), 1.279 (1.15), 1.288 (0.60), 1.298 (1.16), 1.301 (1.04), 1.317 (0.76), 1.323 (0.70), 1.330 (0.57), 1.354 (0.61), 1.363 (0.89), 1.370 (2.00), 1.380 (16.00), 1.398 (0.69), 1.403 (1.06), 1.406 (1.00), 1.408 (1.04), 1.411 (0.78), 1.413 (0.77), 1.416 (0.77), 1.425 (2.31), 1.499 (0.45), 1.505 (0.69), 1.512 (0.65), 1.518 (0.43), 1.533 (0.46), 1.537 (0.60), 1.546 (0.55), 1.551 (0.48), 1.557 (0.44), 1.564 (0.43), 1.571 (0.46), 1.593 (0.72), 1.602 (0.58), 1.615 (0.52), 1.623 (0.53), 1.628 (0.56), 1.635 (0.45), 1.649 (0.40), 1.658 (0.82), 1.663 (0.95), 1.670 (1.00), 1.675 (1.02), 1.681 (0.66), 1.690 (1.04), 1.695 (1.27), 1.701 (1.21), 1.707 (1.17), 1.712 (0.75), 1.724 (1.15), 1.755 (1.41), 1.760 (1.09), 1.765 (0.93), 1.771 (1.00), 1.781 (1.30), 1.786 (1.07), 1.791 (1.09), 1.797 (1.00), 1.802 (0.81), 1.807 (0.61), 1.976 (0.47), 1.987 (0.96), 2.007 (0.43), 2.019 (0.45), 2.283 (0.47), 2.291 (0.54), 2.303 (0.80), 2.315 (0.53), 2.323 (0.79), 2.327 (0.58), 2.331 (0.40), 2.518 (1.55), 2.523 (1.01), 2.669 (0.47), 3.218 (0.72), 3.224 (0.86), 3.248 (0.93), 3.250 (0.95), 3.253 (1.13), 3.255 (1.13), 3.260 (0.66), 3.276 (0.86), 3.280 (1.24), 3.285 (1.19), 3.291 (1.08), 3.296 (0.83), 3.303 (0.87), 3.308 (0.97), 3.319 (1.34), 3.354 (1.04), 3.367 (1.15), 3.371 (1.75), 3.384 (1.70), 3.386 (0.99), 3.400 (0.91), 3.458 (0.63), 3.460 (0.47), 3.470 (0.71), 3.481 (0.59), 3.493 (1.16), 3.500 (0.73), 3.505 (0.88), 3.521 (1.15), 3.532 (1.29), 3.538 (0.69), 3.544 (0.81), 3.548 (0.93), 3.555 (1.15), 3.560 (1.06), 3.566 (0.90), 3.571 (1.08), 3.580 (0.96), 3.584 (0.87), 3.596 (0.55), 3.658 (0.65), 3.663 (1.00), 3.667 (0.47), 3.675 (0.66), 3.678 (0.67), 3.682 (1.55), 3.687 (0.79), 3.694 (0.74), 3.699 (0.83), 3.701 (0.90), 3.709 (0.69), 3.713 (0.72), 3.717 (0.96), 3.722 (0.89), 3.729 (0.76), 3.734 (0.67), 3.745 (0.49), 3.750 (0.54), 3.770 (0.68), 3.774 (0.66), 3.782 (0.66), 3.786 (0.64), 3.799 (0.79), 3.803 (0.77), 3.811 (0.85), 3.815 (0.66), 3.829 (0.49), 3.930 (0.64), 3.941 (0.76), 3.948 (0.70), 3.955 (1.21), 3.964 (1.45), 4.310 (0.96), 4.322 (2.08), 4.335 (1.23), 4.537 (1.51), 4.544 (1.40), 4.587 (0.49), 4.599 (0.51), 4.630 (0.51), 4.644 (0.41), 4.655 (0.48), 4.689 (2.97), 4.701 (2.83), 5.746 (2.64), 8.311 (0.66), 8.318 (5.57), 8.332 (1.56), 8.341 (0.58).

### Intermediate 57

### exo/endo-tert-butyl 2-(8-oxabicyclo[3.2.1]octan-3-yl)hydrazine-1-carboxylate

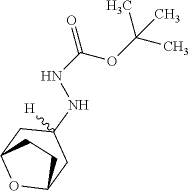 [SMILES: CC(C)(C)OC(=O)NNC1C[C@H]2CC[C@@H](C1)O2]

Step 1:

To a solution of 8-oxabicyclo[3.2.1]octan-3-one (2.0 g, 16 mmol, CAS-RN:[77745-32-], commercially available e.g. ABCR) in 13 mL methanol a solution of tert-butyl hydrazinecarboxylate (1.99 g, 15.1 mmol) in 13 mL methanol was added dropwise. The mixture was then stirred for 1 h at room temperature and directly used in the next step.

Step 2:

In analogy to intermediate 35) the product of the first step (3.80 g, 15.8 mmol) reacted and give without further purification 3.52 g (92% yield) of the desired title compound as raw product.

### Intermediate 58

### exo/endo-(8-oxabicyclo[3.2.1]octan-3-yl)hydrazine-hydrogen chloride (1/1)

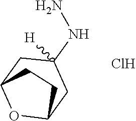

In analogy to intermediate 36) exo/endo-tert-butyl 2-(8-oxabicyclo[3.2.1]octan-3-yl)hydrazine-1-carboxylate (1.50 g, 6.19 mmol, intermediate 57) reacted and give without further purification 1.20 g (109% yield) of the desired title compound as hygroscopic raw product.

### Intermediate 59

### exo/endo-6-cyclopropyl-1-(8-oxabicyclo[3.2.1]octan-3-yl)-1H-pyrazolo[3,4-b]pyrazin-3-amine

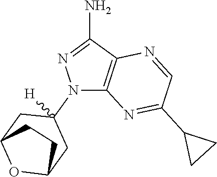 [SMILES: Nc1nn(C2C[C@H]3CC[C@@H](C2)O3)c2nc(C3CC3)cnc12]

In analogy to example 37) 3-chloro-5-cyclopropylpyrazine-2-carbonitrile (275 mg, 1.54 mmol, intermediate 1) and exo/endo-(8-oxabicyclo[3.2.1]octan-3-yl)hydrazine-hydrogen chloride (1/1) (1.10 g, 6.16 mmol, intermediate 58) reacted and give after one purification using two subsequent Biotage chromatography system 434 mg (44% purity, 43% yield) of the desired title compound.

LC-MS (Method 2): Rₜ=0.96 min; MS (ESIpos): m/z=286 [M+H]⁺

¹H-NMR (400 MHz, DMSO-d6) δ [ppm]: 1.002 (0.41), 1.006 (0.49), 1.016 (0.57), 1.023 (0.50), 1.028 (0.58), 1.034 (0.50), 1.042 (0.71), 1.049 (0.90), 1.054 (0.87), 1.061 (0.95), 1.068 (0.64), 1.076 (0.73), 1.083 (0.92), 1.089 (0.53), 1.096 (0.68), 1.103 (0.83), 1.109 (0.45), 1.172 (0.66), 1.259 (0.54), 1.379 (11.80), 1.426 (0.48), 1.434 (0.58), 1.453 (1.21), 1.458 (1.21), 1.476 (0.64), 1.495 (0.77), 1.520 (0.59), 1.523 (0.60), 1.556 (0.89), 1.559 (0.86), 1.562 (0.67), 1.567 (0.73), 1.583 (0.87), 1.600 (0.83), 1.607 (1.08), 1.624 (0.71), 1.636 (0.74), 1.643 (0.66), 1.654 (0.99), 1.669 (1.19), 1.683 (2.47), 1.686 (2.44), 1.698 (2.61), 1.705 (2.02), 1.714 (2.54), 1.733 (1.36), 1.746 (1.48), 1.759 (1.23), 1.770 (1.36), 1.784 (2.05), 1.794 (2.29), 1.803 (1.89), 1.814 (1.49), 1.827 (1.47), 1.838 (1.44), 1.852 (1.49), 1.863 (1.19), 1.876 (2.01), 1.887 (0.88), 1.915 (1.32), 1.936 (0.80), 1.943 (1.41), 1.961 (1.02), 1.966 (1.04), 1.977 (1.00), 1.983 (1.30), 1.987 (1.62), 2.007 (1.71), 2.043 (1.80), 2.153 (0.64), 2.168 (1.34), 2.185 (1.57), 2.206 (0.49), 2.299 (0.43), 2.418 (0.61), 2.427 (0.62), 2.454 (0.52), 2.464 (0.55), 2.518 (0.88), 2.523 (0.69), 2.596 (1.11), 2.634 (0.99), 3.250 (0.56), 3.264 (0.96), 3.279 (0.52), 3.682 (0.97), 4.130 (0.47), 4.186 (1.96), 4.250 (0.48), 4.257 (0.53), 4.283 (0.42), 4.422 (1.72), 4.472 (0.51), 4.478 (0.52), 4.481 (0.54), 4.495 (0.44), 5.427 (1.38), 5.744 (1.25), 5.758 (16.00), 7.822 (0.59), 8.301 (2.54), 8.336 (0.71).

### Intermediate 60

### trans-5-cyclopropyl-3-{[3-(hydroxymethyl)cyclobutyl]amino}pyrazine-2-carbonitrile

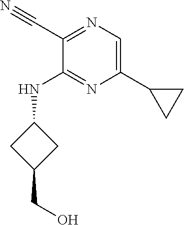 [SMILES: N#Cc1ncc(C2CC2)nc1N[C@H]1C[C@H](CO)C1]

In analogy to 39) 3-chloro-5-cyclopropylpyrazine-2-carbonitrile (250 mg, 1.18 mmol, intermediate 1) and (trans-3-aminocyclobutyl)methanol-hydrogen chloride (1/1) (179 mg, 1.30 mmol, CAS-RN:[1284250-10-7], commercially available e.g. ABCR) reacted and give after purification using a Biotage chromatography 232 mg (90% purity, 72% yield) of the desired title compound.

LC-MS (Method 2): Rₜ=0.95 min; MS (ESIpos): m/z=245 [M+H]⁺

¹H-NMR (400 MHz, DMSO-d6) δ [ppm]: 0.960 (1.14), 0.966 (1.30), 0.970 (2.93), 0.977 (4.75), 0.982 (3.19), 0.984 (2.90), 0.989 (4.05), 0.996 (2.00), 1.008 (0.56), 1.043 (1.65), 1.051 (3.62), 1.058 (2.15), 1.063 (2.64), 1.071 (4.04), 1.078 (2.39), 1.082 (1.32), 1.089 (1.03), 2.010 (0.86), 2.019 (1.16), 2.025 (0.78), 2.031 (1.08), 2.041 (2.45), 2.050 (1.98), 2.055 (1.29), 2.062 (2.32), 2.070 (2.04), 2.075 (1.74), 2.083 (1.45), 2.095 (2.46), 2.107 (2.35), 2.115 (1.77), 2.130 (2.71), 2.149 (1.96), 2.155 (2.07), 2.160 (1.43), 2.175 (0.89), 2.181 (1.02), 2.213 (0.45), 2.221 (0.69), 2.230 (0.94), 2.237 (0.97), 2.246 (0.89), 2.252 (1.05), 2.267 (0.64), 2.327 (0.53), 2.518 (1.52), 2.523 (1.03), 2.669 (0.44), 2.729 (0.66), 2.888 (0.75), 3.433 (4.25), 3.447 (5.02), 3.450 (5.21), 3.463 (4.73), 3.480 (0.42), 3.896 (0.68), 4.287 (0.81), 4.306 (1.32), 4.323 (1.29), 4.342 (0.75), 4.573 (2.91), 4.586 (6.73), 4.599 (2.77), 4.607 (0.41), 5.758 (1.78), 7.579 (2.16), 7.594 (2.10), 7.755 (0.78), 7.900 (16.00).

### Intermediate 61

### [trans-3-(3-amino-6-cyclopropyl-1H-pyrazolo[3,4-b]pyrazin-1-yl)cyclobutyl]methanol

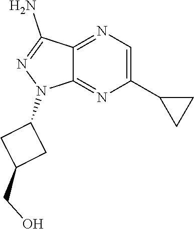 [SMILES: Nc1nn([C@H]2C[C@H](CO)C2)c2nc(C3CC3)cnc12]

In analogy to 42) trans-5-cyclopropyl-3-{[3-(hydroxymethyl)cyclobutyl]amino}pyrazine-2-carbonitrile (232 mg, 950 μmol, intermediate 60) and O-(diphenylphosphinoyl)hydroxylamine (299 mg, 1.28 mmol) reacted and give after purification using a Biotage chromatography 314 mg (90% purity, 115% yield) of the desired title compound.

LC-MS (Method 2): Rₜ=0.80 min; MS (ESIpos): m/z=260 [M+H]⁺

¹H-NMR (400 MHz, DMSO-d6) δ [ppm]: 1.000 (0.55), 1.004 (0.58), 1.014 (0.91), 1.026 (3.05), 1.033 (4.22), 1.039 (4.52), 1.045 (3.99), 1.052 (2.95), 1.062 (2.55), 1.068 (4.02), 1.075 (2.34), 1.082 (3.23), 1.089 (4.50), 1.096 (2.35), 1.108 (1.09), 1.154 (0.42), 1.172 (0.78), 1.230 (0.43), 1.987 (1.32), 2.144 (1.23), 2.152 (1.57), 2.159 (1.08), 2.165 (1.51), 2.175 (2.38), 2.184 (1.68), 2.198 (1.69), 2.205 (1.36), 2.263 (0.67), 2.276 (1.38), 2.284 (1.44), 2.291 (1.65), 2.296 (2.44), 2.304 (1.15), 2.308 (1.53), 2.315 (1.55), 2.323 (1.05), 2.327 (1.48), 2.331 (1.08), 2.337 (0.72), 2.349 (0.68), 2.356 (0.79), 2.363 (0.97), 2.371 (0.80), 2.379 (0.78), 2.387 (0.66), 2.518 (2.39), 2.523 (1.57), 2.584 (1.32), 2.591 (0.84), 2.605 (1.79), 2.609 (2.20), 2.615 (1.84), 2.629 (1.75), 2.635 (2.17), 2.653 (0.79), 2.659 (1.42), 2.665 (1.05), 2.669 (1.07), 2.673 (0.83), 3.159 (0.43), 3.172 (0.43), 3.530 (3.80), 3.544 (4.94), 3.545 (4.80), 3.559 (3.69), 3.957 (0.47), 4.702 (2.46), 4.715 (6.21), 4.728 (2.39), 5.136 (1.35), 5.156 (1.99), 5.177 (1.32), 5.759 (4.52), 5.818 (7.10), 5.843 (0.79), 7.552 (0.59), 7.555 (0.46), 7.561 (0.59), 7.571 (0.46), 7.579 (0.43), 7.607 (0.42), 7.756 (0.72), 7.792 (0.49), 7.796 (0.60), 7.813 (0.52), 7.823 (0.49), 7.826 (0.56), 7.843 (0.48), 8.315 (16.00), 8.324 (1.62).

### Intermediate 62

### cis-5-cyclopropyl-3-{[3-(hydroxymethyl)cyclobutyl]amino}pyrazine-2-carbonitrile

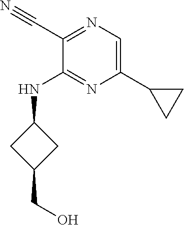 [SMILES: N#Cc1ncc(C2CC2)nc1N[C@H]1C[C@@H](CO)C1]

In analogy to 39) 3-chloro-5-cyclopropylpyrazine-2-carbonitrile (250 mg, 1.18 mmol, intermediate 1) and (cis-3-aminocyclobutyl)methanol-hydrogen chloride (1/1) (132 mg, 1.30 mmol, CAS-RN:[142733-65-1], commercially available e.g. ABCR) reacted and give after purification using a Biotage chromatography 168 mg (95% purity, 55% yield) of the desired title compound.

LC-MS (Method 2): Rₜ=1.01 min; MS (ESIpos): m/z=254 [M+H]⁺

¹H-NMR (400 MHz, DMSO-d6) δ [ppm]: 0.970 (1.16), 0.982 (3.19), 0.988 (5.29), 0.992 (3.34), 0.995 (3.12), 1.000 (4.22), 1.007 (2.12), 1.013 (0.67), 1.031 (0.63), 1.047 (1.87), 1.055 (4.01), 1.062 (2.47), 1.067 (2.89), 1.075 (4.70), 1.082 (2.72), 1.087 (1.35), 1.093 (1.18), 1.748 (1.01), 1.755 (0.77), 1.771 (2.75), 1.778 (2.44), 1.794 (2.63), 1.800 (3.05), 1.817 (0.98), 1.822 (1.36), 1.987 (0.67), 2.037 (0.44), 2.056 (0.85), 2.061 (0.81), 2.066 (1.14), 2.072 (1.11), 2.078 (2.29), 2.086 (1.99), 2.091 (1.50), 2.097 (3.11), 2.109 (1.78), 2.118 (1.65), 2.129 (0.66), 2.211 (1.40), 2.218 (1.16), 2.230 (2.49), 2.237 (2.41), 2.252 (2.17), 2.258 (2.53), 2.270 (0.93), 2.277 (1.16), 2.518 (1.68), 2.523 (1.09), 2.728 (1.24), 2.888 (1.51), 3.345 (4.93), 3.359 (7.31), 3.374 (4.64), 4.144 (0.75), 4.147 (0.76), 4.166 (1.40), 4.182 (1.41), 4.201 (0.74), 4.205 (0.70), 4.453 (3.21), 4.467 (7.37), 4.480 (3.07), 5.758 (0.78), 7.545 (2.30), 7.562 (2.26), 7.897 (16.00).

### Intermediate 63

### [cis-3-(3-amino-6-cyclopropyl-1H-pyrazolo[3,4-b]pyrazin-1-yl)cyclobutyl]methanol

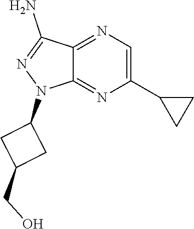 [SMILES: Nc1nn([C@H]2C[C@@H](CO)C2)c2nc(C3CC3)cnc12]

In analogy to 42) cis-5-cyclopropyl-3-{[3-(hydroxymethyl)cyclobutyl]amino}pyrazine-2-carbonitrile (168 mg, 688 μmol, intermediate 62) and O-(diphenylphosphinoyl)hydroxylamine (216 mg, 928 μmol) reacted and give after purification using a Biotage chromatography 55.0 mg (95% purity, 29% yield) of the desired title compound.

LC-MS (Method 2): Rₜ=0.84 min; MS (ESIpos): m/z=260 [M+H]⁺

¹H-NMR (400 MHz, DMSO-d6) δ [ppm]: 1.024 (0.75), 1.036 (2.91), 1.043 (4.51), 1.048 (4.64), 1.055 (4.16), 1.061 (3.07), 1.068 (2.50), 1.074 (4.23), 1.081 (2.21), 1.088 (2.95), 1.094 (4.67), 1.101 (2.41), 1.114 (0.94), 1.171 (0.40), 1.987 (0.59), 2.178 (0.62), 2.184 (0.57), 2.198 (1.04), 2.205 (0.70), 2.213 (0.68), 2.220 (1.10), 2.238 (0.66), 2.267 (0.88), 2.279 (1.53), 2.292 (7.18), 2.299 (3.11), 2.312 (10.81), 2.332 (4.81), 2.474 (0.44), 2.518 (2.04), 2.523 (1.30), 2.664 (0.41), 2.669 (0.56), 2.673 (0.42), 3.428 (3.99), 3.442 (6.77), 3.456 (−3.77), 4.550 (2.61), 4.563 (6.03), 4.577 (2.52), 4.903 (0.47), 4.925 (1.93), 4.946 (2.75), 4.966 (1.66), 4.988 (0.45), 5.758 (4.31), 5.796 (7.13), 8.323 (16.00).

### Intermediate 64

### 5-cyclopropyl-3-{[cis/trans-3-(2-hydroxypropan-2-yl)cyclobutyl]amino}pyrazine-2-carbonitrile

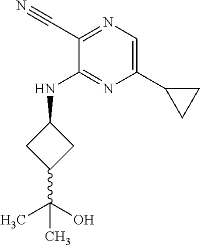

In analogy to 39) 3-chloro-5-cyclopropylpyrazine-2-carbonitrile (250 mg, 1.18 mmol, intermediate 1) and cis/trans-2-(3-aminocyclobutyl)propan-2-ol (168 mg, 1.30 mmol, CAS-RN:[1609546-13-5], commercially available e.g. Enamine) reacted and give after purification using a Biotage chromatography 196 mg (95% purity, 58% yield) of the desired title compound.

LC-MS (Method 2): Rₜ=1.14 min; MS (ESIpos): m/z=273 [M+H]⁺

¹H-NMR (400 MHz, DMSO-d6) δ [ppm]: 0.971 (0.48), 0.982 (1.20), 0.993 (16.00), 1.007 (0.71), 1.030 (1.93), 1.046 (0.59), 1.054 (1.09), 1.061 (0.65), 1.066 (0.83), 1.073 (1.21), 1.081 (0.67), 1.897 (0.46), 1.915 (1.20), 1.931 (1.01), 2.079 (0.52), 2.087 (0.62), 2.090 (0.56), 2.099 (1.07), 2.107 (0.87), 2.110 (0.84), 2.118 (0.82), 2.125 (0.64), 2.518 (0.64), 2.523 (0.41), 2.728 (1.68), 2.888 (2.00), 4.101 (4.07), 4.192 (0.46), 7.472 (0.64), 7.488 (0.63), 7.898 (4.14).

### Intermediate 65

### 2-[cis/trans-3-(3-amino-6-cyclopropyl-1H-pyrazolo[3,4-b]pyrazin-1-yl)cyclobutyl]propan-2-ol

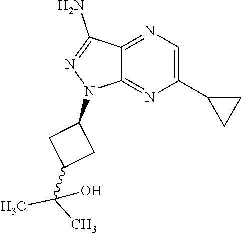

In analogy to 42) 5-cyclopropyl-3-{[cis/trans-3-(2-hydroxypropan-2-yl)cyclobutyl]amino}pyrazine-2-carbonitrile (196 mg, 720 μmol, intermediate 64) and O-(diphenylphosphinoyl)hydroxylamine (227 mg, 972 μmol) reacted and give after purification using a Biotage chromatography 80.0 mg (90% purity, 35% yield) of the desired title compound.

LC-MS (Method 2): Rₜ=1.02 min; MS (ESIpos): m/z=289 [M+H]⁺

¹H-NMR (400 MHz, DMSO-d6) δ [ppm]: 1.030 (0.42), 1.048 (16.00), 1.058 (1.89), 1.064 (1.74), 1.069 (1.66), 1.076 (0.84), 1.083 (2.82), 1.089 (1.63), 1.095 (0.70), 1.171 (0.43), 1.987 (0.88), 2.030 (0.43), 2.118 (0.66), 2.123 (0.71), 2.136 (0.59), 2.142 (0.70), 2.282 (0.42), 2.295 (0.71), 2.326 (0.41), 2.462 (0.40), 2.518 (1.01), 2.522 (0.59), 4.188 (4.18), 4.397 (0.46), 4.796 (0.57), 5.741 (2.08), 5.758 (0.51), 8.306 (0.69), 8.315 (4.70).

### Intermediate 66

### 3-(cyclobutylamino)-5-cyclopropylpyrazine-2-carbonitrile

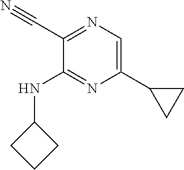 [SMILES: N#Cc1ncc(C2CC2)nc1NC1CCC1]

In analogy to 39) 3-chloro-5-cyclopropylpyrazine-2-carbonitrile (400 mg, 2.23 mmol, intermediate 1) and cyclobutanamine (174 mg, 2.45 mmol) reacted and give after purification using a Biotage chromatography 318 mg (78% purity, 52% yield) of the desired title compound.

LC-MS (Method 2): Rₜ=1.33 min; MS (ESIpos): m/z=215 [M+H]⁺

¹H-NMR (400 MHz, DMSO-d6) δ [ppm]: 0.969 (1.06), 0.974 (1.11), 0.980 (3.11), 0.987 (5.45), 0.991 (3.34), 0.994 (2.82), 0.998 (4.91), 1.005 (2.48), 1.017 (1.38), 1.021 (0.87), 1.029 (1.54), 1.037 (0.53), 1.041 (0.51), 1.047 (1.79), 1.055 (3.90), 1.062 (2.23), 1.067 (2.69), 1.074 (4.47), 1.082 (2.62), 1.086 (1.14), 1.093 (1.07), 1.118 (0.40), 1.126 (0.98), 1.134 (0.65), 1.138 (0.51), 1.147 (1.38), 1.154 (1.66), 1.162 (0.70), 1.174 (1.12), 1.182 (0.72), 1.623 (0.61), 1.642 (1.43), 1.650 (1.33), 1.660 (0.95), 1.667 (2.85), 1.674 (2.14), 1.684 (2.13), 1.693 (1.54), 1.696 (1.21), 1.698 (1.11), 1.708 (1.19), 2.033 (0.47), 2.040 (0.43), 2.056 (1.59), 2.062 (1.84), 2.067 (1.28), 2.079 (3.21), 2.085 (3.01), 2.087 (3.60), 2.099 (2.99), 2.104 (1.91), 2.107 (2.64), 2.110 (3.20), 2.119 (1.39), 2.126 (0.72), 2.131 (0.97), 2.141 (0.46), 2.146 (1.01), 2.153 (0.93), 2.164 (1.74), 2.169 (1.68), 2.173 (1.95), 2.179 (1.36), 2.181 (1.73), 2.187 (1.66), 2.191 (1.61), 2.202 (0.97), 2.209 (0.54), 2.212 (0.61), 2.220 (0.51), 2.245 (0.42), 2.256 (0.73), 2.298 (0.42), 2.318 (0.74), 2.326 (0.51), 2.330 (0.45), 2.518 (0.87), 2.522 (0.55), 3.154 (0.43), 3.189 (0.61), 3.905 (1.03), 4.282 (0.71), 4.301 (1.27), 4.320 (1.28), 4.340 (0.65), 7.503 (0.41), 7.587 (1.58), 7.604 (1.53), 7.760 (0.43), 7.903 (16.00), 8.554 (3.37).

### Intermediate 67

### 1-cyclobutyl-6-cyclopropyl-1H-pyrazolo[3,4-b]pyrazin-3-amine

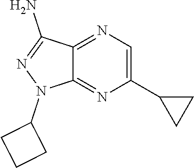 [SMILES: Nc1nn(C2CCC2)c2nc(C3CC3)cnc12]

In analogy to 42) 3-(cyclobutylamino)-5-cyclopropylpyrazine-2-carbonitrile (318 mg, 1.19 mmol, intermediate 66) and O-(diphenylphosphinoyl)hydroxylamine (375 mg, 1.60 mmol) reacted and give after purification using a Biotage chromatography 540 mg (45% purity, 89% yield) of the desired title compound.

LC-MS (Method 2): Rₜ=1.09 min; MS (ESIpos): m/z=230 [M+H]⁺

¹H-NMR (400 MHz, DMSO-d6) δ [ppm]: 1.032 (0.18), 1.039 (0.22), 1.045 (0.22), 1.051 (0.21), 1.057 (0.17), 1.072 (0.22), 1.085 (0.17), 1.092 (0.23), 2.298 (0.20), 2.729 (14.09), 2.888 (16.00), 2.897 (0.18), 5.802 (0.32), 7.903 (0.23), 7.950 (1.96), 8.323 (0.76), 8.555 (0.20).

### Intermediate 68

### 5-cyclopropyl-3-[(trans-4-hydroxycyclohexyl)amino]pyrazine-2-carbonitrile

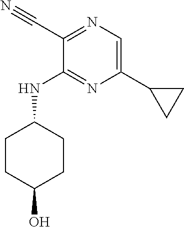 [SMILES: N#Cc1ncc(C2CC2)nc1N[C@H]1CC[C@H](O)CC1]

In analogy to 39) 3-chloro-5-cyclopropylpyrazine-2-carbonitrile (410 mg, 1.94 mmol, intermediate 1) and trans-4-aminocyclohexan-1-ol (246 mg, 2.13 mmol, CAS-RN:[27489-62-9], commercially available e.g. Acros Organics) reacted and give after purification using a Biotage chromatography 360 mg (95% purity, 68% yield) of the desired title compound.

LC-MS (Method 2): Rₜ=0.98 min; MS (ESIpos): m/z=259 [M+H]⁺

¹H-NMR (400 MHz, DMSO-d6) δ [ppm]: 0.958 (0.94), 0.969 (2.73), 0.976 (4.38), 0.979 (2.84), 0.983 (2.50), 0.987 (3.71), 0.995 (1.68), 1.050 (1.50), 1.058 (3.43), 1.066 (2.12), 1.070 (2.37), 1.078 (3.95), 1.085 (2.33), 1.096 (1.01), 1.154 (0.89), 1.172 (1.27), 1.183 (1.61), 1.190 (1.71), 1.215 (1.86), 1.241 (0.98), 1.249 (0.88), 1.364 (0.66), 1.371 (0.72), 1.400 (1.63), 1.433 (1.70), 1.458 (0.68), 1.765 (1.72), 1.808 (1.73), 1.820 (1.71), 1.849 (1.64), 1.987 (1.87), 2.071 (0.54), 2.082 (1.15), 2.090 (1.25), 2.102 (2.20), 2.114 (1.15), 2.122 (1.07), 2.133 (0.44), 2.518 (1.64), 2.522 (1.05), 2.727 (1.11), 2.729 (1.06), 2.888 (1.31), 3.349 (0.81), 3.365 (1.15), 3.375 (1.18), 3.386 (0.62), 3.391 (0.59), 3.402 (0.50), 3.692 (0.41), 3.701 (0.78), 3.711 (0.98), 3.720 (0.78), 3.730 (0.98), 3.739 (0.76), 3.748 (0.40), 3.900 (1.46), 4.017 (0.41), 4.541 (6.38), 4.552 (5.96), 5.758 (16.00), 7.057 (2.20), 7.076 (2.13), 7.485 (0.63), 7.876 (15.20).

### Intermediate 69

### trans-4-(3-amino-6-cyclopropyl-1H-pyrazolo[3,4-b]pyrazin-1-yl)cyclohexan-1-ol

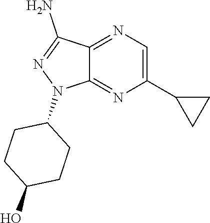 [SMILES: Nc1nn([C@H]2CC[C@H](O)CC2)c2nc(C3CC3)cnc12]

In analogy to 42) 5-cyclopropyl-3-[(trans-4-hydroxycyclohexyl)amino]pyrazine-2-carbonitrile (360 mg, 1.39 mmol, intermediate 68) and O-(diphenylphosphinoyl)hydroxylamine (439 mg, 1.88 mmol) reacted and give after purification using a Biotage chromatography 217 mg (95% purity, 54% yield) of the desired title compound.

LC-MS (Method 2): Rₜ=0.79 min; MS (ESIpos): m/z=274 [M+H]⁺

¹H-NMR (400 MHz, DMSO-d6) δ [ppm]: 1.017 (0.85), 1.030 (3.05), 1.036 (4.01), 1.042 (4.48), 1.048 (3.87), 1.055 (2.48), 1.066 (2.32), 1.073 (3.71), 1.080 (2.16), 1.086 (2.69), 1.093 (4.25), 1.100 (2.26), 1.112 (1.07), 1.307 (0.52), 1.335 (1.58), 1.343 (1.42), 1.370 (1.79), 1.396 (0.73), 1.751 (1.40), 1.774 (2.01), 1.860 (0.76), 1.867 (0.98), 1.900 (3.91), 1.927 (3.67), 1.987 (0.54), 2.262 (0.56), 2.274 (1.10), 2.282 (1.20), 2.291 (1.13), 2.294 (2.01), 2.303 (0.88), 2.306 (1.16), 2.314 (1.07), 2.323 (0.47), 2.327 (0.83), 2.518 (1.53), 2.523 (1.03), 2.669 (0.43), 3.435 (0.49), 3.445 (0.61), 3.461 (1.03), 3.471 (1.05), 3.481 (0.61), 3.488 (0.56), 3.499 (0.48), 3.963 (1.22), 4.337 (0.60), 4.356 (0.84), 4.366 (1.17), 4.376 (0.60), 4.384 (0.56), 4.395 (0.58), 4.647 (5.64), 4.658 (5.46), 5.696 (7.00), 5.758 (3.13), 7.919 (0.71), 8.293 (16.00).

### Intermediate 70

### 5-cyclopropyl-3-[(cis-4-hydroxycyclohexyl)amino]pyrazine-2-carbonitrile

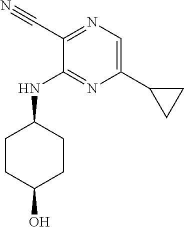 [SMILES: N#Cc1ncc(C2CC2)nc1N[C@H]1CC[C@@H](O)CC1]

In analogy to 39) 3-chloro-5-cyclopropylpyrazine-2-carbonitrile (410 mg, 1.94 mmol, intermediate 1) and cis-4-aminocyclohexan-1-ol-hydrogen chloride (1/1) (324 mg, 2.13 mmol, CAS-RN:[56239-26-0], commercially available e.g. Fluorochem) reacted and give after purification using a Biotage chromatography 290 mg (95% purity, 55% yield) of the desired title compound.

LC-MS (Method 2): Rₜ=1.07 min; MS (ESIpos): m/z=259 [M+H]⁺

¹H-NMR (400 MHz, DMSO-d6) δ [ppm]: 0.953 (0.98), 0.964 (2.90), 0.971 (4.85), 0.976 (3.00), 0.978 (2.71), 0.983 (3.98), 0.990 (1.85), 1.038 (1.62), 1.045 (3.75), 1.052 (2.23), 1.057 (2.58), 1.065 (4.26), 1.073 (2.46), 1.083 (1.02), 1.421 (0.52), 1.430 (0.92), 1.437 (0.71), 1.461 (2.05), 1.499 (2.44), 1.508 (1.64), 1.518 (1.50), 1.529 (1.38), 1.539 (1.96), 1.549 (1.90), 1.622 (1.86), 1.632 (1.76), 1.643 (1.26), 1.655 (1.44), 1.665 (1.33), 1.737 (0.80), 1.744 (0.89), 1.767 (1.89), 1.793 (1.67), 1.800 (1.48), 1.821 (0.63), 1.831 (0.58), 2.066 (0.58), 2.078 (1.21), 2.086 (1.33), 2.098 (2.37), 2.106 (0.95), 2.109 (1.24), 2.118 (1.15), 2.129 (0.48), 2.518 (1.56), 2.522 (0.98), 3.751 (2.15), 3.758 (2.00), 3.776 (1.09), 3.784 (0.78), 3.792 (0.43), 3.891 (1.68), 4.354 (5.10), 4.361 (5.03), 5.758 (0.93), 6.999 (1.95), 7.017 (1.91), 7.487 (0.73), 7.882 (16.00).

### Intermediate 71

### cis-4-(3-amino-6-cyclopropyl-1H-pyrazolo[3,4-b]pyrazin-1-yl)cyclohexan-1-ol

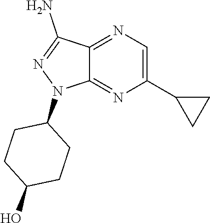 [SMILES: Nc1nn([C@H]2CC[C@@H](O)CC2)c2nc(C3CC3)cnc12]

In analogy to 42) 5-cyclopropyl-3-[(cis-4-hydroxycyclohexyl)amino]pyrazine-2-carbonitrile (290 mg, 1.12 mmol, intermediate 70) and O-(diphenylphosphinoyl)hydroxylamine (353 mg, 1.52 mmol) reacted and give after purification using a Biotage chromatography 206 mg (93% purity, 62% yield) of the desired title compound.

LC-MS (Method 2): Rₜ=0.90 min; MS (ESIpos): m/z=274 [M+H]⁺

¹H-NMR (400 MHz, DMSO-d6) δ [ppm]: 1.009 (0.77), 1.021 (2.83), 1.028 (3.74), 1.033 (4.10), 1.040 (3.58), 1.047 (2.24), 1.059 (2.15), 1.065 (3.47), 1.071 (1.97), 1.078 (2.40), 1.085 (3.95), 1.092 (2.06), 1.104 (1.02), 1.480 (1.42), 1.489 (1.61), 1.499 (1.06), 1.511 (1.58), 1.520 (1.64), 1.539 (0.88), 1.545 (0.94), 1.579 (1.98), 1.612 (1.32), 1.745 (1.79), 1.752 (1.77), 1.775 (1.29), 1.784 (1.28), 1.987 (0.52), 2.244 (0.55), 2.258 (0.84), 2.271 (1.79), 2.278 (2.39), 2.286 (2.40), 2.290 (2.41), 2.303 (2.18), 2.310 (2.22), 2.322 (1.14), 2.326 (0.85), 2.332 (0.71), 2.336 (0.80), 2.344 (0.52), 2.518 (1.83), 2.522 (1.19), 2.668 (0.54), 3.158 (0.89), 3.171 (1.03), 3.851 (1.56), 3.857 (1.56), 3.953 (1.10), 4.365 (0.76), 4.374 (0.54), 4.388 (4.78), 4.394 (5.27), 4.402 (0.97), 4.411 (0.49), 4.421 (0.68), 5.672 (6.31), 5.758 (2.32), 7.917 (0.61), 8.290 (16.00).

### Intermediate 72

### 5-cyclopropyl-3-[(2,2,6,6-tetramethyloxan-4-yl)amino]pyrazine-2-carbonitrile

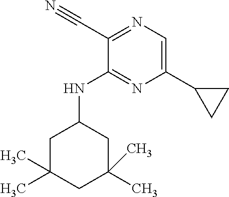 [SMILES: CC1(C)CC(Nc2nc(C3CC3)cnc2C#N)CC(C)(C)C1]

In analogy to 39) 3-chloro-5-cyclopropylpyrazine-2-carbonitrile (400 mg, 2.12 mmol, intermediate 1) and 2,2,6,6-tetramethyloxan-4-amine (366 mg, 2.33 mmol, CAS-RN:[1301178-43-7], commercially available e.g. ABCR) reacted and give after purification using a Biotage chromatography 509 mg (75% purity, 60% yield) of the desired title compound.

LC-MS (Method 2): Rₜ=1.40 min; MS (ESIpos): m/z=302 [M+H]⁺

¹H-NMR (400 MHz, DMSO-d6) δ [ppm]: 0.990 (0.54), 1.001 (1.22), 1.008 (1.85), 1.012 (1.44), 1.014 (1.30), 1.019 (1.74), 1.026 (0.88), 1.055 (0.81), 1.062 (1.71), 1.069 (0.98), 1.075 (1.37), 1.083 (2.32), 1.093 (1.78), 1.100 (1.07), 1.103 (1.01), 1.109 (0.69), 1.126 (16.00), 1.136 (2.22), 1.163 (0.87), 1.197 (1.10), 1.213 (0.44), 1.251 (15.28), 1.263 (0.84), 1.272 (1.80), 1.284 (1.22), 1.291 (0.50), 1.322 (0.87), 1.353 (1.75), 1.383 (0.97), 1.727 (1.28), 1.736 (1.31), 1.758 (1.11), 1.767 (1.05), 2.102 (0.48), 2.110 (0.51), 2.121 (0.90), 2.133 (0.56), 2.141 (0.48), 2.518 (0.42), 2.727 (5.71), 2.888 (6.69), 3.077 (0.57), 3.189 (4.23), 3.919 (1.28), 3.969 (0.54), 7.218 (0.95), 7.236 (0.93), 7.523 (0.52), 7.941 (7.18), 7.950 (0.84), 7.995 (0.57), 8.835 (1.16).

### Intermediate 73

### 6-cyclopropyl-1-(2 ,2,6,6-tetramethyloxan-4-yl)-1H-pyrazolo[3,4-b]pyrazin-3-amine

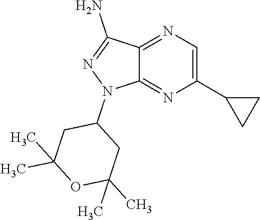 [SMILES: CC1(C)CC(n2nc(N)c3ncc(C4CC4)nc32)CC(C)(C)O1]

In analogy to 42) 5-cyclopropyl-3-[(2,2,6,6-tetramethyloxan-4-yl)amino]pyrazine-2-carbonitrile (509 mg, 1.27 mmol, intermediate 72) and O-(diphenylphosphinoyl)hydroxylamine (400 mg, 1.72 mmol) reacted 2 hours at room temperature and 16 hours at 50° C. and give after purification using a Biotage chromatography 320 mg (80% purity, 64% yield) of the desired title compound.

LC-MS (Method 2): Rₜ=1.22 min; MS (ESIpos): m/z=316 [M+H]⁺

¹H-NMR (400 MHz, DMSO-d6) δ [ppm]: 0.990 (0.73), 0.996 (0.71), 1.001 (0.75), 1.008 (0.52), 1.012 (0.60), 1.016 (0.70), 1.025 (0.65), 1.038 (1.48), 1.044 (1.95), 1.050 (2.10), 1.056 (1.89), 1.063 (1.30), 1.078 (1.35), 1.085 (1.90), 1.093 (2.35), 1.098 (1.78), 1.105 (2.25), 1.112 (1.36), 1.126 (2.99), 1.137 (4.01), 1.154 (2.21), 1.170 (16.00), 1.181 (1.84), 1.189 (1.57), 1.197 (1.62), 1.217 (0.54), 1.230 (0.43), 1.251 (2.38), 1.273 (3.73), 1.295 (0.81), 1.317 (15.19), 1.330 (1.33), 1.736 (1.07), 1.746 (0.93), 1.768 (1.72), 1.778 (1.49), 1.824 (1.27), 1.855 (1.77), 1.886 (0.82), 1.986 (3.30), 2.286 (0.49), 2.294 (0.52), 2.302 (0.49), 2.306 (0.87), 2.318 (0.56), 2.326 (0.72), 2.518 (0.87), 2.522 (0.59), 2.729 (1.30), 2.888 (1.52), 3.189 (0.68), 3.974 (0.92), 4.016 (0.71), 4.034 (0.69), 4.938 (0.69), 5.750 (3.09), 5.758 (1.86), 7.798 (1.43), 7.943 (0.98), 7.951 (0.63), 8.322 (7.37).

### Intermediate 74

### 5-cyclopropyl-3-[(2-oxaspiro[3.3]heptan-6-yl)amino]pyrazine-2-carbonitrile

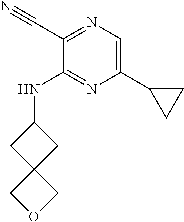 [SMILES: N#Cc1ncc(C2CC2)nc1NC1CC2(COC2)C1]

In analogy to 39) 3-chloro-5-cyclopropylpyrazine-2-carbonitrile (400 mg, 2.23 mmol, intermediate 1) and 2-oxaspiro[3.3]heptan-6-amine (277 mg, 2.45 mmol, CAS-RN:[1363381-78-5], commercially available e.g. Combi-Blocks Inc.) reacted and give after purification using a Biotage chromatography 510 mg (84% purity, 75% yield) of the desired title compound.

LC-MS (Method 2): Rₜ=1.07 min; MS (ESIneg): m/z=255 [M−H]⁻

¹H-NMR (400 MHz, DMSO-d6) δ [ppm]: 0.974 (0.84), 0.981 (1.36), 0.986 (0.86), 0.988 (0.78), 0.992 (1.15), 1.000 (0.55), 1.049 (0.46), 1.056 (1.03), 1.063 (0.63), 1.068 (0.72), 1.076 (1.19), 1.083 (0.72), 2.098 (0.67), 2.253 (0.59), 2.260 (0.40), 2.273 (0.63), 2.278 (0.62), 2.285 (0.76), 2.299 (0.47), 2.306 (0.76), 2.518 (0.92), 2.525 (0.51), 2.563 (0.43), 2.570 (0.57), 2.729 (13.33), 2.888 (16.00), 4.118 (0.51), 4.134 (0.49), 4.488 (4.76), 4.614 (4.81), 7.567 (0.57), 7.582 (0.57), 7.908 (4.52), 7.950 (1.96).

### Intermediate 75

### 6-cyclopropyl-1-(2-oxaspiro[3.3]heptan-6-yl)-1H-pyrazolo[3,4-b]pyrazin-3-amine

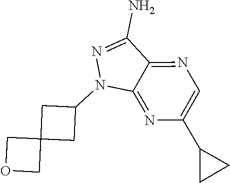 [SMILES: Nc1nn(C2CC3(COC3)C2)c2nc(C3CC3)cnc12]

In analogy to 42) 5-cyclopropyl-3-[(2-oxaspiro[3.3]heptan-6-yl)amino]pyrazine-2-carbonitrile (510 mg, 1.67 mmol, intermediate 74) and O-(diphenylphosphinoyl)hydroxylamine (526 mg, 2.26 mmol) reacted 16 hours at room temperature and 16 hours at 50° C. and give after purification using a Biotage chromatography 200 mg (76% purity, 34% yield) of the desired title compound.

LC-MS (Method 2): Rₜ=0.91 min; MS (ESIpos): m/z=272 [M+H]⁺

¹H-NMR (400 MHz, DMSO-d6) δ [ppm]: 0.991 (0.45), 1.005 (0.87), 1.010 (0.67), 1.017 (2.02), 1.024 (2.45), 1.030 (2.61), 1.036 (2.44), 1.043 (1.35), 1.048 (0.72), 1.060 (1.58), 1.067 (2.21), 1.074 (1.45), 1.080 (1.32), 1.087 (2.49), 1.093 (1.34), 1.105 (0.65), 2.272 (0.69), 2.280 (0.74), 2.288 (0.69), 2.292 (1.16), 2.300 (0.54), 2.304 (0.69), 2.312 (0.65), 2.518 (0.97), 2.523 (0.64), 2.647 (0.45), 2.651 (0.46), 2.676 (1.78), 2.679 (2.13), 2.684 (1.90), 2.686 (1.90), 2.691 (2.25), 2.696 (2.19), 2.699 (2.17), 2.704 (1.91), 2.706 (1.95), 2.711 (2.03), 2.727 (13.89), 2.739 (0.55), 2.888 (16.00), 4.489 (1.62), 4.508 (0.50), 4.573 (8.50), 4.629 (1.72), 4.689 (9.06), 4.946 (1.30), 4.966 (1.99), 4.987 (1.21), 5.827 (3.95), 7.768 (1.26), 7.950 (1.91), 8.326 (9.37).

### Intermediate 76

### 5-cyclopropyl-3-[(oxetan-3-yl)amino]pyrazine-2-carbonitrile

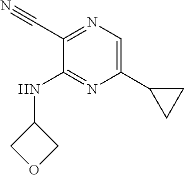 [SMILES: N#Cc1ncc(C2CC2)nc1NC1COC1]

In analogy to 39) 3-chloro-5-cyclopropylpyrazine-2-carbonitrile (400 mg, 2.23 mmol, intermediate 1) and oxetan-3-amine (179 mg, 2.45 mmol) reacted and give after purification using a Biotage chromatography 328 mg (76% purity, 52% yield) of the desired title compound.

LC-MS (Method 2): Rₜ=0.94 min; MS (ESIneg): m/z=215 [M−H]⁻

¹H-NMR (400 MHz, DMSO-d6) δ [ppm]: 0.948 (0.77), 0.955 (1.12), 0.959 (0.76), 0.963 (0.59), 0.967 (1.01), 0.974 (0.44), 1.056 (0.92), 1.064 (0.59), 1.068 (0.56), 1.076 (1.01), 1.083 (0.68), 2.105 (0.59), 2.729 (13.11), 2.888 (16.00), 4.555 (0.97), 4.571 (2.30), 4.588 (1.32), 4.670 (0.95), 4.688 (1.80), 4.703 (1.10), 7.847 (0.47), 7.950 (1.94), 7.988 (3.34), 8.077 (0.43), 8.087 (0.43).

### Intermediate 77

### 6-cyclopropyl-1-(oxetan-3-yl)-1H-pyrazolo[3,4-b]pyrazin-3-amine

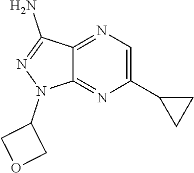 [SMILES: Nc1nn(C2COC2)c2nc(C3CC3)cnc12]

In analogy to 42) 5-cyclopropyl-3-[(oxetan-3-yl)amino]pyrazine-2-carbonitrile (328 mg, 1.15 mmol, intermediate 76) and O-(diphenylphosphinoyl)hydroxylamine (363 mg, 1.56 mmol) reacted 16 hours at room temperature and 16 hours at 50° C. and give after purification using a Biotage chromatography 234 mg (75% purity, 66% yield) of the desired title compound.

LC-MS (Method 2): Rₜ=0.81 min; MS (ESIpos): m/z=232 [M+H]⁺

¹H-NMR (400 MHz, DMSO-d6) δ [ppm]: 2.727 (12.77), 2.729 (14.49), 2.888 (16.00), 2.898 (0.22), 4.972 (0.25), 5.990 (0.17), 7.951 (2.01), 8.371 (0.41).

### Intermediate 78

### tert-butyl 2-(4-methoxybutan-2-ylidene)hydrazine-1-carboxylate

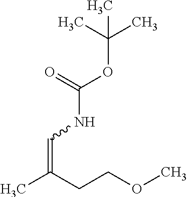

To a solution of 4-methoxybutan-2-one (300 mg, 2.94 mmol) in 2.0 mL methanol was added a solution of tert-butyl hydrazinecarboxylate (369 mg, 2.79 mmol) in 2.0 mL mthanol dropwise. The mixture was stirred for 1 h and directly used in the next step.

### Intermediate 79

### (±)-tert-butyl 2-(4-methoxybutan-2-yl)hydrazine-1-carboxylate

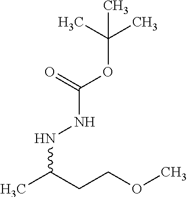

To the reaction mixture of tert-butyl 2-(4-methoxybutan-2-ylidene)hydrazine-1-carboxylate (640 mg, 2.96 mmol, Intermediate 78) 6.0 mL water and 2.0 mL acetic acid were added. The mixture was cooled to 0° C. and sodium cyanoborohydride (223 mg, 3.55 mmol) was added. The mixture was stirred over night at room temperature. The mixture was adjusted with a sodiumhydroxide solution (4.0 M) to pH=7 and extracted with dichloromethane thrice. The combined organic layers were washed with brine, filtered over a hydrophobic filter and concentrated under reduced pressure not to complete dryness (some solvent is left) to give 700 mg (85% purity, 92% yield) of the desired compound

¹H-NMR (400 MHz, DMSO-d6) δ [ppm]: 0.884 (3.38), 0.900 (3.40), 1.363 (0.61), 1.382 (16.00), 1.907 (4.45), 3.330 (1.96), 3.338 (0.66), 3.344 (0.59), 3.358 (0.92), 3.373 (0.58), 3.375 (0.56).

### Intermediate 80

### (±)-(4-methoxybutan-2-yl)hydrazine-hydrogen chloride (1/1)

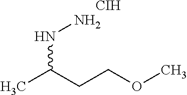

(±)-Tert-butyl 2-(4-methoxybutan-2-yl)hydrazine-1-carboxylate (3.00 g, 13.7 mmol, Intermediate 79) was dissolved in hydrochloric acid in dioxane (34 mL, 4.0 M, 140 mmol) and stirred for 1 hour at 20° C. The mixture was evaporated under reduced pressure. The residue was directly used in the next step.

### Intermediate 81

### (±)-6-cyclopropyl-1-(4-methoxybutan-2-yl)-1H-pyrazolo[3,4-b]pyrazin-3-amine

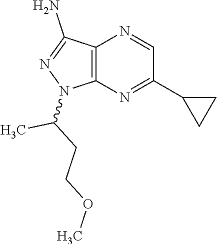 [SMILES: COCCC(C)n1nc(N)c2ncc(C3CC3)nc21]

To a solution of 3-chloro-5-cyclopropylpyrazine-2-carbonitrile (1.00 g, 5.57 mmol, intermediate 1) in 15 mL 1-butanol was added potassium carbonate (3.08 g, 22.3 mmol) and (±)-(4-methoxybutan-2-yl)hydrazine-hydrogen chloride (1/1) (1.29 g, 8.35 mmol, intermediate 80). The mixture was stirred for 4 hours at 120° C. and over night at room temperature. The reaction mixture was diluted with 150 mL ethyl acetate, extracted with water twice, brine, dried using a hydrophobic filter and evaporated to dryness. The residue was adsorbed on Isolute® and this absorbed material was then purified by two subsequent column chromatographies using a Biotage chromatography system (1.55 g snap NH column, hexane/0-100% ethyl acetate then ethyl acetate/0-50% ethanol, 2.55 g snap NH column, hexane/0-100% ethyl acetate then ethyl acetate/0-100% ethanol) to give 529 mg (90% purity, 33% yield) of desired compound.

LC-MS (Method 2): Rₜ=0.99 min; MS (ESIpos): m/z=262 [M+H]⁺

¹H-NMR (400 MHz, DMSO-d6) δ [ppm]: 1.009 (0.42), 1.019 (0.84), 1.026 (0.95), 1.028 (0.82), 1.031 (0.91), 1.038 (0.92), 1.064 (0.60), 1.070 (1.19), 1.080 (0.72), 1.082 (0.80), 1.090 (1.23), 1.098 (0.53), 1.100 (0.49), 1.108 (0.88), 1.124 (0.70), 1.152 (0.55), 1.169 (1.04), 1.187 (0.49), 1.341 (4.19), 1.358 (4.25), 1.367 (0.43), 1.909 (0.40), 1.986 (1.76), 2.019 (0.41), 2.043 (0.47), 2.295 (0.64), 2.929 (0.42), 2.943 (0.48), 2.949 (0.42), 3.061 (16.00), 3.097 (1.13), 3.099 (0.41), 3.114 (0.68), 3.128 (0.43), 3.137 (0.64), 3.198 (0.57), 3.209 (1.52), 3.949 (0.72), 5.743 (2.28), 7.927 (0.41), 8.316 (5.14), 11.207 (0.44).

### Intermediate 82

### 3-(3-amino-6-cyclopropyl-1H-pyrazolo[3,4-b]pyrazin-1-yl)propan-1-ol

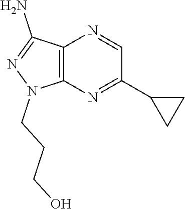 [SMILES: Nc1nn(CCCO)c2nc(C3CC3)cnc12]

To a solution of 3-chloro-5-cyclopropylpyrazine-2-carbonitrile (1.00 g, 5.57 mmol, intermediate 1) in 15 mL 1-butanol was added potassium carbonate (3.08 g, 22.3 mmol) and 3-hydrazinylpropan-1-ol-hydrogen chloride (1/2) (1.36 g, 8.35 mmol, CAS-RN:[1258651-49-8], commercially available e.g. ChemBridge Corp.). The mixture was stirred for 4 hours at 120° C. and over night at room temperature. The reaction mixture was diluted with 150 mL ethyl acetate, extracted with water twice, brine, dried using a hydrophobic filter and evaporated to dryness. The residue was purified by column chromatographiy using a Biotage chromatography system (28 g snap NH column, hexane/0-100% ethyl acetate then ethyl acetate/0-100% ethanol) to give 750 mg (91% purity, 52% yield) of desired compound.

LC-MS (Method 2): Rₜ=0.73 min; MS (ESIpos): m/z=234 [M+H]⁺

¹H-NMR (400 MHz, DMSO-d6) δ [ppm]: 1.014 (0.58), 1.027 (2.08), 1.033 (2.81), 1.039 (3.04), 1.045 (2.72), 1.052 (1.62), 1.064 (1.66), 1.071 (2.61), 1.078 (1.47), 1.084 (1.76), 1.091 (2.99), 1.098 (1.53), 1.109 (0.75), 1.849 (0.61), 1.865 (2.11), 1.883 (2.97), 1.900 (2.24), 1.915 (0.79), 2.277 (0.79), 2.285 (0.84), 2.293 (0.79), 2.297 (1.43), 2.306 (0.60), 2.309 (0.81), 2.317 (0.77), 3.335 (16.00), 3.348 (2.13), 3.351 (3.53), 3.364 (3.62), 3.380 (1.50), 3.955 (1.38), 4.106 (2.35), 4.124 (4.04), 4.133 (0.45), 4.141 (2.27), 4.484 (2.12), 4.497 (4.76), 4.509 (2.09), 5.715 (4.78), 7.923 (0.64), 8.311 (10.22).

### Intermediate 83

### tert-butyl 1-(3-cyano-6-cyclopropylpyrazin-2-yl)hydrazine-1-carboxylate

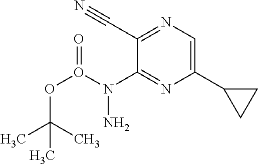

A solution of tert-butyl hydrazinecarboxylate (1.01 g, 7.66 mmol) in 14 mL N,N-dimethylformamide was cooled to 0° C. Sodium hydride (334 mg, 60% purity, 8.35 mmol) was added and the mixture was stirred for 30 minutes at 0° C. 3-chloro-5-cyclopropylpyrazine-2-carbonitrile (1.25 g, 6.96 mmol, intermediate 1) was added and the mixture was stirred for two days at room temperature. The reaction mixture was diluted with 100 mL ethyl acetate, extracted with water twice, brine, dried using a hydrophobic filter and evaporated to dryness. The residue was adsorbed on Isolute® and this absorbed material was then purified by two subsequent column chromatographies using a Biotage chromatography system (1.50 g snap KP-Sil column, hexane/0-100% ethyl acetate then ethyl acetate/0-10% ethanol, 2.50g snap KP-Sil column, hexane/0-100% ethyl acetate then ethyl acetate/0-10% ethanol) to give 0.49 g (25% yield) of desired compound.

1H-NMR (400 MHz, DMSO-d6) δ [ppm]: 1.069 (0.55), 1.076 (0.78), 1.082 (0.64), 1.088 (0.80), 1.118 (0.41), 1.125 (0.74), 1.132 (0.44), 1.138 (0.47), 1.145 (0.81), 1.152 (0.68), 1.170 (0.55), 1.377 (2.66), 1.427 (0.52), 1.568 (16.00), 1.986 (0.93), 5.757 (0.71), 6.457 (1.41), 8.619 (3.03).

### Intermediate 84

### tert-butyl (6-cyclopropyl-1H-pyrazolo[3,4-b]pyrazin-3-yl)carbamate

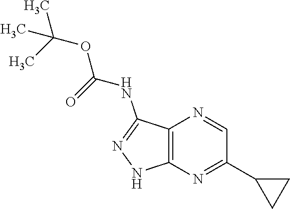 [SMILES: CC(C)(C)OC(=O)Nc1n[nH]c2nc(C3CC3)cnc12]

A solution of tert-butyl 1-(3-cyano-6-cyclopropylpyrazin-2-yl)hydrazine-1-carboxylate (490 mg, 1.78 mmol, intermediate 83) in 3.6 mL N,N-dimethylformamide was cooled to 0° C. Sodium hydride (71.2 mg, 60% purity, 1.78 mmol) was added and the mixture was stirred for 30 minutes at 0° C. followed by two days at room temperature. The reaction mixture was diluted with 100 mL ethyl acetate, extracted with water twice, brine, dried using a hydrophobic filter and evaporated to dryness. The residue was purified by column chromatography using a Biotage chromatography system (50 g snap KP-Sil column, hexane/0-100% ethyl acetate then ethyl acetate/0-10% ethanol) to give 230 mg (90% purity, 42% yield) of the desired compound.

LC-MS (Method 2): Rₜ=0.88 min; MS (ESIpos): m/z=276 [M+H]⁺

¹H-NMR (400 MHz, DMSO-d6) δ [ppm]: 1.038 (0.91), 1.044 (1.25), 1.049 (0.94), 1.052 (0.89), 1.056 (1.14), 1.064 (0.58), 1.077 (0.41), 1.088 (0.45), 1.115 (0.63), 1.123 (1.19), 1.130 (0.88), 1.135 (0.89), 1.143 (1.28), 1.150 (0.90), 1.153 (1.39), 1.161 (0.44), 1.170 (1.62), 1.189 (0.87), 1.427 (16.00), 1.437 (2.07), 1.569 (6.56), 1.609 (1.26), 1.986 (3.01), 2.371 (0.42), 2.382 (0.68), 2.392 (0.43), 2.729 (3.28), 2.888 (3.95), 4.016 (0.67), 4.034 (0.66), 6.459 (0.56), 7.950 (0.47), 8.601 (3.73), 8.620 (1.20), 9.338 (1.24), 13.314 (1.24).

### Intermediate 85

### tert-butyl [6-cyclopropyl-1-(3-methoxypropyl)-1H-pyrazolo[3,4-b]pyrazin-3-yl]carbamate

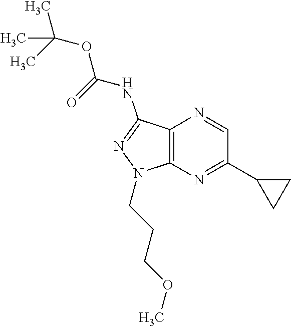 [SMILES: COCCCn1nc(NC(=O)OC(C)(C)C)c2ncc(C3CC3)nc21]

The reaction was performed in two separate batches.

The first batch was prepared as followed :

A solution of tert-butyl (6-cyclopropyl-1H-pyrazolo[3,4-b]pyrazin-3-yl)carbamate (115 mg, 418 μmol, intermediate 84) in 2.2 mL tetrahydrofuran was cooled to 0° C. Sodium hydride (16.7 mg, 60% purity, 418 μmol) was added and the mixture was stirred for 30 min at 0° C. 1-bromo-3-methoxypropane (63.9 mg, 418 μmol) was added and the mixture was stirred over night at room temperature.

The second batch was prepared as followed:

A solution of tert-butyl (6-cyclopropyl-1H-pyrazolo[3,4-b]pyrazin-3-yl)carbamate (115 mg, 418 μmol, intermediate 84) and 1-bromo-3-methoxypropane (63.9 mg, 418 μmol) in 2.0 mL tetrahydrofuran was added dropwise to a mixture of sodium hydride (16.7 mg, 60% purity, 418 μmol) in 3.0 mL tetrahydrofuran at 0° C. The mixture was stirred overnight at room temperature.

The batches were combined, diluted with 50 mL ethyl acetate, extracted with water twice, brine, dried using a hydrophobic filter and evaporated to dryness. The residue was purified by column chromatography using a Biotage chromatography system (10 g snap KP-Sil column, hexane/0-100% ethyl acetate then ethyl acetate/0-100% ethanol) to give 160 mg (57% purity, 32% yield) of the desired compound.

LC-MS (Method 2): Rₜ=1.19 min; MS (ESIpos): m/z=348 [M+H]⁺

¹H-NMR (400 MHz, DMSO-d6) δ [ppm]: 1.045 (0.41), 1.057 (0.45), 1.083 (0.69), 1.089 (0.94), 1.095 (0.86), 1.101 (1.10), 1.108 (0.63), 1.114 (0.44), 1.124 (0.47), 1.135 (0.67), 1.142 (1.12), 1.149 (0.68), 1.154 (0.93), 1.163 (1.04), 1.169 (0.64), 1.172 (0.68), 1.181 (0.43), 1.309 (2.50), 1.321 (0.61), 1.428 (16.00), 1.456 (0.53), 1.473 (0.43), 1.987 (0.56), 2.005 (0.70), 2.021 (1.07), 2.037 (0.69), 2.394 (0.51), 2.518 (0.51), 3.098 (2.60), 3.107 (0.48), 3.110 (0.64), 3.135 (2.50), 3.172 (9.95), 3.222 (0.53), 3.250 (0.88), 3.252 (0.73), 3.265 (1.85), 3.280 (0.81), 3.318 (0.66), 4.315 (0.64), 4.332 (1.25), 4.349 (0.60), 5.758 (7.91), 8.597 (3.12), 8.601 (1.19), 8.649 (0.85), 9.427 (0.84).

### Intermediate 86

### 6-cyclopropyl-1-(3-methoxypropyl)-1H-pyrazolo[3,4-b]pyrazin-3-amine

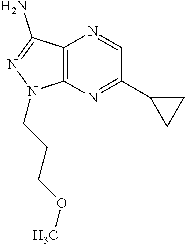 [SMILES: COCCCn1nc(N)c2ncc(C3CC3)nc21]

Tert-butyl [6-cyclopropyl-1-(3-methoxypropyl)-1H-pyrazolo[3,4-b]pyrazin-3-yl]carbamate (160 mg, 461 μmol, intermediate 85) was dissolved in hydrochloric acid in dioxane (1.0 mL, 4.0 M, 4.0 mmol) and stirred for 2 hour at room temperature. The mixture was diluted with 30 mL ethyl acetate, extracted with water twice, brine, dried using a hydrophobic filter and evaporated to dryness. The residue was purified by column chromatography using a Biotage chromatography system (11g NH column, hexane/0-100% ethyl acetate then ethyl acetate/0-100% ethanol) to give 39.0 mg (80% purity, 27% yield) of the desired compound.

LC-MS (Method 2): Rₜ=0.91 min; MS (ESIpos): m/z=248 [M+H]⁺

¹H-NMR (400 MHz, DMSO-d6) δ [ppm]: 0.794 (0.43), 1.005 (0.41), 1.027 (1.17), 1.033 (1.84), 1.040 (1.67), 1.045 (1.48), 1.052 (0.82), 1.068 (1.03), 1.075 (1.47), 1.083 (1.50), 1.089 (0.95), 1.096 (1.71), 1.102 (0.89), 1.114 (0.41), 1.258 (0.85), 1.842 (0.41), 1.934 (1.08), 1.944 (0.64), 1.950 (1.76), 1.959 (0.47), 1.966 (1.11), 2.285 (0.46), 2.293 (0.48), 2.301 (0.44), 2.305 (0.74), 2.317 (0.51), 2.325 (0.48), 2.518 (0.87), 2.523 (0.58), 3.161 (16.00), 3.164 (5.31), 3.171 (0.45), 3.214 (1.55), 3.221 (0.72), 3.228 (7.32), 3.236 (1.10), 3.245 (1.47), 3.252 (0.47), 3.315 (0.42), 3.394 (0.47), 3.410 (0.98), 3.426 (0.43), 4.105 (1.15), 4.122 (2.50), 4.139 (1.32), 5.732 (2.29), 8.304 (1.46), 8.323 (5.31).

### Intermediate 87

### tert-butyl 2[4-(dimethylamino)butan-2-ylidene]hydrazine-1-carboxylate

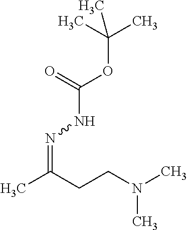 [SMILES: CC(CCN(C)C)=NNC(=O)OC(C)(C)C]

To a solution of 4-(dimethylamino)butan-2-one (3.50 g, 30.4 mmol) in 23 mL methanol was added a solution of tert-butyl hydrazinecarboxylate (3.82 g, 28.9 mmol) in 23 mL methanol dropwise. The mixture was stirred for 1 h and directly used in the next step.

### Intermediate 88

### (±)-tert-butyl 2-[4-(dimethylamino)butan-2-yl]hydrazine-1-carboxylate

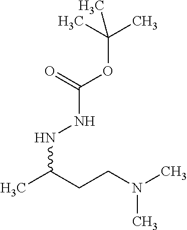 [SMILES: CC(CCN(C)C)NNC(=O)OC(C)(C)C]

To the reaction mixture of tert-butyl 2-[4-(dimethylamino)butan-2-ylidene]hydrazine-1-carboxylate (3.49 g, 15.2 mmol, intermediate 87) 31 mL water and 10 mL acetic acid were added. The mixture was cooled to 0° C. and sodium cyanoborohydride (1.15 g, 18.2 mmol) was added. The mixture was stirred over night at room temperature. The mixture was adjusted with a sodiumhydroxide solution (4.0 M) to pH=7 and extracted with dichloromethane thrice. The combined organic layers were washed with brine, filtered over a hydrophobic filter and concentrated under reduced pressure to give 6.40 g (90% purity, 164% yield) of the desired compound

¹H-NMR (400 MHz, DMSO-d6) δ [ppm]: 0.961 (1.34), 0.977 (1.35), 1.378 (1.96), 1.399 (16.00), 1.746 (1.33), 2.644 (2.39), 5.758 (3.24).

### Intermediate 89

### (±)-3-hydrazinyl-N,N-dimethylbutan-1-amine—hydrogen chloride (1/1)

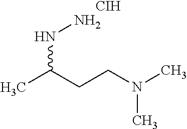

(±)-Tert-butyl 2-[4-(dimethylamino)butan-2-yl]hydrazine-1-carboxylate (3.00 g, 13.0 mmol, intermediate 88) was dissolved in hydrochloric acid in dioxane (32 mL, 4.0 M, 130 mmol) and stirred for 1 hour at 20° C. The suspension was filtered and the solid was washed with small amounts of dioxane. The solid was directly used in the next step.

### Intermediate 90

### (±)-6-cyclopropyl-1-[4-(dimethylamino)butan-2-yl]-1H-pyrazolo[3,4-b]pyrazin-3-amine

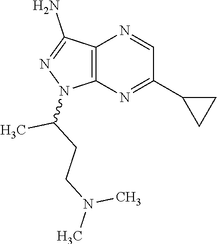 [SMILES: CC(CCN(C)C)n1nc(N)c2ncc(C3CC3)nc21]

To a solution of 3-chloro-5-cyclopropylpyrazine-2-carbonitrile (1.00 g, 5.57 mmol, intermediate 1) in 15 mL 1-butanol was added potassium carbonate (3.08 g, 22.3 mmol) and (±)-3-hydrazinyl-N,N-dimethylbutan-1-amine-hydrogen chloride (1/1) (1.40 g, 8.35 mmol). The mixture was stirred for 4 hours at 120° C. and over night at room temperature. The reaction mixture was diluted with 150 mL ethyl acetate, extracted with water twice, brine, dried using a hydrophobic filter and evaporated to dryness. The residue was purified by column chromatographiy using a Biotage chromatography system (55 g snap NH column, hexane/0-100% ethyl acetate then ethyl acetate/0-100% ethanol) to give 668 mg (80% purity, 35% yield) of desired compound.

LC-MS (Method 2): Rₜ=1.03 min; MS (ESIpos): m/z=276 [M+H]⁺

¹H-NMR (400 MHz, DMSO-d6) δ [ppm]: 0.840 (0.51), 0.858 (1.29), 0.876 (0.67), 0.998 (1.09), 1.002 (0.79), 1.011 (0.83), 1.018 (1.11), 1.023 (0.89), 1.032 (0.60), 1.035 (0.60), 1.067 (0.87), 1.077 (0.72), 1.088 (0.94), 1.096 (0.60), 1.152 (1.64), 1.170 (3.54), 1.187 (1.80), 1.344 (3.18), 1.361 (3.16), 1.389 (1.36), 1.405 (1.28), 1.885 (0.41), 1.893 (0.58), 1.904 (0.41), 1.917 (0.67), 1.928 (0.52), 1.949 (0.76), 1.951 (0.76), 1.954 (0.75), 1.971 (0.75), 1.986 (6.39), 1.993 (0.51), 2.010 (16.00), 2.038 (0.82), 2.071 (6.10), 2.076 (1.10), 2.292 (0.53), 3.682 (0.44), 3.944 (0.42), 3.997 (0.41), 4.015 (1.27), 4.033 (1.28), 4.051 (0.42), 5.730 (1.84), 6.498 (0.64), 8.129 (1.71), 8.310 (3.91).

### Intermediate 91

### 6-cyclopropyl-1-(2-methoxyethyl)-1H-pyrazolo[3,4-b]pyrazin-3-amine

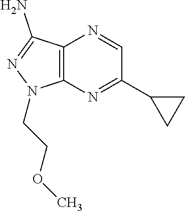 [SMILES: COCCn1nc(N)c2ncc(C3CC3)nc21]

To a solution of 3-chloro-5-cyclopropylpyrazine-2-carbonitrile (300 mg, 1.67 mmol, intermediate 1) in 4.1 mL 1-butanol was added potassium carbonate (923 mg, 6.68 mmol) and (2-methoxyethyl)hydrazine—hydrogen chloride (1/2) (409 mg, 2.51 mmol, CAS-RN:[885330-C₃-0], commercially available e.g. Ark Pharm Inc.). The mixture was stirred for 4 hours at 120° C. and over night at 90° C. To the reaction mixture was added saturated sodiumcarbonate solution. The mixture was extracted with ethyl acetate thrice The combined organic layer were dried using a hydrophobic filter and evaporated to dryness. The residue was purified by column chromatographiy using a Biotage chromatography system (55 g snap NH column, hexane/0-100% ethyl acetate then ethyl acetate/0-100% ethanol) to give 230 mg (98% purity, 58% yield) of desired compound.

LC-MS (Method 2): Rₜ=0.84 min; MS (ESIpos): m/z=234 [M+H]⁺

¹H-NMR (400 MHz, DMSO-d6) δ [ppm]: 1.023 (0.92), 1.030 (1.28), 1.036 (1.07), 1.041 (1.24), 1.049 (0.63), 1.069 (0.66), 1.076 (1.14), 1.082 (0.78), 1.089 (0.67), 1.096 (1.31), 1.103 (0.70), 2.306 (0.65), 2.318 (0.42), 2.326 (0.47), 2.518 (0.55), 3.179 (16.00), 3.210 (0.77), 3.669 (1.13), 3.683 (2.53), 3.697 (1.30), 3.956 (0.59), 4.208 (1.19), 4.221 (2.23), 4.235 (1.05), 5.744 (2.08), 8.326 (4.91).

### Intermediate 92

### 2-(3-amino-6-cyclopropyl-1H-pyrazolo[3,4-b]pyrazin-1-yl)ethan-1-ol

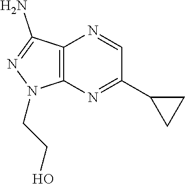 [SMILES: Nc1nn(CCO)c2nc(C3CC3)cnc12]

To a solution of 3-chloro-5-cyclopropylpyrazine-2-carbonitrile (300 mg, 1.67 mmol, intermediate 1) in 4.1 mL 1-butanol was added potassium carbonate (923 mg, 6.68 mmol) and 2-hydrazinylethan-1-ol (170 μL, 2.5 mmol, CAS-RN:[109-84-2], commercially available e.g. Sigma Aldrich). The mixture was stirred for 4 hours at 120° C. and over night at 90° C. To the reaction mixture was added saturated sodium carbonate solution. The mixture was extracted with ethyl acetate thrice The combined organic layer were dried using a hydrophobic filter and evaporated to dryness. The residue was purified by column chromatographiy using a Biotage chromatography system (55 g snap NH column, hexane/0-100% ethyl acetate then ethyl acetate/0-100% ethanol) to give 266 mg (90% purity, 65% yield) of desired compound.

LC-MS (Method 2): Rₜ=0.68 min; MS (ESIpos): m/z=220 [M+H]⁺

¹H-NMR (400 MHz, DMSO-d6) δ [ppm]: 1.014 (1.07), 1.027 (3.75), 1.033 (5.12), 1.039 (5.79), 1.045 (5.13), 1.052 (3.18), 1.065 (3.10), 1.071 (4.68), 1.078 (2.85), 1.085 (3.29), 1.092 (5.49), 1.099 (2.99), 1.110 (1.58), 1.231 (0.47), 2.267 (0.66), 2.279 (1.37), 2.287 (1.49), 2.296 (1.48), 2.299 (2.48), 2.308 (1.17), 2.311 (1.54), 2.319 (1.58), 2.327 (0.98), 2.331 (1.15), 2.518 (2.71), 2.523 (1.74), 2.665 (0.56), 2.669 (0.77), 2.673 (0.55), 2.728 (0.49), 2.888 (0.59), 3.159 (4.98), 3.172 (5.11), 3.699 (2.08), 3.714 (6.47), 3.729 (6.95), 3.744 (2.70), 3.758 (0.49), 3.956 (1.52), 4.084 (0.82), 4.094 (4.53), 4.110 (9.69), 4.125 (3.82), 4.730 (3.24), 4.744 (7.19), 4.759 (3.01), 5.713 (8.32), 5.759 (1.02), 7.923 (0.69), 8.308 (16.00), 8.343 (0.50).

### Intermediate 93

### tert-butyl [6-cyclopropyl-1-(3-methoxy-3-methylbutyl)-1H-pyrazolo[3,4-b]pyrazin-3-yl]carbamate

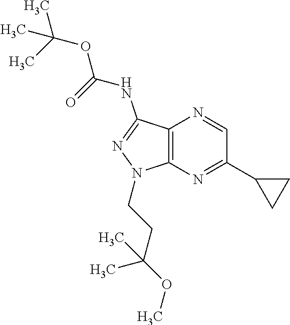 [SMILES: COC(C)(C)CCn1nc(NC(=O)OC(C)(C)C)c2ncc(C3CC3)nc21]

A solution of tert-butyl (6-cyclopropyl-1H-pyrazolo[3,4-b]pyrazin-3-yl)carbamate (150 mg, 545 μmol, intermediate 84) in 3.2 tetrahydrofuran was cooled to 0° C. A solution of lithium diisopropylamide in tetrahydrofuran was added (570 μL, 2.0 M, 1.1 mmol) and the mixtre was stirred for 30 minutes at 0° C. 1-bromo-3-methoxy-3-methylbutane (98.7 mg, 545 pmol) was added and the mixture was stirred at 50° C. for three days. Another portion of the lithiumdiisopropylamid solution (570 μL, 2.0 M, 1.1 mmol) was added and the mixture was stirred for 23 days at 50° C. The reaction mixture was diluted with 50 mL ethyl acetate, extracted with water twice, brine, dried using a hydrophobic filter and evaporated to dryness. The residue was purified by column chromatographiy using a Biotage chromatography system (10g snap KP-Sil column, hexane/0-100% ethyl acetate then ethyl acetate/0-100% ethanol) to give 99.7 mg (72% purity, 35% yield) of desired compound.

LC-MS (Method 2): Rₜ=1.31 min; MS (ESIpos): m/z=376 [M+H]⁺

¹H-NMR (400 MHz, DMSO-d6) δ [ppm]: 1.051 (2.14), 1.063 (0.43), 1.074 (0.79), 1.081 (1.23), 1.088 (0.94), 1.096 (1.76), 1.134 (12.98), 1.142 (1.48), 1.150 (1.15), 1.154 (1.74), 1.162 (1.07), 1.172 (2.26), 1.190 (1.07), 1.231 (0.96), 1.317 (0.81), 1.363 (1.21), 1.427 (16.00), 1.930 (0.64), 1.944 (0.57), 1.950 (0.71), 1.956 (0.62), 1.970 (0.71), 1.987 (3.13), 2.383 (0.44), 2.394 (0.57), 2.518 (0.81), 2.523 (0.54), 3.010 (0.98), 3.043 (1.30), 3.098 (8.79), 4.017 (0.74), 4.035 (0.73), 4.291 (0.64), 4.304 (0.55), 4.310 (0.66), 4.316 (0.55), 4.330 (0.64), 5.759 (8.36), 8.594 (3.25), 8.601 (1.11), 9.416 (0.95).

### Intermediate 94

### 6-cyclopropyl-1-(3-methoxy-3-methylbutyl)-1H-pyrazolo[3,4-b]pyrazin-3-amine

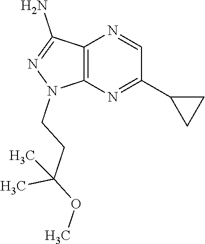 [SMILES: COC(C)(C)CCn1nc(N)c2ncc(C3CC3)nc21]

Tert-butyl [6-cyclopropyl-1-(3-methoxy-3-methylbutyl)-1H-pyrazolo[3,4-b]pyrazin-3-yl]carbamate (99.0 mg, 264 μmol, intermediate 93) was dissolved in hydrochloric acid in dioxane (1.0 mL, 4.0 M, 4.0 mmol) and stirred for 2 hour at room temperature. The mixture was diluted with 30 mL ethyl acetate, extracted with water twice, brine, dried using a hydrophobic filter and evaporated to dryness. The residue was purified by column chromatography using a Biotage chromatography system (11g NH column, hexane/0-100% ethyl acetate then ethyl acetate/0-100% ethanol) to give 38.2 mg (89% purity, 47% yield) of the desired compound.

LC-MS (Method 2): Rₜ=1.04 min; MS (ESIpos): m/z=276 [M+H]⁺

¹H-NMR (400 MHz, DMSO-d6) δ [ppm]: 1.020 (0.85), 1.026 (1.21), 1.033 (1.01), 1.038 (1.19), 1.045 (0.60), 1.052 (0.42), 1.069 (0.61), 1.076 (1.11), 1.082 (0.78), 1.089 (0.81), 1.096 (1.37), 1.109 (16.00), 1.135 (2.04), 1.847 (0.80), 1.861 (0.68), 1.867 (0.89), 1.873 (0.72), 1.886 (0.85), 2.305 (0.56), 2.318 (0.42), 2.323 (0.41), 2.327 (0.54), 2.518 (1.45), 2.523 (0.96), 2.669 (0.41), 3.100 (12.81), 3.125 (1.47), 4.083 (0.85), 4.096 (0.71), 4.103 (0.88), 4.108 (0.73), 4.122 (0.88), 5.717 (1.83), 8.295 (0.44), 8.317 (4.24).

### Intermediate 95

### 6-cyclopropyl-1-[2-(dimethylamino)ethyl]-1 H-pyrazolo[3,4-b]pyrazin-3-amine

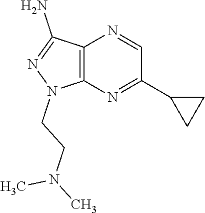 [SMILES: CN(C)CCn1nc(N)c2ncc(C3CC3)nc21]

In analogy to 90) 3-chloro-5-cyclopropylpyrazine-2-carbonitrile (300 mg, 1.67 mmol) and 2-hydrazinyl-N,N-dimethylethan-1-amine-hydrogen chloride (1/2) (441 mg, 2.51 mmol, CAS-RN:[57659-80-0], commercially available e.g. Fluorochem) reacted and give after purification using a Biotage chromatography 118 mg (71% purity, 20% yield) of the desired title compound.

¹H-NMR (400 MHz, DMSO-d6) δ [ppm]: 1.007 (0.43), 1.019 (0.82), 1.025 (1.16), 1.032 (0.93), 1.037 (1.17), 1.044 (0.58), 1.067 (0.59), 1.074 (1.07), 1.081 (0.64), 1.087 (0.63), 1.094 (1.19), 1.101 (0.65), 2.109 (16.00), 2.147 (1.59), 2.163 (2.71), 2.306 (0.57), 2.326 (0.43), 2.518 (0.64), 2.523 (0.40), 2.578 (0.82), 2.594 (1.93), 2.610 (0.92), 2.931 (1.83), 3.682 (0.83), 3.953 (0.96), 4.141 (0.87), 4.156 (1.87), 4.172 (0.83), 5.715 (1.90), 5.759 (0.87), 7.736 (0.51), 7.923 (0.45), 8.322 (4.01).

### Intermediate 96

### 5-cyclopropyl-3-[(2-hydroxy-2-methylpropyl)amino]pyrazine-2-carbonitrile

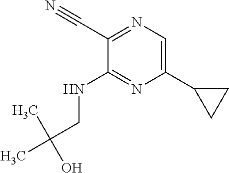 [SMILES: CC(C)(O)CNc1nc(C2CC2)cnc1C#N]

In analogy to 39) 3-chloro-5-cyclopropylpyrazine-2-carbonitrile (400 mg, 2.23 mmol, intermediate 1) and 1-amino-2-methylpropan-2-ol (218 mg, 2.45 mmol) reacted and give after purification using a Biotage chromatography 379 mg (91% purity, 67% yield) of the desired title compound.

LC-MS (Method 2): Rₜ=1.03 min; MS (ESIpos): m/z=233 [M+H]⁺

¹H-NMR (400 MHz, DMSO-d6) δ [ppm]: 0.980 (0.71), 0.987 (1.20), 0.991 (0.72), 0.994 (0.65), 0.998 (0.96), 1.006 (0.47), 1.047 (0.49), 1.055 (1.14), 1.066 (13.82), 1.074 (1.35), 1.081 (0.68), 2.106 (0.55), 2.727 (8.93), 2.888 (10.58), 3.321 (1.90), 3.331 (16.00), 3.336 (2.16), 4.632 (3.69), 6.879 (0.45), 7.905 (3.52), 7.950 (1.27).

### Intermediate 97

### 1-(3-amino-6-cyclopropyl-1H-pyrazolo[3,4-b]pyrazin-1-yl)-2-methylpropan-2-ol

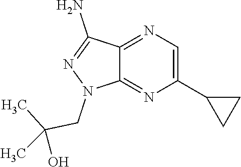 [SMILES: CC(C)(O)Cn1nc(N)c2ncc(C3CC3)nc21]

In analogy to 42) 5-cyclopropyl-3-[(2-hydroxy-2-methyl propyl)amino]pyrazine-2-carbonitrile (379 mg, 1.63 mmol, intermediate 96) and O-(diphenylphosphinoyl)hydroxylamine (514 mg, 2.206 mmol) reacted 16 hours at room temperature and give after purification using a Biotage chromatography 101 mg (80% purity, 20% yield) of the desired title compound.

LC-MS (Method 2): Rₜ=0.84 min; MS (ESIpos): m/z=248 [M+H]⁺

¹H-NMR (400 MHz, DMSO-d6) δ [ppm]: 1.006 (0.48), 1.018 (1.11), 1.024 (1.51), 1.031 (1.23), 1.036 (1.57), 1.043 (0.83), 1.065 (16.00), 1.076 (1.66), 1.083 (1.06), 1.089 (0.98), 1.097 (1.54), 1.103 (0.92), 1.110 (0.47), 1.114 (0.55), 1.119 (1.10), 1.128 (1.32), 1.135 (0.41), 1.171 (0.61), 1.987 (1.09), 2.304 (0.67), 2.316 (0.42), 2.325 (0.46), 2.518 (0.96), 2.522 (0.61), 2.727 (0.48), 2.888 (0.57), 3.993 (4.11), 4.684 (4.34), 5.744 (2.32), 5.758 (0.66), 8.330 (4.72).

### Intermediate 98

### 5-cyclopropyl-3-{[(trans-3-hydroxycyclobutyl)methyl]amino}pyrazine-2-carbonitrile

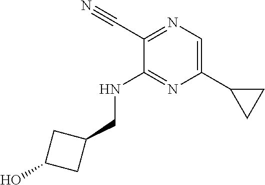 [SMILES: N#Cc1ncc(C2CC2)nc1NC[C@H]1C[C@H](O)C1]

In analogy to 39) 3-chloro-5-cyclopropylpyrazine-2-carbonitrile (250 mg, 1.18 mmol, intermediate 1) and trans-3-(aminomethyl)cyclobutan-1-ol (132 mg, 1.30 mmol, CAS-RN:[1234616-04-6], commercially available e.g. Advanced ChemBlocks Inc.) reacted and give after purification using a Biotage chromatography 134 mg (90% purity, 42% yield) of the desired title compound.

LC-MS (Method 2): Rₜ=0.95 min; MS (ESIpos): m/z=245 [M+H]⁺

¹H-NMR (400 MHz, DMSO-d6) δ [ppm]: 0.960 (1.04), 0.971 (2.76), 0.978 (4.45), 0.982 (2.90), 0.985 (2.57), 0.989 (3.72), 0.997 (1.74), 1.012 (0.41), 1.032 (0.47), 1.047 (1.65), 1.055 (3.56), 1.062 (2.20), 1.067 (2.48), 1.075 (3.89), 1.082 (2.36), 1.093 (0.98), 1.172 (0.79), 1.190 (0.43), 1.805 (0.90), 1.812 (0.66), 1.823 (1.14), 1.829 (2.01), 1.836 (1.77), 1.847 (1.63), 1.854 (2.46), 1.859 (1.79), 1.870 (0.92), 1.877 (1.52), 1.949 (1.45), 1.956 (1.99), 1.967 (1.77), 1.974 (2.53), 1.980 (2.12), 1.987 (2.41), 1.991 (1.12), 1.998 (1.38), 2.005 (1.03), 2.068 (0.57), 2.080 (1.16), 2.088 (1.26), 2.100 (2.25), 2.108 (0.95), 2.111 (1.22), 2.119 (1.10), 2.131 (0.49), 2.284 (0.70), 2.304 (0.97), 2.318 (0.67), 2.323 (0.88), 2.327 (1.11), 2.331 (0.74), 2.518 (1.80), 2.523 (1.18), 2.669 (0.51), 2.728 (0.53), 2.889 (0.61), 3.341 (3.60), 3.356 (3.65), 3.360 (3.50), 3.376 (3.03), 3.898 (1.27), 4.167 (0.85), 4.184 (1.71), 4.201 (1.72), 4.219 (0.89), 4.945 (7.61), 4.953 (0.47), 4.960 (7.19), 7.480 (0.58), 7.505 (0.98), 7.520 (1.95), 7.534 (0.94), 7.876 (16.00).

### Intermediate 99

### trans-3-[(3-amino-6-cyclopropyl-1H-pyrazolo[3,4-b]pyrazin-1-yl)methyl]cyclobutan-1-ol

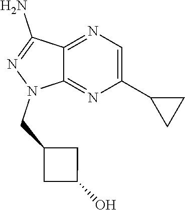 [SMILES: Nc1nn(C[C@H]2C[C@H](O)C2)c2nc(C3CC3)cnc12]

In analogy to 42) 5-cyclopropyl-3-{[(trans-3-hydroxycyclobutyl)methyl]amino}pyrazine-2-carbonitrile (134 mg, 549 μmol, intermediate 98) and O-(diphenylphosphinoyl)hydroxylamine (173 mg, 740 μmol) reacted and give after purification using a Biotage chromatography 98.0 mg (95% purity, 65% yield) of the desired title compound.

LC-MS (Method 2): Rₜ=0.78 min; MS (ESIpos): m/z=260 [M+H]⁺

¹H-NMR (400 MHz, DMSO-d6) δ [ppm]: 1.011 (1.44), 1.023 (4.20), 1.030 (6.21), 1.036 (4.94), 1.042 (5.94), 1.049 (2.86), 1.061 (0.95), 1.070 (1.29), 1.075 (2.74), 1.082 (5.68), 1.089 (3.20), 1.095 (3.36), 1.103 (5.85), 1.109 (3.24), 1.116 (1.20), 1.121 (1.38), 1.153 (0.43), 1.171 (0.76), 1.231 (0.46), 1.820 (1.41), 1.827 (1.02), 1.838 (1.77), 1.844 (3.08), 1.851 (2.77), 1.861 (2.55), 1.868 (3.65), 1.874 (2.56), 1.885 (1.35), 1.892 (2.05), 1.987 (2.81), 1.997 (2.86), 2.007 (2.70), 2.014 (3.69), 2.021 (3.15), 2.029 (1.96), 2.039 (2.07), 2.047 (1.55), 2.277 (0.94), 2.289 (1.74), 2.297 (1.90), 2.309 (2.97), 2.321 (1.92), 2.328 (1.91), 2.340 (0.70), 2.518 (3.00), 2.523 (2.10), 2.665 (0.44), 2.669 (0.58), 2.673 (0.43), 3.959 (1.24), 4.083 (0.66), 4.102 (10.27), 4.120 (9.87), 4.134 (2.78), 4.151 (1.36), 4.956 (9.35), 4.971 (8.61), 5.709 (9.23), 5.758 (2.35), 7.931 (0.52), 8.312 (0.57), 8.324 (16.00).

### Intermediate 100

### 5-cyclopropyl-3-{[(cis-3-hydroxycyclobutyl)methyl]amino}pyrazine-2-carbonitrile

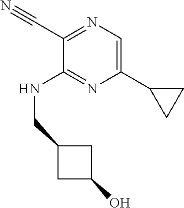 [SMILES: N#Cc1ncc(C2CC2)nc1NC[C@H]1C[C@@H](O)C1]

In analogy to 39) 3-chloro-5-cyclopropylpyrazine-2-carbonitrile (250 mg, 1.18 mmol, intermediate 1) and cis-3-(aminomethyl)cyclobutan-1-ol (132 mg, 1.30 mmol, CAS-RN:[917827-91-9], commercially available e.g. Combi-Blocks Inc.) reacted 16 hours at 100° C. and give after purification using a Biotage chromatography 162 mg (95% purity, 53% yield) of the desired title compound.

LC-MS (Method 2): Rₜ=0.98 min; MS (ESIpos): m/z=245 [M+H]⁺

¹H-NMR (400 MHz, DMSO-d6) δ [ppm]: 0.970 (1.40), 0.982 (3.58), 0.989 (5.40), 0.993 (3.48), 0.995 (3.30), 1.000 (4.37), 1.007 (2.13), 1.029 (0.91), 1.044 (2.24), 1.052 (4.11), 1.059 (2.50), 1.064 (2.91), 1.071 (4.67), 1.079 (2.55), 1.090 (1.04), 1.448 (1.13), 1.455 (0.89), 1.469 (2.34), 1.472 (2.13), 1.475 (2.36), 1.493 (2.46), 1.500 (2.43), 1.514 (1.05), 1.520 (1.39), 1.857 (0.58), 1.874 (0.91), 1.881 (0.94), 1.893 (0.86), 1.899 (1.41), 1.916 (1.00), 1.923 (0.65), 1.940 (0.43), 1.987 (0.40), 2.064 (0.68), 2.076 (1.35), 2.084 (1.48), 2.095 (2.54), 2.107 (1.39), 2.115 (1.24), 2.127 (0.58), 2.145 (1.39), 2.152 (1.25), 2.163 (2.62), 2.169 (2.30), 2.173 (2.04), 2.180 (1.99), 2.183 (2.33), 2.190 (2.49), 2.197 (0.93), 2.201 (1.12), 2.208 (1.24), 2.327 (0.49), 2.518 (1.52), 2.523 (0.94), 2.669 (0.44), 2.728 (1.33), 2.888 (1.60), 3.292 (3.69), 3.307 (6.46), 3.322 (4.25), 3.355 (0.46), 3.811 (1.00), 3.814 (0.87), 3.831 (1.99), 3.848 (2.02), 3.865 (0.91), 3.868 (0.93), 4.919 (7.38), 4.935 (7.12), 4.945 (0.49), 5.758 (0.41), 7.474 (1.25), 7.488 (2.37), 7.503 (1.19), 7.731 (0.46), 7.871 (16.00), 7.875 (1.33).

### Intermediate 101

### cis-3-[(3-amino-6-cyclopropyl-1H-pyrazolo[3,4-b]pyrazin-1-yl)methyl]cyclobutan-1-ol

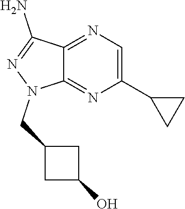 [SMILES: Nc1nn(C[C@H]2C[C@@H](O)C2)c2nc(C3CC3)cnc12]

In analogy to 42) 5-cyclopropyl-3-{[(cis-3-hydroxycyclobutyl)methyl]amino}pyrazine-2-carbonitrile (162 mg, 663 μmol, intermediate 100) and O-(diphenylphosphinoyl)hydroxylamine (209 mg, 895 μmol) reacted and give after purification using a Biotage chromatography 102 mg (90% purity, 53% yield) of the desired title compound.

LC-MS (Method 2): Rₜ=0.79 min; MS (ESIpos): m/z=261 [M+H]⁺

¹H-NMR (400 MHz, DMSO-d6) δ [ppm]: 1.003 (0.45), 1.016 (1.20), 1.027 (2.92), 1.034 (4.05), 1.040 (3.39), 1.045 (3.93), 1.052 (1.93), 1.060 (0.73), 1.074 (2.13), 1.081 (3.70), 1.087 (2.39), 1.094 (2.26), 1.101 (4.15), 1.108 (2.21), 1.116 (0.68), 1.119 (0.96), 1.154 (0.44), 1.171 (0.89), 1.189 (0.44), 1.522 (0.54), 1.528 (0.66), 1.532 (0.68), 1.542 (1.96), 1.548 (1.80), 1.564 (2.06), 1.568 (1.95), 1.586 (0.86), 1.591 (0.91), 1.987 (1.50), 2.070 (0.62), 2.087 (0.84), 2.092 (0.91), 2.107 (1.34), 2.120 (2.74), 2.135 (2.54), 2.140 (2.00), 2.152 (1.64), 2.157 (1.90), 2.167 (0.85), 2.176 (0.78), 2.272 (0.59), 2.284 (1.17), 2.292 (1.26), 2.304 (2.05), 2.313 (0.95), 2.316 (1.25), 2.324 (1.31), 2.332 (0.52), 2.336 (0.63), 2.518 (1.62), 2.522 (1.02), 2.669 (0.47), 3.812 (0.78), 3.831 (1.44), 3.848 (1.40), 3.865 (0.63), 4.034 (0.41), 4.053 (5.78), 4.069 (4.94), 4.102 (0.44), 4.939 (6.95), 4.946 (0.51), 4.957 (6.45), 4.962 (0.42), 5.694 (6.97), 5.724 (0.42), 5.758 (0.89), 7.731 (0.57), 8.316 (16.00), 8.323 (0.95), 8.326 (0.80).

### Intermediate 102

### 5-cyclopropyl-3-{[(1-hydroxycyclobutyl)methyl]amino}pyrazine-2-carbonitrile

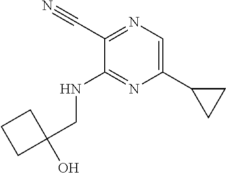 [SMILES: N#Cc1ncc(C2CC2)nc1NCC1(O)CCC1]

In analogy to 39) 3-chloro-5-cyclopropylpyrazine-2-carbonitrile (400 mg, 2.23 mmol, intermediate 1) and 1-(aminomethyl)cyclobutan-1-ol (248 mg, 2.45 mmol, CAS-RN:[180205-28-1], commercially available e.g. Fluorochem) reacted and give after purification using a Biotage chromatography 390 mg (98% purity, 70% yield) of the desired title compound.

LC-MS (Method 2): Rₜ=1.09 min; MS (ESIpos): m/z=245 [M+H]⁺

¹H-NMR (400 MHz, DMSO-d6) δ [ppm]: 0.993 (1.12), 1.000 (1.87), 1.004 (1.19), 1.007 (1.08), 1.012 (1.51), 1.019 (0.73), 1.058 (0.66), 1.066 (1.44), 1.073 (0.84), 1.078 (1.00), 1.086 (1.67), 1.093 (0.94), 1.097 (0.43), 1.429 (0.47), 1.433 (0.40), 1.456 (0.57), 1.886 (0.68), 1.890 (0.68), 1.912 (1.73), 1.916 (1.79), 1.933 (1.45), 1.938 (1.33), 1.944 (0.94), 2.099 (0.49), 2.107 (0.53), 2.119 (0.89), 2.130 (0.49), 2.138 (0.45), 2.518 (0.51), 2.729 (13.40), 2.888 (16.00), 3.509 (2.59), 3.524 (2.65), 5.249 (5.29), 6.882 (0.77), 7.923 (5.73), 7.950 (1.95).

### Intermediate 103

### 1-[(3-amino-6-cyclopropyl-1H-pyrazolo[3,4-b]pyrazin-1-yl)methyl]cyclobutan-1-ol

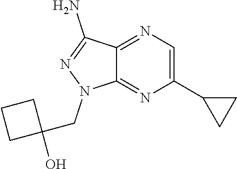 [SMILES: Nc1nn(CC2(O)CCC2)c2nc(C3CC3)cnc12]

In analogy to 40) 5-cyclopropyl-3-{[(1-hydroxycyclobutyl)methyl]amino}pyrazine carbonitrile (390 mg, 1.60 mmol, intermediate 102) and O-(diphenylphosphinoyl)hydroxylamine (503/372 mg, 2.16/1.60 mmol) reacted and give after purification using a Biotage chromatography 83.6 mg (91% purity, 18% yield) of the desired title compound.

LC-MS (Method 2): Rₜ=0.91 min; MS (ESIpos): m/z=260 [M+H]⁺

¹H-NMR (400 MHz, DMSO-d6) δ [ppm]: 1.001 (1.21), 1.004 (0.94), 1.012 (3.16), 1.019 (4.48), 1.026 (3.31), 1.031 (4.04), 1.038 (1.94), 1.052 (0.58), 1.059 (0.81), 1.066 (2.06), 1.073 (4.11), 1.080 (2.28), 1.086 (2.60), 1.093 (4.20), 1.100 (2.38), 1.106 (0.83), 1.111 (1.06), 1.184 (0.70), 1.206 (1.23), 1.211 (0.99), 1.228 (1.15), 1.233 (1.49), 1.256 (0.92), 1.532 (0.41), 1.548 (0.54), 1.556 (1.04), 1.565 (0.61), 1.575 (0.60), 1.584 (1.06), 1.589 (0.58), 1.608 (0.52), 1.872 (0.89), 1.879 (0.70), 1.896 (2.36), 1.903 (2.20), 1.920 (2.14), 1.927 (2.59), 1.951 (0.98), 2.170 (1.23), 2.178 (1.64), 2.185 (1.14), 2.191 (1.70), 2.200 (2.55), 2.209 (1.53), 2.216 (0.92), 2.223 (1.34), 2.231 (0.92), 2.272 (0.61), 2.284 (1.19), 2.292 (1.26), 2.304 (2.13), 2.313 (0.92), 2.316 (1.21), 2.324 (1.27), 2.332 (0.42), 2.336 (0.61), 2.518 (1.16), 2.523 (0.79), 3.524 (0.41), 3.632 (0.42), 3.660 (0.45), 4.129 (12.53), 5.121 (15.89), 5.248 (0.77), 5.722 (7.40), 5.758 (0.62), 7.924 (0.87), 8.329 (16.00).

### Intermediate 104

### 5-cyclopropyl-3-{[(1-hydroxycyclopentyl)methyl]amino}pyrazine-2-carbonitrile

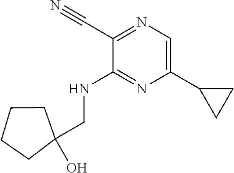 [SMILES: N#Cc1ncc(C2CC2)nc1NCC1(O)CCCC1]

In analogy to 39) 3-chloro-5-cyclopropylpyrazine-2-carbonitrile (400 mg, 2.23 mmol, intermediate 1) and 1-(aminomethyl)cyclopentan-1-ol-hydrogen chloride (1/1) (371 mg, 2.45 mmol, CAS-RN:[76066-27-8], commercially available e.g. Combi-Blocks Inc.) reacted and give after purification using a Biotage chromatography 470 mg (85% purity, 70% yield) of the desired title compound.

LC-MS (Method 2): Rₜ=1.15 min; MS (ESIpos): m/z=259 [M+H]⁺

¹H-NMR (400 MHz, DMSO-d6) δ [ppm]: 0.982 (0.85), 0.989 (1.42), 0.993 (0.85), 0.996 (0.78), 1.000 (1.19), 1.008 (0.56), 1.048 (0.50), 1.055 (1.10), 1.062 (0.63), 1.068 (0.75), 1.076 (1.24), 1.083 (0.76), 1.478 (0.60), 1.486 (1.21), 1.497 (2.57), 1.519 (0.66), 1.532 (0.41), 1.670 (0.54), 1.677 (0.58), 1.689 (0.52), 2.107 (0.69), 2.727 (13.36), 2.888 (16.00), 3.442 (2.29), 3.457 (2.26), 4.592 (4.31), 6.897 (0.59), 7.905 (4.49), 7.950 (1.88).

### Intermediate 105

### 1-[(3-amino-6-cyclopropyl-1H-pyrazolo[3,4-b]pyrazin-1-yl)methyl]cyclopentan-1-ol

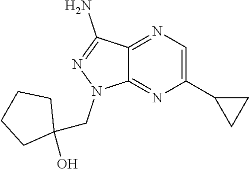 [SMILES: Nc1nn(CC2(O)CCCC2)c2nc(C3CC3)cnc12]

In analogy to 42) 5-cyclopropyl-3-{[(1-hydroxycyclopentyl)methyl]amino}pyrazine-2-carbonitrile (470 mg, 1.82 mmol, intermediate 104) and O-(diphenylphosphinoyl)hydroxylamine (573 mg, 2.46 mmol) reacted and give after purification using a Biotage chromatography 190 mg (92% purity, 35% yield) of the desired title compound.

LC-MS (Method 2): Rₜ=0.98 min; MS (ESIpos): m/z=274 [M+H]⁺

¹H-NMR (400 MHz, DMSO-d6) δ [ppm]: 1.005 (1.08), 1.017 (2.92), 1.023 (4.29), 1.029 (3.28), 1.035 (4.10), 1.042 (1.93), 1.054 (0.65), 1.062 (0.77), 1.068 (1.79), 1.076 (3.95), 1.082 (2.21), 1.088 (2.30), 1.095 (3.98), 1.102 (2.28), 1.109 (0.80), 1.114 (1.00), 1.401 (2.38), 1.420 (0.99), 1.451 (1.08), 1.465 (1.79), 1.473 (1.86), 1.480 (2.02), 1.489 (1.81), 1.497 (1.69), 1.622 (2.58), 1.631 (3.58), 1.640 (5.35), 1.648 (2.98), 2.269 (0.53), 2.281 (1.11), 2.290 (1.21), 2.301 (2.03), 2.310 (0.91), 2.313 (1.16), 2.321 (1.27), 2.326 (0.50), 2.333 (0.55), 2.518 (1.13), 2.522 (0.71), 2.727 (3.01), 2.729 (3.07), 2.887 (3.81), 4.131 (13.53), 4.534 (11.74), 4.542 (0.49), 5.733 (7.36), 5.758 (0.54), 7.950 (0.47), 8.324 (16.00).

### Intermediate 106

### 5-cyclopropyl-3-({[1-(hydroxymethyl)cyclobutyl]methyl}amino)pyrazine-2-carbonitrile

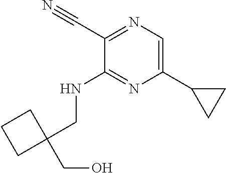 [SMILES: N#Cc1ncc(C2CC2)nc1NCC1(CO)CCC1]

In analogy to 39) 3-chloro-5-cyclopropylpyrazine-2-carbonitrile (400 mg, 2.23 mmol, intermediate 1) and [1-(aminomethyl)cyclobutyl]methanol (282 mg, 2.45 mmol, CAS-RN:[2041-56-7], commercially available e.g. Fluorochem) reacted and give after purification using a Biotage chromatography 340 mg (95% purity, 56% yield) of the desired title compound.

LC-MS (Method 1): Rₜ=1.17 min; MS (ESIpos): m/z=259 [M+H]⁺

¹H-NMR (400 MHz, DMSO-d6) δ [ppm]: 0.997 (1.37), 1.009 (3.35), 1.016 (4.47), 1.022 (4.38), 1.028 (4.17), 1.034 (2.18), 1.038 (1.15), 1.051 (2.63), 1.057 (3.87), 1.064 (2.33), 1.070 (2.39), 1.077 (4.78), 1.083 (2.32), 1.095 (1.05), 1.678 (0.83), 1.687 (1.62), 1.693 (2.27), 1.696 (2.24), 1.710 (6.82), 1.724 (6.66), 1.743 (1.24), 1.748 (0.83), 1.755 (1.28), 1.760 (1.27), 1.769 (1.75), 1.776 (1.82), 1.785 (2.15), 1.795 (1.37), 1.803 (1.57), 1.819 (0.88), 1.824 (0.54), 2.074 (0.60), 2.086 (1.19), 2.094 (1.28), 2.102 (1.22), 2.106 (2.08), 2.114 (0.96), 2.118 (1.24), 2.126 (1.12), 2.137 (0.47), 2.518 (1.16), 2.523 (0.75), 2.729 (12.96), 2.888 (16.00), 3.384 (8.13), 3.398 (8.05), 3.466 (7.11), 3.480 (7.12), 4.799 (2.40), 4.813 (6.12), 4.827 (2.32), 7.247 (1.05), 7.261 (2.13), 7.275 (1.03), 7.890 (15.37), 7.950 (1.95), 8.556 (0.58).

### Intermediate 107

### {1-[(3-amino-6-cyclopropyl-1H-pyrazolo[3,4-b]pyrazin-1-yl)methyl]cyclobutyl}methanol

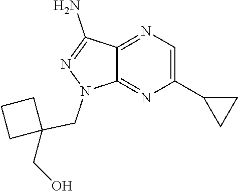 [SMILES: Nc1nn(CC2(CO)CCC2)c2nc(C3CC3)cnc12]

In analogy to 40) 5-cyclopropyl-3-({[1-(hydroxymethyl)cyclobutyl]methyl}amino)pyrazine-2-carbonitrile (340 mg, 1.32 mmol, intermediate 106) and O-(diphenylphosphinoyl)hydroxylamine (414/307 mg, 1.78/1.32 mmol) reacted 16 hours at room temperature and 16 hours at 55° C. and give after purification using a Biotage chromatography 110 mg (43% purity, 13% yield) of the desired title compound.

LC-MS (Method 2): Rₜ=0.97 min; MS (ESIpos): m/z=274 [M+H]⁺

¹H-NMR (400 MHz, DMSO-d6) δ [ppm]: 0.011 (0.85), 0.063 (1.20), 0.067 (0.46), 0.850 (0.57), 0.868 (0.40), 0.946 (0.74), 0.963 (1.14), 0.988 (5.58), 0.996 (4.21), 1.008 (4.90), 1.017 (4.95), 1.024 (7.35), 1.029 (7.40), 1.036 (7.00), 1.043 (4.38), 1.064 (2.39), 1.070 (3.59), 1.078 (6.04), 1.084 (4.73), 1.090 (3.76), 1.097 (5.58), 1.104 (3.99), 1.116 (1.71), 1.154 (1.94), 1.172 (3.64), 1.190 (1.88), 1.230 (1.65), 1.259 (1.65), 1.352 (0.46), 1.613 (0.57), 1.627 (0.97), 1.653 (1.88), 1.670 (2.79), 1.680 (2.68), 1.688 (2.45), 1.702 (2.90), 1.708 (3.13), 1.723 (3.64), 1.740 (7.63), 1.756 (5.30), 1.771 (6.78), 1.781 (5.12), 1.795 (4.10), 1.838 (4.56), 1.850 (4.27), 1.860 (3.47), 1.889 (1.54), 1.987 (6.15), 2.038 (0.46), 2.052 (0.68), 2.056 (0.74), 2.062 (0.68), 2.070 (1.42), 2.084 (1.02), 2.088 (1.08), 2.104 (0.85), 2.118 (0.68), 2.146 (0.46), 2.159 (1.25), 2.276 (0.63), 2.288 (1.31), 2.296 (1.48), 2.308 (2.45), 2.322 (2.90), 2.327 (3.87), 2.331 (2.56), 2.336 (1.20), 2.518 (12.58), 2.522 (7.91), 2.660 (1.02), 2.664 (2.22), 2.668 (3.02), 2.673 (2.16), 2.678 (1.02), 2.888 (0.46), 3.277 (0.80), 3.302 (9.00), 3.316 (11.27), 3.353 (5.41), 3.372 (1.77), 3.385 (2.05), 3.397 (1.54), 3.406 (1.71), 3.419 (1.71), 3.466 (0.68), 3.482 (4.16), 3.497 (4.10), 3.537 (0.51), 3.556 (1.42), 3.570 (1.59), 3.584 (1.65), 3.599 (1.54), 3.615 (0.46), 3.999 (0.46), 4.017 (1.31), 4.034 (1.37), 4.052 (0.46), 4.131 (14.98), 4.399 (0.63), 4.430 (0.80), 4.546 (0.46), 4.570 (0.51), 4.589 (2.68), 4.603 (6.83), 4.617 (2.62), 4.670 (1.59), 4.683 (3.81), 4.696 (1.59), 4.787 (0.51), 4.800 (1.25), 4.813 (0.68), 5.720 (8.65), 5.758 (8.26), 7.459 (1.20), 7.656 (0.63), 7.720 (8.14), 7.811 (0.74), 7.847 (0.97), 7.856 (2.90), 7.880 (0.91), 7.891 (0.85), 7.920 (2.90), 7.983 (1.14), 7.998 (0.40), 8.333 (16.00), 8.340 (0.91), 8.470 (0.57), 8.592 (0.74), 8.639 (0.63), 8.825 (0.63), 8.839 (1.20), 8.854 (0.63), 9.006 (0.57), 9.906 (0.51), 10.537 (1.42), 10.849 (0.57), 11.932 (1.42).

### Intermediate 108

### 3-[(cyclobutylmethyl)amino]-5-cyclopropylpyrazine-2-carbonitrile

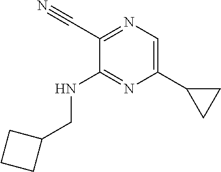 [SMILES: N#Cc1ncc(C2CC2)nc1NCC1CCC1]

In analogy to 39) 3-chloro-5-cyclopropylpyrazine-2-carbonitrile (400 mg, 2.23 mmol, intermediate 1) and 1-cyclobutylmethanamine-hydrogen chloride (1/1) (298 mg, 2.45 mmol) reacted and give after purification using a Biotage chromatography 680 mg (80% purity, 107% yield) of the desired title compound.

LC-MS (Method 2): Rₜ=1.41 min; MS (ESIpos): m/z=229 [M+H]⁺

¹H-NMR (400 MHz, DMSO-d6) δ [ppm]: 0.977 (0.22), 0.984 (0.34), 0.989 (0.25), 0.996 (0.35), 1.003 (0.19), 1.052 (0.25), 1.064 (0.19), 1.072 (0.30), 1.080 (0.17), 1.780 (0.20), 1.801 (0.25), 2.523 (0.18), 2.729 (12.99), 2.888 (16.00), 3.189 (0.41), 3.342 (0.34), 3.345 (0.33), 3.360 (0.26), 7.868 (0.91), 7.950 (1.98), 8.554 (0.22).

### Intermediate 109

### -(cyclobutylmethyl)-6-cyclopropyl-1H-pyrazolo[3,4-b]pyrazin-3-amine

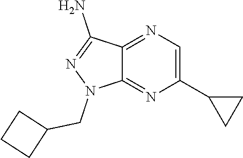 [SMILES: Nc1nn(CC2CCC2)c2nc(C3CC3)cnc12]

In analogy to 42) 3-[(cyclobutylmethyl)amino]-5-cyclopropylpyrazine-2-carbonitrile (680 mg, 2.38 mmol, intermediate 108) and O-(diphenylphosphinoyl)hydroxylamine (750 mg, 3.22 mmol) reacted and give after purification using a Biotage chromatography 300 mg (63% purity, 33% yield) of the desired title compound.

LC-MS (Method 2): Rₜ=1.17 min; MS (ESIpos): m/z=244 [M+H]⁺

¹H-NMR (400 MHz, DMSO-d6) δ [ppm]: 1.030 (0.47), 1.042 (0.45), 1.076 (0.42), 1.097 (0.47), 1.778 (0.40), 2.729 (13.77), 2.888 (16.00), 3.189 (0.57), 4.082 (0.79), 4.100 (0.78), 5.691 (0.72), 7.950 (1.95), 8.313 (1.64).

### Intermediate 110

### 4-(3-amino-6-cyclopropyl-1H-pyrazolo[3,4-b]pyrazin-1-yl)-2-methylbutan-2-ol

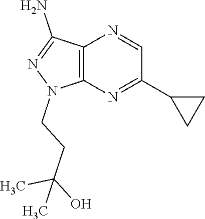 [SMILES: CC(C)(O)CCn1nc(N)c2ncc(C3CC3)nc21]

In analogy to 90) 3-chloro-5-cyclopropylpyrazine-2-carbonitrile (300 mg, 1.67 mmol) and oxalic acid-4-hydrazinyl-2-methylbutan-2-ol (1/1) (522 mg, 2.51 mmol, CAS-RN:[1803581-82-9], commercially available e.g. Enamine) reacted 4 hours at 120° C. and 16 hours at 90° C. and give after purification using a Biotage chromatography 431 mg (95% purity, 94% yield) of the desired title compound.

LC-MS (Method 2): Rₜ=0.87 min; MS (ESIpos): m/z=262 [M+H]⁺

¹H-NMR (400 MHz, DMSO-d6) δ [ppm]: 1.027 (0.88), 1.033 (1.17), 1.040 (1.31), 1.045 (1.14), 1.052 (0.64), 1.068 (0.79), 1.075 (1.21), 1.078 (1.44), 1.082 (0.92), 1.084 (0.88), 1.088 (0.91), 1.095 (1.61), 1.105 (16.00), 1.131 (0.83), 1.153 (0.60), 1.170 (1.10), 1.188 (0.50), 1.800 (0.86), 1.814 (0.69), 1.820 (0.91), 1.826 (0.68), 1.840 (0.92), 1.986 (2.07), 2.300 (0.59), 3.959 (0.48), 4.016 (0.44), 4.034 (0.44), 4.130 (0.92), 4.144 (0.70), 4.150 (0.91), 4.156 (0.69), 4.170 (0.88), 4.393 (4.06), 5.703 (2.04), 5.758 (4.29), 8.302 (4.32).

### Intermediate 111

### 3-chloro-5-methylpyrazine-2-carbonitrile

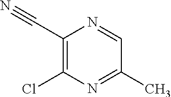

To a mixture of 3,5-dichloropyrazine-2-carbonitrile (1.00 g, 5.75 mmol) and methylboronic acid (413 mg, 6.90 mmol) in 10 mL toluene cesium fluoride (2.62 g, 17.2 mmol) and 1,1′-bis(diphenylphosphino)ferrocenepalladium(II) chloride (210 mg, 287 μmol; CAS-RN:[72287-26-4]). The mixture was stirred for 16 hours at 90° C. After cooling to room temperature the mixture was filtered under suction and the filter cake was washed with ethyl acetate. The filtrate was concentrated and the residue was purified using a Biotage chromatography system to give 105 mg (95% purity, 11% yield) of the disered title compound.

¹H-NMR (400 MHz, DMSO-d6) δ [ppm]: 2.616 (16.00), 4.023 (1.20), 8.773 (3.94).

### Intermediate 112

### 6-methyl-1-(1-methylpiperidin-4-yl)-1H-pyrazolo[3,4-b]pyrazin-3-amine

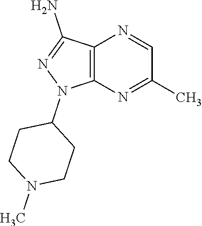 [SMILES: Cc1cnc2c(N)nn(C3CCN(C)CC3)c2n1]

In analogy to 90) 3-chloro-5-methylpyrazine-2-carbonitrile (252 mg, 1.64 mmol, intermediate 111) and 4-hydrazinyl-1-methylpiperidine-hydrogen chloride (1/2) (498 mg, 2.46 mmol) reacted4 hours at 120° C. and 16 hours at 90° C. and give after purification using a Biotage chromatography 65.0 mg (90% purity, 14% yield) of the desired title compound.

LC-MS (Method 2): Rₜ=0.76 min; MS (ESIpos): m/z=247 [M+H]⁺

¹H-NMR (400 MHz, DMSO-d6) δ [ppm]: 0.919 (0.59), 1.231 (0.40), 1.722 (1.13), 1.727 (1.10), 1.754 (1.11), 1.987 (0.67), 2.013 (0.69), 2.043 (1.41), 2.071 (2.81), 2.097 (1.19), 2.104 (1.17), 2.121 (1.32), 2.134 (0.95), 2.156 (0.42), 2.164 (0.79), 2.175 (0.80), 2.181 (0.49), 2.196 (1.43), 2.204 (12.32), 2.323 (0.54), 2.327 (0.80), 2.331 (0.54), 2.346 (0.50), 2.373 (0.66), 2.382 (0.43), 2.395 (1.03), 2.410 (1.79), 2.432 (0.90), 2.450 (1.06), 2.459 (0.58), 2.518 (1.77), 2.523 (1.26), 2.570 (16.00), 2.590 (0.99), 2.641 (0.79), 2.647 (0.74), 2.665 (0.48), 2.669 (0.64), 2.674 (0.46), 2.861 (1.25), 2.886 (1.50), 3.183 (0.58), 3.250 (1.15), 3.957 (0.84), 4.420 (0.50), 4.431 (0.63), 5.759 (0.50), 5.776 (3.49), 7.929 (0.44), 7.970 (0.46), 8.249 (6.08).

### EXAMPLE 1

### N-[(1H-benzimidazol-2-yl)methyl]-6-cyclopropyl-1-(1-methylpiperidin-4-yl)-1H-pyrazolo[3,4-b]pyrazin-3-amine

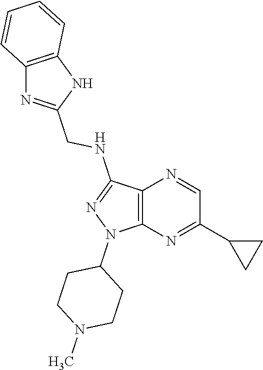 [SMILES: CN1CCC(n2nc(NCc3nc4ccccc4[nH]3)c3ncc(C4CC4)nc32)CC1]

To a solution of 6-cyclopropyl-1-(1-methylpiperidin-4-yl)-1H-pyrazolo[3,4-b]pyrazin-3-amine (150 mg, 551 μmol, intermediate 2) in 4.9 mL methanol was added 1H-benzimidazole-2-carbaldehyde (161 mg, 1.10 mmol, CAS-RN:[3314-30-5], commercially available e.g. Apollo) and 16 μL acetic acid. The mixture was stirred over night at 60° C. After cooling to room temperature sodium cyanoborohydride (69.2 mg, 1.10 mmol) and stirred for two hours at room temperature. Then an additional portion of sodium cyanoborohydride (69.2 mg, 1.10 mmol) and 16 μL acetic acid was added and stirring was continued for additional 2 hours at room temperature. Then the mixture was diluted with 100 mL ethyl acetate, extracted twice with water, brine, filtered using a hydrophobic filter and evaporated to dryness. The residue was purified by HPLC obtaining 46.6 mg (95% purity, 20% yield) of the desired titled compound.

LC-MS (Method 2): Rₜ=1.11 min; MS (ESIpos): m/z=403 [M+H]⁺

¹H-NMR (400 MHz, DMSO-d6) δ [ppm]=1.01-1.07 (m, 2H), 1.07-1.13 (m, 2H), 1.66-1.77 (m, 2H), 1.99-2.05 (m, 2H), 2.07-2.19 (m, 5H), 2.28-2.36 (m, 1H), 2.82 (br d, 2H), 4.33 (tt, 1H), 4.76 (d, 2H), 6.90 (t, 1H), 7.07-7.15 (m, 2H), 7.37-7.44 (m, 1H), 7.49-7.56 (m, 1H), 8.36 (s, 1H), 12.18 (s, 1H).

### EXAMPLE 2

### 6-cyclopropyl-N-[(4-methoxy-1H-benzimidazol-2-yl)methyl]-1-(1-methylpiperidin-4-yl)-1H-pyrazolo[3,4-b]pyrazin-3-amine

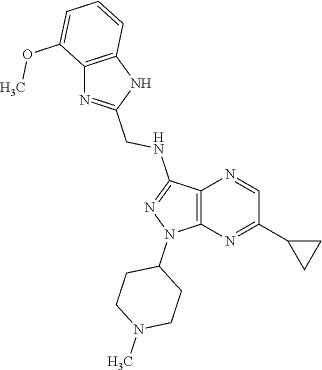 [SMILES: COc1cccc2[nH]c(CNc3nn(C4CCN(C)CC4)c4nc(C5CC5)cnc34)nc12]

To a solution of 6-cyclopropyl-1-(1-methylpiperidin-4-yl)-1H-pyrazolo[3,4-b]pyrazin-3-amine (75.0 mg, 275 μmol, intermediate 2) in 2.5 mL methanol was added 4-methoxy-1H-benzimidazole-2-carbaldehyde (164 mg, 89% purity, 826 μmol, commercially available e.g. Ellanova Laborotories) and 32 μL acetic acid. The mixture was stirred over night at 60° C. After cooling to room temperature sodium cyanoborohydride (69.2 mg, 1.10 mmol) was added and the mixture was stirred for two hours at room temperature. Then the mixture was diluted with 100 mL ethyl acetate, extracted twice with water, brine, filtered using a hydrophobic filter and evaporated to dryness. The residue was purified by two subsequent HPLC obtaining 16.5 mg (95% purity, 13% yield) of the desired titled compound.

LC-MS (Method 2): Rₜ=1.14 min; MS (ESIpos): m/z=433 [M+H]⁺

¹H-NMR (400 MHz, DMSO-d6) δ [ppm]: 1.017 (0.72), 1.029 (1.98), 1.035 (2.80), 1.040 (2.48), 1.047 (2.45), 1.090 (2.36), 1.097 (1.50), 1.109 (2.43), 1.117 (1.50), 1.154 (0.47), 1.172 (0.73), 1.190 (0.40), 1.232 (1.34), 1.708 (1.47), 1.732 (1.59), 1.988 (1.83), 2.021 (1.66), 2.048 (1.19), 2.097 (0.70), 2.119 (1.01), 2.128 (1.45), 2.148 (0.96), 2.157 (1.38), 2.176 (9.48), 2.189 (6.67), 2.285 (0.48), 2.297 (0.71), 2.305 (0.83), 2.318 (1.85), 2.322 (2.07), 2.327 (2.97), 2.332 (2.04), 2.336 (1.34), 2.518 (7.21), 2.523 (4.92), 2.539 (2.61), 2.660 (0.72), 2.664 (1.56), 2.669 (2.22), 2.673 (1.59), 2.678 (0.67), 2.810 (1.45), 2.838 (1.48), 3.898 (16.00), 3.904 (10.31), 4.303 (0.44), 4.321 (0.57), 4.331 (0.85), 4.359 (0.44), 4.715 (3.67), 4.730 (3.62), 5.759 (4.45), 6.615 (1.36), 6.619 (1.45), 6.634 (1.56), 6.637 (1.52), 6.669 (0.50), 6.684 (1.04), 6.700 (0.46), 6.722 (1.16), 6.741 (1.32), 6.846 (0.73), 6.861 (1.57), 6.876 (0.68), 6.983 (0.56), 6.987 (0.98), 7.003 (2.79), 7.007 (2.49), 7.012 (2.92), 7.015 (1.30), 7.029 (2.23), 7.036 (2.01), 7.050 (0.90), 7.055 (1.17), 7.126 (1.52), 7.146 (1.02), 8.358 (10.63), 12.161 (1.40), 12.435 (0.99).

### EXAMPLE 3

### 6-cyclopropyl-N-[(4-fluoro-1H-benzimidazol-2-yl)methyl]-1-(1-methylpiperidin-4-yl)-1H-pyrazolo[3,4-b]pyrazin-3-amine

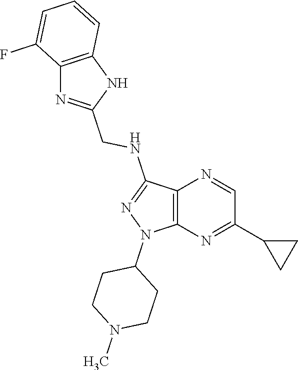 [SMILES: CN1CCC(n2nc(NCc3nc4c(F)cccc4[nH]3)c3ncc(C4CC4)nc32)CC1]

To a solution of 6-cyclopropyl-1-(1-methylpiperidin-4-yl)-1H-pyrazolo[3,4-b]pyrazin-3-amine (100 mg, 367 μmol, intermediate 2) in 3.3 mL methanol was added 4-fluoro-1H-benzimidazole-2-carbaldehyde (121 mg, 734 μmol, intermediate 5) and 2.1 μL acetic acid. The mixture was stirred over night at 60° C. After cooling to room temperature sodium cyanoborohydride (46.1 mg, 734 μmol) was added and the mixture was stirred for 90 minutes at room temperature. Then the mixture was diluted with water, extracted threetimes with ethyl acetate. The combined organic phase was filtered using a hydrophobic filter and evaporated to dryness. The residue was purified via Biotage chromatography system followed by HPLC obtaining 12.7 mg (7.4% yield) of the desired title compound.

LC-MS (Method 2): Rₜ=1.15 min; MS (ESIpos): m/z=422 [M+H]⁺

¹H-NMR (400 MHz, DMSO-d6) δ [ppm]: −0.149 (0.67), 0.146 (0.68), 0.836 (0.65), 0.854 (1.13), 0.869 (0.71), 0.908 (0.49), 1.042 (1.93), 1.054 (4.98), 1.061 (7.08), 1.067 (6.44), 1.073 (6.94), 1.080 (3.44), 1.091 (1.49), 1.106 (3.52), 1.113 (6.87), 1.119 (4.28), 1.125 (4.36), 1.132 (7.06), 1.139 (4.15), 1.151 (1.94), 1.199 (0.77), 1.234 (4.42), 1.355 (0.45), 1.753 (0.50), 1.952 (2.61), 2.104 (0.56), 2.206 (2.45), 2.235 (2.22), 2.310 (1.20), 2.321 (3.12), 2.325 (3.89), 2.329 (5.96), 2.334 (4.13), 2.341 (3.87), 2.353 (2.18), 2.362 (1.87), 2.374 (0.88), 2.521 (13.68), 2.525 (8.87), 2.542 (2.92), 2.589 (4.12), 2.662 (1.62), 2.667 (3.06), 2.672 (4.04), 2.676 (3.06), 2.681 (1.64), 2.698 (1.78), 2.876 (0.92), 3.017 (0.60), 3.233 (2.83), 4.618 (1.23), 4.757 (8.39), 4.772 (8.33), 4.799 (0.71), 6.902 (2.20), 6.921 (2.95), 6.929 (2.37), 6.949 (2.81), 7.077 (3.00), 7.090 (2.99), 7.097 (5.93), 7.109 (6.17), 7.117 (4.28), 7.130 (4.39), 7.233 (5.45), 7.253 (4.08), 7.377 (0.52), 7.396 (0.44), 8.020 (0.73), 8.091 (0.62), 8.351 (0.41), 8.416 (16.00), 12.476 (3.27), 12.846 (0.49).

### EXAMPLE 4

### 6-cyclopropyl-N-[(5-fluoro-1H-benzimidazol-2-yl)methyl]-1-(1-methylpiperidin-4-yl)-1H-pyrazolo[3,4-b]pyrazin-3-amine

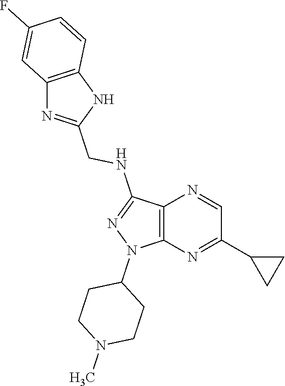 [SMILES: CN1CCC(n2nc(NCc3nc4cc(F)ccc4[nH]3)c3ncc(C4CC4)nc32)CC1]

In analoga to example 2) 6-cyclopropyl-1-(1-methylpiperidin-4-yl)-1H-pyrazolo[3,4-b]pyrazin-3-amine (60.0 mg, 220 μmol, intermediate 2) and 5-fluoro-1H-benzimidazole-2-carbaldehyde (72.3 mg, 441 μmol, intermediate 6) reacted and give after one HPLC purifcation 14.0 mg (14% yield) of the desired title compound.

LC-MS (Method 2): Rₜ=1.21 min; MS (ESIpos): m/z=422 [M+H]⁺

¹H-NMR (400MHz, DMSO-d6): δ [ppm]=1.00-1.14 (m, 4H), 1.71 (m, 2H), 1.98-2.21 (m, 7H), 2.25-2.35 (m, 1H), 2.82 (m, 2H), 4.29-4.37 (m, 1H), 4.71-4.77 (m, 2H), 6.91-7.01 (m, 2H), 7.16-7.23/7.29-7.35 (m, 1H), 7.36-7.41/7.49-7.56 (m, 1H), 8.36 (s, 1H), 12.23-12.32 (m, 1H).

### EXAMPLE 5

### 6-cyclopropyl-1-(1-methylpiperidin-4-yl)-N-{[4-(trifluoromethoxy)-1H-benzimidazol-2-yl]methyl}-1H-pyrazolo[3,4-b]pyrazin-3-amine

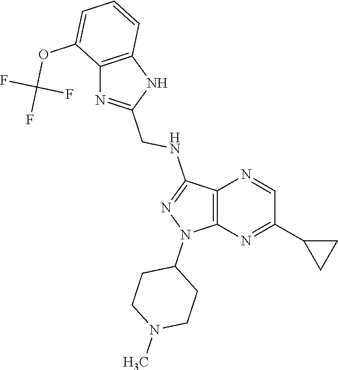 [SMILES: CN1CCC(n2nc(NCc3nc4c(OC(F)(F)F)cccc4[nH]3)c3ncc(C4CC4)nc32)CC1]

In analogy to example 2) 6-cyclopropyl-1-(1-methylpiperidin-4-yl)-1H-pyrazolo[3,4-b]pyrazin-3-amine (75.0 mg, 275 μmol, intermediate 2) and 4-(trifluoromethoxy)-1H-benzimidazole-2-carbaldehyde (147 mg, 551 μmol, intermediate 7) reacted and give after one HPLC purifcation 16.0 mg (11% yield) of the desired title compound.

LC-MS (Method 2): R₁=1.29 min; MS (ESIneg): m/z=485 [M−H]⁻

¹H-NMR (400 MHz, DMSO-d6) δ [ppm]: 1.028 (3.11), 1.035 (4.56), 1.041 (3.81), 1.046 (4.06), 1.054 (2.12), 1.084 (1.94), 1.091 (3.73), 1.098 (2.51), 1.104 (2.72), 1.111 (4.06), 1.118 (2.56), 1.232 (1.32), 1.689 (2.07), 1.718 (2.30), 1.978 (1.42), 2.004 (3.24), 2.033 (2.69), 2.074 (1.27), 2.084 (1.76), 2.097 (2.23), 2.105 (2.33), 2.126 (1.94), 2.135 (2.10), 2.160 (16.00), 2.286 (0.70), 2.298 (1.19), 2.307 (1.37), 2.318 (2.72), 2.323 (2.05), 2.327 (2.80), 2.337 (1.50), 2.350 (0.67), 2.518 (10.80), 2.523 (7.35), 2.593 (0.65), 2.665 (1.09), 2.669 (1.55), 2.673 (1.22), 2.784 (2.61), 2.813 (2.59), 3.462 (0.49), 4.296 (0.83), 4.313 (1.01), 4.324 (1.66), 4.334 (0.98), 4.352 (0.85), 4.776 (6.86), 4.792 (6.96), 5.760 (1.32), 7.016 (1.01), 7.031 (1.94), 7.046 (0.98), 7.092 (1.37), 7.112 (2.17), 7.169 (3.13), 7.188 (4.87), 7.208 (2.23), 7.428 (2.41), 7.448 (2.12), 8.364 (11.60), 8.418 (0.44), 12.585 (1.89).

### EXAMPLE 6

### 6-cyclopropyl-N-[(5-methyl-1H-benzimidazol-2-yl)methyl]-1-(1-methylpiperidin-4-yl)-1H-pyrazolo[3,4-b]pyrazin-3-amine

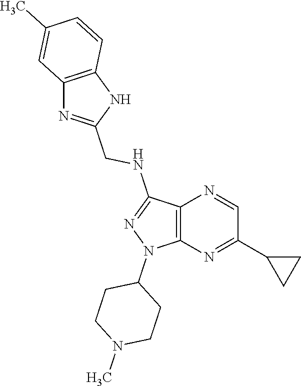 [SMILES: Cc1ccc2[nH]c(CNc3nn(C4CCN(C)CC4)c4nc(C5CC5)cnc34)nc2c1]

In analogy to example 3) 6-cyclopropyl-1-(1-methylpiperidin-4-yl)-1H-pyrazolo[3,4-b]pyrazin-3-amine (100 mg, 367 μmol, intermediate 2) and 5-methyl-1H-benzimidazole-2-carbaldehyde (118 mg, 734 μmol, intermediate 8) reacted and purification via a Biotage chromatography sytem followed by HPLC and followed by thin layer chromatography 8.0 mg (4% yield) of the desired title compound.

LC-MS (Method 2): Rₜ=1.18 min; MS (ESIneg): m/z=415 [M−H]⁻

¹H-NMR (400 MHz, CHLOROFORM-d) δ [ppm]: 0.000 (0.61), 0.811 (0.63), 1.030 (0.94), 1.042 (2.06), 1.049 (3.76), 1.056 (2.11), 1.062 (2.15), 1.069 (3.47), 1.075 (2.37), 1.089 (1.86), 1.096 (3.32), 1.102 (3.94), 1.108 (3.49), 1.115 (2.65), 1.128 (0.84), 1.163 (0.44), 1.184 (2.61), 1.215 (0.63), 1.701 (0.66), 1.899 (1.41), 1.928 (1.56), 2.077 (0.53), 2.089 (1.11), 2.097 (1.10), 2.101 (0.78), 2.110 (1.73), 2.113 (1.12), 2.122 (1.08), 2.129 (1.12), 2.141 (1.39), 2.168 (2.26), 2.198 (1.50), 2.317 (16.00), 2.337 (1.72), 2.346 (1.75), 2.381 (9.90), 2.407 (0.51), 2.976 (1.68), 3.005 (1.56), 3.829 (4.32), 4.481 (0.69), 4.491 (0.44), 4.500 (0.75), 4.510 (1.36), 4.520 (0.72), 4.529 (0.44), 4.539 (0.67), 4.753 (3.75), 4.769 (4.00), 5.134 (0.77), 6.931 (0.74), 6.953 (1.36), 6.959 (1.57), 6.978 (1.67), 7.069 (0.83), 7.438 (0.53), 7.451 (0.44), 7.517 (0.53), 7.538 (0.48), 7.767 (0.89), 7.789 (0.82), 8.140 (11.25), 9.825 (0.98).

### EXAMPLE 7

### 6-cyclopropyl-1-(1-methylpiperidin-4-yl)-N-{[4-(trifluoromethyl)-1H-benzimidazol-2-yl]methyl}-1H-pyrazolo[3,4-b]pyrazin-3-amine

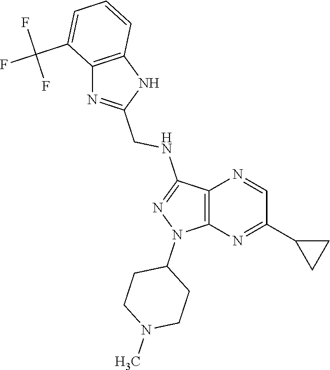 [SMILES: CN1CCC(n2nc(NCc3nc4c(C(F)(F)F)cccc4[nH]3)c3ncc(C4CC4)nc32)CC1]

In analogy to example 2) 6-cyclopropyl-1-(1-methylpiperidin-4-yl)-1H-pyrazolo[3,4-b]pyrazin-3-amine (100 mg, 367 μmol, intermediate 2) and 4-(trifluoromethyl)-1H-benzimidazole-2-carbaldehyde (157 mg, 734 μmol, intermediate 9) reacted and give after one HPLC purifcation 16.7 mg (9% yield) of the desired title compound.

LC-MS (Method 2): Rₜ=1.28 min; MS (ESIpos): m/z=472 [M+H]⁺

¹H-NMR (400 MHz, DMSO-d6) δ [ppm]: 0.850 (0.51), 1.031 (4.79), 1.038 (6.28), 1.043 (5.41), 1.049 (5.01), 1.087 (3.09), 1.094 (5.08), 1.100 (3.44), 1.107 (3.95), 1.114 (5.23), 1.121 (3.04), 1.132 (1.22), 1.188 (0.92), 1.204 (0.98), 1.231 (2.37), 1.703 (2.94), 1.732 (3.17), 2.013 (1.29), 2.040 (2.68), 2.069 (2.62), 2.084 (2.59), 2.117 (3.46), 2.147 (2.99), 2.172 (16.00), 2.190 (4.17), 2.217 (0.85), 2.288 (0.84), 2.301 (1.63), 2.309 (1.87), 2.321 (3.73), 2.326 (2.92), 2.332 (2.87), 2.341 (1.42), 2.352 (0.60), 2.518 (7.12), 2.522 (4.46), 2.664 (1.34), 2.668 (1.82), 2.673 (1.31), 2.804 (3.00), 2.829 (3.15), 4.311 (0.94), 4.329 (1.32), 4.338 (1.87), 4.366 (1.01), 4.810 (8.60), 4.825 (8.75), 6.823 (0.72), 7.047 (1.67), 7.062 (3.65), 7.078 (1.65), 7.258 (1.79), 7.277 (3.93), 7.297 (2.50), 7.322 (0.46), 7.451 (4.00), 7.470 (3.33), 7.502 (0.86), 7.521 (0.70), 7.689 (3.83), 7.710 (3.46), 7.842 (0.76), 7.863 (0.70), 8.369 (14.88), 12.651 (2.89), 12.713 (0.75).

### EXAMPLE 8

### 6-cyclopropyl-N-[(4-methyl-1H-benzimidazol-2-yl)methyl]-1-(1-methylpiperidin-4-yl)-1H-pyrazolo[3,4-b]pyrazin-3-amine

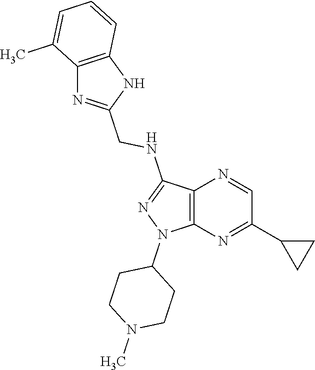 [SMILES: Cc1cccc2[nH]c(CNc3nn(C4CCN(C)CC4)c4nc(C5CC5)cnc34)nc12]

In analogy to example 2) 6-cyclopropyl-1-(1-methylpiperidin-4-yl)-1H-pyrazolo[3,4-b]pyrazin-3-amine (100 mg, 367 μmol, intermediate 2) and 4-methyl-1H-benzimidazole-2-carbaldehyde (118 mg, 734 μmol, intermediate 10) reacted and give after one HPLC purifcation 15.0 mg (9% yield) of the desired title compound.

¹H-NMR (400 MHz, DMSO-d6) δ [ppm]: 0.851 (0.54), 1.033 (3.84), 1.040 (5.48), 1.046 (4.82), 1.052 (5.15), 1.059 (2.58), 1.087 (2.34), 1.094 (4.94), 1.100 (3.00), 1.106 (3.09), 1.113 (5.21), 1.120 (3.03), 1.132 (1.44), 1.232 (2.10), 1.752 (2.67), 2.007 (0.42), 2.164 (4.19), 2.246 (8.27), 2.289 (1.29), 2.301 (1.86), 2.309 (2.01), 2.321 (3.54), 2.327 (2.94), 2.331 (3.06), 2.340 (2.10), 2.522 (8.90), 2.548 (2.28), 2.602 (1.35), 2.629 (1.11), 2.646 (0.72), 2.665 (1.71), 2.669 (2.07), 2.673 (1.56), 2.903 (2.55), 3.416 (1.53), 3.443 (0.78), 3.508 (0.60), 3.533 (0.66), 4.384 (1.08), 4.748 (8.09), 4.764 (8.18), 6.772 (0.48), 6.909 (2.64), 6.927 (3.75), 6.986 (4.10), 7.006 (5.54), 7.024 (3.09), 7.224 (1.05), 7.333 (0.69), 8.368 (16.00), 12.100 (0.72), 12.239 (0.48).

### EXAMPLE 9

### 6-cyclopropyl-N-[(4,5-difluoro-1H-benzimidazol-2-yl)methyl]-1-(1-methylpiperidin-4-yl)-1H-pyrazolo[3,4-b]pyrazin-3-amine

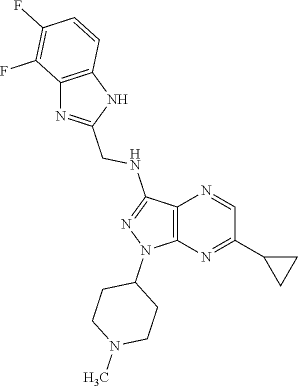 [SMILES: CN1CCC(n2nc(NCc3nc4c(F)c(F)ccc4[nH]3)c3ncc(C4CC4)nc32)CC1]

In analogy to example 2) 6-cyclopropyl-1-(1-methylpiperidin-4-yl)-1H-pyrazolo[3,4-b]pyrazin-3-amine (100 mg, 367 μmol, intermediate 2) and 4,5-difluoro-1H-benzimidazole-2-carbaldehyde (134 mg, 734 μmol, intermediate 11) reacted and give after one HPLC purifcation followed by thin layer chromatography purification 0.9 mg (1% yield) of the desired title compound.

¹H-NMR (400 MHz, DMSO-d6) δ [ppm]: 0.833 (0.59), 0.851 (1.14), 0.867 (0.62), 1.016 (0.83), 1.028 (2.29), 1.034 (3.29), 1.041 (2.67), 1.046 (2.91), 1.053 (1.52), 1.059 (0.59), 1.069 (0.52), 1.074 (0.59), 1.083 (1.39), 1.090 (2.81), 1.097 (1.77), 1.103 (1.84), 1.111 (2.98), 1.118 (1.80), 1.129 (0.80), 1.185 (0.52), 1.231 (5.02), 1.247 (2.94), 1.256 (6.16), 1.278 (0.69), 1.295 (1.84), 1.332 (4.71), 1.688 (1.42), 1.715 (1.52), 1.816 (2.11), 1.975 (1.00), 2.001 (2.25), 2.030 (1.97), 2.056 (0.87), 2.063 (0.90), 2.084 (1.73), 2.092 (1.59), 2.114 (1.28), 2.123 (1.39), 2.145 (1.28), 2.162 (16.00), 2.285 (0.45), 2.297 (0.90), 2.306 (0.97), 2.318 (2.18), 2.323 (1.80), 2.327 (2.74), 2.332 (1.94), 2.337 (1.32), 2.518 (8.94), 2.523 (5.68), 2.660 (0.66), 2.665 (1.45), 2.669 (2.04), 2.673 (1.45), 2.678 (0.66), 2.784 (1.90), 2.813 (1.84), 3.165 (1.32), 3.184 (0.42), 3.377 (1.80), 4.295 (0.55), 4.312 (0.69), 4.323 (1.14), 4.333 (0.62), 4.340 (0.42), 4.350 (0.59), 4.744 (4.71), 4.759 (4.71), 6.791 (0.45), 6.884 (0.48), 6.980 (0.66), 6.994 (1.25), 7.008 (0.66), 7.112 (0.69), 7.131 (0.83), 7.135 (1.18), 7.142 (0.80), 7.153 (1.21), 7.159 (0.83), 7.163 (1.28), 7.181 (1.42), 7.206 (1.14), 7.214 (1.25), 7.227 (0.80), 7.237 (0.66), 8.088 (0.55), 8.362 (11.01), 8.535 (0.52).

### EXAMPLE 10

### (±)-N-[(1H-benzimidazol-2-yl)methyl]-6-cyclopropyl-1-[oxolan-3-yl]-1H-pyrazolo[3,4-b]pyrazin-3-amine

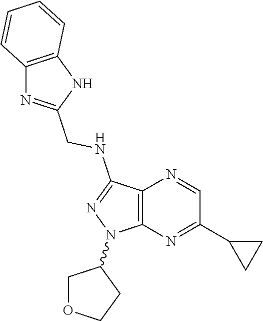 [SMILES: c1ccc2[nH]c(CNc3nn(C4CCOC4)c4nc(C5CC5)cnc34)nc2c1]

In analogy to example 2) (±)-6-cyclopropyl-1-(oxolan-3-yl)-1H-pyrazolo[3,4-b]pyrazin-3-amine (190 mg, 775 μmol, intermediate 4) and 1H-benzimidazole-2-carbaldehyde (226 mg, 1.55 mmol) reacted and give after one HPLC purifcation 65.0 mg (21% yield) of the desired title compound.

LC-MS (Method 2): Rₜ=1.09 min; MS (ESIpos): m/z=376 [M+H]⁺

¹H-NMR (400 MHz, DMSO-d6) δ [ppm]: 1.036 (1.05), 1.048 (3.03), 1.055 (4.30), 1.061 (4.56), 1.066 (4.22), 1.073 (2.33), 1.090 (2.83), 1.097 (4.06), 1.104 (2.83), 1.111 (2.63), 1.117 (4.52), 1.124 (2.49), 1.135 (1.22), 1.154 (3.26), 1.172 (6.34), 1.189 (3.07), 1.231 (0.51), 1.907 (2.74), 1.987 (10.45), 2.206 (2.25), 2.224 (5.13), 2.242 (4.12), 2.253 (1.37), 2.260 (1.37), 2.298 (0.68), 2.311 (1.22), 2.318 (1.61), 2.327 (2.15), 2.330 (2.63), 2.342 (1.38), 2.350 (1.24), 2.362 (0.59), 2.518 (6.26), 2.522 (4.48), 2.664 (0.59), 2.669 (0.84), 2.673 (0.63), 3.763 (1.20), 3.782 (4.51), 3.794 (3.68), 3.800 (2.78), 3.804 (3.51), 3.816 (4.43), 3.905 (1.41), 3.923 (3.36), 3.943 (2.89), 3.955 (3.14), 3.961 (1.32), 3.972 (3.67), 3.977 (2.99), 3.995 (2.57), 3.999 (1.17), 4.017 (2.49), 4.035 (2.46), 4.053 (0.86), 4.745 (6.52), 4.760 (6.89), 5.202 (0.61), 5.215 (1.01), 5.220 (1.33), 5.232 (1.86), 5.246 (1.22), 5.263 (0.59), 7.019 (1.56), 7.034 (3.52), 7.049 (1.60), 7.089 (0.98), 7.099 (5.07), 7.106 (4.69), 7.114 (4.96), 7.122 (5.76), 7.132 (1.25), 7.468 (1.04), 8.399 (16.00), 8.541 (0.78), 12.140 (1.32).

### EXAMPLE 11

### tert-butyl 4-(3-{[(1H-benzimidazol-2-yl)methyl]amino}-6-cyclopropyl-1H-pyrazolo[3,4-b]pyrazin-1-yl)piperidine-1-carboxylate

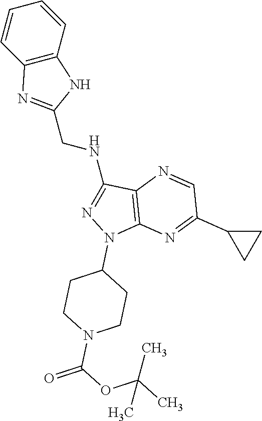 [SMILES: CC(C)(C)OC(=O)N1CCC(n2nc(NCc3nc4ccccc4[nH]3)c3ncc(C4CC4)nc32)CC1]

In analogy to example 2) tert-butyl 4-(3-amino-6-cyclopropyl-1H-pyrazolo[3,4-b]pyrazin-1-yl)piperidine-1-carboxylate (500 mg, 1.39 mmol, intermediate 3) and 1H-benzimidazole-2-carbaldehyde (408 mg, 2.79 mmol) reacted and give after one HPLC purifcation 111 mg (15% yield) of the desired title compound.

LC-MS (Method 2): Rₜ=1.35 min; MS (ESIpos): m/z=489 [M+H]⁺

¹H-NMR (400 MHz, DMSO-d6) δ [ppm]: 1.027 (0.64), 1.033 (0.94), 1.040 (0.81), 1.045 (0.80), 1.052 (0.43), 1.090 (0.75), 1.097 (0.49), 1.103 (0.51), 1.110 (0.80), 1.117 (0.51), 1.154 (0.91), 1.172 (1.83), 1.189 (0.94), 1.404 (16.00), 1.427 (0.55), 1.436 (1.00), 1.448 (1.07), 1.767 (0.49), 1.908 (0.40), 1.919 (0.40), 1.987 (3.46), 2.320 (0.50), 2.331 (0.41), 2.522 (0.71), 3.999 (0.50), 4.017 (0.92), 4.035 (0.85), 4.740 (1.23), 4.755 (1.24), 6.933 (0.71), 7.098 (0.74), 7.105 (0.77), 7.113 (0.80), 7.120 (0.83), 8.375 (2.51).

### EXAMPLE 12

### N-[(1H-benzimidazol-2-yl)methyl]-6-cyclopropyl-1-(piperidin-4-yl)-1H-pyrazolo[3,4-b]pyrazin-3-amine

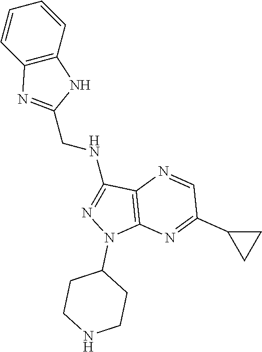 [SMILES: c1ccc2[nH]c(CNc3nn(C4CCNCC4)c4nc(C5CC5)cnc34)nc2c1]

To a solution of tert-butyl 4-(3-{[(1H-benzimidazol-2-yl)methyl]amino}-6-cyclopropyl-1H-pyrazolo[3,4-b]pyrazin-1-yl)piperidine-1-carboxylate (111 mg, 227 μmol, example 11) in 1 mL THF was added 110 μL 4N HCl in dioxan and this mixture was stirred for two hours at room temperature. Then again 110 μL 4N HCl in dioxanwas added and the mixture was stirred at 40° C. over 3 days. After cooling to room temperature the mixture was evaporated to dryness and the residue was purified via two subsequent HPLC to obtain 38.6 mg (42% yield) of the desired titled compound.

LC-MS (Method 2): Rₜ=0.99 min; MS (ESIneg): m/z=387 [M−H]⁻

¹H-NMR (400 MHz, DMSO-d6) δ [ppm]: 1.028 (1.56), 1.040 (4.45), 1.046 (6.17), 1.052 (6.42), 1.057 (6.02), 1.065 (3.04), 1.084 (3.39), 1.091 (5.38), 1.097 (4.17), 1.104 (3.60), 1.111 (6.14), 1.118 (3.50), 1.129 (1.66), 1.154 (1.53), 1.172 (2.99), 1.190 (1.63), 1.228 (1.38), 1.668 (3.09), 1.693 (3.75), 1.902 (1.11), 1.911 (1.26), 1.932 (2.95), 1.941 (3.19), 1.962 (3.04), 1.972 (3.04), 1.988 (6.04), 2.285 (0.83), 2.297 (1.61), 2.305 (1.89), 2.317 (3.19), 2.327 (2.62), 2.337 (1.89), 2.349 (0.78), 2.539 (6.37), 2.572 (5.08), 2.603 (2.89), 2.669 (1.20), 2.989 (4.07), 3.019 (3.59), 3.166 (1.00), 4.000 (0.45), 4.017 (1.26), 4.035 (1.26), 4.053 (0.45), 4.409 (1.16), 4.428 (1.36), 4.438 (2.22), 4.448 (1.36), 4.468 (1.13), 4.756 (9.33), 4.771 (9.48), 6.865 (2.22), 6.880 (4.75), 6.895 (2.19), 7.102 (5.11), 7.109 (5.34), 7.117 (5.59), 7.124 (5.81), 7.423 (1.26), 7.522 (1.28), 8.355 (16.00), 12.181 (1.06).

### EXAMPLE 13

### formic acid—N-[(1H-benzimidazol-2-yl)methyl]-6-cyclopropyl-1-phenyl-1H-pyrazolo[3,4-b]pyrazin-3-amine (1/1)

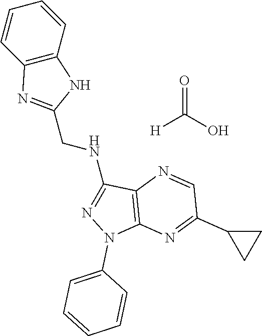

To a solution of 6-cyclopropyl-1-phenyl-1H-pyrazolo[3,4-b]pyrazin-3-amine (100 mg, 99% purity, 0.394 mmol, intermediate 12) in methanol (20 mL) were added 1H-benzimidazole-2-carbaldehyde (115 mg, 0.788 mmol) and titanium(IV) isopropoxide (0.047 mL, 0.160 mmol) at room temperature. The reaction mixture was heated to 60° C. and stirred at 60° C. for 12 hours. After cooling the solution to room temperature, sodium borohydride (74.5 mg, 1.97 mmol) was added to the reaction mixture at room temperature. The reaction mixture was stirred at room temperature for 12 hours. The mixture was filtered and the filtrate was evaporated under reduced pressure to give a residue. The combined residue was purified by preparative TLC (dichlorometane:methanol=10:1) and preparative-HPLC [Instrument:Gilson-281; Column: Phenomenex Gemini 150*25mm*5 pm; eluent A: water (0.225% formic acid), eluent B: acetonitrile; gradient: 0-7.8 min 27-47% B; flow 25 mL/min; temperature: RT; Detector: UV 220/254 nm.) to give the desired titled compound (38.1 mg, 99% purity, 22% yield) as a yellow solid.

LC-MS (Method 5): Rₜ=0.754 min; MS (ESIpos): m/z=382.2 [M+H]⁺.

¹H-NMR (400 MHz, DMSO-d6): δ [ppm]=1.23-1.06 (m, 4H), 2.48-2.39 (m, 1H), 4.86 (d, 2H), 7.24-7.06 (m, 3H), 7.53-7.38 (m, 5H), 8.11 (d, 2H), 8.56 (s, 1H).

### EXAMPLE 14

### N-[(1H-benzimidazol-2-yl)methyl]-6-cyclopropyl-1-(propan-2-yl)-1H-pyrazolo[3,4-b]pyrazin-3-amine

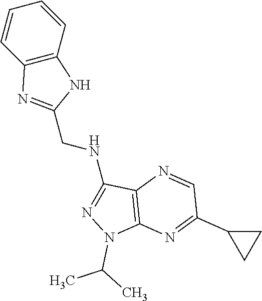 [SMILES: CC(C)n1nc(NCc2nc3ccccc3[nH]2)c2ncc(C3CC3)nc21]

To a solution of 6-cyclopropyl-1-isopropyl-1H-pyrazolo[3,4-b]pyrazin-3-amine (70.0 mg, 99% purity, 0.319 mmol, intermediate 13) in methanol (3.0 mL, 74 mmol) was added 1H-benzimidazole-2-carbaldehyde (93.2 mg, 638 μmol) and acetic acid (1.8 μL, 0.032 mmol) at room temperature. The reaction mixture was heated to 60° C. and stirred at 60° C. for 12 hours. The reaction mixture was cooled to room temperature. Then sodium cyanoborohydride (40.1 mg, 0.638 mmol) was added to the reaction mixture. The reaction mixture was stirred at room temperature for 2 hours. Water was added to the reaction mixture, the solution was extracted with ethyl acetate, the organic layer was evaporated under reduced pressure to give a residue. The residue was purified by preparative-TLC (dichloromethane:methanol=10:1) and preparative-HPLC (Instrument:Gilson-281; Column: Phenomenex Gemini 150*25 mm*5 μm; eluent A: water (0.225% formic acid), eluent B: acetonitrile; gradient: 0-7.8 min 30-50% B; flow 25 mL/min; temperature: RT; Detector: UV 220/254 nm) to give the deired titled compound (55.0 mg, 95% purity, 47% yield) as a yellow solid.

LC-MS (Method 5): Rₜ=0.692 min; MS (ESIpos): m/z=348.2 [M+H]⁺.

¹H-NMR (400 MHz, DMSO-d6): δ [ppm]=1.06-1.03 (m, 2H), 1.11-1.08 (m, 2H), 1.34 (d, 6H), 2.33-2.30 (m, 1H), 4.82-4.77 (m, 3H), 6.87 (t, 1H), 7.13-7.09 (m, 2H), 7.47 (s, 2H), 8.36 (s, 1H), 12.19 (s, 1H).

### EXAMPLE 15

### N-[(1H-benzi midazol-2-yl)methyl]-6-cyclopropyl-1-methyl-1H-pyrazolo[3,4-b]pyrazin-3-amine

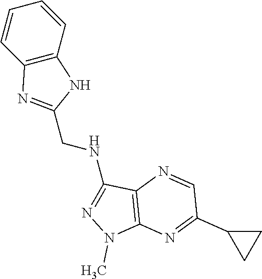 [SMILES: Cn1nc(NCc2nc3ccccc3[nH]2)c2ncc(C3CC3)nc21]

To a solution of 6-cyclopropyl-1-methyl-1H-pyrazolo[3,4-b]pyrazin-3-amine (140 mg, 0.740 mmol, intermediate 14) in methanol (14 mL) was added 1H-benzimidazole-2-carbaldehyde (216 mg, 1.48 mmol) and acetic acid (4.2 μL, 74 μmol) at room temperature. The reaction was heated to 60° C. and stiired at 60° C. for 12 hours. After cooling the solution to room temperature, sodium cyanoborohydride (93.0 mg, 1.48 mmol) was added to the reaction mixture at room temperature. The reaction mixture was stirred at room temperature for 1 hour. Water was added to the reaction mixture. The solution was extracted with ethyl acetate. The organic layer was evaporated under reduced pressure to give a residue. The residue was purified by preparative-TLC (dichloromethane: methanol =10: 1) and preparative-HPLC (Instrument:Gilson-281; Column: Phenomenex Gemini 150*25 mm*5 μm; eluent A: water (0.225% formic acid), eluent B: acetonitrile; gradient: 0-7.8 min 17-37% B; flow 25 mL/min; temperature: RT; Detector: UV 220/254 nm) to give the desired titled compound (6.40 mg, 99% purity, 3% yield) as a yellow solid.

LC-MS (Method 4): Rₜ=0.764 min; MS (ESIpos): m/z=320.3 [M+H]⁺.

¹H-NMR (400 MHz, DMSO-d₆): δ [ppm]=1.11-1.05 (m, 4H), 2.34-2.31 (m, 1H), 3.69 (s, 3H), 4.73 (d, 2H), 6.97 (t, 1H), 7.14-7.05 (m, 2H), 7.46 (s, 2H), 8.37 (s, 1H).

### EXAMPLE 16

### N-[(1H-benzimidazol-2-yl)methyl]-6-cyclopropyl-1-(oxan-4-yl)-1H-pyrazolo[3,4-b]pyrazin-3-amine

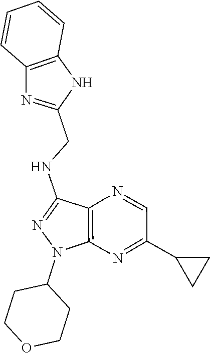 [SMILES: c1ccc2[nH]c(CNc3nn(C4CCOCC4)c4nc(C5CC5)cnc34)nc2c1]

To a solution of 6-cyclopropyl-1-(oxan-4-yl)-1H-pyrazolo[3,4-b]pyrazin-3-amine (230 mg, 0.887 mmol, intermediate 18) and 1H-benzimidazole-2-carbaldehyde (259 mg, 1.77 mmol) in methanol (20 mL) was added acetic acid (5.1 μL, 89 μmol) at room temperature. The reaction mixture was stirred at 60° C. for 16 hours. The mixture was cooled to room temperature. Sodium cyanoborohydride (111 mg, 1.77 mmol) was added to above mixture and the mixture was stirred for 2 hours at room temperature. The mixture was filtered through a pad of celite and the filtrate was concentrated to give a residue. The residue was dissolved in ethyl acetate. The mixture was washed with brine, dried over anhydrous sodium sulfate, filtered and concentrated to give a residue. The residue was purified by preparative HPLC [Instrument:Gilson-281; Column: Boston pH-lex 150*25 10 um; eluent A: water (0.225% formic acid), eluent B: acetonitrile; gradient: 0-10 min 19-49% B; flow 25 mL/min; temperature: RT; Detector: UV 220/254 nm] to give the desired titled compound 81.3 mg (95% purity, 22% yield) as a yellow solid.

LC-MS (Method 4): Rₜ=0.698 min; MS (ESIpos): m/z=390.1 [M+H]⁺.

¹H NMR (400 MHz, DMSO-d₆): δ [ppm]=1.07-1.01 (m, 2H), 1.15-1.08 (m, 2H), 1.64 (dd, 2H), 1.99-1.87 (m, 2H), 2.36-2.29 (m, 1H), 3.47-3.34 (m, 2H), 3.86-3.80 (m, 3H), 4.65-4.53 (m, 1H), 5.00 (s, 2H), 7.51-7.40 (m, 3H), 7.75-7.71 (m, 2H), 8.39 (s, 1H).

### EXAMPLE 17

### (+) or (−)-N-[(1H-benzimidazol-2-yl)methyl]-6-cyclopropyl-1-(oxolan-3-yl)-1H-pyrazolo[3,4-b]pyrazin-3-amine

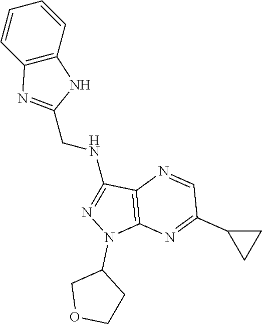 [SMILES: c1ccc2[nH]c(CNc3nn(C4CCOC4)c4nc(C5CC5)cnc34)nc2c1]

Chiral H PLC-separation of racemate example 10)

LC-MS (Method 2): Rₜ=1.09 min; MS (ESIpos): m/z=376 [M+H]⁺

### Example 18

### (-) or (+) -N-[(1H-benzimidazol-2-yl)methyl]-6-cyclopropyl-1-[oxolan-3-yl]-1H-pyrazolo[3,4-b]pyrazin-3-amine

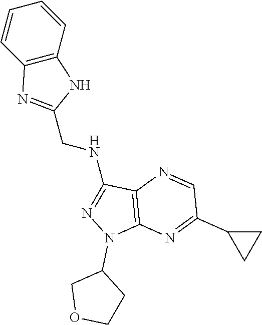 [SMILES: c1ccc2[nH]c(CNc3nn(C4CCOC4)c4nc(C5CC5)cnc34)nc2c1]

Chiral H PLC-separation of racemate example 10)

LC-MS (Method 2): Rₜ=1.09 min; MS (ESIpos): m/z=376 [M+H]⁺

### Example 19

### 6-cyclopropyl-N-[(5,6-dichloro-1H-benzimidazol-2-yl)methyl]-1-(1-methylpiperidin-4-yl)-1H-pyrazolo[3,4-b]pyrazin-3-amine

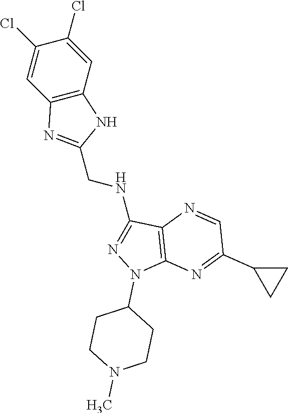 [SMILES: CN1CCC(n2nc(NCc3nc4cc(Cl)c(Cl)cc4[nH]3)c3ncc(C4CC4)nc32)CC1]

In analogy to example 1) 6-cyclopropyl-1-(1-methylpiperidin-4-yl)-1H-pyrazolo[3,4-b]pyrazin-3-amine (100 mg, 367 μmol, intermediate 2) and 5,6-dichloro-1H-benzimidazole-2-carbaldehyde (186 mg, 85% purity, 734 μmol intermediate 19) reacted and give after purification using a Biotage chromatography system followed by a HPLC purifcation 71.0 mg (97% purity, 40% yield) of the desired title compound.

LC-MS (Method 2): Rₜ=1.29 min; MS (ESIpos): m/z=471 [M+H]⁺

¹H-NMR (400 MHz, DMSO-d6) δ [ppm]: 1.014 (0.84), 1.026 (2.51), 1.033 (3.60), 1.039 (3.05), 1.044 (3.19), 1.051 (1.67), 1.073 (0.56), 1.083 (1.43), 1.090 (3.01), 1.096 (1.94), 1.103 (2.07), 1.110 (3.30), 1.117 (2.00), 1.128 (0.85), 1.681 (1.77), 1.708 (1.81), 1.977 (0.97), 2.007 (2.25), 2.035 (2.86), 2.074 (2.29), 2.098 (1.33), 2.105 (1.48), 2.137 (0.57), 2.164 (16.00), 2.242 (0.41), 2.284 (0.46), 2.295 (0.92), 2.303 (1.09), 2.315 (1.83), 2.327 (1.53), 2.335 (1.08), 2.539 (0.51), 2.668 (0.44), 2.787 (2.10), 2.813 (2.29), 4.293 (0.57), 4.310 (0.83), 4.320 (1.17), 4.330 (0.67), 4.348 (0.62), 4.748 (5.05), 4.763 (5.11), 7.001 (1.30), 7.016 (2.97), 7.032 (1.28), 7.649 (0.54), 7.790 (0.54), 8.360 (10.69), 12.484 (1.27).

### EXAMPLE 20

### N-[(4-bromo-1H-benzimidazol-2-yl)methyl]-6-cyclopropyl-1-(1-methylpiperidin-4-yl)-1H-pyrazolo[3,4-b]pyrazin-3-amine

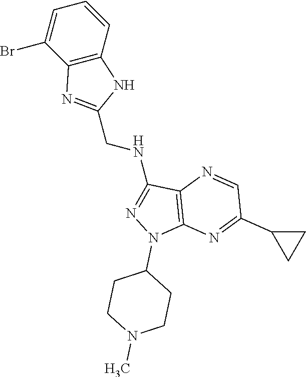 [SMILES: CN1CCC(n2nc(NCc3nc4c(Br)cccc4[nH]3)c3ncc(C4CC4)nc32)CC1]

In analogy to example 2) 6-cyclopropyl-1-(1-methylpiperidin-4-yl)-1H-pyrazolo[3,4-b]pyrazin-3-amine (100 mg, 367 μmol, intermediate 2) and 4-bromo-1H-benzimidazole-2-carbaldehyde (165 mg, 734 μmol, intermediate 20) reacted and give after purification using a Biotage chromatography system followed by a HPLC purifcation 40.0 mg (98% purity, 22% yield) of the desired title compound.

LC-MS (Method 2): Rₜ=1.22 min; MS (ESIpos): m/z=481 [M+H]⁺

¹H-NMR (400 MHz, DMSO-d6) δ [ppm]: 1.028 (2.33), 1.035 (3.38), 1.041 (2.89), 1.047 (3.05), 1.054 (1.55), 1.083 (1.42), 1.090 (2.99), 1.097 (1.85), 1.103 (1.95), 1.110 (3.13), 1.117 (1.92), 1.128 (0.81), 1.699 (1.58), 1.723 (1.75), 1.979 (1.06), 2.005 (2.44), 2.034 (1.91), 2.084 (3.49), 2.111 (1.66), 2.118 (1.72), 2.140 (1.53), 2.149 (1.70), 2.166 (16.00), 2.285 (0.49), 2.297 (0.93), 2.306 (1.04), 2.317 (1.79), 2.327 (1.39), 2.337 (0.98), 2.349 (0.43), 2.669 (0.48), 2.795 (2.06), 2.823 (1.95), 3.166 (3.41), 4.299 (0.62), 4.317 (0.76), 4.327 (1.23), 4.338 (0.73), 4.356 (0.62), 4.765 (4.96), 4.780 (5.05), 6.970 (0.80), 7.041 (2.28), 7.060 (4.62), 7.080 (2.76), 7.327 (3.29), 7.329 (3.47), 7.346 (2.90), 7.348 (2.84), 7.431 (1.54), 7.450 (1.39), 8.364 (10.25).

### EXAMPLE 21

### N-[(5-chloro-4-methyl-1H-benzimidazol-2-yl)methyl]-6-cyclopropyl-1-(1-methylpiperidin-4-yl)-1H-pyrazolo[3,4-b]pyrazin-3-amine

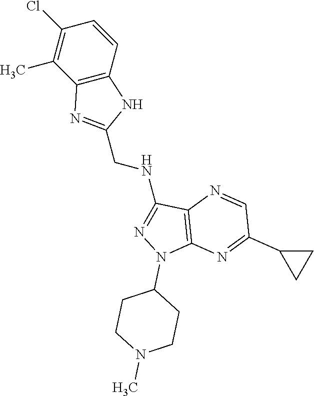 [SMILES: Cc1c(Cl)ccc2[nH]c(CNc3nn(C4CCN(C)CC4)c4nc(C5CC5)cnc34)nc12]

In analogy to example 3) 6-cyclopropyl-1-(1-methylpiperidin-4-yl)-1H-pyrazolo[3,4-b]pyrazin-3-amine (90 mg, 330 μmol, intermediate 2) and 5-chloro-4-methyl-1H-benzimidazole-2-carbaldehyde (129 mg, 661 μmol, intermediate 21) reacted and give after purification using a Biotage chromatography system followed by a HPLC purifcation 56.0 mg (95% purity, 36% yield) of the desired title compound.

LC-MS (Method 2): Rₜ=1.28 min; MS (ESIpos): m/z=451 [M+H]⁺

¹H-NMR (400 MHz, DMSO-d6) δ [ppm]: 1.028 (4.28), 1.034 (6.20), 1.040 (5.30), 1.045 (5.49), 1.053 (2.82), 1.090 (4.88), 1.097 (3.22), 1.103 (3.43), 1.110 (5.36), 1.117 (3.29), 1.231 (0.82), 1.696 (2.93), 1.719 (3.21), 1.979 (1.73), 2.008 (4.04), 2.038 (3.18), 2.081 (1.48), 2.111 (2.98), 2.142 (2.56), 2.167 (16.00), 2.284 (0.71), 2.296 (1.54), 2.304 (1.76), 2.316 (3.01), 2.327 (3.21), 2.336 (1.99), 2.348 (0.69), 2.523 (8.12), 2.535 (14.75), 2.665 (1.14), 2.669 (1.48), 2.798 (3.60), 2.825 (3.42), 3.159 (2.04), 3.172 (2.16), 4.098 (0.45), 4.111 (0.42), 4.299 (1.04), 4.318 (1.26), 4.329 (2.02), 4.356 (0.99), 4.749 (7.56), 4.764 (7.68), 6.799 (0.83), 6.963 (1.07), 6.978 (2.01), 6.993 (1.03), 7.127 (4.81), 7.148 (6.72), 7.228 (2.79), 7.248 (1.91), 7.353 (0.92), 7.375 (0.74), 8.360 (15.11), 12.287 (2.21), 12.463 (0.95).

### EXAMPLE 22

### N-[(5-chloro-1H-benzimidazol-2-yl)methyl]-6-cyclopropyl-1-(1-methylpiperidin-4-yl)-1H-pyrazolo[3,4-b]pyrazin-3-amine

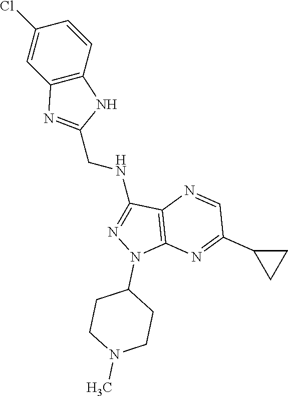 [SMILES: CN1CCC(n2nc(NCc3nc4cc(Cl)ccc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (90 mg, 330 μmol, intermediate 2) and 5-chloro-1H-benzimidazole-2-carbaldehyde (119 mg, 661 μmol, intermediate 22) reacted and give after purification using a Biotage chromatography system followed by a HPLC purifcation 31.0 mg (93% purity, 20% yield) of the desired title compound.

LC-MS (Method 2): Rₜ=1.21 min; MS (ESIpos): m/z=437 [M+H]⁺

¹H-NMR (400 MHz, DMSO-d6) δ [ppm]: 1.016 (0.76), 1.027 (2.32), 1.034 (3.35), 1.040 (2.79), 1.045 (2.97), 1.052 (1.52), 1.075 (0.55), 1.083 (1.43), 1.090 (2.87), 1.097 (1.83), 1.103 (2.05), 1.111 (3.12), 1.118 (1.87), 1.129 (0.80), 1.163 (0.46), 1.230 (0.60), 1.688 (1.53), 1.717 (1.67), 1.973 (1.00), 1.998 (2.36), 2.028 (2.19), 2.055 (0.99), 2.063 (0.97), 2.084 (2.20), 2.092 (1.71), 2.114 (1.30), 2.123 (1.40), 2.163 (16.00), 2.192 (0.60), 2.284 (0.44), 2.297 (0.85), 2.304 (1.03), 2.316 (1.82), 2.327 (1.71), 2.337 (1.10), 2.523 (2.89), 2.665 (0.57), 2.669 (0.79), 2.673 (0.58), 2.789 (2.05), 2.817 (2.00), 4.292 (0.57), 4.310 (0.74), 4.320 (1.22), 4.331 (0.70), 4.349 (0.61), 4.743 (4.67), 4.759 (4.75), 5.759 (1.75), 6.954 (1.15), 6.970 (2.59), 6.984 (1.12), 7.124 (1.73), 7.129 (1.75), 7.145 (1.95), 7.150 (2.00), 7.455 (0.49), 8.360 (10.78), 12.356 (0.61).

### EXAMPLE 23

### 6-cyclopropyl-N-[(4-fluoro-1H-benzimidazol-2-yl)methyl]-1-(oxan-4-yl)-1H-pyrazolo[3,4-b]pyrazin-3-amine

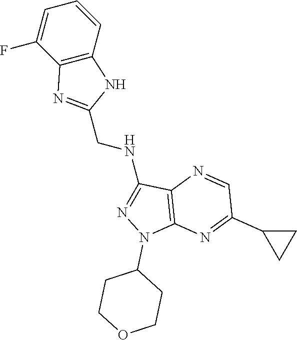 [SMILES: Fc1cccc2[nH]c(CNc3nn(C4CCOCC4)c4nc(C5CC5)cnc34)nc12]

In analogy to example 3) 6-cyclopropyl-1-(oxan-4-yl)-1H-pyrazolo[3,4-b]pyrazin-3-amine (110 mg, 424 μmol, intermediate 18) and 4-fluoro-1H-benzimidazole-2-carbaldehyde (279 mg, 1.70 mmol, intermediate 5) reacted and give after purification using a Biotage chromatography system followed by a HPLC purifcation 22.9 mg (97% purity, 13% yield) of the desired title compound.

LC-MS (Method 2): Rₜ=1.10 min; MS (ESIpos): m/z=408 [M+H]⁺

¹H-NMR (400 MHz, DMSO-d6) δ [ppm]: 1.031 (1.27), 1.043 (3.78), 1.049 (5.27), 1.055 (4.84), 1.061 (5.19), 1.068 (2.32), 1.077 (0.87), 1.090 (2.43), 1.097 (4.65), 1.104 (3.03), 1.110 (2.81), 1.118 (5.15), 1.125 (2.80), 1.136 (1.21), 1.154 (1.18), 1.172 (2.29), 1.190 (1.11), 1.231 (0.66), 1.698 (2.42), 1.703 (2.45), 1.729 (2.93), 1.734 (2.79), 1.988 (4.23), 2.040 (0.90), 2.052 (1.07), 2.071 (2.39), 2.082 (2.58), 2.102 (2.38), 2.113 (2.28), 2.132 (0.97), 2.144 (0.84), 2.294 (0.71), 2.306 (1.42), 2.314 (1.62), 2.326 (3.30), 2.332 (1.56), 2.337 (1.75), 2.345 (1.33), 2.358 (0.56), 2.518 (3.93), 2.523 (2.69), 2.540 (0.45), 2.665 (0.89), 2.669 (1.24), 2.674 (0.85), 3.436 (2.58), 3.461 (4.65), 3.466 (4.65), 3.491 (2.72), 3.896 (2.89), 3.904 (2.98), 3.925 (2.53), 3.932 (2.53), 4.017 (0.93), 4.035 (0.92), 4.582 (0.56), 4.592 (1.12), 4.603 (0.76), 4.610 (1.24), 4.621 (2.20), 4.631 (1.20), 4.639 (0.72), 4.650 (1.06), 4.660 (0.51), 4.763 (9.19), 4.778 (9.24), 6.893 (1.64), 6.913 (2.08), 6.920 (1.73), 6.941 (2.00), 7.003 (1.57), 7.019 (3.28), 7.034 (1.44), 7.072 (2.11), 7.084 (2.23), 7.092 (4.07), 7.104 (4.41), 7.112 (2.34), 7.124 (2.28), 7.237 (4.21), 7.256 (3.28), 8.378 (16.00), 12.493 (2.39), 12.856 (0.41).

### EXAMPLE 24

### 6-cyclopropyl-N-[(4-methoxy-1H-benzimidazol-2-yl)methyl]-1-(oxan-4-yl)-1H-pyrazolo[3,4-b]pyrazin-3-amine

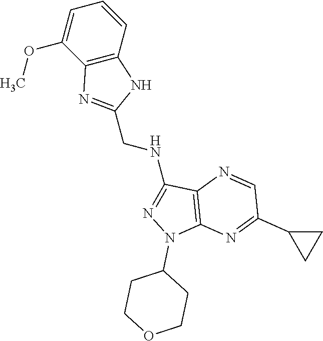 [SMILES: COc1cccc2[nH]c(CNc3nn(C4CCOCC4)c4nc(C5CC5)cnc34)nc12]

In analogy to example 2) 6-cyclopropyl-1-(oxan-4-yl)-1H-pyrazolo[3,4-b]pyrazin-3-amine (110 mg, 424 μmol, intermediate 18) and 4-methoxy-1H-benzimidazole-2-carbaldehyde (299 mg, 1.70 mmol, CAS-RN:[933741-33-4]) reacted and give after three HPLC purifications 56.2 mg (97% purity, 31% yield) of the desired title compound.

LC-MS (Method 2): Rₜ=1.12 min; MS (ESIpos): m/z=420 [M+H]⁺

¹H-NMR (400 MHz, DMSO-d6) δ [ppm]: 1.030 (0.92), 1.042 (2.76), 1.048 (3.90), 1.054 (3.81), 1.059 (3.81), 1.067 (1.83), 1.087 (1.92), 1.094 (3.28), 1.101 (2.43), 1.108 (2.18), 1.115 (3.73), 1.121 (2.09), 1.133 (0.93), 1.231 (0.61), 1.708 (1.95), 1.715 (2.03), 1.741 (2.40), 1.746 (2.29), 2.077 (0.65), 2.097 (1.53), 2.107 (1.65), 2.127 (1.55), 2.137 (1.55), 2.157 (0.61), 2.292 (0.51), 2.304 (1.04), 2.312 (1.18), 2.323 (2.47), 2.331 (1.67), 2.336 (1.46), 2.344 (1.01), 2.356 (0.46), 2.518 (7.32), 2.523 (4.98), 2.665 (0.79), 2.669 (1.10), 2.673 (0.78), 3.445 (1.44), 3.475 (2.86), 3.504 (1.53), 3.900 (16.00), 3.946 (2.01), 4.599 (0.71), 4.617 (0.82), 4.628 (1.40), 4.639 (0.85), 4.656 (0.70), 4.720 (5.62), 4.736 (5.78), 5.760 (7.01), 6.619 (1.03), 6.635 (1.16), 6.721 (1.17), 6.740 (0.90), 6.879 (0.61), 6.894 (1.15), 6.908 (0.58), 6.989 (0.56), 7.013 (3.50), 7.033 (2.94), 7.053 (1.34), 7.126 (0.87), 7.145 (0.60), 8.373 (13.37), 12.168 (1.47), 12.445 (0.95).

### EXAMPLE 25

### 6-cyclopropyl-1-(1-methylpiperidin-4-yl)-N-{[5-(trifluoromethyl)-1H-benzimidazol-2-yl]methyl}-1H-pyrazolo[3,4-b]pyrazin-3-amine

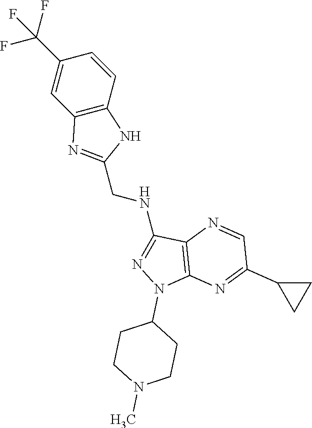 [SMILES: CN1CCC(n2nc(NCc3nc4cc(C(F)(F)F)ccc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (80 mg, 294 μmol, intermediate 2) and 5-(trifluoromethyl)-1H-benzimidazole-2-carbaldehyde (132 mg, 617 μmol, intermediate 23) reacted and give after purification using a Biotage chromatography system followed by a HPLC purifcation 33.0 mg (95% purity, 23% yield) of the desired title compound.

LC-MS (Method 2): Rₜ=1.27 min; MS (ESIpos): m/z=471 [M+H]⁺

¹H-NMR (400 MHz, DMSO-d6) δ [ppm]: 0.797 (0.50), 0.814 (0.51), 0.821 (0.50), 0.904 (0.49), 1.016 (0.86), 1.028 (2.55), 1.035 (5.56), 1.040 (3.07), 1.046 (3.21), 1.052 (5.54), 1.070 (2.70), 1.084 (1.48), 1.091 (2.98), 1.098 (1.98), 1.104 (2.07), 1.111 (3.24), 1.118 (1.97), 1.130 (0.84), 1.229 (0.87), 1.681 (1.63), 1.709 (1.74), 1.970 (0.99), 1.998 (2.40), 2.028 (2.40), 2.045 (1.17), 2.074 (1.71), 2.083 (1.97), 2.103 (1.35), 2.111 (1.43), 2.154 (16.00), 2.286 (0.51), 2.299 (0.90), 2.306 (1.03), 2.318 (2.00), 2.327 (1.62), 2.338 (0.96), 2.350 (0.40), 2.522 (2.43), 2.665 (0.59), 2.669 (0.78), 2.673 (0.60), 2.776 (2.09), 2.804 (2.06), 3.430 (0.58), 3.443 (0.54), 4.292 (0.60), 4.309 (0.79), 4.320 (1.20), 4.330 (0.73), 4.348 (0.80), 4.794 (4.39), 4.809 (4.35), 5.759 (2.71), 7.046 (1.37), 7.438 (1.41), 7.459 (1.75), 7.596 (0.42), 7.736 (0.47), 7.884 (0.56), 8.297 (0.41), 8.366 (10.01), 12.622 (0.73).

### EXAMPLE 26

### 6-cyclopropyl-N-[(5-fluoro-4-methyl-1H-benzimidazol-2-yl)methyl]-1-(1-methylpiperidin-4-yl)-1H-pyrazolo[3,4-b]pyrazin-3-amine

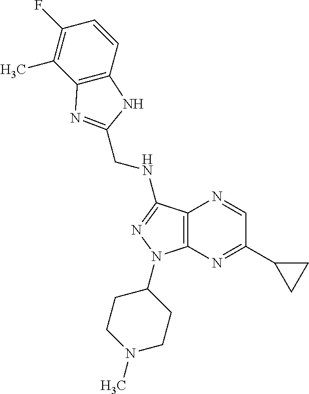 [SMILES: Cc1c(F)ccc2[nH]c(CNc3nn(C4CCN(C)CC4)c4nc(C5CC5)cnc34)nc12]

In analogy to example 3) 6-cyclopropyl-1-(1-methylpiperidin-4-yl)-1H-pyrazolo[3,4-b]pyrazin-3-amine (100 mg, 367 μmol, intermediate 2) and 5-fluoro-4-methyl-1H-benzimidazole-2-carbaldehyde (196 mg, 1.10 mmol, intermediate 24) reacted and give after purification using a Biotage chromatography system followed by a HPLC purifcation 49.0 mg (95% purity, 29% yield) of the desired title compound.

LC-MS (Method 2): Rₜ=1.20 min; MS (ESIneg): m/z=433 [M−H]⁻

¹H-NMR (400 MHz, DMSO-d6) δ [ppm]: 0.797 (0.69), 0.814 (0.70), 0.820 (0.68), 0.904 (0.69), 1.016 (1.26), 1.028 (3.98), 1.035 (12.52), 1.040 (5.05), 1.046 (5.47), 1.052 (16.00), 1.070 (8.40), 1.090 (4.40), 1.096 (2.83), 1.103 (2.97), 1.109 (4.63), 1.116 (2.85), 1.231 (1.02), 1.701 (2.56), 1.730 (2.82), 1.984 (1.36), 2.013 (3.14), 2.041 (2.37), 2.088 (1.29), 2.118 (2.58), 2.148 (2.37), 2.169 (14.55), 2.180 (9.05), 2.284 (0.65), 2.296 (1.28), 2.304 (1.47), 2.316 (2.51), 2.327 (2.48), 2.335 (1.69), 2.347 (0.64), 2.383 (5.76), 2.412 (10.20), 2.522 (5.63), 2.664 (0.77), 2.668 (1.02), 2.673 (0.78), 2.801 (2.71), 2.829 (2.81), 3.404 (0.91), 3.417 (1.04), 3.422 (2.42), 3.434 (2.50), 3.439 (2.51), 3.452 (2.48), 3.457 (1.01), 3.469 (0.85), 4.329 (1.51), 4.346 (2.20), 4.358 (3.40), 4.371 (1.72), 4.733 (2.84), 4.742 (5.21), 4.757 (4.66), 6.762 (0.56), 6.777 (1.21), 6.791 (0.60), 6.906 (1.81), 6.928 (3.26), 6.953 (4.43), 6.968 (1.15), 7.179 (1.36), 7.191 (1.51), 7.202 (1.35), 7.213 (1.25), 7.320 (0.76), 7.331 (0.79), 7.341 (0.74), 7.352 (0.68), 8.360 (11.68), 12.200 (2.22), 12.404 (1.28).

### EXAMPLE 27

### 6-cyclopropyl-1-(1-methylpiperidin-4-yl)-N-({5-[(trifluoromethyl)sulfanyl]-1H-benzimidazol-2-yl}methyl)-1H-pyrazolo[3,4-b]pyrazin-3-amine

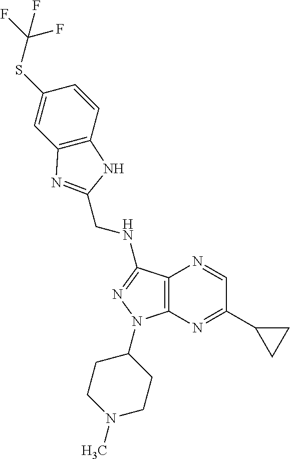 [SMILES: CN1CCC(n2nc(NCc3nc4cc(SC(F)(F)F)ccc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (100 mg, 367 μmol, intermediate 2) and 5-[(trifluoromethyl)sulfanyl]-1H-benzimidazole-2-carbaldehyde (154 mg, 624 μmol, intermediate 25) reacted and give after purification using a Biotage chromatography system followed by a HPLC purifcation 47.0 mg (93% purity, 24% yield) of the desired title compound.

LC-MS (Method 2): Rₜ=1.34 min; MS (ESIpos): m/z=503 [M+H]⁺

¹H-NMR (400 MHz, DMSO-d6) δ [ppm]: 0.850 (0.44), 1.016 (0.88), 1.028 (2.46), 1.035 (5.53), 1.041 (2.83), 1.046 (3.12), 1.053 (6.70), 1.070 (3.07), 1.084 (1.49), 1.091 (3.00), 1.098 (1.93), 1.104 (2.00), 1.111 (3.24), 1.118 (2.00), 1.130 (0.95), 1.172 (0.65), 1.204 (0.45), 1.230 (2.10), 1.684 (1.47), 1.712 (1.57), 1.751 (0.43), 1.904 (0.41), 1.976 (0.96), 2.002 (2.24), 2.031 (2.25), 2.048 (1.07), 2.054 (0.99), 2.076 (1.57), 2.084 (3.08), 2.106 (1.25), 2.114 (1.35), 2.153 (16.00), 2.202 (0.52), 2.285 (0.48), 2.297 (0.90), 2.306 (1.00), 2.318 (2.02), 2.323 (1.12), 2.327 (1.76), 2.337 (1.07), 2.518 (4.05), 2.523 (2.72), 2.665 (0.74), 2.669 (1.05), 2.673 (0.74), 2.774 (1.87), 2.802 (1.89), 3.411 (0.43), 3.429 (0.61), 3.445 (0.56), 3.964 (0.46), 4.293 (0.59), 4.310 (0.75), 4.321 (1.16), 4.331 (0.68), 4.339 (0.54), 4.349 (0.74), 4.359 (0.46), 4.782 (3.87), 4.797 (3.88), 7.032 (1.03), 7.428 (1.03), 7.448 (1.37), 7.905 (0.49), 8.268 (0.73), 8.365 (11.38), 12.616 (0.42).

### EXAMPLE 28

### 6-cyclopropyl-N-[(4,5-dimethyl-1H-benzimidazol-2-yl)methyl]-1-(1-methylpiperidin-4-yl)-1H-pyrazolo[3,4-b]pyrazin-3-amine

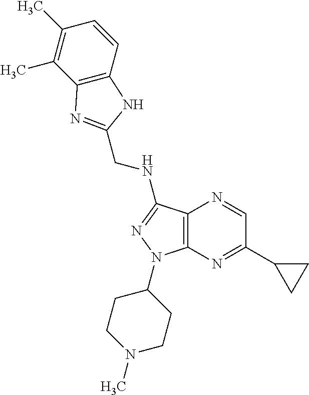 [SMILES: Cc1ccc2[nH]c(CNc3nn(C4CCN(C)CC4)c4nc(C5CC5)cnc34)nc2c1C]

In analogy to example 3) 6-cyclopropyl-1-(1-methylpiperidin-4-yl)-1H-pyrazolo[3,4-b]pyrazin-3-amine (100 mg, 367 μmol, intermediate 2) and 4,5-dimethyl-1H-benzimidazole-2-carbaldehyde (95.9 mg, 551 μmol, intermediate 26) reacted and give after purification using a Biotage chromatography system followed by a HPLC purifcation 43.0 mg (93% purity, 25% yield) of the desired title compound.

LC-MS (Method 2): Rₜ=1.24 min; MS (ESIpos): m/z=431 [M+H]⁺

¹H-NMR (400 MHz, DMSO-d6) δ [ppm]: 1.019 (1.01), 1.031 (2.94), 1.035 (9.11), 1.043 (3.56), 1.049 (4.14), 1.053 (15.07), 1.070 (6.69), 1.083 (1.76), 1.091 (3.78), 1.097 (2.27), 1.104 (2.39), 1.111 (3.91), 1.118 (2.44), 1.129 (1.33), 1.172 (0.59), 1.230 (1.73), 1.729 (1.80), 1.752 (1.88), 2.050 (0.75), 2.080 (1.75), 2.109 (2.30), 2.145 (1.88), 2.176 (1.56), 2.209 (11.13), 2.253 (0.48), 2.292 (16.00), 2.305 (1.86), 2.317 (2.33), 2.322 (1.30), 2.327 (1.95), 2.337 (1.37), 2.349 (0.76), 2.422 (2.23), 2.518 (4.02), 2.523 (2.55), 2.539 (0.58), 2.665 (0.71), 2.669 (0.98), 2.673 (0.73), 2.849 (1.96), 2.874 (2.13), 3.411 (1.06), 3.429 (2.30), 3.446 (2.24), 3.464 (0.82), 4.329 (0.66), 4.357 (1.28), 4.725 (5.84), 4.741 (5.85), 6.903 (3.45), 6.924 (3.86), 7.105 (0.45), 8.178 (0.40), 8.361 (13.99), 11.953 (0.47).

### EXAMPLE 29

### N-[(5-chloro-6-fluoro-1H-benzimidazol-2-yl)methyl]-6-cyclopropyl-1-(1-methylpiperidin-4-yl)-1H-pyrazolo[3,4-b]pyrazin-3-amine

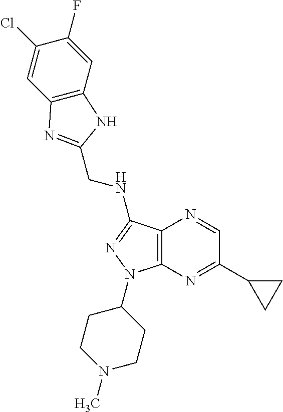 [SMILES: CN1CCC(n2nc(NCc3nc4cc(Cl)c(F)cc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (100 mg, 367 μmol, intermediate 2) and 5-chloro-6-fluoro-1H-benzimidazole-2-carbaldehyde (109 mg, 551 μmol, intermediate 27) reacted and give after purification using a Biotage chromatography system followed by a HPLC purifcation 65.0 mg (90% purity, 35% yield) of the desired title compound.

LC-MS (Method 2): Rₜ=1.24 min; MS (ESIpos): m/z=455 [M+H]⁺

¹H-NMR (400 MHz, DMSO-d6) δ [ppm]: 1.015 (0.79), 1.026 (2.30), 1.035 (9.96), 1.045 (3.02), 1.052 (16.00), 1.070 (8.95), 1.083 (1.49), 1.090 (2.89), 1.097 (1.86), 1.103 (2.06), 1.111 (3.18), 1.117 (1.91), 1.128 (0.89), 1.145 (0.40), 1.163 (0.69), 1.169 (0.64), 1.180 (0.65), 1.189 (0.62), 1.196 (0.48), 1.207 (0.43), 1.229 (0.96), 1.685 (1.34), 1.713 (1.47), 1.976 (0.89), 2.002 (2.08), 2.031 (2.22), 2.047 (1.26), 2.076 (1.47), 2.084 (1.52), 2.105 (1.18), 2.114 (1.30), 2.163 (15.39), 2.195 (0.54), 2.200 (0.64), 2.240 (0.54), 2.283 (0.44), 2.295 (0.86), 2.304 (0.94), 2.315 (1.59), 2.327 (1.60), 2.332 (0.84), 2.335 (0.98), 2.518 (2.70), 2.523 (1.83), 2.539 (1.01), 2.665 (0.50), 2.669 (0.69), 2.673 (0.50), 2.786 (1.87), 2.815 (1.77), 3.411 (0.94), 3.429 (2.28), 3.446 (2.19), 3.464 (0.77), 4.292 (0.54), 4.310 (0.74), 4.320 (1.15), 4.330 (0.74), 4.338 (0.63), 4.348 (0.85), 4.358 (0.61), 4.739 (4.08), 4.754 (4.09), 5.390 (0.60), 6.969 (0.83), 6.984 (1.74), 6.999 (0.81), 8.359 (11.96), 8.635 (0.86), 9.777 (0.82).

### EXAMPLE 30

### 6-cyclopropyl-N-[(5,6-difluoro-1H-benzimidazol-2-yl)methyl]-1-(1-methylpiperidin-4-yl)-1H-pyrazolo[3,4-b]pyrazin-3-amine

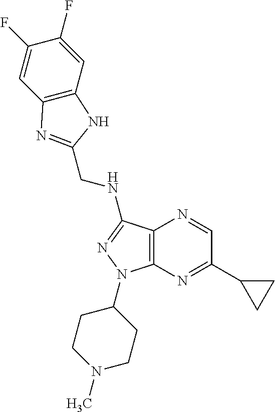 [SMILES: CN1CCC(n2nc(NCc3nc4cc(F)c(F)cc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (100 mg, 367 μmol, intermediate 2) and 5,6-difluoro-1H-benzimidazole-2-carbaldehyde (167 mg, 918 μmol, intermediate 28) reacted and give after purification using a Biotage chromatography system followed by a HPLC purifcation 40.0 mg (95% purity, 24% yield) of the desired title compound.

LC-MS (Method 2): Rₜ=1.19 min; MS (ESIpos): m/z=439 [M+H]⁺

¹H-NMR (400 MHz, DMSO-d6) δ [ppm]: 0.797 (0.41), 0.814 (0.42), 0.821 (0.44), 0.904 (0.45), 1.015 (0.90), 1.026 (2.51), 1.035 (4.21), 1.039 (3.02), 1.045 (3.15), 1.052 (4.54), 1.070 (2.16), 1.083 (1.52), 1.090 (2.99), 1.097 (1.97), 1.103 (2.09), 1.110 (3.20), 1.117 (1.97), 1.128 (0.87), 1.231 (0.62), 1.691 (1.61), 1.719 (1.76), 1.982 (1.01), 2.009 (2.41), 2.038 (2.41), 2.062 (1.14), 2.084 (2.84), 2.092 (1.83), 2.114 (1.39), 2.121 (1.50), 2.168 (16.00), 2.202 (0.64), 2.284 (0.47), 2.296 (0.90), 2.304 (1.03), 2.316 (1.78), 2.327 (1.77), 2.335 (1.14), 2.562 (0.59), 2.665 (0.57), 2.669 (0.79), 2.673 (0.56), 2.727 (4.76), 2.794 (2.05), 2.821 (2.11), 2.888 (5.95), 3.426 (0.42), 4.296 (0.60), 4.313 (0.77), 4.323 (1.24), 4.334 (0.74), 4.351 (0.78), 4.729 (4.80), 4.744 (4.86), 6.941 (1.17), 6.957 (2.66), 6.972 (1.22), 7.422 (0.73), 7.571 (0.70), 7.950 (0.79), 8.358 (10.47), 12.384 (1.26).

### EXAMPLE 31

### N-[(4-chloro-5-fluoro-1H-benzimidazol-2-yl)methyl]-6-cyclopropyl-1-(1-methylpiperidin-4-yl)-1H-pyrazolo[3,4-b]pyrazin-3-amine

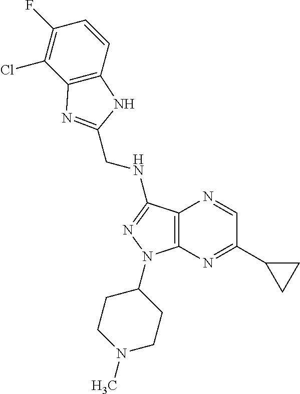 [SMILES: CN1CCC(n2nc(NCc3nc4c(Cl)c(F)ccc4[nH]3)c3ncc(C4CC4)nc32)CC1]

In analogy to example 3) 6-cyclopropyl-1-(1-methylpiperidin-4-yl)-1H-pyrazolo[3,4-b]pyrazin-3-amine (100 mg, 367 μmol, intermediate 2) and 4-chloro-5-fluoro-1H-benzimidazole-2-carbaldehyde (168 mg, 844 μmol, intermediate 29) reacted and give after purification using a Biotage chromatography system followed by a HPLC purifcation 36.0 mg (95% purity, 20% yield) of the desired title compound.

LC-MS (Method 2): Rₜ=1.22 min; MS (ESIpos): m/z=455 [M+H]⁺

¹H-NMR (400 MHz, DMSO-d6) δ [ppm]: 1.016 (0.89), 1.027 (2.59), 1.035 (7.32), 1.040 (3.08), 1.045 (3.36), 1.052 (10.62), 1.070 (4.46), 1.083 (1.57), 1.090 (3.10), 1.097 (1.98), 1.103 (2.12), 1.111 (3.28), 1.117 (2.00), 1.128 (0.89), 1.229 (1.08), 1.343 (0.71), 1.689 (1.52), 1.719 (1.69), 1.976 (1.00), 2.002 (2.36), 2.031 (2.08), 2.062 (0.93), 2.069 (0.91), 2.092 (1.66), 2.099 (1.70), 2.120 (1.42), 2.129 (1.44), 2.161 (16.00), 2.199 (0.71), 2.285 (0.54), 2.297 (0.97), 2.305 (1.08), 2.317 (1.96), 2.327 (1.71), 2.337 (1.16), 2.349 (0.43), 2.518 (5.11), 2.523 (3.53), 2.536 (1.17), 2.665 (0.58), 2.669 (0.81), 2.674 (0.56), 2.785 (1.94), 2.813 (1.89), 3.410 (0.59), 3.428 (1.25), 3.446 (1.23), 3.464 (0.43), 4.294 (0.62), 4.313 (0.79), 4.323 (1.28), 4.334 (0.82), 4.351 (0.83), 4.763 (4.76), 4.778 (4.78), 6.984 (0.42), 7.006 (0.60), 7.140 (2.50), 7.162 (2.91), 7.166 (2.47), 7.188 (2.89), 7.394 (0.66), 8.364 (12.28).

### EXAMPLE 32

### 6-cyclopropyl-N-[(4,5-difluoro-1H-benzimidazol-2-yl)methyl]-1-(oxan-4-yl)-1H-pyrazolo[3,4-b]pyrazin-3-amine

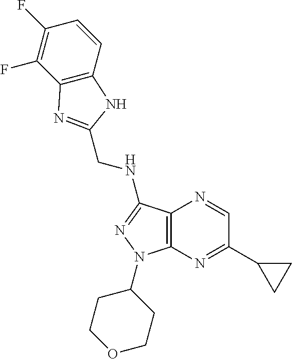 [SMILES: Fc1ccc2[nH]c(CNc3nn(C4CCOCC4)c4nc(C5CC5)cnc34)nc2c1F]

To a solution of 6-cyclopropyl-1-(oxan-4-yl)-1H-pyrazolo[3,4-b]pyrazin-3-amine (100 mg, 386 μmol, intermediate 18) in 3.4 mL methanol was added 4,5-difluoro-1H-benzimidazole-2-carbaldehyde (140 mg, 771 μmol, intermediate 11) and 11 μL acetic acid. The mixture was stirred over night at 60° C. After cooling to room temperature sodium cyanoborohydride (48.5 mg, 771 μmol) was added and the mixture was stirred for two hours at room temperature. Another 9.7 mg of sodium cyanoborohydride (154.2 μmol]) and 2.2 μL of acetic acid were added and the mixture was stirred over night at room temperature. Then the mixture was diluted with 100 mL ethyl acetate, extracted twice with water, brine, filtered using a hydrophobic filter and evaporated to dryness. The residue was purified using a Biotage chromatography system followed by a HPLC purification to give 42.0 mg of the desired compound (97% purity, 25% yield).

LC-MS (Method 2): R₁=1.14 min; MS (ESIpos): m/z=427 [M+H]⁺

¹H-NMR (400 MHz, DMSO-d6) δ [ppm]: 1.030 (1.45), 1.041 (4.13), 1.048 (5.50), 1.054 (4.95), 1.060 (5.66), 1.067 (2.46), 1.077 (1.00), 1.090 (2.56), 1.097 (4.81), 1.104 (3.07), 1.111 (3.02), 1.118 (5.24), 1.124 (2.91), 1.136 (1.25), 1.230 (0.69), 1.316 (0.61), 1.689 (2.48), 1.696 (2.61), 1.721 (3.06), 1.727 (2.99), 2.024 (0.92), 2.035 (1.06), 2.054 (2.33), 2.065 (2.49), 2.085 (2.42), 2.095 (2.27), 2.116 (1.03), 2.126 (0.89), 2.293 (0.72), 2.306 (1.44), 2.313 (1.68), 2.326 (3.53), 2.332 (1.96), 2.337 (1.94), 2.345 (1.35), 2.358 (0.58), 2.518 (5.19), 2.523 (3.55), 2.539 (0.60), 2.660 (0.50), 2.665 (1.15), 2.669 (1.62), 2.673 (1.15), 2.678 (0.49), 3.431 (2.62), 3.461 (4.78), 3.487 (2.62), 3.889 (2.87), 3.897 (3.07), 3.918 (2.68), 3.925 (2.66), 3.976 (0.83), 4.577 (0.65), 4.588 (1.21), 4.599 (0.83), 4.606 (1.29), 4.617 (2.26), 4.627 (1.24), 4.634 (0.72), 4.645 (1.04), 4.656 (0.52), 4.758 (7.23), 4.773 (6.91), 5.759 (15.30), 6.902 (0.45), 7.046 (1.68), 7.061 (3.73), 7.076 (1.59), 7.128 (0.98), 7.145 (1.21), 7.150 (2.01), 7.155 (1.12), 7.167 (2.29), 7.178 (2.26), 7.193 (5.19), 7.203 (4.70), 7.214 (1.47), 7.225 (1.29), 8.365 (0.42), 8.379 (16.00), 12.580 (2.60), 13.062 (0.47).

### EXAMPLE 33

### 6-cyclopropyl-N-[(4-methyl-1H-benzimidazol-2-yl)methyl]-1-(oxan-4-yl)-1H-pyrazolo[3,4-b]pyrazin-3-amine

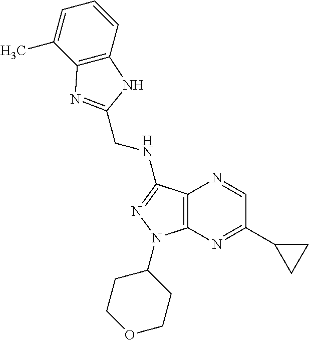 [SMILES: Cc1cccc2[nH]c(CNc3nn(C4CCOCC4)c4nc(C5CC5)cnc34)nc12]

In analogy to example 3) 6-cyclopropyl-1-(oxan-4-yl)-1H-pyrazolo[3,4-b]pyrazin-3-amine (100 mg, 386 μmol, intermediate 18) and 4-methyl-1H-benzimidazole-2-carbaldehyde (124 mg, 771 μmol, intermediate 10) reacted and give after purification using a Biotage chromatography system followed by a HPLC purifcation 36.5 mg (97% purity, 23% yield) of the desired title compound.

LC-MS (Method 2): Rₜ=1.14 min; MS (ESIpos): m/z=404 [M+H]⁺

¹H-NMR (400 MHz, DMSO-d6) δ [ppm]: 1.032 (1.15), 1.044 (3.06), 1.050 (4.24), 1.057 (3.80), 1.062 (4.04), 1.069 (2.01), 1.078 (0.78), 1.090 (1.94), 1.097 (3.51), 1.104 (2.51), 1.110 (2.22), 1.118 (3.94), 1.124 (2.14), 1.136 (0.94), 1.232 (0.85), 1.715 (1.87), 1.720 (1.93), 1.746 (2.26), 1.752 (2.23), 2.075 (0.66), 2.087 (0.79), 2.106 (1.71), 2.116 (1.83), 2.136 (1.72), 2.146 (1.66), 2.166 (0.70), 2.178 (0.64), 2.294 (0.56), 2.306 (1.14), 2.313 (1.28), 2.323 (2.47), 2.326 (3.09), 2.332 (1.82), 2.337 (1.64), 2.345 (1.13), 2.358 (0.46), 2.468 (5.29), 2.518 (6.00), 2.523 (4.29), 2.539 (0.51), 2.550 (0.51), 2.660 (0.68), 2.665 (1.46), 2.669 (1.90), 2.673 (1.30), 2.678 (0.57), 3.448 (1.58), 3.478 (2.99), 3.507 (1.57), 3.923 (2.12), 3.942 (1.93), 3.977 (0.97), 4.605 (0.58), 4.633 (1.05), 4.661 (0.51), 4.754 (6.90), 4.769 (6.77), 5.759 (14.78), 6.781 (0.71), 6.903 (1.10), 6.920 (2.58), 6.934 (2.01), 6.987 (2.24), 7.006 (3.38), 7.024 (1.71), 7.209 (1.33), 7.228 (1.09), 7.335 (0.83), 7.354 (0.73), 8.001 (0.48), 8.376 (16.00), 12.103 (1.30), 12.238 (0.90).

### EXAMPLE 34

### (±)-N-[(1H-benzimidazol-2-yl)methyl]-6-cyclopropyl-1-(1-methylazepan-4-yl)-1H-pyrazolo[3,4-b]pyrazin-3-amine

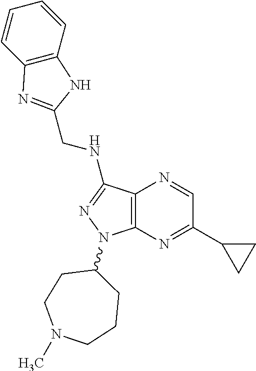 [SMILES: CN1CCCC(n2nc(NCc3nc4ccccc4[nH]3)c3ncc(C4CC4)nc32)CC1]

In analogy to example 32) (±)-6-cyclopropyl-1-(1-methylazepan-4-yl)-1H-pyrazolo[3,4-b]pyrazin-3-amine (434 mg, 758 μmol, intermediate 37) and 1H-benzimidazole-2-carbaldehyde (221 mg, 1.52 mmol) reacted and give after purification using a Biotage chromatography system followed by a HPLC purifcation 49.0 mg (95% purity, 15% yield) of the desired title compound.

LC-MS (Method 2): R₁=1.15 min; MS (ESIpos): m/z=418 [M+H]⁺

¹H-NMR (400 MHz, DMSO-d6) δ [ppm]: 1.021 (0.72), 1.031 (1.76), 1.038 (2.53), 1.044 (2.12), 1.050 (2.32), 1.057 (1.12), 1.067 (0.43), 1.078 (0.67), 1.082 (0.98), 1.089 (2.17), 1.095 (1.35), 1.102 (1.22), 1.109 (2.24), 1.116 (1.32), 1.123 (0.49), 1.127 (0.55), 1.568 (0.47), 1.750 (0.47), 1.758 (0.43), 1.763 (0.48), 1.770 (0.46), 1.810 (0.73), 1.824 (0.43), 1.838 (0.59), 1.844 (0.65), 1.852 (0.70), 1.858 (0.86), 1.865 (0.61), 1.872 (0.77), 1.886 (0.53), 1.907 (0.61), 1.993 (1.11), 2.011 (0.65), 2.016 (0.64), 2.035 (0.40), 2.042 (0.52), 2.069 (0.52), 2.076 (0.54), 2.092 (0.63), 2.099 (0.71), 2.114 (0.53), 2.121 (0.46), 2.127 (0.53), 2.135 (0.56), 2.217 (16.00), 2.228 (0.84), 2.276 (1.10), 2.282 (0.46), 2.294 (0.96), 2.302 (0.78), 2.314 (1.25), 2.323 (1.06), 2.327 (1.41), 2.332 (0.85), 2.335 (0.80), 2.404 (0.42), 2.410 (0.49), 2.427 (0.59), 2.436 (0.97), 2.444 (0.76), 2.459 (1.60), 2.466 (1.71), 2.518 (2.70), 2.523 (1.82), 2.539 (1.13), 2.573 (0.54), 2.581 (0.66), 2.592 (0.63), 2.599 (0.62), 2.606 (0.51), 2.614 (0.45), 2.625 (0.43), 2.665 (0.58), 2.669 (0.84), 2.673 (0.58), 2.852 (0.54), 2.865 (0.54), 3.967 (0.69), 4.696 (0.51), 4.705 (0.60), 4.719 (0.99), 4.732 (0.82), 4.747 (3.97), 4.763 (3.82), 5.759 (1.32), 6.868 (0.85), 6.884 (1.93), 6.899 (0.81), 7.086 (0.59), 7.095 (3.04), 7.103 (2.93), 7.110 (3.05), 7.119 (3.45), 7.128 (0.63), 7.463 (0.64), 7.974 (0.41), 8.272 (0.54), 8.352 (9.61), 12.160 (0.53).

### EXAMPLE 35

### (±)-N-[(1H-benzimidazol-2-yl)methyl]-6-cyclopropyl-1-(1-methylpyrrolidin-3-yl)-1H-pyrazolo[3,4-b]pyrazin-3-amine

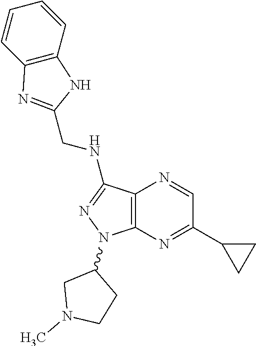 [SMILES: CN1CCC(n2nc(NCc3nc4ccccc4[nH]3)c3ncc(C4CC4)nc32)C1]

In analogy to example 32) (±)-6-cyclopropyl-1-(1-methylpyrrolidin-3-yl)-1H-pyrazolo[3,4-b]pyrazin-3-amine (300 mg, 1.16 mmol, intermediate 38) and 1H-benzimidazole-2-carbaldehyde (339 mg, 2.32 mmol) reacted and give after purification using a Biotage chromatography system followed by a HPLC purifcation 45.0 mg (95% purity, 9% yield) of the desired title compound.

LC-MS (Method 2): Rₜ=1.09 min; MS (ESIneg): m/z=387 [M−H]⁻

¹H-NMR (400 MHz, DMSO-d6) δ [ppm]: 1.030 (0.71), 1.042 (1.89), 1.049 (2.76), 1.054 (2.80), 1.061 (2.59), 1.066 (1.35), 1.086 (1.27), 1.093 (2.46), 1.100 (1.69), 1.106 (1.27), 1.113 (2.68), 1.120 (1.51), 1.129 (0.70), 1.232 (0.59), 2.093 (0.80), 2.106 (1.02), 2.113 (0.61), 2.119 (0.64), 2.126 (0.81), 2.137 (0.66), 2.155 (0.85), 2.173 (0.66), 2.179 (0.96), 2.187 (0.64), 2.197 (0.63), 2.222 (16.00), 2.242 (0.64), 2.247 (0.59), 2.289 (1.04), 2.303 (0.89), 2.310 (1.04), 2.322 (2.15), 2.327 (1.62), 2.331 (1.69), 2.342 (0.78), 2.354 (0.41), 2.518 (6.92), 2.523 (5.57), 2.540 (6.03), 2.565 (2.55), 2.583 (0.76), 2.625 (0.74), 2.637 (0.81), 2.644 (1.02), 2.657 (1.18), 2.665 (1.38), 2.669 (1.46), 2.673 (1.08), 2.861 (0.41), 2.874 (0.44), 2.891 (1.01), 2.911 (1.46), 2.934 (0.92), 4.752 (4.28), 4.767 (4.25), 5.105 (0.54), 5.125 (0.69), 5.144 (0.53), 5.760 (1.68), 6.968 (0.92), 6.984 (2.12), 6.999 (0.91), 7.098 (2.21), 7.106 (2.26), 7.113 (2.33), 7.121 (2.42), 7.419 (0.44), 7.514 (0.45), 8.382 (9.45), 12.142 (0.50).

### EXAMPLE 36

### N-[(1H-benzimidazol-2-yl)methyl]-6-cyclopropyl-1-(4,4-difluorocyclohexyl)-1H-pyrazolo[3,4-b]pyrazin-3-amine

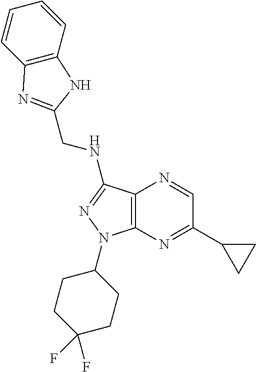 [SMILES: FC1(F)CCC(n2nc(NCc3nc4ccccc4[nH]3)c3ncc(C4CC4)nc32)CC1]

In analogy to example 3) 6-cyclopropyl-1-(4,4-difluorocyclohexyl)-1H-pyrazolo[3,4-b]pyrazin-3-amine (45.0 mg, 153 μmol, intermediate 33) and 1H-benzimidazole-2-carbaldehyde (44.8 mg, 307 μmol) reacted and give after purification using a Biotage chromatography system followed by a HPLC purifcation 6.8 mg (95% purity, 10% yield) of the desired title compound.

LC-MS (Method 2): Rₜ=1.23 min; MS (ESIpos): m/z=424 [M+H]⁺

¹H-NMR (400 MHz, DMSO-d6) δ [ppm]: 0.852 (0.40), 1.029 (1.25), 1.041 (3.36), 1.048 (4.75), 1.054 (4.12), 1.060 (4.65), 1.067 (2.25), 1.079 (0.91), 1.092 (2.14), 1.100 (4.26), 1.106 (2.62), 1.112 (2.69), 1.119 (4.44), 1.126 (2.67), 1.138 (1.22), 1.232 (1.59), 1.844 (1.89), 1.875 (2.07), 1.987 (0.58), 2.011 (1.08), 2.057 (1.51), 2.114 (4.41), 2.133 (2.30), 2.162 (1.61), 2.295 (0.75), 2.307 (1.35), 2.323 (2.97), 2.327 (5.07), 2.332 (2.76), 2.336 (2.03), 2.347 (1.21), 2.359 (0.56), 2.518 (13.51), 2.523 (8.17), 2.539 (2.10), 2.665 (2.05), 2.669 (2.83), 2.673 (2.06), 4.629 (1.22), 4.655 (0.75), 4.749 (6.99), 4.765 (6.95), 5.759 (4.67), 6.926 (1.71), 6.941 (3.74), 6.956 (1.57), 7.083 (0.96), 7.096 (3.46), 7.101 (5.00), 7.110 (5.80), 7.119 (5.49), 7.124 (3.71), 7.138 (1.01), 7.392 (2.59), 7.395 (2.24), 7.401 (1.71), 7.409 (2.06), 7.414 (2.09), 7.513 (2.35), 7.519 (2.07), 7.527 (1.37), 7.535 (2.14), 8.380 (16.00), 12.165 (2.83).

### EXAMPLE 37

### (+) or (−)-N-[(1H-benzimidazol-2-yl)methyl]-6-cyclopropyl-1-[(4R*)-1-methylazepan-4-yl]-1H-pyrazolo[3,4-b]pyrazin-3-amine

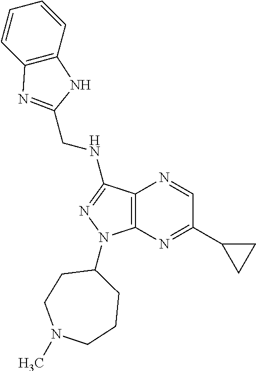 [SMILES: CN1CCCC(n2nc(NCc3nc4ccccc4[nH]3)c3ncc(C4CC4)nc32)CC1]

(±)-N-[(1H-benzimidazol-2-yl)methyl]-6-cyclopropyl-1-(1-methylazepan-4-yl)-1H-pyrazolo[3,4-b]pyrazin-3-amine (49.0 mg, 118 μmol, Example 34) was separated in the two enantiomers using a chiral HPLC (column: SC-50-1803 F11) to give 11.2 mg (95% purity, 22% yield) of the desired compound.

### EXAMPLE 38

### (−) or (+)-N-[(1H-benzimidazol-2-yl)methyl]-6-cyclopropyl-1-[(4R*)-1-methylazepan-4-yl]-1H-pyrazolo[3,4-b]pyrazin-3-amine

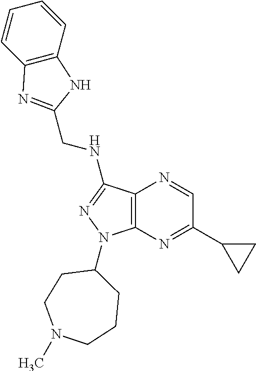 [SMILES: CN1CCCC(n2nc(NCc3nc4ccccc4[nH]3)c3ncc(C4CC4)nc32)CC1]

(±)-N-[(1H-benzimidazol-2-yl)methyl]-6-cyclopropyl-1-(1-methylazepan-4-yl)-1H-pyrazolo[3,4-b]pyrazin-3-amine (49.0 mg, 118 μmol, Example 34) was separated in the two enantiomers using a chiral HPLC (column : SC-50-1803 F11) to give 13.0 mg (95% purity, 25% yield) of the desired compound.

### EXAMPLE 39

### N-[(1H-benzimidazol-2-yl)methyl]-1-cyclopentyl-6-cyclopropyl-1H-pyrazolo[3,4-b]pyrazin-3-amine

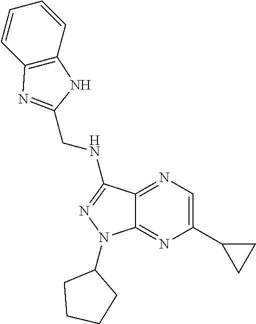 [SMILES: c1ccc2[nH]c(CNc3nn(C4CCCC4)c4nc(C5CC5)cnc34)nc2c1]

In analogy to example 3) 1-cyclopentyl-6-cyclopropyl-1H-pyrazolo[3,4-b]pyrazin-3-amine (137 mg, 563 μmol, Intermediate 40) and 1H-benzimidazole-2-carbaldehyde (165 mg, 1.13 mmol) reacted and give after purification using a Biotage chromatography system followed by a HPLC purifcation 8.00 mg (95% purity, 4% yield) of the desired title compound.

LC-MS (Method 2): Rₜ=1.31 min; MS (ESIpos): m/z=374 [M+H]⁺

¹H-NMR (400 MHz, DMSO-d6) δ [ppm]: 0.781 (0.51), 0.851 (0.63), 1.019 (1.27), 1.031 (3.37), 1.038 (4.57), 1.044 (4.20), 1.049 (4.54), 1.056 (2.22), 1.065 (1.09), 1.078 (2.36), 1.085 (4.08), 1.092 (2.91), 1.098 (2.75), 1.105 (4.66), 1.112 (2.80), 1.123 (1.57), 1.170 (2.12), 1.231 (3.21), 1.547 (1.26), 1.557 (2.21), 1.569 (2.39), 1.574 (1.93), 1.585 (1.74), 1.605 (0.61), 1.614 (0.60), 1.700 (0.55), 1.728 (1.66), 1.745 (2.25), 1.753 (2.13), 1.765 (1.42), 1.784 (0.61), 1.797 (0.44), 1.824 (0.51), 1.842 (0.87), 1.861 (1.76), 1.878 (3.00), 1.893 (3.68), 1.898 (3.25), 1.912 (2.98), 1.930 (1.52), 1.944 (0.75), 2.284 (0.63), 2.296 (1.25), 2.304 (1.33), 2.316 (2.29), 2.327 (2.26), 2.331 (1.33), 2.336 (1.48), 2.348 (0.50), 2.518 (4.81), 2.523 (3.13), 2.540 (12.24), 2.665 (0.85), 2.669 (1.19), 2.673 (0.83), 2.994 (0.41), 4.747 (6.92), 4.762 (6.94), 4.920 (0.57), 4.939 (1.61), 4.957 (2.49), 4.976 (1.55), 4.993 (0.52), 6.885 (1.57), 6.900 (3.51), 6.915 (1.52), 7.079 (0.63), 7.093 (3.01), 7.098 (4.50), 7.106 (4.13), 7.115 (4.96), 7.120 (3.21), 7.134 (0.70), 7.389 (2.06), 7.398 (1.32), 7.405 (1.61), 7.411 (1.68), 7.511 (1.81), 7.516 (1.65), 7.523 (1.16), 7.532 (1.74), 8.360 (16.00), 8.637 (0.48), 12.154 (2.12).

### EXAMPLE 40

### cis-3-(3-{[(1H-benzimidazol-2-yl)methyl]amino}-6-cyclopropyl-1H-pyrazolo[3,4-b]pyrazin-1-yl)cyclobutan-1-ol

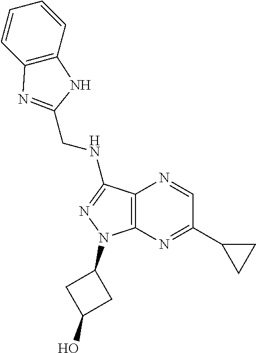 [SMILES: O[C@H]1C[C@@H](n2nc(NCc3nc4ccccc4[nH]3)c3ncc(C4CC4)nc32)C1]

To a solution of cis-3-(3-amino-6-cyclopropyl-1H-pyrazolo[3,4-b]pyrazin-1-yl)cyclobutan-1-ol (357 mg, 1.46 mmol, intermediate 42) in 6.7 mL methanol was added 1H-benzimidazole-2-carbaldehyde (425 mg, 2.91 mmol) and 120 μL acetic acid. The mixture was stirred over night at 60° C. After cooling to room temperature sodium cyanoborohydride (183 mg, 2.91 mmol) was added and the mixture was stirred for two hours at room temperature. Then additional 91 mg of sodium cyanoborohydride (1.45 mmol]) and 5 drops of trifluoroacetic acid were added and the mixture was stirred 3 hours at 50° C. and over night at room temperature. Then again an additional 46 mg of sodium cyanoborohydride (0.735 mmol]) and 5 drops of trifluoroacetic acid were added and the mixture was stirred at 50° C. for 3 hours and over night at room temperature. Then the mixture was diluted with 100 mL ethyl acetate, extracted twice with water, brine, filtered using a hydrophobic filter and evaporated to dryness. The residue was purified using a Biotage chromatography system followed by a HPLC purification to give 108 mg of the desired compound (97% purity, 19% yield).

LC-MS (Method 2): Rₜ=0.98 min; MS (ESIpos): m/z=376 [M+H]⁺

¹H-NMR (400MHz, DMSO-d₆): δ [ppm]=1.02-1.13 (m, 4H), 2.28-2.36 (m, 1H), 2.40-2.49 (m, 2H), 2.52-2.60 (m, 2H), 3.91-4.03 (m, 1H), 4.55-4.67 (m, 1H), 4.81 (d, 2H), 5.23 (d, 1H), 6.96 (t, 1H), 7.09-7.15 (m, 2H), 7.48 (br s, 2H), 8.37 (s, 1H), 12.19 (br s, 1H).

### EXAMPLE 41

### (±)-cis/trans-N-[(1H-benzimidazol-2-yl)methyl]-6-cyclopropyl-1-(1,2-dimethylpiperidin-4-yl)-1H-pyrazolo[3,4-b]pyrazin-3-amine

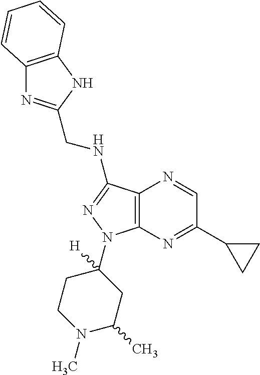 [SMILES: CC1CC(n2nc(NCc3nc4ccccc4[nH]3)c3ncc(C4CC4)nc32)CCN1C]

In analogy to example 3) (±)-cis/trans-6-cyclopropyl-1-(1,2-dimethylpiperidin-4-yl)-1H-pyrazolo[3,4-b]pyrazin-3-amine (277 mg, 967 μmol, intermediate 46) and 1H-benzimidazole-2-carbaldehyde (283 mg, 1.93 mmol) reacted and give after purification using a Biotage chromatography system followed by a HPLC purifcation 46.8 mg (95% purity, 11% yield) of the desired title compound.

LC-MS (Method 2): Rₜ=1.17 min; MS (ESIneg): m/z=416 [M−H]⁻

¹H-NMR (400MHz, DMSO-d₆): δ [ppm]=0.97-1.14 (m, 7H), 1.64-1.86 (m, 3H), 1.96-2.04 (m, 1H), 2.06-2.21 (m, 5H), 2.26-2.40 (m, 1H), 2.78-2.92 (m, 1H), 4.36-4.46 (m, 1H), 4.71-4.80 (m, 2H), 6.89 (t, 1H), 7.05-7.17 (m, 2H), 7.36-7.47 (m, 1H), 7.45-7.58 (m, 1H), 8.34-8.38 (m, 1H), 12.13-12.22 (m, 1H).

### EXAMPLE 42

### (±)-cis/trans-N-[(1H-benzimidazol-2-yl)methyl]-6-cyclopropyl-1-(3-methyloxan-4-yl)-1H-pyrazolo[3,4-b]pyrazin-3-amine

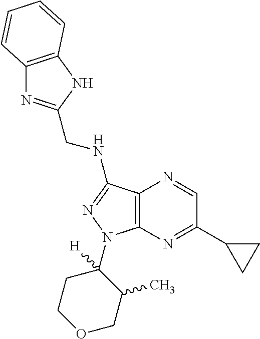 [SMILES: CC1COCCC1n1nc(NCc2nc3ccccc3[nH]2)c2ncc(C3CC3)nc21]

In analogy to example 3) (±)-cis/trans-6-cyclopropyl-1-(3-methyloxan-4-yl)-1H-pyrazolo[3,4-b]pyrazin-3-amine (366 mg, 1.34 mmol, intermediate 50) and 1H-benzimidazole-2-carbaldehyde (391 mg, 2.68 mmol) reacted and give after purification using a Biotage chromatography system followed by a HPLC purifcation 12.4 mg (93% purity, 2% yield) of the desired title compound.

LC-MS (Method 2): Rₜ=1.12 min; MS (ESIneg): m/z=402 [M−H]⁻

¹H-NMR (400 MHz, DMSO-d6) δ [ppm]: 0.351 (11.59), 0.368 (11.64), 0.435 (5.30), 0.452 (5.35), 0.851 (0.55), 1.023 (2.48), 1.035 (3.20), 1.042 (3.26), 1.055 (1.88), 1.080 (1.38), 1.088 (2.70), 1.097 (4.69), 1.108 (3.48), 1.117 (4.41), 1.124 (2.65), 1.172 (0.66), 1.232 (2.26), 1.353 (0.39), 1.652 (0.99), 1.662 (0.99), 1.684 (1.16), 1.694 (1.10), 1.849 (0.44), 1.988 (0.94), 2.090 (0.77), 2.101 (0.88), 2.121 (1.27), 2.131 (1.32), 2.152 (1.38), 2.157 (1.43), 2.163 (1.43), 2.185 (1.43), 2.195 (0.99), 2.213 (0.77), 2.289 (0.66), 2.301 (1.32), 2.309 (1.54), 2.323 (4.30), 2.327 (4.25), 2.331 (3.97), 2.340 (1.43), 2.352 (0.55), 2.518 (12.74), 2.523 (8.50), 2.540 (11.14), 2.660 (1.16), 2.665 (2.54), 2.669 (3.53), 2.673 (2.43), 2.678 (1.10), 3.071 (1.66), 3.099 (3.20), 3.127 (1.60), 3.430 (0.77), 3.439 (1.10), 3.447 (1.16), 3.458 (1.43), 3.473 (2.15), 3.502 (1.38), 3.562 (0.94), 3.576 (0.94), 3.590 (0.66), 3.604 (0.61), 3.789 (1.27), 3.801 (1.38), 3.818 (1.32), 3.830 (1.21), 3.912 (1.05), 3.924 (1.16), 3.941 (0.99), 3.951 (0.94), 4.017 (0.61), 4.030 (0.61), 4.046 (0.44), 4.195 (0.77), 4.206 (0.88), 4.224 (1.32), 4.233 (1.32), 4.252 (0.83), 4.262 (0.72), 4.733 (2.54), 4.754 (7.23), 4.769 (6.79), 4.785 (1.05), 4.797 (0.50), 5.760 (4.14), 6.920 (1.77), 6.935 (3.92), 6.951 (1.82), 7.065 (0.44), 7.079 (2.26), 7.082 (2.76), 7.092 (5.63), 7.098 (5.63), 7.101 (3.97), 7.106 (6.34), 7.115 (5.41), 7.121 (3.48), 7.134 (0.88), 7.368 (1.16), 7.371 (0.99), 7.377 (0.83), 7.382 (1.10), 7.391 (3.14), 7.393 (2.10), 7.400 (1.71), 7.407 (1.77), 7.412 (1.88), 7.495 (1.10), 7.502 (1.05), 7.511 (2.32), 7.517 (2.65), 7.523 (1.27), 7.533 (1.88), 8.374 (16.00), 8.388 (8.77), 12.142 (1.32), 12.180 (2.48).

### EXAMPLE 43

### trans-3-(3-{[(1H-benzimidazol-2-yl)methyl]amino}-6-cyclopropyl-1H-pyrazolo[3,4-b]pyrazin-1-yl)cyclobutan-1-ol

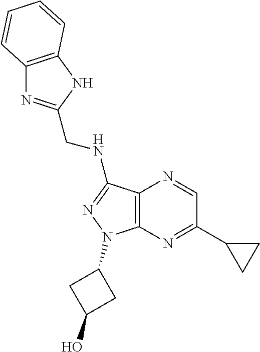 [SMILES: O[C@H]1C[C@H](n2nc(NCc3nc4ccccc4[nH]3)c3ncc(C4CC4)nc32)C1]

In analogy to example 3) trans-3-(3-amino-6-cyclopropyl-1H-pyrazolo[3,4-b]pyrazin-1-yl)cyclobutan-1-ol (266 mg, 1.08 mmol, intermediate 52) and 1H-benzimidazole-2-carbaldehyde (317 mg, 2.17 mmol) reacted and give after purification using a Biotage chromatography system followed by a HPLC purifcation 85.2 mg (95% purity, 20% yield) of the desired title compound.

LC-MS (Method 2): Rₜ=0.92 min; MS (ESIpos): m/z=377 [M+H]⁺

¹H-NMR (400 MHz, DMSO-d6) δ [ppm]: 1.026 (0.94), 1.039 (2.69), 1.045 (3.69), 1.051 (3.67), 1.057 (3.54), 1.064 (1.80), 1.070 (0.77), 1.082 (2.05), 1.089 (3.28), 1.096 (2.38), 1.103 (2.04), 1.109 (3.78), 1.116 (2.05), 1.128 (0.88), 2.212 (1.29), 2.221 (1.41), 2.233 (1.65), 2.244 (2.84), 2.254 (1.87), 2.266 (1.69), 2.275 (1.53), 2.288 (0.56), 2.300 (1.04), 2.308 (1.15), 2.319 (1.96), 2.328 (1.17), 2.331 (1.38), 2.340 (1.02), 2.352 (0.43), 2.518 (1.56), 2.523 (1.04), 2.587 (1.47), 2.596 (0.81), 2.605 (2.32), 2.612 (1.43), 2.620 (2.81), 2.628 (1.38), 2.635 (2.03), 2.644 (0.71), 2.652 (1.26), 2.669 (0.47), 4.416 (1.11), 4.426 (1.09), 4.792 (5.69), 4.807 (5.72), 5.125 (2.35), 5.137 (2.24), 5.204 (0.91), 5.209 (0.90), 5.224 (1.62), 5.241 (0.89), 5.245 (0.87), 5.759 (13.95), 6.989 (1.23), 7.004 (2.63), 7.018 (1.17), 7.106 (0.63), 7.116 (6.42), 7.124 (5.42), 7.131 (5.65), 7.139 (6.77), 7.149 (0.80), 7.460 (0.69), 7.468 (4.69), 7.476 (3.81), 7.483 (3.76), 7.491 (4.05), 7.499 (0.57), 8.379 (16.00).

### EXAMPLE 44

### (±)-cis/trans-N-[(1H-benzimidazol-2-yl)methyl]-6-cyclopropyl-1-(2-methyloxan-4-yl)-1H-pyrazolo[3,4-b]pyrazin-3-amine

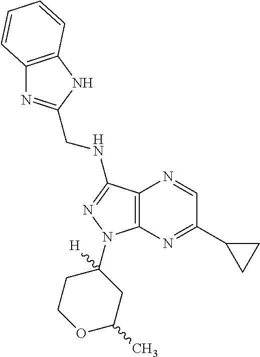 [SMILES: CC1CC(n2nc(NCc3nc4ccccc4[nH]3)c3ncc(C4CC4)nc32)CCO1]

In analogy to example 3) (±)-6-cyclopropyl-1-(cis/trans-2-methyloxan-4-yl)-1H-pyrazolo[3,4-b]pyrazin-3-amine (310 mg, 873 μmol, intermediate 56) and 1H-benzimidazole-2-carbaldehyde (255 mg, 1.75 mmol) reacted and give after purification using a Biotage chromatography system followed by a HPLC purifcation 201 mg (97% purity, 55% yield) of the desired title compound.

LC-MS (Method 2): Rₜ=1.14 min; MS (ESIpos): m/z=404 [M+H]⁺

¹H-NMR (400 MHz, DMSO-d6) δ [ppm]: 1.029 (1.29), 1.041 (3.45), 1.047 (4.86), 1.053 (4.64), 1.059 (4.83), 1.066 (2.54), 1.086 (14.79), 1.096 (6.89), 1.102 (16.00), 1.116 (5.04), 1.123 (2.86), 1.134 (1.24), 1.231 (0.51), 1.675 (1.23), 1.706 (1.54), 1.720 (1.67), 1.746 (4.53), 1.765 (3.17), 1.977 (0.46), 1.988 (0.60), 2.008 (1.27), 2.019 (1.32), 2.039 (1.21), 2.050 (1.21), 2.069 (0.46), 2.290 (0.63), 2.302 (1.26), 2.310 (1.47), 2.322 (2.90), 2.332 (1.84), 2.342 (1.24), 2.354 (0.55), 2.664 (0.69), 2.668 (0.90), 2.673 (0.69), 3.473 (1.20), 3.499 (2.33), 3.515 (1.26), 3.530 (2.60), 3.548 (1.51), 3.564 (0.97), 3.903 (1.47), 3.911 (1.56), 3.931 (1.33), 3.940 (1.29), 4.604 (0.48), 4.634 (1.12), 4.644 (1.12), 4.655 (1.12), 4.674 (0.66), 4.685 (0.48), 4.753 (7.16), 4.768 (7.24), 5.758 (1.76), 6.903 (1.78), 6.919 (3.84), 6.934 (1.73), 7.101 (4.07), 7.109 (4.20), 7.116 (4.35), 7.124 (4.55), 7.425 (0.85), 7.513 (0.87), 8.369 (13.73), 12.184 (2.80).

### EXAMPLE 45

### exo/endo-N-[(1H-benzimidazol-2-yl)methyl]-6-cyclopropyl-1-(8-oxabicyclo[3.2.1]octan-3-yl)-1H-pyrazolo[3,4-b]pyrazin-3-amine

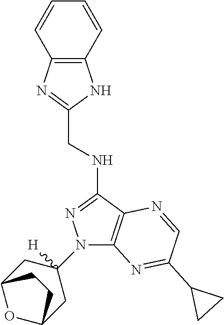 [SMILES: c1ccc2[nH]c(CNc3nn(C4C[C@H]5CC[C@@H](C4)O5)c4nc(C5CC5)cnc34)nc2c1]

In analogy to example 3) exo/endo-6-cyclopropyl-1-(8-oxabicyclo[3.2.1]octan-3-yl)-1H-pyrazolo[3,4-b]pyrazin-3-amine (434 mg, 760 μmol, intermediate 59) and 1H-benzimidazole-2-carbaldehyde (222 mg, 1.52 mmol) reacted and give after purification using a Biotage chromatography system followed by a HPLC purifcation 10.5 mg (95% purity, 3% yield) of the desired title compound.

LC-MS (Method 2): Rₜ=1.12 min; MS (ESIpos): m/z=417 [M+H]⁺

¹H-NMR (400 MHz, DMSO-d6) δ [ppm]: 1.040 (0.96), 1.052 (2.88), 1.059 (3.98), 1.065 (4.28), 1.071 (3.71), 1.078 (1.97), 1.093 (2.43), 1.100 (3.59), 1.107 (2.33), 1.114 (2.24), 1.121 (4.13), 1.128 (2.16), 1.139 (0.96), 1.232 (0.64), 1.629 (1.82), 1.643 (1.89), 1.662 (2.04), 1.675 (1.97), 1.808 (0.57), 1.821 (2.29), 1.840 (3.61), 1.870 (2.31), 1.880 (2.61), 1.902 (1.40), 2.167 (1.35), 2.176 (1.52), 2.198 (2.46), 2.205 (2.46), 2.229 (1.35), 2.237 (1.20), 2.288 (0.57), 2.300 (1.11), 2.308 (1.20), 2.320 (2.38), 2.327 (2.04), 2.332 (2.11), 2.340 (1.11), 2.352 (0.47), 2.518 (5.21), 2.522 (3.24), 2.660 (0.47), 2.664 (1.03), 2.669 (1.43), 2.673 (1.06), 2.678 (0.47), 3.839 (0.42), 4.400 (3.39), 4.753 (6.29), 4.768 (6.29), 4.793 (0.74), 4.808 (0.98), 4.822 (1.33), 4.836 (0.96), 4.851 (0.66), 5.759 (5.82), 6.904 (1.40), 6.919 (3.10), 6.934 (1.33), 7.097 (0.93), 7.106 (5.65), 7.114 (5.28), 7.121 (5.48), 7.129 (6.24), 7.139 (1.08), 7.471 (1.92), 8.355 (16.00), 12.231 (0.84).

### EXAMPLE 46

### [trans-3-(3-{[(1H-benzimidazol-2-yl)methyl]amino}-6-cyclopropyl-1H-pyrazolo[3,4-b]pyrazin-1-yl)cyclobutyl]methanol

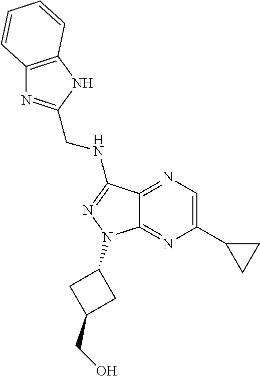 [SMILES: OC[C@H]1C[C@H](n2nc(NCc3nc4ccccc4[nH]3)c3ncc(C4CC4)nc32)C1]

In analogy to example 3) [trans-3-(3-amino-6-cyclopropyl-1H-pyrazolo[3,4-b]pyrazin-1-yl)cyclobutyl]methanol (150 mg, 521 μmol, intermediate 61) and 1H-benzimidazole-2-carbaldehyde (152 mg, 1.04 mmol) reacted and give after purification using a Biotage chromatography system followed by a HPLC purifcation 28.0 mg (95% purity, 13% yield) of the desired title compound.

LC-MS (Method 2): Rₜ=0.98 min; MS (ESIpos): m/z=391 [M+H]⁺

¹H-NMR (400 MHz, DMSO-d6) δ [ppm]: 1.027 (0.90), 1.035 (4.11), 1.039 (2.78), 1.046 (3.79), 1.052 (10.48), 1.057 (3.59), 1.064 (1.95), 1.070 (4.51), 1.080 (2.29), 1.086 (3.32), 1.093 (2.08), 1.100 (2.14), 1.107 (3.91), 1.114 (2.10), 1.126 (1.01), 1.231 (1.16), 1.751 (0.51), 1.907 (0.58), 2.084 (1.15), 2.099 (1.00), 2.107 (1.37), 2.114 (0.88), 2.121 (1.30), 2.130 (2.20), 2.139 (1.50), 2.152 (1.52), 2.160 (1.22), 2.286 (0.62), 2.298 (1.30), 2.306 (1.63), 2.318 (2.72), 2.322 (1.93), 2.326 (2.67), 2.330 (2.44), 2.337 (1.88), 2.351 (0.92), 2.518 (4.13), 2.522 (2.61), 2.573 (1.16), 2.579 (0.73), 2.597 (2.03), 2.604 (1.73), 2.617 (1.63), 2.623 (1.93), 2.641 (0.54), 2.648 (0.86), 2.660 (0.41), 2.664 (0.85), 2.668 (1.11), 2.673 (0.83), 3.404 (0.49), 3.417 (0.49), 3.422 (1.39), 3.435 (1.41), 3.439 (1.26), 3.452 (1.26), 3.457 (0.47), 3.469 (0.49), 3.502 (3.32), 3.515 (4.43), 3.531 (3.31), 4.343 (0.83), 4.356 (1.63), 4.369 (0.79), 4.673 (2.14), 4.686 (5.15), 4.700 (2.07), 4.795 (5.97), 4.810 (5.92), 5.121 (1.28), 5.141 (1.88), 5.161 (1.22), 6.950 (1.43), 6.965 (3.23), 6.980 (1.35), 7.084 (0.54), 7.097 (2.82), 7.102 (4.21), 7.111 (3.66), 7.120 (4.62), 7.126 (2.93), 7.138 (0.60), 7.398 (1.78), 7.407 (1.11), 7.414 (1.37), 7.420 (1.46), 7.518 (1.58), 7.524 (1.41), 7.530 (0.96), 7.540 (1.50), 8.367 (16.00), 10.210 (1.15), 12.182 (2.22).

### EXAMPLE 47

### [cis-3-(3-{[(1H-benzimidazol-2-yl)methyl]amino}-6-cyclopropyl-1H-pyrazolo[3,4-b]pyrazin-1-yl)cyclobutyl]methanol

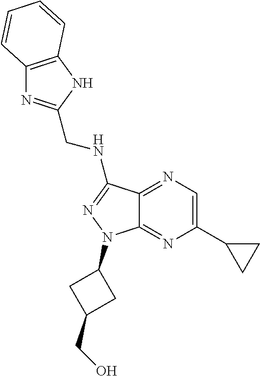 [SMILES: OC[C@H]1C[C@@H](n2nc(NCc3nc4ccccc4[nH]3)c3ncc(C4CC4)nc32)C1]

In analogy to example 3) [cis-3-(3-amino-6-cyclopropyl-1H-pyrazolo[3,4-b]pyrazin-1-yl)cyclobutyl]methanol (55.0 mg, 201 μmol, intermediate 63) and 1H-benzimidazole-2-carbaldehyde (58.9 mg, 403 μmol) reacted and give after purification using a Biotage chromatography system followed by a HPLC purifcation 20.0 mg (90% purity, 23% yield) of the desired title compound.

LC-MS (Method 2): Rₜ=1.00 min; MS (ESIpos): m/z=391 [M+H]⁺

¹H-NMR (400 MHz, DMSO-d6) δ [ppm]: 0.833 (0.66), 0.850 (1.12), 1.036 (2.23), 1.049 (6.70), 1.055 (9.30), 1.061 (10.31), 1.067 (8.48), 1.074 (5.49), 1.088 (5.79), 1.093 (8.79), 1.100 (5.43), 1.106 (6.15), 1.113 (9.65), 1.120 (5.28), 1.132 (2.79), 1.169 (2.18), 1.194 (1.57), 1.230 (5.49), 1.751 (1.27), 1.907 (0.97), 2.084 (9.55), 2.152 (1.57), 2.169 (2.29), 2.176 (1.98), 2.193 (2.18), 2.209 (1.32), 2.268 (1.12), 2.295 (9.90), 2.318 (13.97), 2.326 (11.83), 2.331 (9.96), 2.340 (5.69), 2.371 (0.91), 2.518 (10.87), 2.522 (7.16), 2.660 (1.02), 2.664 (2.18), 2.668 (3.00), 2.673 (2.13), 3.159 (4.17), 3.172 (3.91), 3.408 (7.21), 3.423 (11.99), 3.437 (7.06), 3.490 (0.61), 3.504 (1.02), 3.518 (0.56), 4.096 (0.97), 4.109 (0.91), 4.546 (3.76), 4.560 (8.08), 4.573 (3.66), 4.667 (0.86), 4.781 (5.64), 4.793 (5.54), 4.875 (0.81), 4.897 (2.84), 4.917 (4.22), 4.939 (2.90), 4.960 (0.81), 5.758 (0.81), 5.796 (0.46), 6.948 (2.54), 7.107 (6.35), 7.120 (6.35), 7.270 (0.46), 7.331 (0.41), 7.352 (0.56), 7.372 (0.46), 7.422 (1.93), 7.528 (1.93), 8.323 (0.97), 8.376 (16.00), 8.777 (1.78), 9.796 (0.81), 12.171 (2.08).

### EXAMPLE 48

### 2-[cis/trans-3-(3-{[(1H-benzimidazol-2-yl)methyl]amino}-6-cyclopropyl-1H-pyrazolo[3,4-b]pyrazin-1-yl)cyclobutyl]propan-2-ol

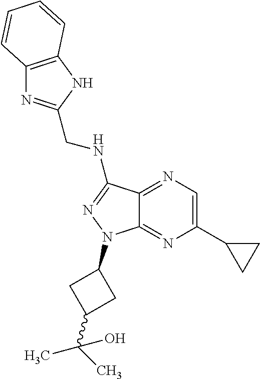

In analogy to example 32) 2-[cis/trans-3-(3-amino-6-cyclopropyl-1H-pyrazolo[3,4-b]pyrazin-1-yl)cyclobutyl]propan-2-ol (80.0 mg, 251 μmol, intermediate 65) and 1H-benzimidazole-2-carbaldehyde (73.2 mg, 501 μmol) reacted and give after purification using a Biotage chromatography system followed by a HPLC purifcation 39.0 mg (93% purity, 35% yield) of the desired title compound.

LC-MS (Method 2): Rₜ=1.19 min; MS (ESIpos): m/z=419 [M+H]⁺

¹H-NMR (400 MHz, DMSO-d6) δ [ppm]: 1.036 (16.00), 1.049 (0.87), 1.052 (1.11), 1.059 (1.80), 1.064 (1.63), 1.071 (1.67), 1.078 (1.46), 1.083 (1.73), 1.090 (0.83), 1.097 (0.91), 1.104 (1.57), 1.110 (0.77), 1.231 (0.51), 2.042 (0.50), 2.061 (0.42), 2.141 (0.75), 2.147 (0.78), 2.160 (0.68), 2.166 (0.78), 2.299 (0.43), 2.312 (0.72), 2.323 (0.59), 2.327 (0.58), 2.331 (0.63), 2.518 (1.59), 2.523 (1.50), 4.275 (2.26), 4.764 (1.82), 4.779 (1.82), 4.809 (0.66), 5.759 (10.95), 6.939 (0.44), 6.954 (0.94), 6.969 (0.42), 7.105 (0.97), 7.112 (1.03), 7.119 (1.06), 7.127 (1.07), 8.364 (5.25), 12.108 (0.57).

### EXAMPLE 49

### N-[(1H-benzimidazol-2-yl)methyl]-1-cyclobutyl-6-cyclopropyl-1H-pyrazolo[3,4-b]pyrazin-3-amine

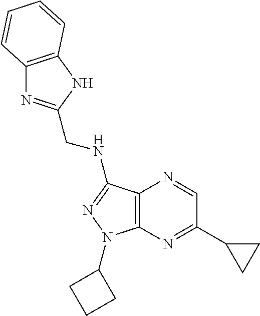 [SMILES: c1ccc2[nH]c(CNc3nn(C4CCC4)c4nc(C5CC5)cnc34)nc2c1]

In analogy to example 3) 1-cyclobutyl-6-cyclopropyl-1H-pyrazolo[3,4-b]pyrazin-3-amine (250 mg, 491 μmol, intermediate 67) and 1H-benzimidazole-2-carbaldehyde (143 mg, 981 μmol) reacted and give after purification using a Biotage chromatography system followed by a HPLC purifcation 17.3 mg (95% purity, 9% yield) of the desired title compound.

LC-MS (Method 2): Rₜ=1.23 min; MS (ESIpos): m/z=360 [M+H]⁺

¹H-NMR (400 MHz, DMSO-d6) δ [ppm]: 1.033 (1.30), 1.045 (4.51), 1.051 (6.05), 1.058 (6.92), 1.063 (5.87), 1.070 (3.27), 1.085 (3.77), 1.091 (5.75), 1.098 (3.40), 1.104 (3.64), 1.111 (6.55), 1.118 (3.40), 1.130 (1.61), 1.231 (1.11), 1.710 (0.86), 1.724 (1.98), 1.736 (2.59), 1.749 (4.02), 1.760 (2.29), 1.765 (1.92), 1.774 (2.22), 1.791 (0.80), 2.227 (2.59), 2.233 (2.90), 2.241 (2.78), 2.253 (2.41), 2.289 (0.86), 2.301 (1.61), 2.309 (1.85), 2.322 (4.82), 2.327 (4.26), 2.332 (3.83), 2.341 (1.73), 2.354 (0.68), 2.518 (16.00), 2.523 (11.00), 2.563 (3.52), 2.569 (3.34), 2.592 (2.22), 2.617 (0.68), 2.660 (1.17), 2.665 (2.59), 2.669 (3.58), 2.673 (2.47), 2.678 (1.17), 4.788 (4.57), 4.802 (4.45), 5.036 (0.56), 5.057 (1.79), 5.078 (2.72), 5.099 (1.73), 5.120 (0.49), 5.760 (2.10), 6.967 (2.04), 7.101 (4.02), 7.108 (4.32), 7.116 (4.51), 7.122 (4.32), 7.420 (1.11), 8.376 (11.49), 8.535 (0.86), 12.190 (1.73).

### EXAMPLE 50

### trans-4-(3-{[(1H-benzimidazol-2-yl)methyl]amino}-6-cyclopropyl-1H-pyrazolo[3,4-b]pyrazin-1-yl)cyclohexan-1-ol

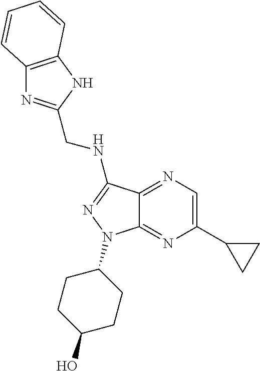 [SMILES: O[C@H]1CC[C@H](n2nc(NCc3nc4ccccc4[nH]3)c3ncc(C4CC4)nc32)CC1]

In analogy to example 3) trans-4-(3-amino-6-cyclopropyl-1H-pyrazolo[3,4-b]pyrazin-1-yl)cyclohexan-1-ol (100 mg, 348 μmol, intermediate 69) and 1H-benzimidazole-2-carbaldehyde (102 mg, 695 μmol) reacted and give after purification using a Biotage chromatography system followed by a HPLC purifcation 16.0 mg (98% purity, 11% yield) of the desired title compound.

LC-MS (Method 2): Rₜ=0.98 min; MS (ESIpos): m/z=405 [M+H]⁺

¹H-NMR (400 MHz, DMSO-d6) δ [ppm]: 1.026 (1.29), 1.038 (3.75), 1.045 (5.27), 1.051 (5.25), 1.056 (5.10), 1.064 (2.51), 1.071 (1.14), 1.083 (2.71), 1.090 (4.61), 1.097 (3.42), 1.103 (2.94), 1.111 (5.22), 1.117 (2.94), 1.129 (1.32), 1.232 (1.27), 1.277 (0.91), 1.307 (2.48), 1.341 (2.54), 1.366 (0.89), 1.378 (0.81), 1.737 (2.21), 1.762 (2.89), 1.877 (3.90), 1.904 (6.64), 1.933 (2.46), 1.966 (0.89), 2.282 (0.71), 2.294 (1.39), 2.302 (1.57), 2.314 (2.61), 2.323 (2.33), 2.327 (2.92), 2.332 (1.93), 2.346 (0.61), 2.518 (9.86), 2.523 (6.24), 2.665 (1.12), 2.669 (1.55), 2.673 (1.12), 3.159 (0.58), 3.173 (0.58), 3.421 (0.68), 3.447 (1.39), 3.457 (1.39), 3.484 (0.68), 4.324 (0.89), 4.343 (1.12), 4.354 (1.77), 4.363 (1.09), 4.372 (0.74), 4.382 (0.91), 4.623 (6.19), 4.634 (6.03), 4.746 (7.45), 4.761 (7.51), 6.846 (1.88), 6.861 (4.06), 6.877 (1.83), 7.086 (0.81), 7.099 (3.45), 7.105 (5.22), 7.113 (5.25), 7.122 (5.73), 7.128 (3.85), 7.141 (0.91), 7.398 (2.64), 7.407 (1.70), 7.414 (2.16), 7.420 (2.21), 7.517 (2.33), 7.523 (2.18), 7.530 (1.55), 7.539 (2.23), 8.345 (16.00), 12.170 (3.40).

### EXAMPLE 51

### cis-4-(3-{[(1H-benzimidazol-2-yl)methyl]amino}-6-cyclopropyl-1H-pyrazolo[3,4-b]pyrazin-1-yl)cyclohexan-1-ol

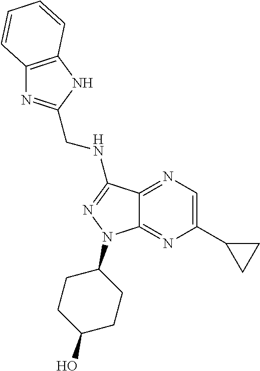 [SMILES: O[C@H]1CC[C@@H](n2nc(NCc3nc4ccccc4[nH]3)c3ncc(C4CC4)nc32)CC1]

In analogy to example 3) cis-4-(3-amino-6-cyclopropyl-1H-pyrazolo[3,4-b]pyrazin-1-yl)cyclohexan-1-ol (100 mg, 348 μmol, intermediate 71), 1H-benzimidazole-2-carbaldehyde (102 mg, 695 μmol) and in addition some drops of N,N-dimethylformamide as cosolvent reacted 3 days at 60° C. and give after purification using a Biotage chromatography system followed by a HPLC purifcation 4.80 mg (80% purity, 3% yield) of the desired title compound.

LC-MS (Method 2): Rₜ=1.07 min; MS (ESIpos): m/z=405 [M+H]⁺

¹H-NMR (400 MHz, METHANOL-d4) δ [ppm]: 1.106 (0.66), 1.122 (0.49), 1.127 (1.34), 1.130 (0.58), 1.135 (0.53), 1.139 (0.76), 4.880 (1.90), 4.888 (16.00), 5.493 (1.10), 7.172 (0.63), 7.180 (0.55), 7.187 (0.59), 7.195 (0.69), 8.263 (1.08).

### EXAMPLE 52

### N-[(1H-benzimidazol-2-yl)methyl]-6-cyclopropyl-1-(2,2,6,6-tetramethyloxan-4-yl)-1H-pyrazolo[3,4-b]pyrazin-3-amine

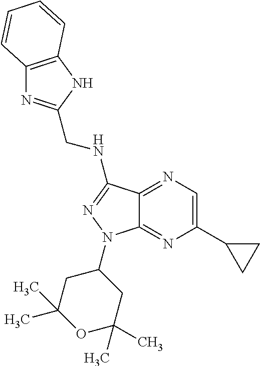 [SMILES: CC1(C)CC(n2nc(NCc3nc4ccccc4[nH]3)c3ncc(C4CC4)nc32)CC(C)(C)O1]

In analogy to example 3) 6-cyclopropyl-1-(2,2,6,6-tetramethyloxan-4-yl)-1H-pyrazolo[3,4-b]pyrazin-3-amine (150 mg, 357 μmol, intermediate 73) and 1H-benzimidazole carbaldehyde (104 mg, 713 μmol) reacted and give after purification using a Biotage chromatography system followed by a HPLC purifcation 50.0 mg (90% purity, 28% yield) of the desired title compound.

LC-MS (Method 2): Rₜ=1.34 min; MS (ESIpos): m/z=447 [M+H]⁺

¹H-NMR (400 MHz, DMSO-d6) δ [ppm]: 1.032 (0.45), 1.044 (1.28), 1.051 (1.86), 1.057 (1.56), 1.062 (1.78), 1.070 (0.86), 1.096 (0.82), 1.103 (1.72), 1.109 (1.09), 1.115 (1.20), 1.123 (2.30), 1.135 (16.00), 1.172 (0.40), 1.218 (1.09), 1.232 (0.76), 1.293 (15.16), 1.377 (1.02), 1.386 (0.53), 1.695 (0.94), 1.705 (1.02), 1.727 (1.47), 1.737 (1.37), 1.826 (1.16), 1.858 (1.84), 1.889 (0.80), 2.307 (0.52), 2.314 (0.58), 2.318 (0.50), 2.323 (0.87), 2.327 (1.52), 2.331 (0.71), 2.336 (0.61), 2.347 (0.46), 2.518 (2.86), 2.523 (2.02), 2.665 (0.50), 2.669 (0.69), 2.673 (0.47), 4.760 (2.17), 4.776 (2.17), 4.928 (0.71), 5.760 (2.00), 6.898 (0.54), 6.913 (1.16), 6.928 (0.52), 7.098 (1.12), 7.105 (1.53), 7.113 (1.68), 7.120 (1.63), 7.127 (1.27), 7.400 (0.83), 7.408 (0.51), 7.417 (0.68), 7.422 (0.66), 7.518 (0.67), 7.539 (0.65), 8.375 (6.85), 12.190 (0.91).

### EXAMPLE 53

### 6-cyclopropyl-N-[(5-fluoro-1H-benzimidazol-2-yl)methyl]-1-(oxan-4-yl)-1H-pyrazolo[3,4-b]pyrazin-3-amine

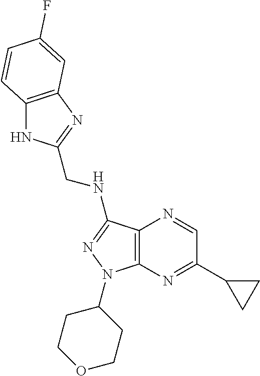 [SMILES: Fc1ccc2[nH]c(CNc3nn(C4CCOCC4)c4nc(C5CC5)cnc34)nc2c1]

In analogy to example 3) 6-cyclopropyl-1-(oxan-4-yl)-1H-pyrazolo[3,4-b]pyrazin-3-amine (100 mg, 386 μmol, intermediate 18) and 5-fluoro-1H-benzimidazole-2-carbaldehyde (104 mg, 713 μmol, intermediate 6) reacted and give after purification using a Biotage chromatography system followed by a HPLC purifcation 70.0 mg (95% purity, 42% yield) of the desired title compound.

LC-MS (Method 2): Rₜ=1.12 min; MS (ESIpos): m/z=409 [M+H]⁺

¹H-NMR (400 MHz, DMSO-d6) δ [ppm]: 1.031 (1.24), 1.042 (3.63), 1.049 (5.11), 1.055 (4.49), 1.060 (5.24), 1.068 (2.31), 1.077 (0.83), 1.091 (2.28), 1.098 (4.59), 1.104 (2.88), 1.111 (2.83), 1.118 (5.08), 1.125 (2.88), 1.131 (0.91), 1.136 (1.19), 1.231 (0.44), 1.701 (2.26), 1.732 (2.65), 2.041 (0.65), 2.049 (0.73), 2.070 (1.79), 2.084 (2.93), 2.101 (1.84), 2.111 (1.79), 2.130 (0.70), 2.140 (0.62), 2.293 (0.67), 2.305 (1.37), 2.313 (1.56), 2.325 (3.19), 2.332 (1.92), 2.337 (1.92), 2.345 (1.35), 2.357 (0.54), 2.518 (7.11), 2.523 (4.77), 2.660 (0.52), 2.665 (1.17), 2.669 (1.61), 2.674 (1.12), 2.679 (0.52), 3.435 (2.20), 3.464 (4.20), 3.492 (2.31), 3.906 (2.70), 3.925 (2.36), 4.582 (0.44), 4.592 (0.86), 4.610 (0.96), 4.621 (1.63), 4.631 (0.96), 4.649 (0.80), 4.660 (0.41), 4.737 (2.90), 4.748 (4.30), 4.762 (3.19), 5.759 (3.27), 6.929 (0.80), 6.936 (1.17), 6.955 (3.01), 6.958 (3.29), 6.975 (3.06), 6.980 (2.13), 6.982 (2.26), 6.998 (1.01), 7.005 (0.93), 7.184 (0.99), 7.190 (0.99), 7.207 (1.01), 7.213 (0.96), 7.305 (1.01), 7.310 (1.06), 7.329 (1.04), 7.335 (1.01), 7.374 (1.04), 7.387 (1.12), 7.397 (1.04), 7.409 (0.93), 7.504 (0.83), 7.517 (0.91), 7.527 (0.86), 7.539 (0.78), 8.375 (16.00), 12.273 (1.37), 12.291 (1.45).

### EXAMPLE 54

### N-[(1H-benzimidazol-2-yl)methyl]-6-cyclopropyl-1-(2-oxaspiro[3.3]heptan-6-yl)-1H-pyrazolo[3,4-b]pyrazin-3-amine

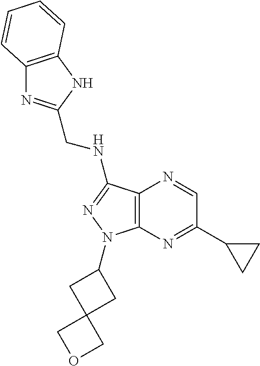 [SMILES: c1ccc2[nH]c(CNc3nn(C4CC5(COC5)C4)c4nc(C5CC5)cnc34)nc2c1]

In analogy to example 3) 6-cyclopropyl-1-(2-oxaspiro[3.3]heptan-6-yl)-1H-pyrazolo[3,4-b]pyrazin-3-amine (200 mg, 737 μmol, intermediate 75) and 1H-benzimidazole-2-carbaldehyde (215 mg, 1.47 mmol) reacted and give after purification using a Biotage chromatography system followed by a HPLC purifcation 85.0 mg (97% purity, 28% yield) of the desired title compound.

LC-MS (Method 2): Rₜ=1.06 min; MS (ESIpos): m/z=403 [M+H]⁺

¹H-NMR (400 MHz, DMSO-d6) δ [ppm]: 1.010 (0.97), 1.022 (2.97), 1.029 (4.38), 1.035 (3.56), 1.041 (4.10), 1.048 (1.95), 1.061 (0.58), 1.069 (0.75), 1.075 (1.77), 1.082 (4.02), 1.089 (2.26), 1.095 (2.38), 1.102 (4.12), 1.109 (2.41), 1.121 (1.04), 2.280 (0.55), 2.292 (1.12), 2.300 (1.27), 2.313 (2.13), 2.325 (1.44), 2.332 (1.46), 2.344 (0.47), 2.518 (2.99), 2.523 (2.09), 2.582 (0.46), 2.612 (7.88), 2.632 (7.99), 2.665 (0.87), 2.669 (0.78), 2.674 (0.50), 4.489 (15.14), 4.617 (16.00), 4.753 (5.23), 4.768 (5.25), 4.884 (0.52), 4.904 (2.22), 4.924 (3.42), 4.943 (2.15), 4.963 (0.48), 5.759 (14.09), 6.999 (1.26), 7.014 (2.70), 7.029 (1.23), 7.083 (0.44), 7.096 (2.19), 7.102 (3.33), 7.110 (2.78), 7.118 (3.58), 7.124 (2.56), 7.137 (0.52), 7.393 (1.43), 7.409 (1.29), 7.513 (1.35), 7.532 (1.25), 8.378 (14.54), 12.158 (2.22).

### EXAMPLE 55

### N-[(1H-benzimidazol-2-yl)methyl]-6-cyclopropyl-1-(oxetan-3-yl)-1H-pyrazolo[3,4-b]pyrazin-3-amine

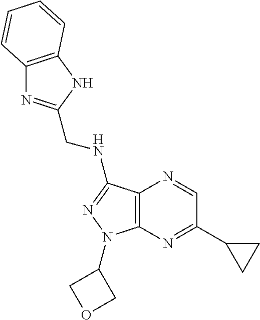 [SMILES: c1ccc2[nH]c(CNc3nn(C4COC4)c4nc(C5CC5)cnc34)nc2c1]

In analogy to example 3) 6-cyclopropyl-1-(oxetan-3-yl)-1H-pyrazolo[3,4-b]pyrazin-3-amine (234 mg, 1.01 mmol, intermediate 77) and 1H-benzimidazole-2-carbaldehyde (296 mg, 2.02 mmol) reacted and give after purification using a Biotage chromatography system followed by a HPLC purifcation 52.5 mg (97% purity, 14% yield) of the desired title compound.

LC-MS (Method 2): Rₜ=1.00 min; MS (ESIpos): m/z=362 [M−H]⁺

¹H-NMR (400 MHz, DMSO-d6) δ [ppm]: 1.041 (0.92), 1.054 (3.41), 1.060 (4.60), 1.066 (5.19), 1.072 (4.35), 1.079 (2.52), 1.092 (2.68), 1.099 (4.23), 1.105 (2.43), 1.112 (2.76), 1.119 (4.85), 1.126 (2.52), 1.137 (1.16), 2.084 (2.10), 2.302 (0.61), 2.314 (1.28), 2.322 (1.91), 2.334 (2.51), 2.346 (1.32), 2.354 (1.23), 2.366 (0.53), 2.518 (3.71), 2.523 (2.55), 2.665 (0.59), 2.669 (0.82), 2.673 (0.58), 4.821 (6.11), 4.828 (5.86), 4.836 (6.34), 4.844 (7.00), 4.848 (5.94), 4.863 (5.47), 4.950 (5.47), 4.966 (9.87), 4.983 (4.64), 5.746 (0.73), 5.760 (9.30), 5.765 (1.74), 5.782 (3.07), 5.798 (1.56), 5.801 (1.59), 5.818 (0.61), 7.100 (2.59), 7.105 (3.73), 7.114 (2.97), 7.123 (4.10), 7.128 (3.07), 7.138 (1.82), 7.154 (2.90), 7.169 (1.32), 7.399 (1.50), 7.414 (1.33), 7.523 (1.39), 7.544 (1.31), 8.426 (16.00), 12.179 (2.24).

### EXAMPLE 56

### trans-4-(6-cyclopropyl-3-{[(5-fluoro-1H-benzimidazol-2-yl)methyl]amino}-1H-pyrazolo[3,4-b]pyrazin-1-yl)cyclohexan-1-ol

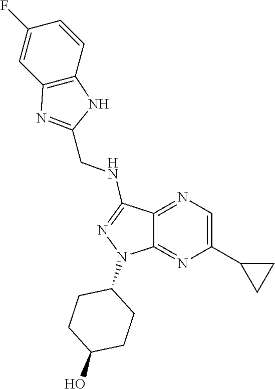 [SMILES: O[C@H]1CC[C@H](n2nc(NCc3nc4cc(F)ccc4[nH]3)c3ncc(C4CC4)nc32)CC1]

In analogy to example 3) trans-4-(3-amino-6-cyclopropyl-1H-pyrazolo[3,4-b]pyrazin-1-yl)cyclohexan-1-ol (100 mg, 348 μmol, intermediate 69) and 5-fluoro-1H-benzimidazole-2-carbaldehyde (114 mg, 695 μmol, intermediate 6) reacted and give after purification using a Biotage chromatography system followed by a HPLC purifcation 84.0 mg (98% purity, 56% yield) of the desired title compound.

LC-MS (Method 2): Rₜ=1.01 min; MS (ESIpos): m/z=423 [M+H]⁺

¹H-NMR (400 MHz, DMSO-d6) δ [ppm]: 1.025 (1.09), 1.036 (3.03), 1.043 (4.25), 1.049 (3.98), 1.054 (4.14), 1.062 (1.98), 1.070 (0.80), 1.083 (2.08), 1.090 (3.81), 1.096 (2.64), 1.103 (2.35), 1.110 (4.27), 1.117 (2.37), 1.128 (1.09), 1.231 (1.11), 1.277 (0.72), 1.304 (1.79), 1.337 (1.79), 1.363 (0.68), 1.374 (0.56), 1.728 (1.57), 1.752 (2.16), 1.865 (2.43), 1.892 (3.86), 1.918 (2.06), 1.951 (0.56), 2.281 (0.62), 2.293 (1.15), 2.301 (1.32), 2.313 (2.16), 2.323 (1.81), 2.326 (1.98), 2.332 (1.69), 2.345 (0.52), 2.518 (5.07), 2.523 (3.46), 2.665 (0.87), 2.669 (1.22), 2.673 (0.85), 3.159 (5.28), 3.172 (5.90), 3.438 (0.93), 3.448 (0.95), 3.474 (0.45), 4.084 (0.45), 4.097 (1.24), 4.110 (1.30), 4.124 (0.43), 4.320 (0.64), 4.339 (0.74), 4.348 (1.24), 4.358 (0.80), 4.376 (0.62), 4.622 (4.19), 4.633 (4.12), 4.726 (2.80), 4.737 (4.08), 4.752 (3.05), 6.870 (0.66), 6.886 (1.63), 6.905 (1.75), 6.921 (0.72), 6.930 (0.74), 6.936 (0.76), 6.952 (1.40), 6.959 (1.65), 6.977 (1.69), 6.984 (1.69), 6.999 (0.82), 7.006 (0.87), 7.182 (1.11), 7.188 (1.13), 7.205 (1.13), 7.211 (1.09), 7.303 (1.09), 7.309 (1.11), 7.328 (1.11), 7.334 (1.07), 7.373 (1.22), 7.385 (1.28), 7.395 (1.18), 7.407 (1.09), 7.503 (0.93), 7.515 (0.97), 7.524 (0.95), 7.537 (0.89), 8.344 (16.00), 12.263 (1.46), 12.281 (1.57).

### EXAMPLE 57

### (±)-N-[(1H-benzimidazol-2-yl)methyl]-6-cyclopropyl-1-(4-methoxybutan-2-yl)-1H-pyrazolo[3,4-b]pyrazin-3-amine

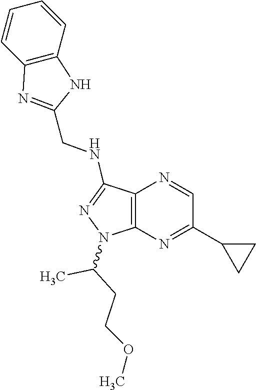 [SMILES: COCCC(C)n1nc(NCc2nc3ccccc3[nH]2)c2ncc(C3CC3)nc21]

In analogy to example 3) (±)-6-cyclopropyl-1-(4-methoxybutan-2-yl)-1H-pyrazolo[3,4-b]pyrazin-3-amine (529 mg, 2.02 mmol, intermediate 81) and 1H-benzimidazole-2-carbaldehyde (592 mg, 4.05 mmol) reacted and give after purification using a Biotage chromatography system followed by a HPLC purifcation 180 mg (92% purity, 21% yield) of the desired title compound.

LC-MS (Method 2): R₁=1.16 min; MS (ESIneg): m/z=390 [M−H]⁻

¹H-NMR (400 MHz, DMSO-d6) δ [ppm]: 1.016 (0.46), 1.026 (0.96), 1.033 (0.91), 1.035 (0.89), 1.037 (0.88), 1.045 (0.83), 1.087 (1.26), 1.096 (0.63), 1.100 (0.64), 1.107 (1.18), 1.115 (0.60), 1.322 (3.92), 1.339 (3.98), 2.014 (0.44), 2.316 (0.67), 2.328 (0.52), 2.518 (0.64), 2.523 (0.43), 2.540 (0.94), 2.863 (0.43), 2.877 (0.48), 2.882 (0.42), 2.980 (16.00), 3.020 (0.45), 3.027 (0.73), 3.042 (0.43), 3.052 (0.61), 4.752 (2.24), 4.767 (2.38), 5.759 (0.46), 6.894 (0.48), 6.909 (1.08), 6.924 (0.46), 7.094 (1.96), 7.102 (1.81), 7.109 (1.89), 7.117 (2.11), 7.460 (0.57), 8.370 (5.10).

### EXAMPLE 58

### (−)-N-[(1H-benzimidazol-2-yl)methyl]-6-cyclopropyl-1-[4-methoxybutan-2-yl]-1H-pyrazolo[3,4-b]pyrazin-3-amine

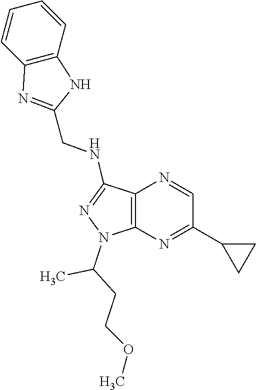 [SMILES: COCCC(C)n1nc(NCc2nc3ccccc3[nH]2)c2ncc(C3CC3)nc21]

(±)-N-[(1H-benzimidazol-2-yl)methyl]-6-cyclopropyl-1-(4-methoxybutan-2-yl)-1H-pyrazolo[3,4-b]pyrazin-3-amine (180 mg, 460 μmol, example 57) was purified using a chiral HPLC (column: IG-30-2006) to give 41.8 mg (95% purity, 22% yield) of the desired compound.

[α]₅₈₉²⁰ =−3.8 (c=1.0000 g/100 mL, DMSO)

### EXAMPLE 59

### (+)-N-[(1H-benzimidazol-2-yl)methyl]-6-cyclopropyl-1-[4-methoxybutan-2-yl]-1H-pyrazolo[3,4-b]pyrazin-3-amine

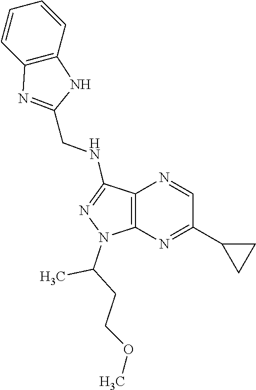 [SMILES: COCCC(C)n1nc(NCc2nc3ccccc3[nH]2)c2ncc(C3CC3)nc21]

(±)-N-[(1 H-benzimidazol-2-yl)methyl]-6-cyclopropyl-1-(4-methoxybutan-2-yl)-1H-pyrazolo[3,4-b]pyrazin-3-amine (180 mg, 460 μmol, example 57) was purified using a chiral HPLC (column: IG-30-2006) to give 46.8 mg (95% purity, 25% yield) of the desired compound.

[α]₅₈₉²⁰ =−3.8 (c=1.0000 g/100 mL, DMSO)

### EXAMPLE 60

### 3-(3-{[(1H-benzimidazol-2-yl)methyl]amino}-6-cyclopropyl-1H-pyrazolo[3,4-b]pyrazin-1-yl)propan-1-ol

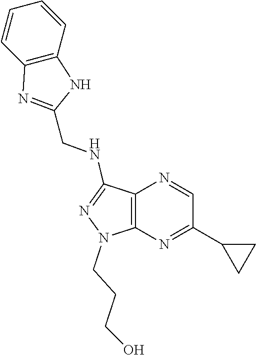 [SMILES: OCCCn1nc(NCc2nc3ccccc3[nH]2)c2ncc(C3CC3)nc21]

In analogy to example 3) 3-(3-amino-6-cyclopropyl-1H-pyrazolo[3,4-b]pyrazin-1-yl)propan-1-ol (650 mg, 2.79 mmol, intermediate 82) and 1H-benzimidazole carbaldehyde (814 mg, 5.57 mmol) reacted and give after purification using a Biotage chromatography system followed by a HPLC purifcation 500 mg (100% purity, 49% yield) of the desired title compound.

LC-MS (Method 2): Rₜ=0.92 min; MS (ESIpos): m/z=364 [M+H]⁺

¹H-NMR (400 MHz, DMSO-d6) δ [ppm]: 1.030 (0.93), 1.042 (2.83), 1.049 (3.83), 1.055 (4.10), 1.060 (3.77), 1.067 (1.97), 1.073 (0.93), 1.085 (2.39), 1.092 (3.51), 1.099 (2.33), 1.104 (2.18), 1.111 (4.05), 1.118 (2.17), 1.130 (1.02), 1.815 (0.83), 1.831 (2.71), 1.848 (3.75), 1.865 (2.81), 1.881 (0.88), 2.290 (0.53), 2.302 (1.07), 2.311 (1.17), 2.323 (2.33), 2.327 (0.96), 2.332 (1.25), 2.335 (1.25), 2.342 (1.05), 2.354 (0.45), 2.518 (2.07), 2.523 (1.43), 2.669 (0.53), 3.159 (2.64), 3.173 (2.95), 3.313 (1.87), 3.341 (5.82), 3.357 (1.87), 4.098 (0.66), 4.111 (0.63), 4.131 (2.99), 4.149 (4.95), 4.167 (2.90), 4.470 (1.79), 4.482 (3.75), 4.495 (1.70), 4.737 (6.58), 4.752 (6.58), 6.928 (1.46), 6.943 (3.32), 6.958 (1.41), 7.090 (0.87), 7.100 (5.35), 7.109 (4.75), 7.116 (5.06), 7.123 (5.89), 7.133 (1.11), 7.464 (1.08), 8.366 (16.00), 12.165 (1.41).

### EXAMPLE 61

### N-[(1H-benzimidazol-2-yl)methyl]-6-cyclopropyl-1-(3-methoxypropyl)-1H-pyrazolo[3,4-b]pyrazin-3-amine

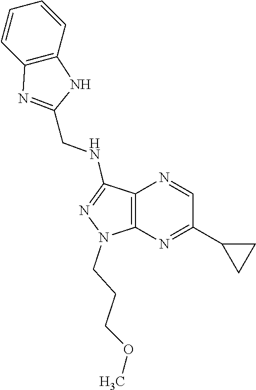 [SMILES: COCCCn1nc(NCc2nc3ccccc3[nH]2)c2ncc(C3CC3)nc21]

In analogy to example 3) 6-cyclopropyl-1-(3-methoxypropyl)-1H-pyrazolo[3,4-b]pyrazin-3-amine (39.0 mg, 126 μmol, intermediate 86) and 1H-benzimidazole-2-carbaldehyde (36.9 mg, 252 μmol) reacted and give after purification using a Biotage chromatography system followed by a HPLC purifcation 10.9 mg (90% purity, 21% yield)of the desired title compound.

LC-MS (Method 2): Rₜ=1.08 min; MS (ESIneg): m/z=376 [M−H]⁻

¹H-NMR (400 MHz, DMSO-d6) δ [ppm]: 1.039 (0.92), 1.045 (1.26), 1.051 (1.09), 1.057 (1.23), 1.064 (0.58), 1.086 (0.59), 1.093 (1.11), 1.099 (0.71), 1.106 (0.68), 1.113 (1.23), 1.120 (0.70), 1.870 (0.90), 1.886 (1.47), 1.902 (0.94), 2.317 (0.47), 2.323 (0.55), 2.328 (0.93), 2.331 (0.50), 2.337 (0.40), 2.518 (1.74), 2.523 (1.14), 2.669 (0.46), 3.076 (16.00), 3.142 (1.32), 3.157 (2.91), 3.162 (1.07), 3.173 (1.24), 3.189 (0.67), 3.212 (1.29), 3.330 (1.81), 4.118 (0.93), 4.135 (1.96), 4.151 (0.88), 4.729 (2.00), 4.745 (2.01), 6.955 (0.45), 6.970 (1.01), 6.985 (0.43), 7.094 (1.34), 7.102 (1.29), 7.109 (1.34), 7.117 (1.47), 8.380 (5.02).

### EXAMPLE 62

### (±)-N-[(1H-benzimidazol-2-yl)methyl]-6-cyclopropyl-1-[4-(dimethylamino)butan-2-yl]-1H-pyrazolo[3,4-b]pyrazin-3-amine

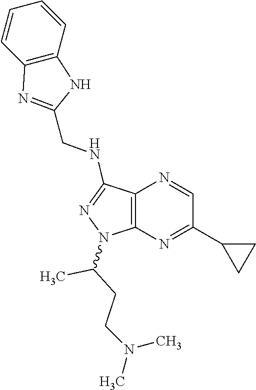 [SMILES: CC(CCN(C)C)n1nc(NCc2nc3ccccc3[nH]2)c2ncc(C3CC3)nc21]

In analogy to example 3) (±)-6-cyclopropyl-1-[4-(dimethylamino)butan-2-yl]-1H-pyrazolo[3,4-b]pyrazin-3-amine (330 mg, 1.20 mmol, intermediate 90) and 1H-benzimidazole-2-carbaldehyde (352 mg, 2.41 mmol) reacted and give after purification using a Biotage chromatography system followed by a HPLC purifcation 110 mg (92% purity, 21% yield) of the desired title compound.

LC-MS (Method 2): Rₜ=1.13 min; MS (ESIpos): m/z=406 [M+H]⁺

¹H-NMR (400 MHz, DMSO-d6) δ [ppm]: 1.011 (0.47), 1.022 (0.83), 1.031 (0.80), 1.041 (0.63), 1.085 (1.06), 1.093 (0.64), 1.104 (1.03), 1.113 (0.61), 1.323 (3.14), 1.340 (3.19), 1.820 (0.43), 1.910 (0.42), 1.916 (0.69), 1.931 (16.00), 1.942 (0.74), 1.962 (0.51), 2.022 (0.60), 2.049 (0.43), 2.055 (0.70), 2.313 (0.55), 2.518 (0.42), 4.749 (1.60), 4.765 (1.61), 6.870 (0.42), 6.886 (0.93), 7.090 (0.75), 7.097 (0.82), 7.105 (0.87), 7.112 (0.84), 8.364 (4.20).

### EXAMPLE 63

### (+) or (−)-N-[(1H-benzimidazol-2-yl)methyl]-6-cyclopropyl-1-[4-(dimethylamino)butan-2-yl]-1H-pyrazolo[3,4-b]pyrazin-3-amine

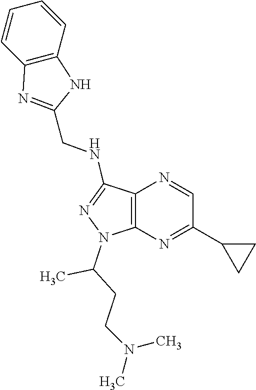 [SMILES: CC(CCN(C)C)n1nc(NCc2nc3ccccc3[nH]2)c2ncc(C3CC3)nc21]

(±)-N-[(1 H-benzimidazol-2-yl)methyl]-6-cyclopropyl-1-[4-(dimethylamino)butan-2-yl]-1H-pyrazolo[3,4-b]pyrazin-3-amine (90.0 mg, 222 μmol, example 62) was purified using a chiral HPLC (column: YMC Cellulose SC 5 μ, 250×30) to give 16.6 mg (95% purity, 18% yield) of the desired compound.

¹H-NMR (400 MHz, DMSO-d6) δ [ppm]: 1.021 (0.67), 1.031 (0.68), 1.040 (0.56), 1.085 (0.96), 1.093 (0.57), 1.105 (0.95), 1.113 (0.55), 1.323 (3.00), 1.340 (3.02), 1.817 (0.40), 1.911 (0.57), 1.927 (16.00), 1.939 (0.74), 2.313 (0.50), 2.326 (0.40), 2.518 (0.72), 2.522 (0.47), 4.748 (1.45), 4.764 (1.50), 6.884 (0.87), 7.091 (0.62), 7.097 (0.65), 7.105 (0.68), 7.110 (0.71), 7.114 (0.62), 8.363 (4.14).

### EXAMPLE 64

### (−) or(+)-N-[(1H-benzimidazol-2-yl)methyl]-6-cyclopropyl-1-[4-(dimethylamino)butan-2-yl]-1H-pyrazolo[3,4-b]pyrazin-3-amine

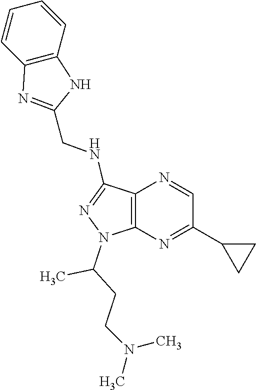 [SMILES: CC(CCN(C)C)n1nc(NCc2nc3ccccc3[nH]2)c2ncc(C3CC3)nc21]

(±)-N-[(1 H-benzimidazol-2-yl)methyl]-6-cyclopropyl-1-[4-(dimethylamino)butan-2-yl]-1H-pyrazolo[3,4-b]pyrazin-3-amine (90.0 mg, 222 μmol, example 62) was purified using a chiral HPLC (column: YMC Cellulose SC 5 μ, 250×30) to give 19.4 mg (95% purity, 20% yield) of the desired compound.

¹H-NMR (400 MHz, DMSO-d6) δ [ppm]: 1.021 (0.65), 1.031 (0.66), 1.040 (0.54), 1.085 (0.93), 1.093 (0.55), 1.105 (0.92), 1.113 (0.53), 1.323 (2.93), 1.340 (2.95), 1.910 (0.58), 1.927 (16.00), 1.938 (0.70), 1.959 (0.49), 2.083 (0.41), 2.313 (0.49), 2.518 (0.58), 4.748 (1.44), 4.764 (1.46), 6.884 (0.85), 7.088 (0.54), 7.091 (0.63), 7.097 (0.64), 7.105 (0.67), 7.110 (0.69), 7.114 (0.60), 8.363 (4.13).

### EXAMPLE 65

### N-[(1H-benzimidazol-2-yl)methyl]-6-cyclopropyl-1-(2-methoxyethyl)-1H-pyrazolo[3,4-b]pyrazin-3-amine

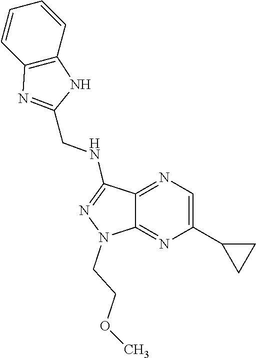 [SMILES: COCCn1nc(NCc2nc3ccccc3[nH]2)c2ncc(C3CC3)nc21]

In analogy to example 3) 6-cyclopropyl-1-(2-methoxyethyl)-1H-pyrazolo[3,4-b]pyrazin-3-amine (230 mg, 986 μmol, intermediate 91) and 1H-benzimidazole-2-carbaldehyde (288 mg, 1.97 mmol) reacted and give after purification using a Biotage chromatography system followed by a HPLC purifcation 66.0 mg (95% purity, 17% yield) of the desired title compound.

LC-MS (Method 2): Rₜ=1.06 min; MS (ESIpos): m/z=365 [M+H]⁺

¹H-NMR (400 MHz, DMSO-d6) δ [ppm]: 1.033 (0.80), 1.040 (1.16), 1.046 (0.93), 1.052 (1.12), 1.059 (0.53), 1.085 (0.50), 1.092 (1.05), 1.099 (0.60), 1.104 (0.63), 1.112 (1.10), 1.119 (0.64), 2.084 (0.51), 2.327 (0.76), 2.518 (0.73), 2.523 (0.49), 3.098 (16.00), 3.618 (0.94), 3.632 (2.00), 3.645 (1.03), 4.222 (0.91), 4.236 (1.71), 4.249 (0.83), 4.733 (1.84), 4.748 (1.84), 5.760 (0.57), 6.968 (0.41), 6.983 (0.93), 6.998 (0.40), 7.094 (0.75), 7.100 (1.07), 7.108 (0.84), 7.117 (1.20), 7.122 (0.82), 7.393 (0.45), 7.512 (0.40), 8.380 (4.75), 12.168 (0.58).

### EXAMPLE 66

### 2-(3-{[(1H-benzimidazol-2-yl)methyl]amino}-6-cyclopropyl-1H-pyrazolo[3,4-b]pyrazin-1-yl)ethan-1-ol

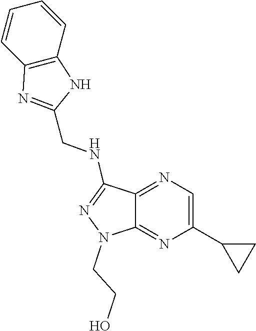 [SMILES: OCCn1nc(NCc2nc3ccccc3[nH]2)c2ncc(C3CC3)nc21]

In analogy to example 32) 2-(3-amino-6-cyclopropyl-1H-pyrazolo[3,4-b]pyrazin-1-yl)ethan-1-ol (266 mg, 1.21 mmol, intermediate 92) and 1H-benzimidazole-2-carbaldehyde (355 mg, 2.43 mmol) reacted and give after purification using a Biotage chromatography system followed by a HPLC purifcation 171 mg (95% purity, 38% yield) of the desired title compound.

LC-MS (Method 2): Rₜ=0.89 min; MS (ESIpos): m/z=350 [M+H]⁺

¹H-NMR (400 MHz, DMSO-d6) δ [ppm]: 1.030 (1.22), 1.035 (6.76), 1.042 (3.42), 1.049 (4.97), 1.053 (16.00), 1.060 (4.56), 1.070 (7.72), 1.083 (2.71), 1.090 (4.10), 1.097 (2.68), 1.103 (2.50), 1.110 (4.67), 1.117 (2.49), 1.129 (1.13), 1.231 (0.48), 2.291 (0.60), 2.303 (1.19), 2.311 (1.33), 2.322 (2.51), 2.334 (1.47), 2.342 (1.18), 2.354 (0.50), 2.522 (1.24), 2.669 (0.52), 3.405 (0.91), 3.418 (1.04), 3.423 (2.79), 3.435 (2.93), 3.440 (2.74), 3.453 (2.72), 3.457 (1.04), 3.470 (0.93), 3.678 (1.73), 3.692 (5.40), 3.707 (5.70), 3.721 (1.98), 4.132 (3.62), 4.147 (7.24), 4.162 (3.22), 4.347 (2.02), 4.360 (3.92), 4.372 (1.88), 4.735 (7.60), 4.741 (4.22), 4.750 (8.09), 4.755 (7.38), 4.769 (2.48), 6.955 (1.80), 6.970 (3.99), 6.985 (1.73), 7.086 (0.69), 7.099 (3.00), 7.104 (4.87), 7.113 (4.67), 7.122 (5.24), 7.127 (3.39), 7.140 (0.78), 7.396 (2.38), 7.405 (1.43), 7.412 (1.84), 7.419 (1.96), 7.518 (2.13), 7.524 (1.87), 7.531 (1.32), 7.540 (2.02), 8.365 (14.21), 12.135 (2.94).

### EXAMPLE 67

### N-[(1H-benzimidazol-2-yl)methyl]-6-cyclopropyl-1-(3-methoxy-3-methylbutyl)-1H-pyrazolo[3,4-b]pyrazin-3-amine

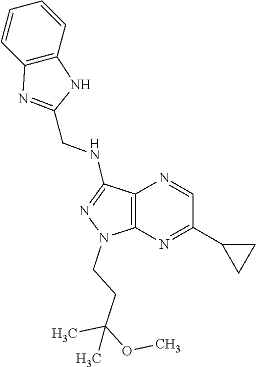 [SMILES: COC(C)(C)CCn1nc(NCc2nc3ccccc3[nH]2)c2ncc(C3CC3)nc21]

In analogy to example 3) 6-cyclopropyl-1-(3-methoxy-3-methylbutyl)-1H-pyrazolo[3,4-b]pyrazin-3-amine (38.0 mg, 138 μmol, intermediate 94) and 1H-benzimidazole-2-carbaldehyde (40.3 mg, 276 μmol) reacted and give after purification using a Biotage chromatography system followed by a HPLC purifcation 10.5 mg (92% purity, 17% yield) of the desired title compound.

LC-MS (Method 2): Rₜ=1.19 min; MS (ESIneg): m/z=404 [M−H]⁻

¹H-NMR (400 MHz, DMSO-d6) δ [ppm]: 1.035 (16.00), 1.045 (1.41), 1.053 (0.66), 1.084 (0.51), 1.091 (0.98), 1.098 (0.62), 1.104 (0.69), 1.111 (1.15), 1.118 (0.67), 1.167 (0.75), 1.776 (0.78), 1.790 (0.71), 1.795 (0.87), 1.800 (0.69), 1.814 (0.80), 2.323 (0.59), 2.327 (1.09), 2.331 (0.53), 2.336 (0.42), 2.518 (2.26), 2.523 (1.48), 2.669 (0.56), 3.017 (11.59), 4.092 (0.77), 4.106 (0.71), 4.111 (0.83), 4.117 (0.71), 4.131 (0.76), 4.729 (1.74), 4.745 (1.75), 6.933 (0.41), 6.949 (0.92), 7.092 (0.76), 7.097 (1.11), 7.106 (0.91), 7.115 (1.23), 7.120 (0.82), 7.386 (0.48), 7.506 (0.43), 7.528 (0.42), 8.373 (4.16), 12.154 (0.62).

### EXAMPLE 68

### N-[(1H-benzimidazol-2-yl)methyl]-6-cyclopropyl-1-[2-(dimethylamino)ethyl]-1H-pyrazolo[3,4-b]pyrazin-3-amine

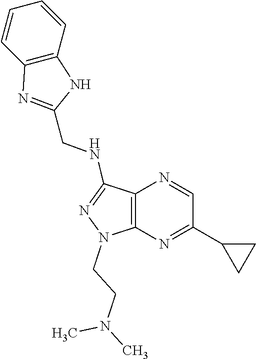 [SMILES: CN(C)CCn1nc(NCc2nc3ccccc3[nH]2)c2ncc(C3CC3)nc21]

In analogy to example 3) 6-cyclopropyl-1-[2-(dimethylamino)ethyl]-1H-pyrazolo[3,4-b]pyrazin-3-amine (118 mg, 479 μmol, intermediate 95) and 1H-benzimidazole-2-carbaldehyde (140 mg, 958 μmol) reacted and give after purification using a Biotage chromatography system followed by a HPLC purifcation 16.9 mg (85% purity, 8% yield) of the desired title compound.

LC-MS (Method 2): Rₜ=1.05 min; MS (ESIneg): m/z=375 [M−H]⁻

¹H-NMR (400 MHz, DMSO-d6) δ [ppm]: 1.016 (0.47), 1.027 (1.14), 1.033 (1.70), 1.040 (1.36), 1.045 (1.52), 1.052 (0.77), 1.074 (0.43), 1.081 (0.76), 1.088 (1.48), 1.095 (0.99), 1.102 (0.99), 1.109 (1.70), 1.116 (0.97), 1.127 (0.44), 2.041 (16.00), 2.173 (0.49), 2.188 (0.90), 2.305 (0.43), 2.313 (0.50), 2.325 (0.96), 2.337 (0.56), 2.345 (0.44), 2.465 (0.94), 2.518 (1.27), 2.523 (1.08), 2.527 (1.00), 2.543 (1.87), 2.558 (0.90), 2.664 (0.51), 3.696 (1.47), 4.151 (1.00), 4.166 (2.12), 4.182 (0.99), 4.725 (2.80), 4.740 (2.87), 5.759 (0.48), 6.934 (0.55), 6.950 (1.25), 6.965 (0.56), 7.093 (2.07), 7.102 (2.02), 7.109 (2.08), 7.116 (2.34), 7.126 (0.50), 7.459 (0.45), 7.740 (0.44), 8.375 (5.76).

### EXAMPLE 69

### 1-(3-{[(1H-benzimidazol-2-yl)methyl]amino}-6-cyclopropyl-1H-pyrazolo[3,4-b]pyrazin-1-yl)-2-methylpropan-2-ol

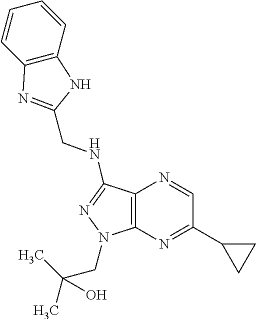 [SMILES: CC(C)(O)Cn1nc(NCc2nc3ccccc3[nH]2)c2ncc(C3CC3)nc21]

In analogy to example 3) 1-(3-amino-6-cyclopropyl-1H-pyrazolo[3,4-b]pyrazin-1-yl)-2-methylpropan-2-ol (101 mg, 408 μmol, intermediate 97) and 1H-benzimidazole-2-carbaldehyde (119 mg, 817 μmol) reacted and give after purification using a Biotage chromatography system followed by a HPLC purifcation 17.6 mg (97% purity, 11% yield) of the desired title compound.

LC-MS (Method 2): Rₜ=1.01 min; MS (ESIpos): m/z=378 [M+H]⁺

¹H-NMR (400 MHz, DMSO-d6) δ [ppm]: 0.967 (16.00), 1.017 (0.52), 1.029 (1.36), 1.035 (2.00), 1.041 (1.61), 1.047 (1.74), 1.054 (0.91), 1.085 (0.81), 1.092 (1.70), 1.099 (1.03), 1.105 (1.14), 1.112 (1.91), 1.119 (1.11), 1.130 (0.54), 1.161 (0.40), 2.305 (0.50), 2.313 (0.58), 2.318 (0.60), 2.327 (1.36), 2.332 (0.89), 2.337 (0.79), 2.345 (0.48), 2.518 (3.84), 2.523 (2.60), 2.665 (0.60), 2.669 (0.83), 2.674 (0.58), 4.033 (3.57), 4.696 (2.58), 4.718 (1.14), 5.759 (2.94), 7.036 (0.51), 7.097 (1.32), 7.109 (1.29), 8.381 (3.07), 12.131 (0.42).

### EXAMPLE 70

### trans-3-[(3-{[(1H-benzimidazol-2-yl)methyl]amino}-6-cyclopropyl-1H-pyrazolo[3,4-b]pyrazin-1-yl)methyl]cyclobutan-1-ol

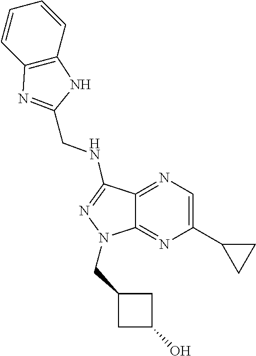 [SMILES: O[C@H]1C[C@H](Cn2nc(NCc3nc4ccccc4[nH]3)c3ncc(C4CC4)nc32)C1]

In analogy to example 3) trans-3-[(3-amino-6-cyclopropyl-1H-pyrazolo[3,4-b]pyrazin-1-yl)methyl]cyclobutan-1-ol (98.0 mg, 359 μmol, intermediate 99) and 1H-benzimidazole-2-carbaldehyde (105 mg, 718 μmol) reacted and give after purification using a Biotage chromatography system followed by a HPLC purifcation 37.0 mg (95% purity, 25% yield) of the desired title compound.

LC-MS (Method 2): Rₜ=0.95 min; MS (ESIpos): m/z=391 [M+H]⁺

¹H-NMR (400 MHz, DMSO-d6) δ [ppm]: 1.024 (1.00), 1.035 (9.37), 1.041 (4.21), 1.048 (3.48), 1.053 (16.00), 1.060 (2.02), 1.070 (6.86), 1.082 (0.66), 1.092 (1.65), 1.100 (3.33), 1.106 (2.09), 1.112 (2.26), 1.119 (3.68), 1.126 (2.22), 1.138 (1.01), 1.230 (0.82), 1.752 (0.48), 1.773 (0.89), 1.779 (0.62), 1.790 (1.14), 1.796 (2.01), 1.804 (1.77), 1.813 (1.64), 1.821 (2.31), 1.827 (1.54), 1.838 (0.81), 1.844 (1.30), 1.967 (1.39), 1.975 (1.81), 1.985 (1.79), 1.993 (2.43), 1.999 (2.07), 2.007 (1.35), 2.017 (1.36), 2.025 (1.03), 2.299 (0.54), 2.311 (1.05), 2.319 (1.36), 2.331 (2.36), 2.342 (1.08), 2.351 (0.97), 2.363 (0.42), 2.518 (2.78), 2.522 (1.91), 2.664 (0.47), 2.668 (0.64), 2.673 (0.45), 3.405 (0.93), 3.418 (0.98), 3.422 (2.63), 3.435 (2.65), 3.440 (2.64), 3.452 (2.66), 3.457 (0.84), 3.470 (0.78), 4.100 (0.94), 4.121 (5.93), 4.140 (5.63), 4.345 (1.75), 4.357 (3.38), 4.370 (1.63), 4.725 (6.01), 4.741 (6.00), 4.921 (5.70), 4.936 (5.43), 6.917 (1.48), 6.932 (3.32), 6.947 (1.41), 7.083 (0.57), 7.096 (2.82), 7.101 (4.32), 7.110 (3.84), 7.119 (4.74), 7.124 (3.08), 7.137 (0.64), 7.393 (1.87), 7.402 (1.19), 7.409 (1.45), 7.415 (1.56), 7.512 (1.70), 7.518 (1.48), 7.525 (1.05), 7.534 (1.59), 8.378 (14.59), 10.211 (0.60), 12.158 (2.37).

### EXAMPLE 71

### cis-3-[(3-{[(1H-benzimidazol-2-yl)methyl]amino}-6-cyclopropyl-1H-pyrazolo[3,4-b]pyrazin-1-yl)methyl]cyclobutan-1-ol

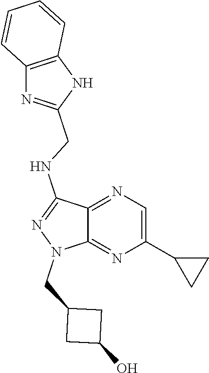 [SMILES: O[C@H]1C[C@@H](Cn2nc(NCc3nc4ccccc4[nH]3)c3ncc(C4CC4)nc32)C1]

In analogy to example 3) cis-3-[(3-amino-6-cyclopropyl-1H-pyrazolo[3,4-b]pyrazin-1-yl)methyl]cyclobutan-1-ol (102 mg, 354 μmol, intermediate 101) and 1H-benzimidazole-2-carbaldehyde (103 mg, 708 μmol) reacted and give after purification using a Biotage chromatography system followed by a HPLC purifcation 18.0 mg (93% purity, 12% yield) of the desired title compound.

LC-MS (Method 2): Rₜ=0.95 min; MS (ESIpos): m/z=391 [M+H]⁺

¹H-NMR (400 MHz, DMSO-d6) δ [ppm]: 1.029 (0.50), 1.040 (1.43), 1.047 (2.07), 1.053 (1.77), 1.059 (1.95), 1.066 (0.89), 1.092 (0.84), 1.099 (1.87), 1.105 (1.07), 1.112 (1.14), 1.119 (1.93), 1.126 (1.10), 1.137 (0.49), 1.503 (0.42), 1.518 (1.13), 1.523 (1.03), 1.539 (1.14), 1.565 (0.50), 2.039 (0.46), 2.055 (0.73), 2.065 (1.57), 2.080 (1.50), 2.095 (0.82), 2.101 (0.97), 2.307 (0.53), 2.316 (0.62), 2.327 (1.34), 2.337 (0.59), 2.339 (0.60), 2.347 (0.50), 2.518 (1.90), 2.523 (1.27), 3.160 (13.69), 3.173 (16.00), 3.774 (0.71), 3.791 (0.68), 4.070 (2.48), 4.085 (3.39), 4.098 (3.83), 4.111 (3.75), 4.124 (1.25), 4.722 (1.96), 4.737 (1.95), 4.901 (3.11), 4.919 (2.99), 6.918 (0.54), 6.933 (1.07), 6.948 (0.51), 7.097 (1.26), 7.101 (2.04), 7.110 (2.06), 7.119 (2.17), 7.123 (1.41), 7.393 (0.78), 7.409 (0.66), 7.415 (0.64), 7.515 (0.67), 7.535 (0.64), 7.732 (0.74), 8.374 (5.70), 12.146 (0.82).

### EXAMPLE 72

### 1-[(3-{[(1H-benzimidazol-2-yl)methyl]amino}-6-cyclopropyl-1H-pyrazolo[3,4-b]pyrazin-1-yl)methyl]cyclobutan-1-ol

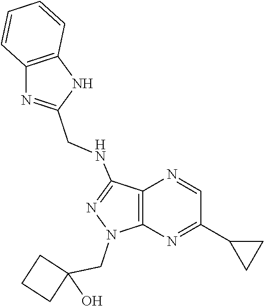 [SMILES: OC1(Cn2nc(NCc3nc4ccccc4[nH]3)c3ncc(C4CC4)nc32)CCC1]

In analogy to example 3) 1-[(3-amino-6-cyclopropyl-1H-pyrazolo[3,4-b]pyrazin-1-yl)methyl]cyclobutan-1-ol (83.6 mg, 322 μmol, intermediate 103) and 1H-benzimidazole-2-carbaldehyde (94.2 mg, 645 μmol) reacted and give after purification using a Biotage chromatography system followed by a HPLC purifcation 19.1 mg (97% purity, 15% yield) of the desired title compound.

LC-MS (Method 2): Rₜ=1.08 min; MS (ESIpos): m/z=390 [M+H]⁺

¹H-NMR (400 MHz, DMSO-d6) δ [ppm]: 1.024 (6.26), 1.030 (9.42), 1.037 (8.35), 1.042 (8.42), 1.049 (4.77), 1.071 (1.48), 1.083 (3.87), 1.090 (7.52), 1.097 (5.35), 1.103 (5.71), 1.110 (8.52), 1.117 (5.55), 1.128 (2.45), 1.160 (1.39), 1.185 (2.45), 1.208 (2.87), 1.231 (2.77), 1.453 (0.87), 1.479 (2.29), 1.504 (2.35), 1.530 (0.94), 1.789 (1.87), 1.818 (4.87), 1.843 (5.35), 1.866 (2.03), 1.988 (0.45), 2.053 (3.68), 2.076 (5.84), 2.099 (2.97), 2.294 (1.10), 2.306 (2.29), 2.314 (2.77), 2.326 (5.61), 2.331 (3.42), 2.337 (3.45), 2.345 (2.32), 2.357 (1.03), 2.665 (1.32), 2.669 (1.94), 2.673 (1.55), 4.174 (16.00), 4.710 (6.35), 4.721 (6.42), 5.185 (9.87), 5.759 (4.39), 7.045 (3.00), 7.099 (6.94), 7.113 (6.61), 7.423 (2.48), 7.512 (2.55), 8.381 (15.87), 12.119 (2.48).

### EXAMPLE 73

### 1-[(3-{[(1H-benzimidazol-2-yl)methyl]amino}-6-cyclopropyl-1H-pyrazolo[3,4-b]pyrazin-1-yl)methyl]cyclopentan-1-ol

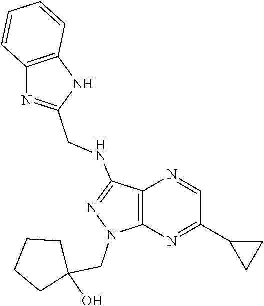 [SMILES: OC1(Cn2nc(NCc3nc4ccccc4[nH]3)c3ncc(C4CC4)nc32)CCCC1]

In analogy to example 3) 1-[(3-amino-6-cyclopropyl-1H-pyrazolo[3,4-b]pyrazin-1-yl)methyl]cyclopentan-1-ol (95.0 mg, 348 μmol, intermediate 105) and 1H-benzimidazole-2-carbaldehyde (102 mg, 695 μmol) reacted and give after purification using a Biotage chromatography system followed by a HPLC purifcation 8.90 mg (95% purity, 6% yield) of the desired title compound.

LC-MS (Method 2): Rₜ=1.12 min; MS (ESIpos): m/z=404 [M+H]⁺

¹H-NMR (400 MHz, DMSO-d6) δ [ppm]: 1.013 (1.41), 1.024 (4.14), 1.031 (6.15), 1.038 (4.84), 1.043 (5.33), 1.050 (2.99), 1.072 (0.87), 1.083 (2.50), 1.090 (5.17), 1.097 (3.21), 1.102 (3.54), 1.110 (5.66), 1.117 (3.43), 1.128 (1.47), 1.154 (0.44), 1.172 (0.65), 1.190 (0.49), 1.257 (3.21), 1.265 (3.27), 1.294 (1.25), 1.312 (1.69), 1.346 (2.88), 1.452 (1.47), 1.466 (2.83), 1.480 (2.83), 1.506 (5.12), 1.532 (2.29), 1.544 (1.41), 1.987 (0.93), 2.290 (0.76), 2.302 (1.58), 2.310 (1.80), 2.322 (5.39), 2.326 (4.19), 2.332 (3.70), 2.342 (1.52), 2.354 (0.65), 2.518 (16.00), 2.522 (10.78), 2.660 (1.09), 2.664 (2.50), 2.668 (3.43), 2.673 (2.39), 2.678 (1.09), 4.155 (14.42), 4.529 (11.21), 4.568 (0.44), 4.695 (5.39), 4.710 (5.28), 5.759 (6.59), 7.025 (1.41), 7.039 (2.72), 7.053 (1.41), 7.088 (4.46), 7.095 (3.81), 7.105 (4.68), 7.110 (3.43), 7.378 (1.69), 7.392 (1.58), 7.502 (1.69), 8.376 (15.95), 8.763 (0.54), 12.110 (2.45).

### EXAMPLE 74

### {1-[(3-{[(1H-benzimidazol-2-yl)methyl]amino}-6-cyclopropyl-1H-pyrazolo[3,4-b]pyrazin-1-yl)methyl]cyclobutyl}methanol

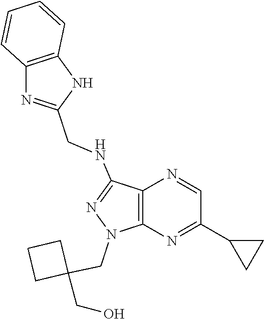 [SMILES: OCC1(Cn2nc(NCc3nc4ccccc4[nH]3)c3ncc(C4CC4)nc32)CCC1]

In analogy to example 3) {1-[(3-amino-6-cyclopropyl-1H-pyrazolo[3,4-b]pyrazin-1-yl)methyl]cyclobutyl}methanol (110 mg, 181 μmol, intermediate 107) and 1H-benzimidazole-2-carbaldehyde (52.9 mg, 362 μmol) reacted and give after purification using a Biotage chromatography system followed by a HPLC purifcation 10.9 mg (90% purity, 13% yield) of the desired title compound.

LC-MS (Method 1): Rₜ=0.92 min; MS (ESIpos): m/z=404 [M+H]⁺

¹H-NMR (400 MHz, DMSO-d6) δ [ppm]: 1.013 (1.10), 1.024 (3.14), 1.031 (4.60), 1.038 (3.56), 1.043 (3.87), 1.050 (2.20), 1.071 (0.68), 1.083 (1.93), 1.091 (3.71), 1.097 (2.35), 1.104 (2.56), 1.111 (4.08), 1.118 (2.51), 1.129 (1.20), 1.233 (0.94), 1.491 (0.58), 1.514 (1.25), 1.535 (0.73), 1.555 (0.58), 1.597 (1.05), 1.602 (1.05), 1.620 (4.29), 1.625 (3.61), 1.643 (2.72), 1.659 (0.78), 1.749 (0.73), 1.772 (2.04), 1.786 (2.46), 1.804 (1.41), 1.809 (1.41), 1.820 (0.89), 2.295 (0.52), 2.307 (1.15), 2.318 (1.83), 2.323 (3.14), 2.327 (5.49), 2.332 (2.93), 2.337 (2.04), 2.347 (1.10), 2.359 (0.47), 2.518 (16.00), 2.523 (10.82), 2.660 (1.05), 2.665 (2.35), 2.669 (3.29), 2.674 (2.30), 2.679 (1.05), 3.248 (5.86), 3.262 (5.91), 3.365 (0.47), 4.140 (11.61), 4.475 (0.47), 4.606 (1.67), 4.620 (3.82), 4.634 (1.62), 4.712 (5.80), 4.727 (5.65), 5.760 (3.19), 6.959 (1.36), 6.974 (2.93), 6.989 (1.31), 7.090 (3.45), 7.097 (3.56), 7.105 (3.71), 7.112 (3.87), 7.122 (0.89), 7.407 (0.78), 7.503 (0.78), 8.384 (15.74), 8.785 (0.63), 9.796 (0.58), 12.135 (1.62).

### EXAMPLE 75

### N-[(1H-benzimidazol-2-yl)methyl]-1-(cyclobutylmethyl)-6-cyclopropyl-1H-pyrazolo[3,4-b]pyrazin-3-amine

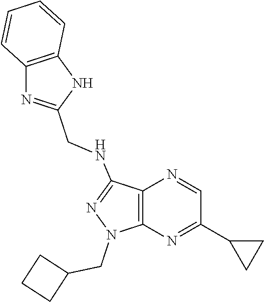 [SMILES: c1ccc2[nH]c(CNc3nn(CC4CCC4)c4nc(C5CC5)cnc34)nc2c1]

In analogy to example 3) 1-(cyclobutylmethyl)-6-cyclopropyl-1H-pyrazolo[3,4-b]pyrazin-3-amine (150 mg, 388 μmol, intermediate 109) and 1H-benzimidazole-2-carbaldehyde (114 mg, 777 μmol) reacted and give after purification using a Biotage chromatography system followed by a HPLC purifcation 22.9 mg (95% purity, 15% yield) of the desired title compound.

LC-MS (Method 2): Rₜ=1.27 min; MS (ESIpos): m/z=374 [M+H]⁺

¹H-NMR (400 MHz, DMSO-d6) δ [ppm]: 1.021 (1.79), 1.032 (5.32), 1.038 (7.95), 1.045 (6.27), 1.050 (7.17), 1.057 (3.50), 1.071 (0.98), 1.078 (1.28), 1.085 (3.23), 1.092 (7.24), 1.099 (4.04), 1.105 (4.28), 1.112 (7.48), 1.119 (4.31), 1.131 (1.89), 1.231 (0.77), 1.652 (1.58), 1.670 (3.10), 1.687 (7.24), 1.700 (9.97), 1.712 (3.91), 1.718 (3.20), 1.730 (1.99), 1.741 (1.68), 1.755 (1.01), 1.771 (0.77), 1.782 (1.11), 1.791 (2.53), 1.798 (2.12), 1.812 (3.47), 1.816 (3.33), 1.827 (4.11), 1.844 (1.35), 2.292 (1.01), 2.304 (2.05), 2.312 (2.32), 2.323 (4.72), 2.332 (2.96), 2.335 (2.73), 2.344 (1.99), 2.356 (0.84), 2.518 (9.30), 2.522 (6.23), 2.628 (0.71), 2.647 (1.68), 2.664 (3.60), 2.668 (3.50), 2.673 (2.26), 2.678 (1.62), 2.683 (1.82), 2.701 (0.71), 4.088 (10.58), 4.105 (10.31), 4.718 (5.59), 4.733 (5.49), 5.759 (1.15), 6.932 (2.69), 6.946 (1.48), 7.093 (4.85), 7.100 (5.25), 7.109 (5.32), 7.115 (5.25), 7.406 (1.38), 7.511 (1.38), 8.369 (16.00), 12.157 (2.22).

### EXAMPLE 76

### 4-(3-{[(1H-benzimidazol-2-yl)methyl]amino}-6-cyclopropyl-1H-pyrazolo[3,4-b]pyrazin-1-yl)-2-methylbutan-2-ol

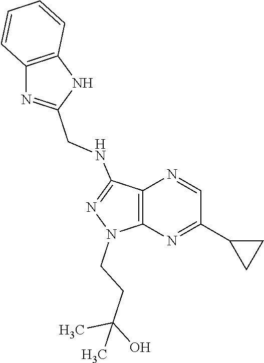 [SMILES: CC(C)(O)CCn1nc(NCc2nc3ccccc3[nH]2)c2ncc(C3CC3)nc21]

In analogy to example 40) 4-(3-amino-6-cyclopropyl-1H-pyrazolo[3,4-b]pyrazin-1-yl)-2-methylbutan-2-ol (200 mg, 765 μmol, intermediate 110) and 1H-benzimidazole-2-carbaldehyde (224 mg, 1.53 mmol) reacted and give after purification using a Biotage chromatography system followed by a HPLC purifcation 36.0 mg (97% purity, 12% yield) of the desired title compound.

LC-MS (Method 2): Rₜ=1.01 min; MS (ESIneg): m/z=390 [M−H]⁻

¹H-NMR (400 MHz, DMSO-d6) δ [ppm]: 1.041 (0.92), 1.047 (1.44), 1.058 (16.00), 1.086 (0.63), 1.093 (1.04), 1.099 (0.76), 1.105 (0.64), 1.112 (1.16), 1.119 (0.65), 1.758 (0.80), 1.773 (0.69), 1.778 (0.88), 1.783 (0.70), 1.797 (0.83), 2.322 (0.65), 2.518 (0.51), 4.155 (0.80), 4.169 (0.67), 4.175 (0.85), 4.180 (0.69), 4.194 (0.78), 4.355 (4.01), 4.737 (1.84), 4.753 (1.84), 5.759 (1.57), 6.908 (0.45), 6.923 (1.01), 6.938 (0.42), 7.098 (0.85), 7.105 (0.91), 7.114 (0.95), 7.121 (0.93), 8.356 (4.18), 12.158 (0.72).

### EXAMPLE 77

### N-[(1H-benzimidazol-2-yl)methyl]-6-methyl-1-(1-methylpiperidin-4-yl)-1H-pyrazolo[3,4-b]pyrazin-3-amine

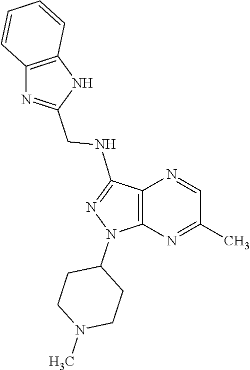 [SMILES: Cc1cnc2c(NCc3nc4ccccc4[nH]3)nn(C3CCN(C)CC3)c2n1]

In analogy to example 3) 6-methyl-1-(1-methylpiperidin-4-yl)-1H-pyrazolo[3,4-b]pyrazin-3-amine (65.0 mg, 264 μmol, intermediate 112) and 1H-benzimidazole-2-carbaldehyde (77.1 mg, 528 μmol) reacted and give after purification using a Biotage chromatography system followed by a HPLC purifcation 8.00 mg (93% purity, 7% yield) of the desired title compound.

LC-MS (Method 2): Rₜ=0.96 min; MS (ESIpos): m/z=377 [M+H]⁺

¹H-NMR (400 MHz, DMSO-d6) δ [ppm]: 1.052 (0.51), 1.172 (0.63), 1.231 (1.86), 1.705 (1.12), 1.729 (1.23), 1.751 (0.84), 1.890 (1.39), 1.995 (0.72), 2.021 (1.66), 2.050 (1.42), 2.087 (0.60), 2.094 (0.63), 2.116 (1.12), 2.125 (1.18), 2.146 (1.00), 2.155 (1.13), 2.177 (12.43), 2.518 (3.04), 2.523 (1.98), 2.540 (5.02), 2.588 (16.00), 2.817 (1.46), 2.844 (1.37), 3.165 (2.30), 4.398 (0.45), 4.416 (0.52), 4.426 (0.86), 4.437 (0.49), 4.455 (0.44), 4.764 (3.82), 4.779 (3.83), 6.926 (0.87), 6.941 (1.94), 6.956 (0.84), 7.100 (1.46), 7.105 (1.87), 7.122 (2.08), 7.127 (1.51), 7.403 (0.70), 7.418 (0.68), 7.524 (0.72), 7.539 (0.66), 8.300 (7.01), 12.205 (0.88).

### EXAMPLE 78

### (+) or (−)-N-[(1H-benzimidazol-2-yl)methyl]-6-cyclopropyl-1-(1-methylpyrrolidin-3-yl)-1H-pyrazolo[3,4-b]pyrazin-3-amine

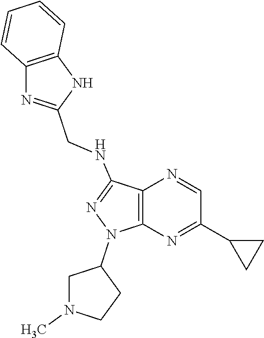 [SMILES: CN1CCC(n2nc(NCc3nc4ccccc4[nH]3)c3ncc(C4CC4)nc32)C1]

(±)-N-[(1H-benzimidazol-2-yl)methyl]-6-cyclopropyl-1-(1-methylpyrrolidin-3-yl)-1H-pyrazolo[3,4-b]pyrazin-3-amine (45.0 mg, 116 μmol, example 35) was separated in the two enantiomers using a chiral HPLC (column : Chiralpak IG-30 1909 F-11) and again purified using a Biotage chromatography system to give 4.90 mg (80% purity, 9% yield) of the desired compound.

### EXAMPLE 79

### (−) or (+)-N-[(1H-benzimidazol-2-yl)methyl]-6-cyclopropyl-1-(1-methylpyrrolidin-3-yl)-1H-pyrazolo[3,4-b]pyrazin-3-amine

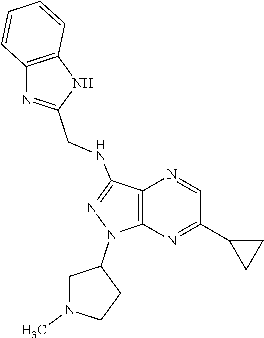 [SMILES: CN1CCC(n2nc(NCc3nc4ccccc4[nH]3)c3ncc(C4CC4)nc32)C1]

(±)-N-[(1H-benzimidazol-2-yl)methyl]-6-cyclopropyl-1-(1-methylpyrrolidin-3-yl)-1H-pyrazolo[3,4-b]pyrazin-3-amine (45.0 mg, 116 μmol, example 35) was separated in the two enantiomers using a chiral HPLC (column: Chiralpak IG-30 1909 F-11) and again purified using a Biotage chromatography system to give 3.20 mg (80% purity, 6% yield) of the desired compound.

### REFERENCE EXAMPLE 1

### N-[(1H-benzimidazol-2-yl)methyl]-8-bromo-2-(morpholin-4-yl)pyrazolo[1,5-a][1,3,5]triazin-4-amine

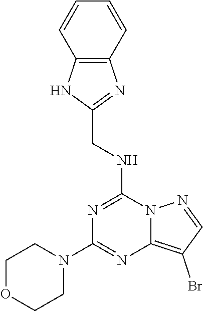 [SMILES: Brc1cnn2c(NCc3nc4ccccc4[nH]3)nc(N3CCOCC3)nc12]

Step 1:

1H-pyrazol-5-amine (58.7 g, 706 mmol; CAS 1820-80-0) was dissolved in ethyl acetate (420 mL), under nitrogen, and stirred at 75° C. Ethyl carbonisothiocyanatidate (88 mL, 750 mmol; CAS 16182-04-0) was added dropwise at 75° C. and the mixture was stirred for 1 h at 75° C. The mixture was cooled to 0° C., filtered, washed with ethyl acetate and the solid was dried under reduced pressure at 50° C. to give 124 g (77% yield) of ethyl [(1H-pyrazol-5-yl)carbamothioyl]carbamate.

Step 2:

Ethyl [(1H-pyrazol-5-yl)carbamothioyl]carbamate (124 g, 580 mmol) was stirred in sodium hydroxide (550 mL, 2.0 M, 1.1 mol) for 3 h at rt. The mixture was cooled to 0° C. and sulfuric acid (580 mL, 2.0 M, 1.2 mol) was added dropwise. The suspension was filtered, washed with water and the solid was dried under reduced pressure at 50° C. to give 85.2 g (87% yield) of 2-sulfanylpyrazolo[1,5-a][1,3,5]triazin-4-ol.

Step 3:

2-sulfanylpyrazolo[1,5-a][1,3,5]triazin-4-ol (85.2 g, 507 mmol) was dissolved in ethanol (2.0 I) and sodium hydroxide (580 mL, 1.7 M, 1.0 mol). lodomethane (32 mL, 510 mmol; CAS 74-88-4) was added dropwise at rt and the mixture was stirred for 2 h at rt. The mixture was cooled to 0° C., sulfuric acid (510 mL, 1.0 M, 510 mmol) was added dropwise and the mixture was stirred for 1 h at rt. The precipitate was collected by filtration, washed with water dried under reduced pressure at 50° C. The solid was stirred 2 times in acetonitrile, liquid phases were filtered off and the soid was washed with hexane and dried to give 60.5 g (65% yield) of 2-(methylsulfanyl)pyrazolo[1,5-a][1,3,5]triazin-4-ol.

Step 4:

2-(methylsulfanyl)pyrazolo[1,5-a][1,3,5]triazin-4-ol (59.0 g, 324 mmol) was dissolved in DMF (690 mL), cooled to 0° C., NBS (63.4 g, 356 mmol; CAS 128-08-5) dissolved in DMF (200 mL) was added dropwise and the mixture was stirred for 1 h at 0° C. The mixture was poured into water, stirred for 15 min, filtered and washed with water, acetonitrile and hexane. The solid was dried under reduced pressure at 50° C. to give 71.7 g (85% yield) of 8-bromo-2-(methylsulfanyl)pyrazolo[1,5-a][1,3,5]triazin-4-ol.

Step 5:

8-bromo-2-(methylsulfanyl)pyrazolo[1,5-a][1,3,5]triazin-4-ol (33.3 g, 128 mmol) was dissolved in phosphorus oxychloride (170 mL, 1.8 mol; CAS 10025-87-3) and N,N-dimethylaniline (16 mL, 130 mmol; CAS 121-69-7) was added. The mixture was stirred for 3 h at 105° C. The mixture was poured carefully into ice water and neutralized with sodium bicarbonate. The suspension was filtered and washed with water and hexane to give 24.0 g (67% yield) of 8-bromo-4-chloro-2-(methylsulfanyl)pyrazolo[1,5-a][1,3,5]triazine.

Step 6:

8-bromo-4-chloro-2-(methylsulfanyl)pyrazolo[1,5-a][1,3,5]triazine (977 mg, 3.49 mmol) and 1-(1H-benzimidazol-2-yl)methanamine dihydrochloride (1.15 g, 5.24 mmol, CAS 5993-91-9) were dissolved in acetonitrile (11 mL), N,N-diisopropylethylamine (2.9 mL, 17 mmol) was added and the mixture was stirred overnight at 50° C. The mixture was evaporated, diluted with a mixture of dichloromethane and 2-propanol (4:1), washed with sodium hydroxide (2M) and brine. The organic layer was filtered. The solid was dried under reduced pressure to give 192 mg (95% purity, 13% yield) of N-[(1H-benzimidazol-2-yl)methyl]-8-bromo-2-(methylsulfanyl)pyrazolo[1,5-a][1,3,5]triazin-4-amine.

Step 7:

N-[(1H-benzi midazol-2-yl)methyl]-8-bromo-2-(methylsulfanyl)pyrazolo[1,5-a][1,3,5]triazin-4-amine (650 mg, 1.67 mmol) was dissolved in dichloromethane (13 mL), cooled to 0° C., mCPBA (1.23 g, 70% purity, 5.00 mmol) was added and the mixture was stirred for 2 h at rt. The mixture was diluted with dichloromethane and washed with sat. sodium bicarbonate solution. The aqueous layer was extracted 2 times with a mixture of dichloromethane and 2-propanol and the combined organic layers were washed with water, dried and concentrated under reduced pressure to give 780 mg N-[(1H-benzimidazol-2-yl)methyl]-8-bromo-2-(methanesulfonyl)pyrazolo[1,5-a][1,3,5]triazin amine.

Step 8:

N-[(1H-benzimidazol-2-yl)methyl]-8-bromo-2-(methanesulfonyl)pyrazolo[1,5-a][1,3,5]triazin-4-amine (143 mg, 339 μmol) and morpholine (89 μL, 1.0 mmol; CAS 110-91-8) were dissolved in acetonitrile (3.2 mL), N,N-diisopropylethylamine (180 μL, 1.0 mmol) was added and the mixture was stirred overnight at 70° C. The mixture was purified by preparative HPLC (HT basic) to give 91.0 mg (62% yield) of the title compound.

LC-MS (Method 2): Rₜ=1.01 min; MS (ESIneg): m/z=427 [M−H]⁻

¹H-NMR (400 MHz, DMSO-d6) δ [ppm]: 2.336 (0.65), 2.518 (8.31), 2.522 (5.70), 2.539 (6.28), 2.678 (0.65), 3.305 (0.44), 3.490 (2.94), 3.620 (4.46), 4.867 (3.30), 4.876 (3.27), 7.100 (0.47), 7.114 (1.92), 7.118 (1.49), 7.124 (2.32), 7.131 (2.54), 7.136 (2.58), 7.143 (1.56), 7.147 (2.10), 7.162 (0.51), 7.393 (1.56), 7.410 (1.38), 7.527 (1.45), 7.544 (1.38), 8.037 (16.00), 9.138 (1.12), 12.253 (1.92).

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

An empty field in any of the following tables means that the respective compound has not been tested in that Assay.

1. Expression and Purification of the CDK12/CycK and CDK13/CycK used in the CDK12 and CDK13 Kinase Activity Assays

1.1 Cloning of CDK 12/13, CycK and CAK1 in Insect Destination Vectors

The cDNAs encoding the following protein sequences were codon optimized for expression in Sf9/Hi-5 insect cells and synthesized by the GeneArt Technology at Thermo Fischer Scientific.

The human CDK12 wt/DN (Acc. Q9NYV4), CDK13 (Q14004), CycK (075909) and the saccharomyces cerevisiae CAK1 (P43568) full length sequence were used for cloning. These cDNAs also encoded att-site sequences at the 5′and 3′ ends for subcloning into the following destination vectors using the Gateway Technology.

By using of baculovirus vectors with a strong polyhedrin promoter provides an N-terminal fusion of a His-tag with a Tobacco Edge virus cleavage site to the integrated gene of interest. Only the saccharomyces cerevisiae CAK1 (P43568) full length sequence was cloned in an insect vector which provides a tag-free gene of interest.

1.2 Sequence
His-CDK12 (aa Q696-S1082)
MTSHHHHHHS SMGSRTSLYK KAGSDYDIPT TENLYFQGQP
YKKRPKICCP RYGERRQTES DWGKRCVDKF DIIGIIGEGT
YGQVYKAKDK DTGELVALKK VRLDNEKEGF PITAIREIKI
LRQLIHRSVV NMKEIVTDKQ DALDFKKDKG AFYLVFEYMD
HDLMGLLESG LVHFSEDHIK SFMKQLMEGL EYCHKKNFLH
RDIKCSNILL NNSGQIKLAD FGLARLYNSE ESRPYTNKVI
TLWYRPPELL LGEERYTPAI DVWSCGCILG ELFTKKPIFQ
ANLELAQLEL ISRLCGSPCP AVWPDVIKLP YFNTMKPKKQ
YRRRLREEFS FIPSAALDLL DHMLTLDPSK RCTAEQTLQS
DFLKDVELSK MAPPDLPHWQ DCHELWSKKR RRQRQSGVVV
EEPPPSKTSR KETTSGTSTE PVKNS
His-CDK12-DN (aa Q696-S1082; K756A; D877N)
MTSHHHHHHS SMGSRTSLYK KAGSDYDIPT TENLYFQGQP
YKKRPKICCP RYGERRQTES DWGKRCVDKF DIIGIIGEGT
YGQVYKAKDK DTGELVALAK VRLDNEKEGF PITAIREIKI
LRQLIHRSVV NMKEIVTDKQ DALDFKKDKG AFYLVFEYMD
HDLMGLLESG LVHFSEDHIK SFMKQLMEGL EYCHKKNFLH
RDIKCSNILL NNSGQIKLAN FGLARLYNSE ESRPYTNKVI
TLWYRPPELL LGEERYTPAI DVWSCGCILG ELFTKKPIFQ
ANLELAQLEL ISRLCGSPCP AVWPDVIKLP YFNTMKPKKQ
YRRRLREEFS FIPSAALDLL DHMLTLDPSK RCTAEQTLQS
DFLKDVELSK MAPPDLPHWQ DCHELWSKKR RRQRQSGVVV
EEPPPSKTSR KETTSGTSTE PVKNS
His-CDK13 (aa Q673-P1059)
MTSHHHHHHS SMGSRTSLYK KAGSDYDIPT TENLYFQGQL
HSKRRPKICG PRYGETKEKD IDWGKRCVDK FDIIGIIGEG
TYGQVYKARD KDTGEMVALK KVRLDNEKEG FPITAIREIK
ILRQLTHQSI INMKEIVTDK EDALDFKKDK GAFYLVFEYM
DHDLMGLLES GLVHFNENHI KSFMRQLMEG LDYCHKKNFL
HRDIKCSNIL LNNRGQIKLA DFGLARLYSS EESRPYTNKV
ITLWYRPPEL LLGEERYTPA IDVWSCGCIL GELFTKKPIF
QANQELAQLE LISRICGSPC PAVWPDVIKL PYFNTMKPKK
QYRRKLREEF VFIPAAALDL FDYMLALDPS KRCTAEQALQ
CEFLRDVEPS KMPPPDLPLW QDCHELWSKK RRRQKQMGMT
DDVSTIKAPR KDLSLGLDDS RTNTP
His-CDK13-DN (aa Q673-P1059; K734A; D855N)
MTSHHHHHHS SMGSRTSLYK KAGSDYDIPT TENLYFQGQL
HSKRRPKICG PRYGETKEKD IDWGKRCVDK FDIIGIIGEG
TYGQVYKARD KDTGEMVALA KVRLDNEKEG FPITAIREIK
ILRQLTHQSI INMKEIVTDK EDALDFKKDK GAFYLVFEYM
DHDLMGLLES GLVHFNENHI KSFMRQLMEG LDYCHKKNFL
HRDIKCSNIL LNNRGQIKLA NFGLARLYSS EESRPYTNKV
ITLWYRPPEL LLGEERYTPA IDVWSCGCIL GELFTKKPIF
QANQELAQLE LISRICGSPC PAVWPDVIKL PYFNTMKPKK
QYRRKLREEF VFIPAAALDL FDYMLALDPS KRCTAEQALQ
CEFLRDVEPS KMPPPDLPLW QDCHELWSKK RRRQKQMGMT
DDVSTIKAPR KDLSLGLDDS RTNTP
His-CycK (aa M1-S300)
MTSHHHHHHS SMGSRTSLYK KAGSDYDIPT TENLYFQGMK
ENKENSSPSV TSANLDHTKP CWYWDKKDLA HTPSQLEGLD
PATEARYRRE GARFIFDVGT RLGLHYDTLA TGIIYFHRFY
MFHSFKQFPR YVTGACCLFL AGKVEETPKK CKDIIKTARS
LLNDVQFGQF GDDPKEEVMV LERILLQTIK FDLQVEHPYQ
FLLKYAKQLK GDKNKIQKLV QMAWTFVNDS LCTTLSLQWE
PEIIAVAVMY LAGRLCKFEI QEWTSKPMYR RWWEQFVQDV
PVDVLEDICH QILDLYSQGK QQMPHHTPHQ LQQPPSLQPT
PQVPQVQQSQ PSQSSEPS
CAK1 (aa M1-P368)
MKLDSIDITH CQLVKSTRTA RIYRSDTYAI KCLALDFDIP
PHNAKFEVSI LNKLGNKCKH ILPLLESKAT DNNDLLLLFP
FEEMNLYEFM QMHYKRDRRK KNPYYDLLNP SIPIVADPPV
QKYTNQLDVN RYSLSFFRQM VEGIAFLHEN KIIHRDIKPQ
NIMLTNNTST VSPKLYIIDF GISYDMANNS QTSAEPMDSK
VTDISTGIYK APEVLFGVKC YDGGVDVWSL LIIISQWFQR
ETSRMGHVPA MIDDGSDDMN SDGSDFRLIC SIFEKLGIPS
IQKWEEVAQH GSVDAFVGMF GADGDGKYVL DQEKDVQISI
VERNMPRLDE IADVKVKQKF INCILGMVSF SPNERWSCQR
ILQELEKP

1.3 Expression of the CDK12-CycK and CDK13-CycK Complex

The Hi-5 insect cells were cultivated in Insect Xpress Medium (Lonza # BE12-730Q) and for co-infection the following baculovirus with multiplicity of infection (MOI) was using for the expression of the complex: CDK12 and CDK13 with MOI 1.0; CycK and CAK1 with MOI 0.5.

The complex formation was performed by co-infection of Hi-5 cells grown in suspension to a density of 2×10⁶ cells/mL in 8 L waver for 72 h. The cells were harvested by centrifugation (10 min., 170 g, 4° C.) and the cell pellets stored at −80° C.

1.4 Purification of the CDK12 and CDK13 Complex

Purification of the His-CDK12/His-CycK/CAK1 or His-CDK13/His-CycK/CAK1 complex was achieved by affinity chromatography using Ni-Sepharose High Performance (GE Healthcare #17-5268-02) or HisTrapTMHP (GE Healthcare #17-5247-01/05)

Cell pellets were resuspended in lysis buffer (50 millimol/L Hepes pH 7.5, 500 millimol/L NaCl, 40 millimol/L Imidazol, 10% Glycerol; 0.5% NP40, Benzonase (150 U/10 g cell pellet), 1 millimol/L DTT and 1× Complete EDTA-free protease inhibitor cocktail (Roche #1873580)).

The lysate was incubated on ice for 30 minutes and clarified by centrifugation (1 h, 4° C., 27500×g). Proteins were captured overnight at 4° C. using Ni-Sepharose or HisTrap HP material, washed with CDK12/13 wash buffer (50 millimol/L Hepes pH 7.5, 500 millimol/L NaCl, 40 millimol/L imidazole, 10% Glycerol, 1 millimol/L DTT) and eluted with wash buffer by using gradient of imidazole (40-500 millimol/L).

For removal of imidazole the eluted protein complexes were desalted with ZebaTM Desalt Spin Columns (Pierce #89893) against CDK12/13 DS buffer (50 millimol/L Hepes pH 7.5, 500 millimol/L NaCl, 10% Glycerol, 1 millimol/L DTT).

The final concentration was calculated densitrometrically using BSA as a standard in a Coomassie stained gel. Elution fractions were aliquoted and shock frozen using liquid nitrogen.

The in vitro activity of the compounds of the present invention can be demonstrated in the following assays:

2. Biochemical Kinase Assays

2.1 CDK12/CycK low ATP Kinase Assay

CDK12/CycK-inhibitory activity of compounds of the present invention at 10 micromol/L adenosine-tri-phosphate (ATP) was quantified employing the TR-FRET (TR-FRET =Time Resolved Fluorescence Energy Transfer) based CDK12/CycK activity inhibition assay as described in the following paragraphs.

A complex of human recombinant CDK12 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK12/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (16.7 micromol/L=>final conc. in the 5 microL assay volume is 10 micromol/L) and substrate (1.67 micromol/L=>final conc. in the 5 microL assay volume is 1 micromol/L) in assay buffer and the resulting mixture was incubated for a reaction time of 60 min at 22° C. The concentration of CDK12/CycK was adjusted depending of the activity of the enzyme lot and was chosen appropriate to have the assay in the linear range, typical concentrations were about 2 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata ScreenerTM software.

2.2 CDK12/CycK high ATP Kinase Assay

In the context of the present invention, the term “IC50 CDK12 hATP” refers to the ICso values obtained according to the assay described in this section (2.2) herein below, i.e. the IC50 values for the inhibition of CDK12 at high ATP.

CDK12/CycK -inhibitory activity of compounds of the present invention at 2 millimol/L adenosine-tri-phosphate (ATP) was quantified employing the TR-FRET (TR-FRET=Time Resolved Fluorescence Energy Transfer) based CDK12/CycK activity inhibition assay as described in the following paragraphs.

A complex of human recombinant CDK12 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK12/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (3.33 millimol/L=>final conc. in the 5 microL assay volume is 2 millimol/L) and substrate (1.67 micromol/L=>final conc. in the 5 microL assay volume is 1 micromol/L) in assay buffer and the resulting mixture was incubated for a reaction time of 60 min at 22° C. The concentration of CDK12/CycK was adjusted depending of the activity of the enzyme lot and was chosen appropriate to have the assay in the linear range, typical concentrations were about 0.75 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

TABLE 1
CDK12/CyclinK, 2 mM ATP (high ATP), IC₅₀ - [mol/l] (median)

| | CDK12/CyclinK, 2 mM |
|---|---|
| Example | ATP (high ATP), |
| No | IC50 - [mol/l] (median) |
| 1 | >2.00E−5 |
| 2 | >2.00E−5 |
| 3 | ≥1.16E−5 |
| 4 | >2.00E−5 |
| 5 | >2.00E−5 |
| 6 | >2.00E−5 |
| 7 | >2.00E−5 |
| 8 | >2.00E−5 |
| 9 | >2.00E−5 |
| 10 | >2.00E−5 |
| 11 | >2.00E−5 |
| 12 | >2.00E−5 |
| 13 | >2.00E−5 |
| 14 | >2.00E−5 |
| 15 | >2.00E−5 |
| 16 | >2.00E−5 |
| 17 | >2.00E−5 |
| 18 | >2.00E−5 |
| 19 | >2.00E−5 |
| 20 | >2.00E−5 |
| 21 | >2.00E−5 |
| 22 | >2.00E−5 |
| 23 | 1.23E−5 |
| 24 | 1.82E−5 |
| 25 | 1.73E−5 |
| 26 | >2.00E−5 |
| 27 | 2.02E−6 |
| 28 | >2.00E−5 |
| 29 | >2.00E−5 |
| 30 | >2.00E−5 |
| 31 | >2.00E−5 |
| 32 | 1.38E−5 |
| 33 | >2.00E−5 |
| 34 | >2.00E−5 |
| 35 | >2.00E−5 |
| 36 | >2.00E−5 |
| 37 | >2.00E−5 |
| 38 | >2.00E−5 |
| 39 | >2.00E−5 |
| 40 | >2.00E−5 |
| 41 | >2.00E−5 |
| 42 | >2.00E−5 |
| 43 | |
| 44 | |
| 45 | |
| 46 | >2.00E−5 |
| 47 | 1.28E−5 |
| 48 | 1.37E−5 |
| 49 | >2.00E−5 |
| 50 | 6.17E−6 |
| 51 | >2.00E−5 |
| 52 | >2.00E−5 |
| 53 | >1.00E−5 |
| 54 | 1.11E−05 |
| 55 | >2.00E−5 |
| 56 | >1.81E−5 |
| 57 | >2.00E−5 |
| 58 | >2.00E−5 |
| 59 | >2.00E−5 |
| 60 | >2.00E−5 |
| 61 | >2.00E−5 |
| 62 | >2.00E−5 |
| 63 | >2.00E−5 |
| 64 | >2.00E−5 |
| 65 | >2.00E−5 |
| 66 | >2.00E−5 |
| 67 | >2.00E−5 |
| 68 | >2.00E−5 |
| 69 | >1.00E−5 |
| 70 | >2.00E−5 |
| 71 | >2.00E−5 |
| 72 | >1.00E−5 |
| 73 | >1.00E−5 |
| 74 | >1.00E−5 |
| 75 | >2.00E−5 |
| 76 | >2.00E−5 |
| 77 | >2.00E−5 |
| 78 | >2.00E−5 |
| 79 | >2.00E−5 |

2.3 CDK13/CycK Low ATP Kinase Assay

CDK13/CycK -inhibitory activity of compounds of the present invention at 10 micromol/L adenosine-tri-phosphate (ATP) was quantified employing the TR-FRET (TR-FRET=Time Resolved Fluorescence Energy Transfer) based CDK13/CycK activity inhibition assay as described in the following paragraphs.

A complex of human recombinant CDK13 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK13/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (16.7 micromol/L=>final conc. in the 5 microL assay volume is 10 micromol/L) and substrate (1.67 micromol/L=>final conc. in the 5 microL assay volume is 1 micromol/L) in assay buffer and the resulting mixture was incubated for a reaction time of 60 min at 22° C. The concentration of CDK13/CycK was adjusted depending of the activity of the enzyme lot and was chosen appropriate to have the assay in the linear range, typical concentrations were about 5 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata ScreenerTM software.

2.4 CDK2/CycE Kinase Assay

CDK2/CycE-inhibitory activity of compounds of the present invention was quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, were purchased from ProQinase GmbH (Freiburg, Germany). As substrate for the kinase reaction biotinylated peptide biotin-Ttds-YISPLKSPYKISEG (C-terminus in amid form) was used which can be purchased e.g. form the company JERINI peptide technologies (Berlin, Germany).

For the assay 50 nanoL of a 100 fold concentrated solution of the test compound in DMSO was pipetted into a black low volume 384 well microtiter plate or a black 1536 well microtiter plate (both Greiner Bio-One, Frickenhausen, Germany), 2 microL of a solution of CDK2/CycE in aqueous assay buffer [50 millimol/L Tris/HCl pH 8.0, 10 millimol/L MgCl₂, 1.0 millimol/L dithiothreitol, 0.1 millimol/L sodium ortho-vanadate, 0.01% (v/v) Nonidet-P40 (Sigma)] were added and the mixture was incubated for 15 min at 22° C. to allow pre-binding of the test compounds to the enzyme before the start of the kinase reaction. Then the kinase reaction was started by the addition of 3 microL of a solution of adenosine-tri-phosphate (ATP, 3.33 millimol/L=>final conc. in the 5 microL assay volume is 2 millimol/L) and substrate (1.25 micromol/L=>final conc. in the 5 microL assay volume is 0.75 micromol/L) in assay buffer and the resulting mixture was incubated for a reaction time of 25 min at 22° C. The concentration of CDK2/CycE was adjusted depending of the activity of the enzyme lot and was chosen appropriate to have the assay in the linear range, typical concentrations were in the range of 10 ng/mL. The reaction was stopped by the addition of 3 microL of a solution of TR-FRET detection reagents (0.333 micromol/L streptavidine-XL665 [Cisbio Bioassays, Codolet, France] and 1.67 nanomol/L anti-RB(pSer807/pSer811)-antibody from BD Pharmingen [#558389] and 2 nanomol/L LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (167 millimol/L EDTA, 0.2% (w/v) bovine serum albumin in 100 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC50 values were calculated using Genedata Screener™ software.

TABLE 2
CDK2/CyclinE, 2 mM ATP (high ATP), IC₅₀ - [mol/l]

| | CDK2/CyclinE, 2 mM ATP |
|---|---|
| Example | (high ATP) IC50 - [mol/l] |
| No | (median) |
| 1 | >2.00E−5 |
| 2 | >2.00E−5 |
| 3 | >2.00E−5 |
| 4 | >2.00E−5 |
| 5 | |
| 6 | >2.00E−5 |
| 7 | >2.00E−5 |
| 8 | >2.00E−5 |
| 9 | >2.00E−5 |
| 10 | >2.00E−5 |
| 11 | >2.00E−5 |
| 12 | >2.00E−5 |
| 13 | >2.00E−5 |
| 14 | >2.00E−5 |
| 15 | >2.00E−5 |
| 16 | >2.00E−5 |
| 17 | >2.00E−5 |
| 18 | >2.00E−5 |
| 19 | >2.00E−5 |
| 20 | >2.00E−5 |
| 21 | >2.00E−5 |
| 22 | >2.00E−5 |
| 23 | 1.64E−5 |
| 24 | 1.79E−5 |
| 25 | >2.00E−5 |
| 26 | >2.00E−5 |
| 27 | >2.00E−5 |
| 28 | >2.00E−5 |
| 29 | >2.00E−5 |
| 30 | >2.00E−5 |
| 31 | >2.00E−5 |
| 32 | >2.00E−5 |
| 33 | >2.00E−5 |
| 34 | >2.00E−5 |
| 35 | >2.00E−5 |
| 36 | >2.00E−5 |
| 37 | >2.00E−5 |
| 38 | >2.00E−5 |
| 39 | >2.00E−5 |
| 40 | >2.00E−5 |
| 41 | >2.00E−5 |
| 42 | >2.00E−5 |
| 43 | >2.00E−5 |
| 44 | >2.00E−5 |
| 45 | >1.82E−5 |
| 46 | >2.00E−5 |
| 47 | >2.00E−5 |
| 48 | >2.00E−5 |
| 49 | >2.00E−5 |
| 50 | >2.00E−5 |
| 51 | >2.00E−5 |
| 52 | >2.00E−5 |
| 53 | >2.00E−5 |
| 54 | >2.00E−5 |
| 55 | >2.00E−5 |
| 56 | >2.00E−5 |
| 57 | >2.00E−5 |
| 58 | >2.00E−5 |
| 59 | >2.00E−5 |
| 60 | >2.00E−5 |
| 61 | >2.00E−5 |
| 62 | >2.00E−5 |
| 63 | >2.00E−5 |
| 64 | >2.00E−5 |
| 65 | >2.00E−5 |
| 66 | >2.00E−5 |
| 67 | >2.00E−5 |
| 68 | >2.00E−5 |
| 69 | >2.00E−5 |
| 70 | >2.00E−5 |
| 71 | >2.00E−5 |
| 72 | >2.00E−5 |
| 73 | >2.00E−5 |
| 74 | >2.00E−5 |
| 75 | >2.00E−5 |
| 76 | >2.00E−5 |
| 77 | >2.00E−5 |
| 78 | >2.00E−5 |
| 79 | >2.00E−5 |

2.5 CDK9/CycT1 High ATP Kinase Assay

CDK9/CycT1 -inhibitory activity of compounds of the present invention at a high ATP concentration after preincubation of enzyme and test compounds was quantified employing the CDK9/CycT1 TR-FRET assay as described in the following paragraphs.

Recombinant full-length His-tagged human CDK9 and CycT1, expressed in insect cells and purified by Ni-NTA affinity chromatography, were purchased from Life Technologies (Cat. No PV4131). As substrate for the kinase reaction biotinylated peptide biotin-Ttds-YISPLKSPYKISEG (C-terminus in amide form) was used which can be purchased e.g. form the company JERINI peptide technologies (Berlin, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK9/CycT1 in aqueous assay buffer [50 millimol/L Tris/HCl pH 8.0, 10 millimol/L MgCl₂, 1.0 millimol/L dithiothreitol, 0.1 millimol/L sodium ortho-vanadate, 0.01% (v/v) Nonidet-P40 (Sigma)] were added and the mixture was incubated for 15 min at 22° C. to allow pre-binding of the test compounds to the enzyme before the start of the kinase reaction. Then the kinase reaction was started by the addition of 3 microL of a solution of adenosine-tri-phosphate (ATP, 3.3 millimol/L=>final conc. in the 5 microL assay volume is 2 millimol/L) and substrate (1.25 micromol/L =>final conc. in the 5 microL assay volume is 0.75 micromol/L) in assay buffer and the resulting mixture was incubated for a reaction time of 25 min at 22° C. The concentration of CDK9/CycT1 was adjusted depending of the activity of the enzyme lot and was chosen appropriate to have the assay in the linear range, typical concentrations were in the range of 0.5 microg/mL. The reaction was stopped by the addition of 3 microL of a solution of TR-FRET detection reagents (0.33 micromol/L streptavidine-XL665 [Cisbio Bioassays, Codolet, France] and 1.67 nanomol/L anti-RB(pSer807/pSer811)-antibody from BD Pharmingen [#558389] and 2 nanomol/L LANCE EU-W1024 labeled anti-mouse IgG antibody [Perkin-Elmer, product no. AD0077]) in an aqueous EDTA-solution (167 millimol/L EDTA, 0.2% (w/v) bovine serum albumin in 100 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀-values were calculated using Genedata Screener™ software.

TABLE 3
CDK9/CyclinT1, 2 mM ATP (high ATP), IC₅₀ - [mol/l]

| | CDK9/CyclinT1, 2 mM |
|---|---|
| Example | ATP (high ATP) |
| No | IC50 - [mol/l] (median) |
| 1 | >2.00E−5 |
| 2 | >2.00E−5 |
| 3 | >2.00E−5 |
| 4 | >2.00E−5 |
| 5 | |
| 6 | ≥1.27E−5 |
| 7 | >2.00E−5 |
| 8 | >2.00E−5 |
| 9 | >2.00E−5 |
| 10 | >2.00E−5 |
| 11 | >2.00E−5 |
| 12 | >2.00E−5 |
| 13 | >2.00E−5 |
| 14 | >2.00E−5 |
| 15 | >2.00E−5 |
| 16 | >2.00E−5 |
| 17 | >2.00E−5 |
| 18 | >2.00E−5 |
| 19 | >2.00E−5 |
| 20 | >2.00E−5 |
| 21 | >2.00E−5 |
| 22 | >2.00E−5 |
| 23 | >2.00E−5 |
| 24 | >2.00E−5 |
| 25 | >2.00E−5 |
| 26 | >2.00E−5 |
| 27 | >2.00E−5 |
| 28 | >2.00E−5 |
| 29 | >2.00E−5 |
| 30 | >2.00E−5 |
| 31 | >2.00E−5 |
| 32 | >2.00E−5 |
| 33 | >2.00E−5 |
| 34 | >2.00E−5 |
| 35 | >2.00E−5 |
| 36 | >2.00E−5 |
| 37 | >2.00E−5 |
| 38 | >2.00E−5 |
| 39 | >2.00E−5 |
| 40 | >2.00E−5 |
| 41 | >2.00E−5 |
| 42 | >2.00E−5 |
| 43 | >2.00E−5 |
| 44 | >2.00E−5 |
| 45 | >2.00E−5 |
| 46 | >2.00E−5 |
| 47 | >2.00E−5 |
| 48 | >2.00E−5 |
| 49 | >2.00E−5 |
| 50 | >2.00E−5 |
| 51 | >2.00E−5 |
| 52 | >2.00E−5 |
| 53 | >2.00E−5 |
| 54 | >2.00E−5 |
| 55 | >2.00E−5 |
| 56 | >2.00E−5 |
| 57 | >2.00E−5 |
| 58 | >2.00E−5 |
| 59 | >2.00E−5 |
| 60 | >2.00E−5 |
| 61 | >2.00E−5 |
| 62 | >2.00E−5 |
| 63 | >2.00E−5 |
| 64 | >2.00E−5 |
| 65 | >2.00E−5 |
| 66 | >2.00E−5 |
| 67 | >2.00E−5 |
| 68 | >2.00E−5 |
| 69 | >2.00E−5 |
| 70 | >2.00E−5 |
| 71 | >2.00E−5 |
| 72 | >2.00E−5 |
| 73 | >2.00E−5 |
| 74 | >2.00E−5 |
| 75 | >2.00E−5 |
| 76 | >2.00E−5 |
| 77 | >2.00E−5 |
| 78 | >2.00E−5 |
| 79 | >2.00E−5 |

3. qRT-PCR Assay: BRCA1, ATR, MCL1 in MDA-MB-231, CAL-120

Tissue cultured human MDA-MB-231 human breast cancer cells were plated in 500 microL per well at 200,000 cells/well in a 24 well microtiter plate. After 24 h, the cells were exposed continuously for 24 h to test substances (substances were added with Tecan HP D300 Dispenser). RNA was prepared using Qiagen RNeasy MiniKit (#74106), RNA was quantified using a NanoDrop Equipment, and 600 nano gamms of RNA was coverted to cDNA using a SuperScript VILO kit (Thermofisher #11755050) followed by qPCR amplification. BRCA1 and ATR gene expression was measured by RT-qPCR and normalised to GAPDH housekeeping gene expression. qPCR primer sets have been purchased from Thermo Fisher Scientific/Applied Biosystems: BRCA1, # Hs 01556193; ATR, #Hs 00992123; GAPDH, #Hs 03929097.

TABLE 4
Inhibition of BRCA1 mRNA expression
in MDA-MB-231 and CAL-120 cells.

| | RTqPCR-MDA-MB-231 | RTqPCR-CAL-120 |
|---|---|---|
| Example | IC50 [mol/l] | IC50 [mol/l] |
| No | BRCA1 (median) | BRCA1 (median) |
| 1 | 1.37E−8 | |
| 2 | 8.55E−9 | |
| 3 | 8.64E−9 | |
| 4 | 1.71E−8 | |
| 5 | | |
| 6 | | |
| 7 | | |
| 8 | | |
| 9 | | |
| 10 | | |
| 11 | | |
| 12 | | |
| 13 | 3.90E−8 | |
| 14 | 4.29E−8 | |
| 15 | 1.31E−7 | |
| 16 | 1.29E−8 | 6.43E−8 |
| 17 | 1.09E−8 | |
| 18 | >1.07E−8 | |
| 19 | 8.59E−9 | |
| 20 | 2.06E−8 | |
| 21 | 2.61E−8 | |
| 22 | 9.36E−9 | |
| 23 | 3.09E−8 | |
| 24 | 4.77E−9 | |
| 25 | 5.88E−9 | |
| 26 | 2.00E−8 | |
| 27 | | |
| 28 | 1.68E−8 | |
| 29 | 9.47E−9 | |
| 30 | 1.17E−8 | |
| 31 | 4.99E−9 | |
| 32 | 2.90E−8 | |
| 33 | 1.69E−8 | |
| 34 | 2.25E−8 | |
| 35 | 3.02E−8 | |
| 36 | | |
| 37 | 1.13E−8 | |
| 38 | 2.18E−8 | |
| 39 | 1.07E−7 | |
| 40 | 8.65E−9 | |
| 41 | 2.06E−8 | |
| 42 | | |
| 43 | 2.89E−8 | |
| 44 | 2.42E−8 | |
| 45 | 7.60E−9 | |
| 46 | 4.02E−9 | |
| 47 | 1.38E−8 | |
| 48 | 3.95E−8 | |
| 49 | | |
| 50 | 1.75E−8 | |
| 51 | | |
| 52 | | |
| 53 | 1.09E−8 | |
| 54 | | |
| 55 | | |
| 56 | | |
| 57 | | |
| 58 | 2.82E−8 | |
| 59 | 2.48E−7 | |
| 60 | | |
| 61 | | |
| 62 | | |
| 63 | | |
| 64 | | |
| 65 | | |
| 66 | | |
| 67 | 7.80E−8 | |
| 68 | | |
| 69 | | |
| 70 | | |
| 71 | 3.01E−9 | |
| 72 | | |
| 73 | | |
| 74 | | |
| 75 | | |
| 76 | 7.40E−9 | |
| 77 | | |
| 78 | 1.56E−8 | |
| 79 | | |

4. Proliferation assay: MDA-MB-231, CAL-120

Human tumour cells were originally obtained from the American Type Culture Collection (ATCC), or from the Deutsche Sammlung von Mikroorganismen and Zellkulturen (DSMZ, German Collection of Microorganisms and Cell Cultures). Cultivated tumour cells (CAL-120, human breast adenocarcinoma cells, DSMZ ACC-459; MDA-M B-231, human breast cancer cells, ATCC HTB-26) were plated at a density of 4,000 cells/well in a 96-well multititer plate in 200 microL of their respective growth medium supplemented 10% fetal calf serum. After 24 hours, the cells of one plate (zero-point plate) were stained with crystal violet (see below), while the medium of the other plates was supplemented with the test substances in various concentrations (0 micromol/L, as well as in the range of 0.01-10 micromol/L; the final concentration of the solvent dimethyl sulfoxide was adjusted to 0.1%) using a Tecan HP D300 Digital Dispenser. The cells were incubated for 4 days in the presence of test substances. Cell proliferation was determined by staining the cells with crystal violet: the cells were fixed by adding 20 microL/measuring point of an 11% glutaric aldehyde solution for 15 minutes at room temperature. After three washing cycles of the fixed cells with water, the plates were dried at room temperature. The cells were stained by adding 100 microL/measuring point of a 0.1% crystal violet solution (pH 3.0). After three washing cycles of the stained cells with water, the plates were dried at room temperature. The dye was dissolved by adding 100 microL/measuring point of a 10% acetic acid solution. The extinction was determined by photometry at a wavelength of 595 nm. The change of cell number, in percent, was calculated by normalization of the measured values to the extinction values of the zero-point plate(=0%) and the extinction of the untreated (0 μM) cells(=100%). The IC50 values (inhibitory concentration at 50% of maximal effect) were determined by means of a 4 parameter fit.

TABLE 5
Antiproliferative data for the compounds of the present invention.

| Example | Prolif-MDA MB231 | Prolif-CAL-120_{—} |
|---|---|---|
| No | IC50 - [mol/l] (median) | IC50 - [mol/l] (median) |
| 1 | 2.49E−8 | 3.28E−8 |
| 2 | 1.08E−8 | 1.10E−8 |
| 3 | 3.73E−9 | 3.30E−9 |
| 4 | 5.13E−8 | 1.63E−8 |
| 5 | | |
| 6 | | |
| 7 | 3.91E−8 | 1.05E−8 |
| 8 | 3.96E−8 | 1.42E−8 |
| 9 | 4.12E−8 | 6.78E−9 |
| 10 | 1.51E−7 | 6.57E−8 |
| 11 | 1.76E−7 | 8.14E−8 |
| 12 | 3.83E−8 | 6.49E−9 |
| 13 | 1.68E−7 | |
| 14 | | 9.64E−8 |
| 15 | | |
| 16 | 7.02E−8 | 1.03E−7 |
| 17 | 1.37E−7 | 7.03E−8 |
| 18 | 1.51E−7 | 7.19E−8 |
| 19 | 4.83E−9 | 3.61E−9 |
| 20 | 1.44E−8 | 7.27E−9 |
| 21 | 1.13E−8 | 7.93E−9 |
| 22 | 8.02E−9 | 4.21E−9 |
| 23 | 2.86E−8 | 1.54E−8 |
| 24 | 1.24E−8 | 1.00E−8 |
| 25 | 5.07E−9 | 3.14E−9 |
| 26 | 2.22E−8 | 1.01E−8 |
| 27 | | 2.66E−7 |
| 28 | 1.99E−8 | 9.54E−9 |
| 29 | 1.45E−8 | 9.75E−9 |
| 30 | 2.99E−8 | 1.34E−8 |
| 31 | 1.82E−8 | 7.09E−9 |
| 32 | 5.52E−9 | 2.12E−9 |
| 33 | 3.70E−8 | 2.25E−8 |
| 34 | 3.88E−8 | 7.70E−9 |
| 35 | 5.95E−8 | 1.17E−8 |
| 36 | 1.14E−7 | 4.88E−8 |
| 37 | 1.51E−8 | 4.14E−9 |
| 38 | 4.65E−8 | 9.85E−9 |
| 39 | 3.15E−7 | 8.88E−8 |
| 40 | 8.39E−8 | 3.46E−8 |
| 41 | 6.71E−8 | 2.04E−8 |
| 42 | 1.48E−7 | 8.45E−8 |
| 43 | 6.22E−8 | 4.19E−8 |
| 44 | 3.79E−8 | 2.35E−8 |
| 45 | 1.68E−8 | 9.86E−9 |
| 46 | 4.72E−8 | 2.01E−8 |
| 47 | 5.08E−8 | 2.75E−8 |
| 48 | 6.61E−8 | 4.25E−8 |
| 49 | 2.00E−7 | 1.22E−7 |
| 50 | 1.50E−8 | 1.10E−8 |
| 51 | | |
| 52 | | |
| 53 | 2.33E−8 | 1.97E−8 |
| 54 | | |
| 55 | | |
| 56 | | |
| 57 | 2.41E−7 | 1.05E−7 |
| 58 | 2.13E−7 | 1.56E−7 |
| 59 | 8.16E−7 | 4.40E−7 |
| 60 | 2.35E−7 | 1.28E−7 |
| 61 | 3.35E−7 | 6.06E−8 |
| 62 | 8.73E−7 | 3.01E−7 |
| 63 | 1.78E−6 | 8.78E−7 |
| 64 | 5.36E−7 | 2.95E−7 |
| 65 | 4.37E−7 | 1.47E−7 |
| 66 | 3.86E−7 | 1.60E−7 |
| 67 | 2.68E−7 | 7.97E−8 |
| 68 | 6.45E−7 | 2.66E−7 |
| 69 | 3.11E−7 | 1.99E−7 |
| 70 | 1.74E−7 | 7.18E−8 |
| 71 | 7.08E−8 | 5.24E−8 |
| 72 | 1.09E−7 | 6.06E−8 |
| 73 | 1.41E−7 | 1.10E−7 |
| 74 | 2.51E−7 | 4.67E−8 |
| 75 | 3.76E−7 | 2.68E−7 |
| 76 | 1.24E−7 | 5.57E−8 |
| 77 | 3.00E−6 | 1.04E−6 |
| 78 | 3.47E−8 | 1.12E−8 |
| 79 | 2.17E−7 | 7.14E−8 |

5. CDK12 protein level: Protein Simple (CDK12, CAL-120), Westernblot (CDK9, 12, 13)

CAL-120 human breast cancer cells (DSMZ ACC 459) were seeded at 300,000 cells/well in 6-well plates containing 2 mL of growth medium (DMEM, 10%FCS, glutamine) and incubated for 24 h at 37° C. in an humidified incubator. Test compounds were added at various concentrations, control wells received solvent (DMSO), and the plates were incubated for another 18h at 37° C. Cells were washed 2 times with PBS and lysed in 75 microL lysis buffer (MSD-Puffer (MSD, #R6OTX-2), +1% SDS+PhosSTOP (Roche #04906837001)+complete mini (Roche #04693159001)) by scraping. The lysates were pushed 2 times through Qiashedders followed by centrifugation at 14,000 rpm for 30-50 sec. The supernatant was stored at -20° C. Proteins were separated by applying 0.4 microgramms of protein lysate to Protein Simple 66-440 kDa (Protein Simple #SM-5002) size assay columns on a PEGGY SUE or SALLY SUE equipment according to the supplier's manual. CDK12 and HSP90 (loading control) were detected using anti-human CDK12 antibody (Cell Signaling Technologies (CST) #11793) at 1:25 dilution and anti-human HSP90 antibody (CST #4877) at 1:5,000 dilution. CDK12 and HSP90 peak areas were determined using Protein Simple Compass software. Ratio of CDK12/HSP90 peak areas were calculated for each sample, and DC50 values (degrading concentration to achieve 50% reduction relative to vehicle treated control) were determined by means of a 4 parameter fit.

Westernblot analyses were performed according to standard protocols. 40 microgramms of protein lysates per lane were subjected to polyacrylamide gel electrophoresis using NuPAGE 3-8% tris acetate gels (ThermoFisher) for detection of CDK12 and CDK13 or using NuPAGE 4-12% bis-tris gels (ThermoFisher) for detection of CDK9 followed by protein transfer to nitrocellulose membranes using a BioRad Transblot Turbo equipment. Membranes were probed with rabbit anti CDK12 antibodies (CST # 11793), rabbit anti-CDK13 antibodies (Novus #NB 100-68268), anti-CDK9 antibodies (CST #2316), and anti HSP90 (Becton Dickinson #610419) or anti-GAPDH (Zytomed #RGM2-6C5) antibodies for loading control.

TABLE 6
CDK12 protein level in CAL-120 breast
cancer cells, DC₅₀ - [mol/l]

| | CDK12 protein level in CAL-120 |
|---|---|
| Example | breast ca cells DC50 - [mol/l] |
| No | (median) |
| 16 | 3.11E−8 |

6. CDK12 Nuclear Protein Level: Immunofluorescence/High Content Analysis

In the context of the present invention, the term “DC50 CDK12” refers to the DC₅₀ values obtained according to the assay described in this section (6) herein below, i.e. the DC₅₀ values for the degradation of CDK12.:

CAL-120 human breast cancer cells (DSMZ ACC 459) are seeded in 1536-well microtiter plates (800 cells per well) containing 50 nanoL of compounds in Dose-Response. Control wells received DMSO or Reference Example 1. Plates are then incubated for 24 h at 37° C. in an humidified incubator and fixed with 4% PFA for 10 min. Then immunofluorescence (IF) against CDK12 (CellSignalling CDK12 Antibody #11973, rabbit, 1:100 dilution) is performed using standard IF protocols. Cells are then stained with Hoechst 33342 (Life Technologies, H-1399, 0.1 microg/mL) and imaged on an automated confocal microscopy system (e.g. Perkin Elmer Opera Phenix). Nuclear and cytoplasmic intensity as well as the nuclear/cytoplasmic intensity ratio is determined by automated image analysis using custom generated scripts (MetaXpress). Data is then transferred to Genedata Screener software, normalized to DMSO and control and DC50 values (degrading concentration to achieve 50% reduction of nuclear CDK12 staining intensity relative to controls) are reported.

TABLE 7
Immunofluorescence CDK12 degradation - DC₅₀ [mol/L]

| Example | CDK12 degradation IF |
|---|---|
| No | DC50 - [mol/l] (median) |
| 1 | 4.20E−9 |
| 2 | 6.09E−9 |
| 3 | 1.92E−9 |
| 4 | 4.30E−9 |
| 5 | 8.34E−8 |
| 6 | 1.31E−8 |
| 7 | 1.44E−8 |
| 8 | 8.53E−9 |
| 9 | 8.07E−9 |
| 10 | 4.08E−8 |
| 11 | 7.49E−8 |
| 12 | 8.53E−9 |
| 13 | 4.39E−8 |
| 14 | 4.96E−8 |
| 15 | 1.55E−7 |
| 16 | 8.40E−8 |
| 17 | 2.48E−8 |
| 18 | 1.75E−8 |
| 19 | 4.66E−9 |
| 20 | 2.22E−8 |
| 21 | 2.87E−8 |
| 22 | 1.18E−8 |
| 23 | 3.17E−8 |
| 24 | 1.20E−8 |
| 25 | 8.43E−9 |
| 26 | 1.60E−8 |
| 27 | 4.91E−8 |
| 28 | 1.06E−8 |
| 29 | 1.75E−8 |
| 30 | 1.71E−8 |
| 31 | 1.44E−8 |
| 32 | 1.15E−8 |
| 33 | 8.65E−9 |
| 34 | 1.42E−8 |
| 35 | 1.91E−8 |
| 36 | 1.62E−7 |
| 37 | 1.26E−8 |
| 38 | 1.32E−8 |
| 39 | 3.82E−8 |
| 40 | 2.55E−9 |
| 41 | 4.05E−9 |
| 42 | 1.11E−8 |
| 43 | 9.53E−9 |
| 44 | 3.49E−8 |
| 45 | 8.11E−9 |
| 46 | 1.38E−8 |
| 47 | 2.28E−8 |
| 48 | 7.12E−9 |
| 49 | 2.92E−8 |
| 50 | 3.15E−9 |
| 51 | 1.07E−8 |
| 52 | 4.93E−8 |
| 53 | 3.20E−8 |
| 54 | 1.31E−8 |
| 55 | 4.69E−8 |
| 56 | 7.11E−9 |
| 57 | 1.70E−7 |
| 58 | 2.72E−8 |
| 59 | 3.24E−8 |
| 60 | 4.12E−8 |
| 61 | 1.63E−7 |
| 62 | 6.76E−7 |
| 63 | 9.12E−7 |
| 64 | 8.26E−8 |
| 65 | 2.05E−8 |
| 66 | 1.94E−8 |
| 67 | 2.10E−8 |
| 68 | 2.91E−8 |
| 69 | 9.94E−8 |
| 70 | 1.08E−8 |
| 71 | 2.77E−8 |
| 72 | 1.87E−8 |
| 73 | 1.12E−7 |
| 74 | 1.51E−8 |
| 75 | 1.35E−7 |
| 76 | 2.17E−9 |
| 77 | 1.27E−7 |
| 78 | 3.36E−8 |
| 79 | 4.12E−8 |

TABLE 8
IC₅₀ CDK12 high ATP to Degradation DC₅₀ CDK12 ratio

| | (IC₅₀ CDK12 high ATP) |
|---|---|
| Example | to (Degradation DC₅₀ |
| No | CDK12) ratio |
| 1 | >2380 |
| 2 | >3284 |
| 3 | ≥6041 |
| 4 | >4651 |
| 5 | >239 |
| 6 | >1526 |
| 7 | >1388 |
| 8 | >2344 |
| 9 | >2478 |
| 10 | >4901 |
| 11 | >267 |
| 12 | >2344 |
| 13 | >455 |
| 14 | >403 |
| 15 | >129 |
| 16 | >238 |
| 17 | >807 |
| 18 | >1142 |
| 19 | >4294 |
| 20 | >1050 |
| 21 | >697 |
| 22 | >1695 |
| 23 | 388 |
| 24 | 1614 |
| 25 | 2193 |
| 26 | >509 |
| 27 | 41 |
| 28 | >1880 |
| 29 | >1145 |
| 30 | >1376 |
| 31 | >1388 |
| 32 | 1206 |
| 33 | >2312 |
| 34 | >1412 |
| 35 | >1046 |
| 36 | >124 |
| 37 | >1589 |
| 38 | >1516 |
| 39 | >534 |
| 40 | >7840 |
| 41 | >4938 |
| 42 | >2322 |
| 43 | |
| 44 | |
| 45 | |
| 46 | >1451 |
| 47 | 562 |
| 48 | 1923 |
| 49 | >685 |
| 50 | 1963 |
| 51 | >1865 |
| 52 | >406 |
| 53 | >313 |
| 54 | 853 |
| 55 | >426 |
| 56 | ≥2539 |
| 57 | >118 |
| 58 | >735 |
| 59 | >618 |
| 60 | >102 |
| 61 | >123 |
| 62 | >30 |
| 63 | >22 |
| 64 | >242 |
| 65 | >978 |
| 66 | >1116 |
| 67 | >937 |
| 68 | >687 |
| 69 | >101 |
| 70 | >1849 |
| 71 | >722 |
| 72 | >536 |
| 73 | >90 |
| 74 | >660 |
| 75 | >148 |
| 76 | >9202 |
| 77 | >133 |
| 78 | >596 |
| 79 | >128 |

7. CYCLIN K Nuclear Protein Level: Immunofluorescence/High Content Analysis

In the context of the present invention, the term “DC50 Cyclin K” refers to the DC₅₀ values obtained according to the assay described in this section (7) herein below, i.e. the DC₅₀ values for the degradation of Cyclin K.

CAL-120 human breast cancer cells (DSMZ ACC 459) are seeded in 1536-well microtiter plates (800 cells per well) containing 50 nanoL of compounds in Dose-Response. Control wells received DMSO or Reference Example 1. Plates are then incubated for 24 h at 37° C. in an humidified incubator and fixed with 4% PFA for 10 min. Then immunofluorescence (IF) against CYCLIN K (ThermoFisher Scientific CCNK Antibody # PA5-85020, rabbit, 1:200 dilution) is performed using standard IF protocols. Cells are then stained with Hoechst 33342 (Life Technologies, H-1399, 0.1 microg/mL) and imaged on an automated confocal microscopy system (e.g. Perkin Elmer Opera Phenix). Nuclear and cytoplasmic intensity as well as the nuclear/cytoplasmic intensity ratio is determined by automated image analysis using custom generated scripts (MetaXpress). Data is then transferred to Genedata Screener software, normalized to DMSO and control and DC50 values (degrading concentration to achieve 50% reduction of nuclear CCNK staining intensity relative to controls) are reported.

TABLE 9
Immunofluorescence CYCLIN K degradation - DC₅₀ [mol/L]

| | CYCLIN K degradation |
|---|---|
| Example | IF, DC50- [mol/l] |
| No | (median) |
| 1 | 7.10E−9 |
| 2 | 7.04E−10 |
| 3 | 1.15E−9 |
| 4 | 9.14E−10 |
| 5 | 3.46E−9 |
| 6 | 2.63E−10 |
| 7 | 2.62E−9 |
| 8 | 6.22E−9 |
| 9 | |
| 10 | 1.59E−7 |
| 11 | |
| 12 | 2.16E−9 |
| 13 | 3.11E−9 |
| 14 | |
| 15 | |
| 16 | >6.77E−7 |
| 17 | |
| 18 | |
| 19 | 3.39E−10 |
| 20 | |
| 21 | |
| 22 | |
| 23 | |
| 24 | 1.22E−9 |
| 25 | 5.43E−10 |
| 26 | |
| 27 | |
| 28 | |
| 29 | |
| 30 | |
| 31 | |
| 32 | 8.77E−10 |
| 33 | |
| 34 | |
| 35 | |
| 36 | |
| 37 | 1.72E−9 |
| 38 | |
| 39 | 3.87E−8 |
| 40 | 5.35E−9 |
| 41 | 2.30E−9 |
| 42 | 1.72E−8 |
| 43 | 4.79E−9 |
| 44 | 1.35E−8 |
| 45 | 2.29E−9 |
| 46 | 4.87E−9 |
| 47 | 6.29E−9 |
| 48 | 1.13E−8 |
| 49 | 2.57E−8 |
| 50 | 3.27E−9 |
| 51 | 4.46E−9 |
| 52 | 9.26E−8 |
| 53 | 6.41E−9 |
| 54 | 3.31E−9 |
| 55 | 1.25E−8 |
| 56 | 1.28E−9 |
| 57 | |
| 58 | 1.56E−8 |
| 59 | 3.86E−8 |
| 60 | |
| 61 | |
| 62 | |
| 63 | |
| 64 | |
| 65 | 3.74E−8 |
| 66 | 2.03E−8 |
| 67 | 1.56E−8 |
| 68 | 2.07E−8 |
| 69 | 3.41E−8 |
| 70 | 1.16E−8 |
| 71 | 8.83E−9 |
| 72 | 2.34E−8 |
| 73 | 5.32E−8 |
| 74 | 1.36E−8 |
| 75 | 1.24E−7 |
| 76 | 8.11E−9 |
| 77 | 1.18E−7 |
| 78 | |
| 79 | |

8. In Vivo Xenotransplantation Models

The anti-tumor activity of test compound was examined in murine xenotransplantation models of human cancer. For this purpose, mice were implanted subcutaneously or orthotopically with specific human tumor cells. At a mean tumor size of 20-30 mm² animals were randomized into treatment and control groups (n=10 animals/group) and treatment started with vehicle only or Compound (formulation: 80% PEG400/20% Water; application route: p.o./per os, orally; dose/schedule: 5 mg/kg daily (QD), 5 mg/kg twice daily (2QD) for 2 days on/5 days off). The oral application volume was 10 mL/kg. The time interval between two applications per day was 6-7 h. The experiment was ended when the untreated control group had tumors of area 225 mm². The tumor size and the body weight were determined three times weekly. Changes in the body weight were a measure of treatment-related toxicity (>10%=critical body weight loss and stop of treatment until recovery, >20%=toxic, termination). The tumor area was detected by means of an electronic caliper gauge [length (mm)×width (mm)]. In vivo anti-tumor efficacy is presented as T/C ratio (Treatment/Control) calculated with tumor areas at study end by the formula [(tumor area of treatment group at day x)−(tumor area of treatment group at day before first treatment)]/[(tumor area of control group at day x)−(tumor area of control group at day before first treatment)]. A compound having a T/C below 0.5 is defined as active (effective). Statistical analysis was assessed using SigmaStat software. A one-way analysis of variance was performed and differences to the control were compared by a pair-wise comparison procedure (Dunn's method).

9. CYP Inhibition Assay

Use of in vitro assays to evaluate the inhibition potential of new drug candidates towards CYP-mediated metabolism has been shown to be effective as part of a strategy to minimize the chances of drug interactions with co-administered drugs.

The inhibitory potency of the test compound towards 5 human cytochrome P450 isoforms (CYP1A2, 2C8, 2C9, 2D6, and 3A4) was determined during the lead optimization phase. In case of CYP3A4 also time dependent inhibitory potential was tested by applying a 30 min pre-incubation time of the test compound in metabolically active incubation system. Human liver microsomes (pooled, >30 male and female donors) were incubated with individual CYP isoform-selective standard probes (phenacetin, amodiaquine, diclofenac, dextromethorphan and midazolam) in the absence and presence of increasing concentrations of the test compound. Furthermore, the inhibitory potency of standard inhibitors was included as positive controls (fluvoxamine for CYP1A2, montelukast for CYP2C8, sulfaphenazole for CYP2C9, fluoxetine for CYP2D6, ketoconazole for CYP3A4 and mibefradil for CYP3A4-preincubation). Incubation conditions (protein and substrate concentration, incubation time) were optimized with regard to linearity and metabolite turnover. Incubation medium consists of 50 millimol/L potassium phosphate buffer (pH 7.4) containing 1 millimol/L EDTA, NADPH regenerating system (1 millimol/L NADP, 5 millimol/L glucose 6-phosphate, glucose 6-phosphate dehydrogenase (1.5 U/mL). Sequential dilutions and incubations were performed on a Freedom Evo Workstation (Tecan, Crailsheim, FRG) in 96-well plates at 37° C. A final incubation volume of 200 μL was used. Reactions were stopped by addition of 100 μL acetonitrile containing the respective internal standard. After centrifugation the supernatants were analyzed by LC-MS/MS. The LC-MS/MS system for quantification of paracetamol (CYP1A2), desethylamodiaquine (CYP2C8), 4′-hydroxydiclofenac (CYP2C9), dextrorphan (CYP2D6), and 1′-hydroxymidazolam (CYP3A4) comprised a QTRAP 6500® LC-MS/MS system (Applied Biosystems, MDS Sciex, Ontario, Canada) equipped with an electrospray ionization (ESI) interface (Turboionspray® interface) used to generate positive [M+H]+ ions, an Agilent HP 1290 liquid chromatograph (Agilent Technologies, Waldbronn, Germany) and a HTS PAL autosampler (CTC Analytics, Zwingen, Switzerland).

Data analysis: The CYP-mediated activities in the presence of test compounds (inhibitors) were expressed as percentages of the corresponding no inhibitor control samples. A sigmoid-shaped curve was fitted to the data, and the enzyme inhibition parameter IC50 was calculated using a nonlinear least-squares regression analysis of the plot of percent control activity versus concentration of the test inhibitor.

10. Investigation of In Vitro Metabolic Stability in Rat Hepatocytes

Hepatocytes from Han Wistar rats were freshly isolated via a 2-step perfusion method. After perfusion, the liver was carefully removed from the rat: the liver capsule was opened and the hepatocytes were gently shaken out into a Petri dish with ice-cold Williams' medium E (WME). The resulting cell suspension was filtered through sterile gaze in 50 mL falcon tubes and centrifuged at 50×g for 3 min at room temperature. The cell pellet was resuspended in 30 mL WME and centrifuged through a Percoll® gradient for 2 times at 100×g. The hepatocytes were washed again with WME and resuspended in medium containing 5% FCS. Cell viability was determined by trypan blue exclusion.

For the metabolic stability assay liver cells were distributed in WME containing 5% FCS to glass vials at a density of 1.0×10⁶ vital cells/mL. The test compound was added to a final concentration of 1 micromol/L. During incubation, the hepatocyte suspensions were continuously shaken at 580 rpm and aliquots were taken at 2, 8, 16, 30, 45 and 90 min, to which equal volumes of cold methanol were immediately added. Samples were frozen at −20° C. over night, after subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, amount of liver cells in vivo and in vitro. The hepatic in vivo blood clearance (CLblood) and the maximal oral bioavailability (Fmax) was calculated using the following formulae: CL'intrinsic [mL/(min*kg)]=kel [1/min]/((cellno/volume of incubation [mL])*fu,inc)*(cellno/liver weight [g])*(specific liver weight [g liver/kg body weight]); CLblood well-stirred [L/(h*kg)]=(QH [L/(h*kg)]*fu,blood*CL′intrinsic [L/(h*kg)])/(QH [L/(h*kg)]+fu,blood*CL′intrinsic [L/(h*kg)]); Fmax=1-CLblood/QH and using the following parameter values: Liver blood flow (QH)−4.2 L/h/kg rat; specific liver weight−32 g/kg rat body weight; liver cells in vivo—1.1×108 cells/g liver, liver cells in vitro—1.0×106/mL; fu,inc and fu,blood is taken as 1.

11. PXR Nuclear Receptor Activation

DPX2 cells (hepatoma cell line stably-cotransfected with a vector for human PXR and a Luciferase reporter gene under the control of two human CYP3A4 promotors, Puracyp, Carlsbad, CA) were cultivated according to manufacturer's instructions with following modifications: Cells were seeded in a 384 well plate and cultivated at 37° C./5% CO2 in humidified air. 24 h prior read-out the cells were treated with compound in a ten point serial dilution of ˜1:3 starting at the highest test concentration of 49.8 micromol/L and ending at 2 nanomol/L. Rifampicin was incubated in the same manner as positive control. In addition, for the normalization of the luminescence signal cells were incubated with Rifampicin at a concentration of 16.7 micromol/L corresponding to 100% activation, as well as DMSO for background luminescence corresponding to 0% activation (n=32 wells each). Cells were lyzed and incubated with the Luciferase substrate ONE-Glo™ Reagent (Promega, Madison Wis., USA) according to manufacturer's instructions and luminescence signal was detected in a plate reader. A concentration-dependent increase of the luciferase activity above 10% of Rifampicin control was classified as PXR transactivation

12. In Vivo Pharmacokinetics in Rats

For in vivo pharmacokinetic experiments test compounds were administered to male Wistar rats intravenously at doses of 0.3 to 1 mg/kg and intragastral at doses of 0.5 to 10 mg/kg formulated as solutions using solubilizers such as PEG400 in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 6 h, 8 h and 24 h after dosing. Depending on the expected half-life additional samples were taken at later time points (e.g. 48 h, 72 h). For pharmacokinetics after intragastral administration test compounds were given intragastral to fasted rats and blood samples were taken at 5 min, 15 min, 30 min, 45 min, 1 h, 2 h, 4 h, 6 h, 8 h and 24 h after dosing. Depending on the expected half-life additional samples were taken at later time points (e.g. 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitril and frozen at −20° C. over night. Samples were subsequently thawed and centrifuged at 3000 rpm, 4° C. for 20 minutes. Aliquots of the supernatants were taken for analytical testing using an Agilent 1200 H PLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0-tlast)norm: Area under the concentration-time curve from t=Oh to the last time point for which plasma concentrations could be measured divided by the administered dose (in kg*h/L); t1/2: terminal half-life (in h); F: oral bioavailability: AUCnorm after intragastral administration divided by AUCnorm after intravenous administration (in %).

1. A compound of formula (I): 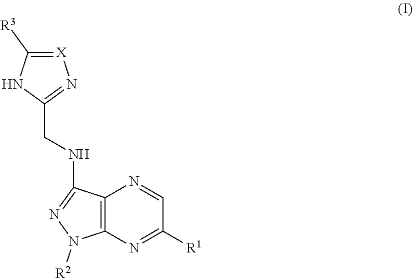
wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group,a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

2. The compound of formula (I) according to claim 1, wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-haloalkyl group, a C₃-C₈-cycloalkyl group, a phenyl group, a heteroaryl group and a heterocycloalkyl group,
wherein said heterocycloalkyl group is connected to the rest of the molecule via a carbon atom of said heterocycloalkyl group,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

3. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-haloalkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a heteroaryl group, a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

4. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group, heteroaryl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

5. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group and a heterocycloalkyl group,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

6. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-hydroxyalkyl group, a (C₁-C₃-alkoxy)-(C₁-C₅-alkyl)- group, a (C₃-C₈-cycloalkyl)-(C₁-C₂-alkyl)- group, a (R⁵R⁶N—)—(C₁-C₅-alkyl)- group, a heterocycloalkyl group, a 7- to 9-membered bridged compound and a 7- to 11-membered spiro compound,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

7. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

8. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a 4- to 7-membered heterocycloalkyl group, a 7- to 9-membered bridged compound and a 7- to 11-membered spiro compound,
wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group, 7- to 9-membered bridged compound or 7- to 11-membered spiro compound,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

9. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₃-C₈-cycloalkyl group, a phenyl group and a heterocycloalkyl group,
wherein said heterocycloalkyl group is connected to the rest of the molecule via a carbon atom of said heterocycloalkyl group,
wherein said phenyl group or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

10. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-hydroxyalkyl group, a (C₁-C₃-alkoxy)-(C₁-C₅-alkyl)- group, a C₃-C₈-cycloalkyl group, a (C₃-C₈-cycloalkyl)-(C₁-C₂-alkyl)- group, a (R⁵R⁶N—)—(C₁-C₅-alkyl)- group, a phenyl group, a 4- to 7-membered heterocycloalkyl group, a 7- to 9-membered bridged compound and a 7- to 11-membered spiro compound,
wherein said 4- to 7-membered heterocycloalkyl group, 7- to 9-membered bridged compound or 7- to 11-membered spiro compound is connected to the rest of the molecule via a carbon atom of said 4- to 7-membered heterocycloalkyl group, 7- to 9-membered bridged compound or 7- to 11-membered spiro compound,
wherein said 7- to 9-membered bridged compound or 7- to 11-membered spiro compound each optionally contains one heteroatom independently selected from nitrogen and oxygen,
wherein said C₃-C₈-cycloalkyl group, phenyl group or 4- to 7-membered heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
and R³ and R⁴, together with the carbon atoms to which they are attached form a phenyl group, wherein said phenyl group is optionally substituted one, two or three times, each substituent independently selected from a halogen atom and a C₁-C₃-haloalkyl group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C1-C4-alkyl group;
or a tautomer, or an N-oxide, or a salt thereof, or a salt of a tautomer, or a salt of an N-oxide, or a mixture of same.

11. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a heterocycloalkyl group, a 7- to 9-membered bridged compound and a 7- to 11-membered spiro compound,
wherein said heterocycloalkyl group is selected from tetrahydrofuranyl-, pyrrolidinyl-, tetrahydropyranyl- and piperidinyl-,
wherein said heterocycloalkyl group, 7- to 9-membered bridged compound or 7- to 11-membered spiro compound is connected to the rest of the molecule via a carbon atom of said heterocycloalkyl group,
wherein said 7- to 9-membered bridged compound or 7- to 11-membered spiro compound each optionally contains one heteroatom independently selected from nitrogen and oxygen,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
and R³ and R⁴, together with the carbon atoms to which they are attached form a phenyl group, wherein said phenyl group is optionally substituted one or two times, each substituent independently selected from a halogen atom and a C₁-C₃-haloalkyl group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₄-alkyl group;
or a tautomer, or an N-oxide, or a salt thereof, or a salt of a tautomer, or a salt of an N-oxide, or a mixture of same.

12. The compound of formula (I) according to claim 1, wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a heterocycloalkyl group selected from tetrahydrofuranyl-, pyrrolidinyl-, tetrahydropyranyl- and piperidinyl-,
wherein said heterocycloalkyl group is connected to the rest of the molecule via a carbon atom of said heterocycloalkyl group,
wherein said heterocycloalkyl group is optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a R⁷OOC— group and a R⁵R⁶N— group;
X is a CR⁴ group;
and R³ and R⁴, together with the carbon atoms to which they are attached form a phenyl group, wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)— (C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

13. The compound of formula (I) according to claim 1, selected from the group consisting of:
N-[(1H-benzimidazol-2-yl)methyl]-6-cyclopropyl-1-(1-methylpiperidin-4-yl)-1H-pyrazolo[3,4-b]pyrazin-3-amine,
6-cyclopropyl-N-[(4-methoxy-1H-benzimidazol-2-yl)methyl]-1-(1-methylpiperidin-4-yl)-1H-pyrazolo[3,4-b]pyrazin-3-amine,
6-cyclopropyl-N-[(4-fluoro-1H-benzimidazol-2-yl)methyl]-1-(1-methylpiperidin-4-yl)-1H-pyrazolo[3,4-b]pyrazin-3-amine,
6-cyclopropyl-N-[(5-fluoro-1H-benzimidazol-2-yl)methyl]-1-(1-methylpiperidin-4-yl)-1H-pyrazolo[3,4-b]pyrazin-3-amine,
6-cyclopropyl-1-(1-methylpiperidin-4-yl)-N-{[4-(trifluoromethoxy)-1H-benzimidazol-2-yl]methyl}-1H-pyrazolo [3,4-b]pyrazin-3-amine,
6-cyclopropyl-N-[(5-methyl-1H-benzimidazol-2-yl)methyl]-1-(1-methylpiperidin-4-yl)-1H-pyrazolo[3,4-b]pyrazin-3-amine,
6-cyclopropyl-1-(1-methylpiperidin-4-yl)-N-{[4-(trifluoromethyl)-1H-benzimidazol-2-yl]methyl}-1H-pyrazolo[3,4-b]pyrazin-3-amine,
6-cyclopropyl-N-[(4-methyl-1H-benzimidazol-2-yl)methyl]-1-(1-methylpiperidin-4-yl)-1H-pyrazolo[3,4-b]pyrazin-3-amine,
6-cyclopropyl-N-[(4,5-difluoro-1H-benzimidazol-2-yl)methyl]-1-(1-methylpiperidin-4-yl)-1H-pyrazolo[3,4-b]pyrazin-3-amine,
(±)-N-[(1H-benzimidazol-2-yl)methyl]-6-cyclopropyl-1-[oxolan-3-yl]-1H-pyrazolo[3,4-b]pyrazin-3-amine,
tert-butyl 4-(3-{[(1H-benzimidazol-2-yl)methyl]amino}-6-cyclopropyl-1H-pyrazolo[3,4-b]pyrazin-1-yl)piperidine-1-carboxylate,
N-[(1H-benzimidazol-2-yl)methyl]-6-cyclopropyl-1-(piperidin-4-yl)-1H-pyrazolo [3,4-b]pyrazin-3-amine,
formic acid-N-[(1H-benzimidazol-2-yl)methyl]-6-cyclopropyl-1-phenyl-1H-pyrazolo [3,4-b]pyrazin-3-amine (1/1),
N-[(1H-benzimidazol-2-yl)methyl]-6-cyclopropyl-1-(propan-2-yl)-1H-pyrazolo[3,4-b]pyrazin-3-amine,
N-[(1H-benzimidazol-2-yl)methyl]-6-cyclopropyl-1-methyl-1H-pyrazolo[3,4-b]pyrazin-3-amine,
N-[(1H-benzimidazol-2-yl)methyl]-6-cyclopropyl-1-(oxan-4-yl)-1H-pyrazolo [3,4-b]pyrazin-3-amine,
(+) or (−)-N-[(1H-benzimidazol-2-yl)methyl]-6-cyclopropyl-1-(oxolan-3-yl)-1H-pyrazolo[3,4-b]pyrazin-3-amine,
(−) or (+)-N-[(1H-benzimidazol-2-yl)methyl]-6-cyclopropyl-1-[oxolan-3-yl]-1H-pyrazolo[3,4-b]pyrazin-3-amine,
6-cyclopropyl-N-[(5,6-dichloro-1H-benzimidazol-2-yl)methyl]-1-(1-methylpiperidin-4-yl)-1H-pyrazolo[3,4-b]pyrazin-3-amine,
N-[(4-bromo-1H-benzimidazol-2-yl)methyl]-6-cyclopropyl-1-(1-methylpiperidin-4-yl)-1H-pyrazolo[3,4-b]pyrazin-3-amine,
N-[(5-chloro-4-methyl-1H-benzimidazol-2-yl)methyl]-6-cyclopropyl-1-(1-methylpiperidin-4-yl)-1H-pyrazolo[3,4-b]pyrazin-3-amine,
N-[(5-chloro-1H-benzimidazol-2-yl)methyl]-6-cyclopropyl-1-(1-methylpiperidin-4-yl)-1H-pyrazolo[3,4-b]pyrazin-3-amine,
6-cyclopropyl-N-[(4-fluoro-1H-benzimidazol-2-yl)methyl]-1-(oxan-4-yl)-1H-pyrazolo[3,4-b]pyrazin-3-amine,
6-cyclopropyl-N-[(4-methoxy-1H-benzimidazol-2-yl)methyl]-1-(oxan-4-yl)-1H-pyrazolo[3,4-b]pyrazin-3-amine,
6-cyclopropyl-1-(1-methylpiperidin-4-yl)-N-{[5-(trifluoromethyl)-1H-benzimidazol-2-yl]methyl}-1H-pyrazolo[3,4-b]pyrazin-3-amine,
6-cyclopropyl-N-[(5-fluoro-4-methyl-1H-benzimidazol-2-yl)methyl]-1-(1-methylpiperidin-4-yl)-1H-pyrazolo[3,4-b]pyrazin-3 -amine,
6-cyclopropyl-1-(1-methylpiperidin-4-yl)-N-({5-[(trifluoromethyl)sulfanyl]-1H-benzimidazol-2-yl}methyl)-1H-pyrazolo[3,4-b]pyrazin-3-amine,
6-cyclopropyl-N-[(4,5-dimethyl-1H-benzimidazol-2-yl)methyl]-1-(1-methylpiperidin-4-yl)-1H-pyrazolo[3,4-b]pyrazin-3-amine,
N-[(5-chloro-6-fluoro-1H-benzimidazol-2-yl)methyl]-6-cyclopropyl-1-(1-methylpiperidin-4-yl)-1H-pyrazolo[3,4-b]pyrazin-3 -amine,
6-cyclopropyl-N-[(5,6-difluoro-1H-benzimidazol-2-yl)methyl]-1-(1-methylpiperidin-4-yl)-1H-pyrazolo[3,4-b]pyrazin-3-amine,
N-[(4-chloro-5-fluoro-1H-benzimidazol-2-yl)methyl]-6-cyclopropyl-1-(1-methylpiperidin-4-yl)-1H-pyrazolo[3,4-b]pyrazin-3 -amine,
6-cyclopropyl-N-[(4,5-difluoro-1H-benzimidazol-2-yl)methyl]-1-(oxan-4-yl)-1H-pyrazolo[3,4-b]pyrazin-3-amine,
6-cyclopropyl-N-[(4-methyl-1H-benzimidazol-2-yl)methyl]-1-(oxan-4-yl)-1H-pyrazolo [3,4-b]pyrazin-3 -amine,
(±)-N-[(1H-benzimidazol-2-yl)methyl]-6-cyclopropyl-1-(1-methylazepan-4-yl)-1H-pyrazolo[3,4-b]pyrazin-3-amine,
(±)-N-[(1H-benzimidazol-2-yl)methyl]-6-cyclopropyl-1-(1-methylpyrrolidin-3 -yl)-1H-pyrazolo[3,4-b]pyrazin-3-amine,
N-[(1H-benzimidazol-2-yl)methyl]-6-cyclopropyl-1-(4,4-difluorocyclohexyl)-1H-pyrazolo[3,4-b]pyrazin-3-amine,
(+) or (—)-N-[(1H-benzimidazol-2-yl)methyl]-6-cyclopropyl-1-[(4R*)-1-methylazepan-4-yl]-1H-pyrazolo[3,4-b]pyrazin-3-amine,
(−) or (+)-N-[(1H-benzimidazol-2-yl)methyl]-6-cyclopropyl-1-[(4R*)-1-methylazepan-4-yl]-1H-pyrazolo[3,4-b]pyrazin-3-amine,
N-[(1H-benzimidazol-2-yl)methyl]-1-cyclopentyl-6-cyclopropyl-1H-pyrazolo[3,4-b]pyrazin-3-amine,
cis-3-(3-{[(1H-benzimidazol-2-yl)methyl]amino}-6-cyclopropyl-1H-pyrazolo[3,4-b]pyrazin-1-yl)cyclobutan-1-ol,
(±)-cis/trans-N-[(1H-benzimidazol-2-yl)methyl]-6-cyclopropyl-1-(1,2-dimethylpiperidin-4-yl)-1H-pyrazolo[3,4-b]pyrazin-3-amine,
(±)-cis/trans-N-[(1H-benzimidazol-2-yl)methyl]-6-cyclopropyl-1-(3-methyloxan-4-yl)-1H-pyrazolo[3,4-b]pyrazin-3-amine,
trans-3-(3-{[(1H-benzimidazol-2-yl)methyl]amino}-6-cyclopropyl-1H-pyrazolo[3,4-b]pyrazin-1-yl)cyclobutan-1-ol,
(±)-cis/trans-N-[(1H-benzimidazol-2-yl)methyl]-6-cyclopropyl-1-(2-methyloxan-4-yl)-1H-pyrazolo[3,4-b]pyrazin-3-amine,
exo/endo-N-[(1H-benzimidazol-2-yl)methyl]-6-cyclopropyl-1-(8-oxabicyclo[3.2.1]octan-3-yl)-1H-pyrazolo[3,4-b]pyrazin-3-amine,
[trans-3-(3-{[(1H-benzimidazol-2-yl)methyl]amino}-6-cyclopropyl-1H-pyrazolo[3,4-b]pyrazin-1-yl)cyclobutyl]methanol,
[cis-3-(3-{[(1H-benzimidazol-2-yl)methyl]amino}-6-cyclopropyl-1H-pyrazolo[3,4-b]pyrazin-1-yl)cyclobutyl]methanol,
2-[cis/trans-3-(3-{[(1H-benzimidazol-2-yl)methyl]amino}-6-cyclopropyl-1H-pyrazolo[3,4-b]pyrazin-1-yl)cyclobutyl]propan-2-ol,
N-[(1H-benzimidazol-2-yl)methyl]-1-cyclobutyl-6-cyclopropyl-1H-pyrazolo[3,4-b]pyrazin-3-amine,
trans-4-(3-{[(1H-benzimidazol-2-yl)methyl]amino}-6-cyclopropyl-1H-pyrazolo[3,4-b]pyrazin-1-yl)cyclohexan-1-ol,
cis-4-(3-{[(1H-benzimidazol-2-yl)methyl]amino}-6-cyclopropyl-1H-pyrazolo[3,4-b]pyrazin-1-yl)cyclohexan-1-ol,
N-[(1H-benzimidazol-2-yl)methyl]-6-cyclopropyl-1-(2,2,6,6-tetramethyloxan-4-yl)-1H-pyrazolo[3,4-b]pyrazin-3-amine,
6-cyclopropyl-N-[(5-fluoro-1H-benzimidazol-2-yl)methyl]-1-(oxan-4-yl)-1H-pyrazolo [3,4-b]pyrazin-3-amine,
N-[(1H-benzimidazol-2-yl)methyl]-6-cyclopropyl-1-(2-oxaspiro[3.3]heptan-6-yl)-1H-pyrazolo[3,4-b]pyrazin-3-amine,
N-[(1H-benzimidazol-2-yl)methyl]-6-cyclopropyl-1-(oxetan-3-yl)-1H-pyrazolo [3,4-b]pyrazin-3-amine,
trans-4-(6-cyclopropyl-3-{[(5-fluoro-1H-benzimidazol-2-yl)methyl]amino}-1H-pyrazolo [3,4-b]pyrazin-1-yl)cyclohexan-1-ol,
(±)-N-[(1H-benzimidazol-2-yl)methyl]-6-cyclopropyl-1-(4-methoxybutan-2-yl)-1H-pyrazolo[3,4-b]pyrazin-3-amine,
(−)-N-[(1H-benzimidazol-2-yl)methyl]-6-cyclopropyl-1-[4-methoxybutan-2-yl]-1H-pyrazolo [3,4-b]pyrazin-3-amine,
(+)-N-[(1H-benzimidazol-2-yl)methyl]-6-cyclopropyl-1-[4-methoxybutan-2-yl]-1H-pyrazolo[3,4-b]pyrazin-3-amine,
3-(3-{[(1H-benzimidazol-2-yl)methyl]amino}-6-cyclopropyl-1H-pyrazolo[3,4-b]pyrazin-1-yl)propan-1-ol,
N-[(1H-benzimidazol-2-yl)methyl]-6-cyclopropyl-1-(3-methoxypropyl)-1H-pyrazolo [3,4-b]pyrazin-3-amine,
(±)-N-[(1H-benzimidazol-2-yl)methyl]-6-cyclopropyl-1-[4-(dimethylamino)butan-2-yl]-1H-pyrazolo[3,4-b]pyrazin-3-amine,
(+) or (−)-N-[(1H-benzimidazol-2-yl)methyl]-6-cyclopropyl-1-[4-(dimethylamino)butan-2-yl]-1H-pyrazolo[3,4-b]pyrazin-3-amine,
(−) or(+)-N-[(1H-benzimidazol-2-yl)methyl]-6-cyclopropyl-1-[4-(dimethylamino)butan-2-yl]-1H-pyrazolo[3,4-b]pyrazin-3-amine,
N-[(1H-benzimidazol-2-yl)methyl]-6-cyclopropyl-1-(2-methoxyethyl)-1H-pyrazolo [3,4-b]pyrazin-3-amine,
2-(3-{[(1H-benzimidazol-2-yl)methyl]amino}-6-cyclopropyl-1H-pyrazolo[3,4-b]pyrazin-1-yl)ethan-1-ol,
N-[(1H-benzimidazol-2-yl)methyl]-6-cyclopropyl-1-(3-methoxy-3-methylbutyl)-1H-pyrazolo[3,4-b]pyrazin-3-amine,
N-[(1H-benzimidazol-2-yl)methyl]-6-cyclopropyl-1-[2-(dimethylamino)ethyl]-1H-pyrazolo [3,4-b]pyrazin-3-amine,
1-(3-{[(1H-benzimidazol-2-yl)methyl]amino}-6-cyclopropyl-1H-pyrazolo[3,4-b]pyrazin-1-yl)-2-methylpropan-2-ol,
trans-3-[(3-{[(1H-benzimidazol-2-yl)methyl]amino}-6-cyclopropyl-1H-pyrazolo [3,4-b]pyrazin-1-yl)methyl]cyclobutan-1-ol,
cis-3-[(3-{[(1H-benzimidazol-2-yl)methyl]amino}-6-cyclopropyl-1H-pyrazolo [3,4-b]pyrazin-1-yl)methyl]cyclobutan-1-ol,
1-[(3-{[(1H-benzimidazol-2-yl)methyl]amino}-6-cyclopropyl-1H-pyrazolo[3,4-b]pyrazin-1-yl)methyl]cyclobutan-1-ol,
1-[(3-{[(1H-benzimidazol-2-yl)methyl]amino}-6-cyclopropyl-1H-pyrazolo[3,4-b]pyrazin-1-yl)methyl]cyclopentan-1-ol,
{1-[(3-{[(1H-benzimidazol-2-yl)methyl]amino}-6-cyclopropyl-1H-pyrazolo[3,4-b]pyrazin-1-yl)methyl]cyclobutyl }methanol,
N-[(1H-benzimidazol-2-yl)methyl]-1-(cyclobutylmethyl)-6-cyclopropyl-1H-pyrazolo [3,4-b]pyrazin-3-amine,
4-(3-{[(1H-benzimidazol-2-yl)methyl]amino}-6-cyclopropyl-1H-pyrazolo[3,4-b]pyrazin-1-yl)-2-methylbutan-2-ol,
N-[(1H-benzimidazol-2-yl)methyl]-6-methyl-1-(1-methylpiperidin-4-yl)-1H-pyrazolo [3,4-b]pyrazin-3-amine,
(+) or (−)-N-[(1H-benzimidazol-2-yl)methyl]-6-cyclopropyl-1-(1-methylpyrrolidin-3-yl)-1H-pyrazolo[3,4-b]pyrazin-3-amine and
(−) or (+)-N-[(1H-benzimidazol-2-yl)methyl]-6-cyclopropyl-1-(1-methylpyrrolidin-3-yl)-1H-pyrazolo[3,4-b]pyrazin-3-amine;
or a tautomer, or an N-oxide, or a salt thereof, or a salt of a tautomer, or a salt of an N-oxide, or a mixture of same.

14. A compound of formula (I) according to claim 1, having a (IC50 CDK12 hATP)/(DC50 CDK12) ratio which is equal or greater than 500 and/or a (DC50 CDK12) value which is equal or lower than 200 nM.

15. (canceled)

16. A method for the treatment and/or prophylaxis of a hyperproliferative disease in a subject in need thereof, comprising administering to the subject a compound of formula (I) according to claim 1, or a stereoisomer, a tautomer, an N-oxide, a hydrate, a solvate, or a salt thereof, particularly a pharmaceutically acceptable salt thereof, or a mixture of same.

17. The method of claim 16, wherein the hyperproliferative disease is breast cancer, liver cancer, lung cancer, ovarian cancer, endometrial cancer, cervical cancer, colorectal cancer, gastric cancer, esophageal cancer, bladder cancer, prostate cancer, Ewing sarcoma, glioblastoma or acute myeloid leukemia.

18. The method of claim 16, wherein the hyperproliferative disease is lung cancer, breast cancer, liver cancer, colorectal cancer, gastric cancer, prostate cancer or leukemia.

19. The method of claim 16, wherein the hyperproliferative disease is wherein the hyperproliferative disease is cancer.

20. A method for the treatment and/or prophylaxis of a hyperproliferative disease in a subject in need thereof, comprising administering to the subject a compound of formula (I) according to claim 13, or a stereoisomer, a tautomer, an N-oxide, a hydrate, a solvate, or a salt thereof, particularly a pharmaceutically acceptable salt thereof, or a mixture of same.

21. The method of claim 20, wherein the hyperproliferative disease is lung cancer, breast cancer, liver cancer, colorectal cancer, gastric cancer, prostate cancer or leukemia.

22. A pharmaceutical composition comprising a compound of formula (I) according to claim 1, or a stereoisomer, a tautomer, an N-oxide, a hydrate, a solvate, or a salt thereof, particularly a pharmaceutically acceptable salt thereof, or a mixture of same, and a pharmaceutically acceptable carrier.

23. A method for the treatment and/or prophylaxis of a hyperproliferative disease in a subject in need thereof, comprising administering to the subject a pharmaceutical composition according to claim 22.

24. A pharmaceutical combination comprising:
One or more first active ingredients selected from a compound of general formula (I) according to claim 1, and
One or more second active ingredients selected from chemotherapeutic anti-cancer agents.

25. A process for the preparation of a compound of formula (I), wherein R¹, R², R³ and X are as defined for the compounds of formula (I) according to claim 1, 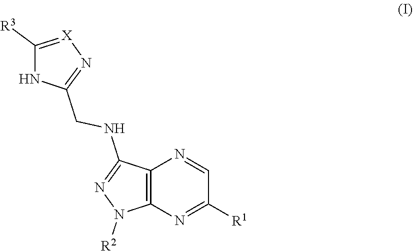
said process comprising the step of allowing an intermediate compound of general formula (II) 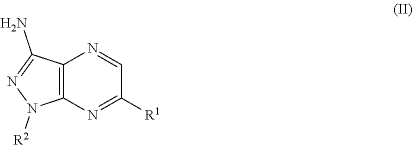
wherein R¹ and R² are as defined for the compounds of formula (I) according to claim 1,
to react with a compound of formula (III) 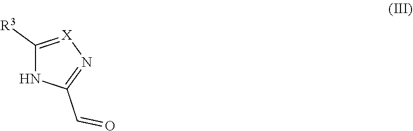
wherein R³ and X are as defined for the compounds of formula (I) according to claim 1,
thereby giving a compound of formula (I).

26. A process for the preparation of a compound of formula (I), wherein R¹, R², R³ and X are as defined for the compounds of formula (I) according to claim 1, said process comprising reacting a compound of general formula (II) 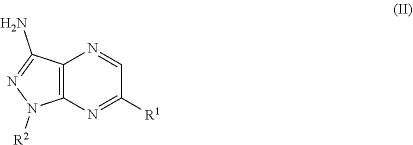
wherein R¹ and R² are as defined for the compounds of formula (I) according to claim 1.


SEQUENCE LISTING (8 sequences)

SEQ ID NO: 1 - protein, 425 aa, Homo sapiens
  His-CDK12
MTSHHHHHHSSMGSRTSLYKKAGSDYDIPTTENLYFQGQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNS

SEQ ID NO: 2 - protein, 425 aa, Homo sapiens
  His-CDK12-DN
MTSHHHHHHSSMGSRTSLYKKAGSDYDIPTTENLYFQGQPYKKRPKICCPRYGERRQTESDWGKRCVDKFDIIGIIGEGTYGQVYKAKDKDTGELVALAKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLANFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNS

SEQ ID NO: 3 - protein, 425 aa, Homo sapiens
  His-CDK13
MTSHHHHHHSSMGSRTSLYKKAGSDYDIPTTENLYFQGQLHSKRRPKICGPRYGETKEKDIDWGKRCVDKFDIIGIIGEGTYGQVYKARDKDTGEMVALKKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLADFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTP

SEQ ID NO: 4 - protein, 425 aa, Homo sapiens
  His-CDK13-DN
MTSHHHHHHSSMGSRTSLYKKAGSDYDIPTTENLYFQGQLHSKRRPKICGPRYGETKEKDIDWGKRCVDKFDIIGIIGEGTYGQVYKARDKDTGEMVALAKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLANFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTP

SEQ ID NO: 5 - protein, 338 aa, Homo sapiens
  His-CycK
MTSHHHHHHSSMGSRTSLYKKAGSDYDIPTTENLYFQGMKENKENSSPSVTSANLDHTKPCWYWDKKDLAHTPSQLEGLDPATEARYRREGARFIFDVGTRLGLHYDTLATGIIYFHRFYMFHSFKQFPRYVTGACCLFLAGKVEETPKKCKDIIKTARSLLNDVQFGQFGDDPKEEVMVLERILLQTIKFDLQVEHPYQFLLKYAKQLKGDKNKIQKLVQMAWTFVNDSLCTTLSLQWEPEIIAVAVMYLAGRLCKFEIQEWTSKPMYRRWWEQFVQDVPVDVLEDICHQILDLYSQGKQQMPHHTPHQLQQPPSLQPTPQVPQVQQSQPSQSSEPS

SEQ ID NO: 6 - protein, 368 aa, Saccharomyces cerevisiae
  CAK1
MKLDSIDITHCQLVKSTRTARIYRSDTYAIKCLALDFDIPPHNAKFEVSILNKLGNKCKHILPLLESKATDNNDLLLLFPFEEMNLYEFMQMHYKRDRRKKNPYYDLLNPSIPIVADPPVQKYTNQLDVNRYSLSFFRQMVEGIAFLHENKIIHRDIKPQNIMLTNNTSTVSPKLYIIDFGISYDMANNSQTSAEPMDSKVTDISTGIYKAPEVLFGVKCYDGGVDVWSLLIIISQWFQRETSRMGHVPAMIDDGSDDMNSDGSDFRLICSIFEKLGIPSIQKWEEVAQHGSVDAFVGMFGADGDGKYVLDQEKDVQISIVERNMPRLDEIADVKVKQKFINCILGMVSFSPNERWSCQRILQELEKP

SEQ ID NO: 7 - protein, 18 aa, Homo sapiens
  biotinylated peptide biotin-Ahx, C-terminus in amid form
KFELLPTPPLSPSRRSGL

SEQ ID NO: 8 - protein, 14 aa, Homo sapiens
  biotinylated peptide biotin-Ttds, C-terminus in amid form
YISPLKSPYKISEG